SEQUENCE LISTING (1 sequence)

SEQ ID NO: 1 - protein, 24 aa, artificial sequence
  Fragment from GS
ITGTNAEVMPAQWEFQIGPCEGIR

(19) United States
(12) Patent Application Publication
(10) Pub. No.: US 2009/0209474 A1
(43) Pub. Date: Aug. 20, 2009

(54) NOVEL USES FOR DRUGS TARGETING GLUTAMINE SYNTHETASE

(75) Inventors: Jean-Christophe Roegel, Niederentzen (FR); Pierre Eftekhari, Strasbourg (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: NEWTHERA, Niederentzen (FR)

(21) Appl. No.: 12/063,360

(22) PCT Filed: Aug. 8, 2006
(86) PCT No.: PCT/IB2006/003374

§ 371 Date: Feb. 8, 2008

(30) Foreign Application Priority Data
Aug. 8, 2005 (EP) 05291692

(51) Int. Cl. A61K 31/7004; A61K 31/554; A61K 31/675; A61K 31/661; A61K 31/535; C12Q 1/25; G01N 33/00; G01N 33/573

(52) U.S. Cl. 514/23; 514/211.13; 514/89; 514/237.8; 514/118; 514/119; 435/4; 436/90; 436/111; 435/7.93

(57) ABSTRACT

The present invention relates to novel therapeutic uses of tianeptine, salts, isomers, pro-drugs, metabolites and structural analogs thereof. Furthermore, the present invention relates to the use of tianeptine, salts, isomers, pro-drugs, metabolites and structural analogs thereof, in obtaining methods of screening and of developing drugs. Finally, the present invention relates to the novel therapeutic use of other glutamine synthetase (GS) ligands and to the use of these ligands in obtaining methods for screening and developing drugs.

### FIELD OF THE INVENTION

The present invention first relates to novel therapeutic uses of tianeptine, salts, isomers, pro-drugs, metabolites and structural analogs thereof. Furthermore, the present invention relates to the use of tianeptine, salts, isomers, pro-drugs, metabolites and structural analogs thereof, in obtaining methods of screening and of developing drugs comprising tests on the activity of the native glutamine synthetase (GS). Finally, the present invention relates to novel therapeutic uses of other GS ligands with regulating properties on native GS activity, and to the use of these GS regulating ligands in obtaining methods for screening and developing drugs comprising tests on the activity of the native GS.

### BACKGROUND OF THE INVENTION

Tianeptine, which has the systematic name 7-[(3-chloro-6,11-dihydro-6-methyl-dibenzo[c,f][1,2] thiazepin-11-yl) amino] heptanoic acid S,S-dioxide (C₂₁H₂₄ClN₂NaO₄S; F.W. 458.9), is a tricyclic anti-depressant of the dibenzothiazepine type.

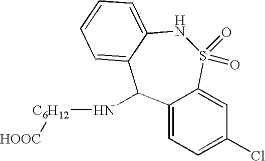

Tianeptine has been patented as a drug for use in the treatment of psychoneurotic disorders, pain and cough (FR 2 104 728); stress (FR 2 635 461), mnemo-cognitive disorders (FR 2 716 623), neurodegenerative diseases (U.S. Pat. No. 6,599,896), and irritable bowel syndrome and nonulcer dyspepsia (U.S. Pat. No. 6,683,072). It is used to treat depression (major depression with or without melancholia or dysthymic disorders, without psychotic features, depressed bipolar disorders, . . . ), neurotic or reactive states of depression, anxiodepressive states with somatic complaints such as digestive problems, anxiodepressive states observed in alcoholic detoxification, and asthma (for asthma: see Lechin et al., 1998). Despite of these therapeutic uses, its target remains unknown. The emerging pharmacological profile suggests that tianeptine serves to balance glutamatergic neurotransmission, in particular in conditions of high glucocorticoid exposure and stress (Plaisant et al., 2003; Reagan et al., 2004). Tianeptine might provide an original way of action in treating functional as well as structural changes that are associated with stress. For instance, tianeptine can both counteract behavioural and biochemical consequences of stress (Luine, 1992; Conrad et al., 1996; Delbende et al., 1991), and the dendritic atrophy of CA3 pyramidal neurons induced by stress (Watanabe et al., 1992). Atrophy of hippocampal dendrites in animal models of stress may be of the same nature than atrophy of the human hippocampus associated with recurrent depressive illness (Sheline et al., 1996), post-traumatic stress disorder (Bremner et al., 1995; Gurvits et al., 1996), mild cognitive impairment in aging (Convit et al., 1997), etc. Furthermore, tianeptine effects are most often observed to be not “dose-dependent” i.e. they do not simply decrease or increase with the dose (see for instance U.S. Pat. No. 6,599,896 B1 and the recent paper by Ceyhan et al. reporting an inhibitory effect of tianeptine on PTZ-induced seizures). There may be J-shaped or inverted U-shaped dose responses with tianeptine (an hormetic effect of tianeptine is questionable).

L-methionine sulfoximine (L-MSO or; MS), which has the systematic name 2-amino-4-(S-methylsulfonimidosyl)butanoic acid or L-s-[3-amino-3-carboxypropyl]-s-methylsulfoximine (C₅H₁₂N₂O₃S; F.W. 180.22), is a rare amino acid with a structural resemblance to glutamate. It occurs in nature and as a by-product of some forms of food processing. A notable example of the latter is a former method for bleaching wheat flour, using nitrogen trichloride, the “a gene process”, in use for most of the first 50 years of the previous century. “Agenized flour” was found to be responsible for various neurological disorders in animals, and MS was identified as the toxic agent. The agene process was subsequently discontinued.

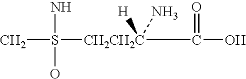

MS is believed to act as an inhibitor of the synthesis of glutathione (GSH) by blocking γ-glutamylcysteine synthetase (Meister and Tate, 1976) and as an inhibitor of the conversion of glutamate to glutamine by blocking glutamine synthetase (GS; EC 6.3.1.2) (Meister and Tate, 1976; Griffith et al., 1979; Meister, 1985). It is probable that the known actions of MS on the nervous system arise mostly from alterations in the activity, amount and distribution of these target proteins. The consequence of the former would be a decrease in GSH status (Kosower and Kosower, 1978) and a subsequent increase in oxidative stress (Richie et al., 1987; Reiter, 1995), a factor to which neurons may be particularly vulnerable (Evans, 1993; Bains and Shaw, 1997). Consequences of the latter could include a decrease in the control of ammonia, glutamate, glutamine, . . . (Finch and Cohen, 1997). Ammonia has been shown to be toxic to neurons and has been implicated as one causal factor in neurological disorders (Seiler, 1993; Hoyer, 1994). Any increase in extracellular glutamate can (potentially) be (excito)toxic, directly or indirectly, upon its binding on glutamatergic ionotropic (GluR) and/or glutamatergic metabotropic (mGluR) receptors. Furthermore, MS has a direct effect on glutamatergic neurotransmission. In particular, it has been reported to increase glutamate release (Shaw et al., 1999), and to increase the neurotoxic effects of kainate and NMDA (Kollegger et al., 1991). Finally, glutamine serves as a fuel source, and as the most important carrier of nitrogen and one of the major build stones in protein synthesis. It is involved in crucial homeostatic functions (see below).

Administration of MS was observed to produce neurological disorders, in particular seizures, in a number of animal species (Lodin and Kolousek, 1956; Lodin, 1958; Hrebicek and Kolousek, 1968) at doses as low as 2 mg/kg body weight (bw) (Campbell et al., 1950; Wada et al., 1967). Tested in patients with far-advanced non resectable cancer, it was observed to impact the central nervous system in the form of hallucinations, disorientation and marked agitation, while no evidence of tumor regression was noted (Krakoff et al., 1961). On the contrary, MS was observed to protect both the cerebral cortex from ischaemia after middle cerebral artery occlusion (Swanson et al., 1990; this effect was proposed to be due to an impaired glutamate neuronal release through GS inhibition and/or to an increase of brain glycogen stores by MS) and from hepatic encephalopathy (see below). MS was also proposed to prevent/treat the Huntington's and other polyglutamine disorders caused by expanded genomic CAG nucleotides (preferably intravenously or orally at a dose between 2 and 10 mg/kg per 6-10 days; US 2004/0152778 A1).

Glufosinate ammonium (GF), which has the systematic name ammonium 4-[hydroxy (methyl)phosphinoyl]-DL-homoalaninate or 2-amino-4-(hydroxymethylphosphinyl) butyric acid ammonium salt (C₅H₁₅N₂O₄P; F.W. 198.16), is a phosphinic acid analog of glutamic acid. It is also denominated phosphinothrycin (PPT). It is produced in nature as the tripeptide L-phosphinothricyl-L-alanyl-L-alanine (bialaphos/bialophos) by the bacteria Streptomyces hygroscopicus and Streptomyces viridochromogenes.

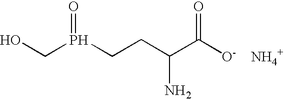

GF is used as an active ingredient in non-selective herbicides (Basta®; Liberty®). Its action is related to inhibition of plant's glutamine synthetase, which is involved in critical homeostatic functions (see below). After application on foliage, GF provides effective weed control in three to seven days. The symptoms appear as chlorotic lesions followed by necrosis. There is limited translocation of GF after application. GF has no soil activity.

GF is non-selective except to crops that carry the gene coding for phosphinothricin tolerance (bar), which encodes the enzyme phosphinothricin acetyltransferase (PAT) (PAT was also derived from Streptomyces hygroscopicus and Streptomyces viridochromogenes; EP 275957 filed in 1987 by Aventis CropScience (former AgrEvo)). At low (herbicidal) rates, GF improves even the yield of crop plants which are resistant to glutamine synthetase inhibitors (U.S. Pat. No. 5,739,082); the mechanism underlying the latter is unknown.

GF can inhibit glutamine synthetase in animals. It has also been observed to behave as an agonist on glutamate receptors (Nakaki et al., 2000; Matsumura et al., 2001; Lapouble et al., 2002). However, under physiological conditions in healthy subjects, only at high (sublethal) doses, glutamate, ammonia and glutamine levels in brain, liver and kidney are affected (Hack et al., 1994; Ohtake et al., 2001).

GF was proposed for the prevention/treatment of hepatic encephalopathy (see below), and of Huntington's and other polyglutamine disorders caused by expanded genomic CAG nucleotides (intrathecal administration at a dose between 1 and 5 mg/kg per 6-10 days; US 2004/0152778 A1). It was also reported to exhibit antimicrobial activity against Gram-positive and Gram-negative bacteria and some fungi in chemically defined minimal media; this antimicrobial activity was abolished by the presence of L-glutamine and there was no antimicrobial activity in complex organic media (Uri et al., 1988).

The toxicokinetics of GF has been investigated in rats and dogs as well as in livestock. The substance is rapidly excreted in all these species regardless of the route of administration. About 80-90% of an oral dose of GF remains unabsorbed and is eliminated unchanged in the faeces over 48 hours, while about 10-15% is eliminated in the urine. The main metabolite of GF found in urine and in faeces is 3-[hydroxy(methyl)phosphinoyl] propionic acid.

GF shows slight to moderate acute oral toxicity in mice, rats and dogs. In mice, the no observed adverse effect level (NOAEL) is 80 ppm (parts per million), equal to 17 and 19 mg/kg bw/day (bw: body weight) in males and females, respectively, based upon increased plasma potassium levels at the next highest tested dose (67 mg/kg bw/day). In rats, effects on kidney weight are seen at dose levels as low as 0.52 mg/kg bw/day. These effects on kidney weight are not found in long-term bioassays. In dogs, the NOAEL is 4.5 mg/kg bw/day, based on decreased body weight at higher doses.

GF is not teratogenic in rabbits and rats. The NOAELs for maternal and embryo/fetal toxicity is 6.3 mg/kg bw/day in rabbits and 2.2 mg/kg bw/day in rats.

GF is not carcinogenic in long-term/carcinogenicity studies in mice and rats. In mice fed 0, 20, 80 or 160 ppm (males) and 0, 20, 80 or 320 ppm (females), the NOAEL is 80 ppm (equal to 11 mg/kg bw/day) with increased mortality at higher dietary concentrations. In rats fed 0, 40, 140 or 500 ppm, the NOAEL is 40 ppm (equal to 2.1 mg/kg bw/day) with increased kidney weight at 140 ppm. At higher doses, a reduction in glutathione level is noted in liver and blood.

Thus, estimate of oral acceptable daily intake (ADI) of this non-selective herbicide has been fixed for man to 0-0.02 mg/kg bw (based upon the NOAEL determined from the long-term study in rats using a 100-fold safety factor).

For review of toxicological evaluation of glufosinate ammonium see:

Website address: http://www.inchem.org/documents/jmpr/jmponono/v91pr12.htm
Website address: http://www.inchem.org/documents/jmpr/jmpmono/v99pr06.htm

GF and MS exhibit the kinetic properties of a slow tight-binding inhibitor and undergo phosphorylation during the course of inactivation (ATP-dependent) (Logusch et al., 1990; Abell and Villafranca, 1991; Abell et al., 1995; Eisenberg et al., 2000). The rate-limiting step in the inhibition reaction is the binding of the inhibitor and/or the conformational change associated with its binding. During the course of the inactivation, progressively slower rates for binding and phosphoryl transfer are observed (see below).

Finally, while GF and MS, and analogs thereof, exhibit herbicidal activity at “high concentrations” i.e. concentrations which in foliage inhibit glutamine synthetase (excess ammonium and glutamine deficiency act in concert to cause plant death), concentrations of MS and GF, and derivatives thereof, below those which exhibit herbicidal activity, were observed by one russian group to have an opposite effect: glutamine synthetase was activated with concomitant stimulation of plant growth and productivity (Evstigneeva et al., 2003).

### SUMMARY OF THE INVENTION

The Inventors have first discovered that tianeptine, an atypical antidepressant with neuroprotective properties, is a selective ligand of the enzyme glutamine synthetase (GS; EC 6.3.1.2). To investigate the effects of tianeptine on GS activity, an usual method used to investigate GS activity (Meister 1985) had to be amended. In fact, tianeptine lost its activity in presence of the reducing agent 2β-mercaptoethanol (β-ME) due to its interaction with SO₂. Thus, the effects of tianeptine on GS activity were first tested in absence of β-ME i.e. on whole GS enzyme instead on GS subunits (see below); β-ME was replaced by dithiothreitol (DTT) in the experiments investigating the effects on GS subunits (monomers) i.e. in presence of a reducing agent. Transferase activity was tested instead of synthetase activity due to its high sensitivity and low background activity. Surprisingly, depending on the concentration/dose and experimental or (patho)physiological condition, tianeptine could both activate and inhibit GS activity in animals and bacteria. In short, i) at “low concentrations”, depending on the experimental or (patho)physiological condition, tianeptine can behave as a GS activator or a GS inhibitor, while ii) at “high concentrations”, tianeptine behaves strictly as a GS inhibitor. In addition to the above mentioned effects, tianeptine was observed to protect GS from oxidation. It could also influence GS protein expression. The dose/effect relationships differed according to the cell, tissue, organ, species, . . . , and changed over time (in particular due to changes in the expression of the enzyme). Thus, GS and its regulation have to be approached by considering their multidimensional nature and in native conditions. These “regulating” effects of tianeptine at “low doses” on GS activity may likely underlie its already proposed clinical applications contrary to its unclear effects on 5-HT uptake that are usually proposed. They can be reconciled with the numerous other previous speculations about its mode of action and pharmacological profile; this includes the emerging one which suggest that tianeptine balance glutamatergic neurotransmission (McEwen et al, 1993 and 2004; Plaisant et al., 2003; Reagan et al., 2004).

Accordingly, the present invention concerns a method for regulating GS activity in a subject, comprising administering to said subject an efficient amount of tianeptine or a salt thereof, an isomer thereof, a pro-drug thereof, a metabolite thereof or a structural analog thereof. The present invention also concerns a method for protecting GS from oxidation in a subject comprising administering to the subject an effective amount of tianeptine, salts, isomers, pro-drugs, metabolites and structural analogs thereof. The present invention further concerns a method for modulating the expression of GS in a subject, comprising administering to the subject an effective amount of tianeptine, salts, isomers, pro-drugs, metabolites and structural analogs thereof.

The present invention relates to enlarge the use of tianeptine, salts, isomers, pro-drugs, metabolites and structural analogs thereof, in obtaining regulators of GS activity intended for the activation, inhibition and/or regulation of functions which involve GS activity or can be influenced by GS activity, directly or indirectly, and the prevention and/or treatment of all types of conditions and disorders which involve or can be influenced by GS activity, directly or indirectly. In particular, the invention concerns the prevention and/or treatment of conditions and disorders related to absolute or relative excess of ammonia, and/or absolute or relative deficiency or excess of glutamate, and/or absolute or relative deficiency or excess of glutamine, in vertebrate animals including humans. Therefore, the present invention concerns the use of tianeptine, salts thereof, isomers thereof, pro-drugs thereof, metabolites thereof and structural analogs thereof, in the manufacture of medicaments intended for prevention and/or treatment of all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, with the exception of the already claimed or proposed clinical applications of tianeptine. In particular, said conditions and disorders which involve or can be influenced by GS activity are selected from those disclosed in Tables 1-4; potential factors and mechanisms which can underlie these conditions and disorders are disclosed in Table 4, without to be limited thereto. The already claimed or proposed clinical applications of tianeptine are disclosed in Table 5.

The present invention also concerns a method for prevention and/or treatment of any condition or disorder which involve or can be influenced by GS activity, directly or indirectly, in particular those related to absolute or relative excess of ammonia, those related to absolute or relative deficiency or excess of glutamate, and those related to absolute or relative deficiency or excess of glutamine, with the exception of the already claimed or proposed clinical applications of tianeptine, comprising administering an efficient amount of tianeptine or a salt thereof, an isomer thereof, a pro-drug thereof, a metabolite thereof or a structural analog thereof, thereby preventing and/or treating said condition or disorder. In a preferred embodiment, said conditions and disorders are selected from those disclosed in Tables 1-4, preferably in Tables 1, 2 and 3, more preferably in Table 1, with the exception of the conditions and disorders disclosed in Table 5.

In a particular embodiment, said metabolites or structural analogs of tianeptine are selected from the group consisting of the compounds in Table 8. Optionally, tianeptine or a salt, an isomer, a pro-drug, a metabolite or a structural analog thereof, can be used in combination with an other drug intended to prevent/treat hyperammon(emi)a or resulting in hyperammon(em)ia, an other drug intended to prevent/treat conditions and disorders related to deficiency or excess of glutamate or resulting in deficiency or excess of glutamate, or an other drug intended to prevent/treat conditions and disorders related to deficiency or excess of glutamine or resulting in deficiency or excess of glutamine. In particular, these drugs include: i) drugs intended to prevent/treat hyperammon(em)ia and/or hyperammon(em)ia symptoms e.g. flumazenil, zinc, sodium benzoate, sodium phenylacetate and arginine hydrochloride, ii) drugs resulting in hyperammon(em)ia e.g. anti-cancer therapies (5-fluorouracil, asparaginase, . . . ) and antiepileptics (sodium valproate, primidone, . . . ), iii) drugs altering glutamate receptor(s) function(s) e.g. ampakines, glutamate release inhibitors (lamotrigine, riluzole, . . . ), glutamate receptor antagonists (D-AP5, ketamine, memantine, MK-801, . . . ) and glycine, iv) drugs intended to prevent/treat disorders where GS protein/activity is altered or relatively ineffective e.g. anti-epileptic treatments (benzodiazepines, carbamazepine, ethosuximide, hydantoins, gabapentin, lamotrigin, phenobarbital, primidone, valproate, . . . ) and antibiotics (aminoglycosides, cephalosporins, chloramphenicol, macrolides, penicillins, quinolones, rifampicine, sulfonamides, tetracyclines, trimethoprim-sulfamethoxazole, . . . ) in certain forms of epilepsy and critical sepsis, respectively, v) drugs intended to combat neoplasms and/or inflammatory diseases e.g. anti-proliferative (5-fluorouracil, asparaginase, . . . ) and vi) anti-inflammatory drugs or drugs preventing from the occurrence of inflammation, in particular glucocorticoids.

In a first preferred embodiment, said conditions and disorders are related to or result in a local excess of ammonia (hyperammonia) or a systemic excess of ammonia (hyperammonemia) i.e. hyperammon(em)ia. Preferably said hyperammon(em)ia is related to a severe liver disease (resulting from cirrhosis, hepatitis, intoxication, . . . ), an inborn error of metabolism (urea cycle disorder, . . . ) and/or a porto-systemic shunt, but various other conditions and disorders, alone or combined, (can) result in a localized ammonia increase or a systemic ammonia increase (see below, and Tables 1, 2-3 (the double-underlined conditions and disorders) and 4). In particular, said conditions and disorders are selected from the group consisting of severe hepatic disorders (resulting from cirrhosis, hepatitis, intoxication, . . . ), errors of metabolism (urea cycle disorder, . . . ), porto-systemic shunts, gastrointestinal haemorrhages, transplant rejections and catabolic states. In particular, said conditions and disorders can be selected from the group consisting of hyperammonemia and i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders.

In a second preferred embodiment, said conditions and disorders are related to or result in (absolute or relative) deficiency or excess of glutamate and/or in (absolute or relative) deficiency or excess of glutamine, and/or sensitive to glutamine deprivation or glutamine supplementation. A myriad of conditions and disorders are prone to be associated with a localized deficiency or excess of glutamine (see below). De novo glutamine synthesis is more or less crucial depending on the cell type and (patho)physiological condition. Thus, said conditions and disorders are preferably selected from those disclosed in Tables 2 (in particular those marked in “italic”) and 3, with the exception of those disclosed in Table 5. In particular, said conditions and disorders can be selected in the group consisting of i) Gastrointestinal inflammatory conditions, Gastrointestinal ulceration and perforation, and Malabsorption conditions, ii) General disorders and administration site conditions such as Mucosal findings abnormal and Tissue disorders, particularly Trophic disorders e.g. Atrophy and Denervation atrophy, iii) Autoimmune disorders and Immune disorders, iv) Infections and infestations, v) Injury, poisoning and procedural complications such as Administration site reactions, Chemical injury, Injuries by physical agents, and other Procedural and device related injuries and complications, vi) Metabolism and nutrition disorders, particularly Catabolic state and Hypercatabolism, vii) Bone disorders, particularly Osteoporosis, Connective tissue disorders, particularly Lupus erythematosus, Joint disorders, Muscle disorders, and Tendon, ligament and cartilage disorders, xiii) Neoplasms benign, malignant and unspecified, particularly Gliomas benign xix) Respiratory tract inflammatory and immunologic conditions, and Bronchitis chronic xx) Arteriosclerosis, Circulatory collapse and shock, Vascular injuries and Venous varices.

The present invention also relates to the use of tianeptine, salts thereof, isomers thereof, pro-drugs thereof, metabolites thereof or structural analogs thereof, in the preparation of compositions to be used to activate, inhibit and/or regulate GS activity intended for the activation, inhibition and/or regulation of functions which involve GS activity or can be influenced by GS activity, directly or indirectly, in non-vertebrate organisms with eukaryotic cells such as invertebrate animals (arthropods, protozoa, shellfish, worms, . . . ), algae, fungi (yeasts, molds, . . . ) or plants, but also prokaryotes e.g. algae and bacteria, aiming to facilitate their growth or protect them from stresses and disorders or, inversely, aiming to combat their growth. Thus, the present invention concerns methods to facilitate the growth of non-vertebrate organisms with eukaryotic cells such as invertebrate animals (arthropods, protozoa, shellfish, worms, . . . ), algae, fungi (yeasts, molds, . . . ) or plants, but also of prokaryotes e.g. algae or bacteria, either to protect them from stresses and disorders, or to combat them, comprising administering an efficient amount of tianeptine or a salt thereof, an isomer thereof, a pro-drug thereof, a metabolite thereof or a structural analog thereof. In a particular embodiment, said metabolites or structural analogs of tianeptine are selected from the group consisting of the compounds disclosed in Table 8. Therefore, the invention concerns the use of tianeptine or a salt, an isomer, a pro-drug, a metabolite or a structural analog thereof, for the preparation of a composition aiming to facilitate the growth of non-vertebrate organisms with eukaryotic cells such as invertebrate animals (arthropods, protozoa, shellfish, worms, . . . ), algae, fungi (yeasts, molds, . . . ) or plants, and prokaryotes e.g. algae or bacteria, or to protect them from stresses and disorders, or inversely, aiming to combat their growth.

In addition, the Inventors also identified the target of naftazone (NF). Indeed, NF is a selective ligand of the enzyme glutamine synthetase (GS; EC 6.3.1.2). The present invention also concerns a method for regulating GS activity in a subject, comprising administering to said subject an efficient amount of NF or a salt thereof, an isomer thereof, a pro-drug thereof, a metabolite thereof or a structural analog thereof. Then, the present invention relates to enlarge the use of NF, salts, isomers, pro-drugs, metabolites and structural analogs thereof, in obtaining regulators of GS activity intended for the activation, inhibition and/or regulation of functions which involve GS activity or can be influenced by GS activity, directly or indirectly, and the prevention and/or treatment of all types of conditions and disorders which involve or can be influenced by GS activity, directly or indirectly. In particular, the invention concerns the prevention and/or treatment of conditions and disorders related to absolute or relative excess of ammonia, and/or absolute or relative deficiency or excess of glutamate, and/or absolute or relative deficiency or excess of glutamine, in vertebrate animals including humans.

Therefore, the present invention concerns the use of NF, salts thereof, isomers thereof, pro-drugs thereof, metabolites thereof and structural analogs thereof, in the manufacture of medicaments intended for prevention and/or treatment of all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, with the exception of the already claimed or proposed clinical applications of NF. In particular, said conditions and disorders which involve or can be influenced by GS activity are selected from those disclosed in Tables 1-4; potential factors and mechanisms which can underlie these conditions and disorders are disclosed in Table 4, without to be limited thereto. The already claimed or proposed clinical applications of tianeptine are disclosed in Table 14.

The present invention also concerns a method for prevention and/or treatment of any condition or disorder all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, with the exception of the already claimed or proposed clinical applications of NF, comprising administering an efficient amount of NF or a salt thereof, an isomer thereof, a pro-drug thereof, a metabolite thereof or a structural analog thereof, thereby preventing and/or treating said condition or disorder. In a preferred embodiment, said conditions and disorders are selected from those disclosed in Tables 1-4, preferably in Tables 1, 2 and 3, more preferably in Table 1, with the exception of the conditions and disorders disclosed in Table 14.

In a particular embodiment, said metabolites or structural analogs of NF are selected from the group consisting of the compounds in Table 15. Optionally, NF or a salt, an isomer, a pro-drug, a metabolite or a structural analog thereof, can be used in combination with an other drug intended to prevent/treat hyperammon(emi)a or resulting in hyperammon(em)ia, an other drug intended to prevent/treat conditions and disorders related to deficiency or excess of glutamate or resulting in deficiency or excess of glutamate, or an other drug intended to prevent/treat conditions and disorders related to deficiency or excess of glutamine or resulting in deficiency or excess of glutamine. In particular, these drugs include: i) drugs intended to prevent/treat hyperammon(em)ia and/or hyperammon(em)ia symptoms e.g. flumazenil, zinc, sodium benzoate, sodium phenylacetate and arginine hydrochloride, ii) drugs resulting in hyperammon(em)ia e.g. anti-cancer therapies (5-fluorouracil, asparaginase, . . . ) and antiepileptics (sodium valproate, primidone, . . . ), iii) drugs altering glutamate receptor(s) function(s) e.g. ampakines, glutamate release inhibitors (lamotrigine, riluzole, . . . ), glutamate receptor antagonists (D-AP5, ketamine, memantine, MK-801, . . . ) and glycine, iv) drugs intended to prevent/treat disorders where GS protein/activity is altered or relatively ineffective e.g. anti-epileptic treatments (benzodiazepines, carbamazepine, ethosuximide, hydantoins, gabapentin, lamotrigin, phenobarbital, primidone, valproate, . . . ) and antibiotics (aminoglycosides, cephalosporins, chloramphenicol, macrolides, penicillins, quinolones, rifampicine, sulfonamides, tetracyclines, trimethoprim-sulfamethoxazole, . . . ) in certain forms of epilepsy and critical sepsis, respectively, v) drugs intended to combat neoplasms and/or inflammatory diseases e.g. anti-proliferative (5-fluorouracil, asparaginase, . . . ) and vi) anti-inflammatory drugs or drugs preventing from the occurrence of inflammation, in particular glucocorticoids.

In a first preferred embodiment, said conditions and disorders are related to or result in a local excess of ammonia (hyperammonia) or a systemic excess of ammonia (hyperammonemia) i.e. hyperammon(em)ia. Preferably said hyperammon(em)ia is related to a severe liver disease (resulting from cirrhosis, hepatitis, intoxication, . . . ), an inborn error of metabolism (urea cycle disorder, . . . ) and/or a porto-systemic shunt, but various other conditions and disorders, alone or combined, (can) result in a localized ammonia increase or a systemic ammonia increase (see below, and Tables 1, 2-3 (the double-underlined conditions and disorders) and 4). In particular, said conditions and disorders are selected from the group consisting of severe hepatic disorders (resulting from cirrhosis, hepatitis, intoxication, . . . ), errors of metabolism (urea cycle disorder, . . . ), porto-systemic shunts, gastrointestinal haemorrhages, transplant rejections and catabolic states.

In particular, said conditions and disorders can be selected in the group consisting of hyperammonemia and i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders.

In a second preferred embodiment, said conditions and disorders are related to or result in (absolute or relative) deficiency or excess of glutamate and/or in (absolute or relative) deficiency or excess of glutamine, and/or sensitive to glutamine deprivation or glutamine supplementation. Thus, said conditions and disorders are preferably selected from those disclosed in Tables 2 (in particular those marked in “italic”) and 3, with the exception of those disclosed in Table 14. In particular, said conditions and disorders can be selected in the group consisting of i) Adrenal gland disorders, particularly cortical hyperfunctions and hypofunctions (incl iatrogenic), ii) Gastrointestinal inflammatory conditions, particularly Inflammatory bowel disease, Gastrointestinal motility and defaecation conditions e.g. Irritable bowel syndrome, Dyspeptic signs and symptoms e.g. Dyspepsia and Flatulence, bloating and distension, Gastrointestinal ulceration and perforation, and Malabsorption conditions, iii) Allergic conditions, more particularly Allergic bronchitis and Asthma, Autoimmune disorders and Immune disorders, iv) Infections and infestations, v) Metabolism and nutrition disorders such as Appetite and general nutritional disorders, Cushing's syndrome, Hypercatabolism and Hypercorticoidism, vi) Bone disorders, particularly Osteoporosis, Connective tissue disorders, particularly Lupus erythematosus, Joint disorders, Muscle disorders, and Tendon, ligament and cartilage disorders, vii) Adjustment disorders, Anxiety disorders and symptoms, more particularly Obsessive compulsive disorder and Stress disorders, Cognitive and attention disorders and disturbances, Depressed mood disorders and disturbances, Manic and bipolar mood disorders and disturbances, Mood disorders and disturbances, more particularly Affect alterations, Emotional and mood disturbances and Mood disorders due to a general medical condition, Mental disorders due to a general medical condition, xiii) Neoplasms benign, malignant and unspecified, particularly Gliomas benign viii) Bronchial conditions e.g. Allergic bronchitis and Bronchitis chronic and Bronchospasm e.g. Asthma, Cough.

The present invention also relates to the use of NF, salts thereof, isomers thereof, pro-drugs thereof, metabolites thereof or structural analogs thereof, in the preparation of compositions to be used to activate, inhibit and/or regulate GS activity intended for the activation, inhibition and/or regulation of functions which involve GS activity or can be influenced by GS activity, directly or indirectly, in non-vertebrate organisms with eukaryotic cells such as invertebrate animals (arthropods, protozoa, shellfish, worms, . . . ), algae, fungi (yeasts, molds, . . . ) or plants, but also prokaryotes e.g. algae and bacteria, aiming to facilitate their growth or protect them from stresses and disorders or, inversely, aiming to combat their growth. Thus, the present invention concerns methods to facilitate the growth of non-vertebrate organisms with eukaryotic cells such as invertebrate animals (arthropods, protozoa, shellfish, worms, . . . ), algae, fungi (yeasts, molds, . . . ) or plants, but also of prokaryotes e.g. algae or bacteria, either to protect them from stresses and disorders, or to combat them, comprising administering an efficient amount of NF or a salt thereof, an isomer thereof, a pro-drug thereof, a metabolite thereof or a structural analog thereof. Therefore, the invention concerns the use of NF or a salt, an isomer, a pro-drug, a metabolite or a structural analog thereof, for the preparation of a composition aiming to facilitate the growth of non-vertebrate organisms with eukaryotic cells such as invertebrate animals (arthropods, protozoa, shellfish, worms, . . . ), algae, fungi (yeasts, molds, . . . ) or plants, and prokaryotes e.g. algae or bacteria, or to protect them from stresses and disorders, or inversely, aiming to combat their growth.

Furthermore, resulting from the use of tests on the native GS enzyme i.e. without reducing agent (due to our experience with tianeptine), the present invention relates to novel pharmacological effects/therapeutic uses of already known, classical GS ligands such as glufosinate (GF; phosphinothrycin (PPT)) and L-methionine sulfoximine (L-MSO or MS), but also hydrazines and bisphosphonates (by extension Mg²⁺ analogs like Sr²⁺), and salts thereof, isomers thereof, pro-drugs thereof, metabolites thereof and structural analogs thereof, for which regulating properties on native GS activity have been discovered by the Inventors. In fact, depending on the dose/concentration and experimental or (patho)physiological condition, the Inventors demonstrated that all these ligands can activate and inhibit GS activity: i) at “low concentrations”, depending on the experimental or (patho)physiological condition, they could behave both as GS activators or GS inhibitors, while ii) at “high concentrations”, they behaved strictly as GS inhibitors. By “regulator” or “regulating” is referred herein to a drug having an activating or inhibiting effect on GS depending on the target site (cell, tissue, or organ) and on the (patho)physiological conditions. The effect is to be opposed to the strictly GS inhibitory effect that the drug has at a high dose. When a high dose is used, the drug has an inhibitory effect on GS whatever the target site is or the (patho)physiological conditions are.

These “other GS regulating ligands” are claimed to be used for treating all the conditions and disorders already proposed to be sensitive to tianeptine or NF, and/or for screening and developing drugs to be used in all the conditions and disorders already proposed to be sensitive to tianeptine or NF, with the exception of: if said GS ligand is GF or MS, the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is a hydrazine, the conditions and disorders disclosed in Table 6; and if said GS ligand is a bisphosphonate, the conditions and disorders disclosed in Table 7. More particularly, the conditions and disorders already proposed to be sensitive to tianeptine or salts thereof, isomers thereof, pro-drugs thereof, metabolites thereof or structural analogs thereof, are selected from the group consisting of the conditions and disorders disclosed in Table 5. More particularly, the conditions and disorders already proposed to be sensitive to NF or salts thereof, isomers thereof, pro-drugs thereof, metabolites thereof or structural analogs thereof, are selected from the group consisting of the conditions and disorders disclosed in Table 14. In particular, these conditions and disorders include: i) asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of AIDS, . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke, and ii) Vascular disorders from Arteriosclerosis, stenosis, vascular insufficiency and necrosis, Embolism and thrombosis, Vascular disorders NEC, Vascular haemorrhagic disorders, Vascular inflammations and Venous varices groups, and Nervous system disorders from the Central nervous system vascular disorders, Encephalopathies, Mental impairment disorders, Movement disorders (incl Parkinsonism), Neurological disorders of the eye, Neuromuscular disorders and Seizures (incl subtypes) groups such as acute and chronic neurodegenerative diseases, Alzheimer's, Huntington's, Parkinson's diseases, multiple sclerosis, amyotrophic lateral sclerosis, spinal muscular atrophy, retinopathy, and traumatic brain injury, drug-induced neurotoxicity, pain, hormonal balance, blood pressure, thermoregulation, respiration, learning, pattern recognition, memory, and disorders subsequent to hypoxia or hypoglycaemia with the exception of: if said GS ligand is GF or MS, the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is a hydrazine, the conditions and disorders disclosed in Table 6; and if said GS ligand is a bisphosphonate, the conditions and disorders disclosed in Table 7. Then, the present invention concerns the use of a GS regulating ligand, with the exception of tianeptine and NF, for the preparation of a medicament for the prevention and/or treatment of a condition or a disorder selected from the group consisting of asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of AIDS, . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke, and ii) Vascular disorders from Arteriosclerosis, stenosis, vascular insufficiency and necrosis, Embolism and thrombosis, Vascular disorders NEC, Vascular haemorrhagic disorders, Vascular inflammations and Venous varices groups, and Nervous system disorders from the Central nervous system vascular disorders, Encephalopathies, Mental impairment disorders, Movement disorders (incl Parkinsonism), Neurological disorders of the eye, Neuromuscular disorders and Seizures (incl subtypes) groups such as acute and chronic neurodegenerative diseases, Alzheimer's, Huntington's, Parkinson's diseases, multiple sclerosis, amyotrophic lateral sclerosis, spinal muscular atrophy, retinopathy, and traumatic brain injury, drug-induced neurotoxicity, pain, hormonal balance, blood pressure, thermoregulation, respiration, learning, pattern recognition, memory, and disorders subsequent to hypoxia or hypoglycaemia, with the exception of: if said GS ligand is GF or MS, the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain; and, if said GS ligand is a hydrazine, the conditions and disorders disclosed in Table 6; and if said GS ligand is a bisphosphonate, the conditions and disorders disclosed in Table 7. Preferably, said “other GS regulating ligand” is selected from the group consisting of GF, MS, hydrazines and bisphosphonates, and salts, isomers, pro-drugs, metabolites and structural analogs thereof. In one preferred embodiment, said GS ligand is GF or a salt, an isomer, a pro-drug, a metabolite or a structural analog thereof. In another preferred embodiment, said GS ligand is MS or a salt, an isomer, a pro-drug, a metabolite or a structural analog thereof. In a further preferred embodiment, said GS ligand is a hydrazine or a salt, an isomer, a pro-drug, a metabolite or a structural analog thereof. In an additional preferred embodiment, said GS ligand is a bisphosphonate or a salt, an isomer, a pro-drug, a metabolite or a structural analog thereof.

These “other GS regulating ligands” can be used for regulating all types of functions which involve GS activity or can be influenced by GS activity, directly or indirectly, and/or for preventing and/or treating all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, either alone or combined.

In fact, the Inventors have discovered that these “other GS regulating ligands”, which were for certain, like GF and MS, unanimously believed to be GS inhibitors in animals, can be used to regulate GS activity i.e. to activate or inhibit GS activity when they are used at concentrations/doses lower as compared to those which inhibit GS activity systematically. In short, i) at low concentrations, depending on the experimental or (patho)physiological condition, these GS ligands can behave both as GS activators or GS inhibitors while ii) at high concentrations, they behave strictly as GS inhibitors.

Therefore, the present invention concerns the use of these “other GS regulating ligands” for the preparation of medicaments having a regulating effect on GS, wherein said GS ligands are used at “low doses” i.e. doses resulting in activation or inhibition of GS activity in the target cells/tissues/organs/organism, without any strict inhibition, thus no secondary safety and tolerability issues. In a most preferred embodiment, the present invention concerns the use of these “other GS regulating ligands” for the preparation of medicaments having a regulating effect on GS, wherein said GS ligands are used at doses resulting in activation of the activity of GS in the target cells/tissues/organs/organism without any strict GS inhibition.

Therefore, the present invention concerns the use of a GS ligand, with the exception of tianeptine and NF, for the preparation of a medicament to prevent and/or treat all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, wherein the GS ligand is used at doses resulting in activation or inhibition of GS activity in the target cells/tissues/organs/organism, without any strict inhibition Preferably, the GS ligand is used at doses resulting in activation of GS activity in the target cells/tissues/organs/organism, without any strict inhibition. In particular, said conditions and disorders are selected from those disclosed in Tables 1-4, with the exception of the already known indications of these ligands i.e. if said GS ligand is GF or MS, the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is a hydrazine, the conditions and disorders disclosed in Table 6; and, if said GS ligand is a bisphosphonate, the conditions and disorders disclosed in Table 7. The present invention also concerns a method for prevention and/or treatment of any condition or disorder selected from those disclosed in Tables 1-4, comprising administering an efficient amount of a GS ligand or a salt thereof, an isomer thereof, a pro-drug thereof, a metabolite thereof or a structural analog thereof at doses resulting in activation or inhibition of GS activity in the target cells/tissues/organs/organism, without any strict inhibition, thereby preventing or treating said condition or disorder, with the exception of: if said GS ligand is GF or MS, the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is a hydrazine, the conditions and disorders disclosed in Table 6; and, if said GS ligand is a bisphosphonate, the conditions and disorders disclosed in Table 7. Preferably, the GS ligand is used at doses resulting in activation of GS activity in the target cells/tissues/organs/organism, without any strict inhibition. Potential factors and mechanisms which can underlie these conditions and disorders are disclosed in Table 4, without to be limited thereto. In a preferred embodiment, said conditions and disorders are selected from those disclosed in Tables 2 and 3. In another preferred embodiment, said conditions and disorders are related to hyperammon(em)ia (see Tables 1 and 2-3 (the double-underlined conditions and disorders)). In a particular embodiment, said GS ligand is selected from the group consisting of GF, MS, hydrazines and bisphosphonates, and salts, isomers, pro-drugs, metabolites and structural analogs thereof. In one preferred embodiment, said GS ligand is GF or a salt, an isomer, a pro-drug, a metabolite or a structural analog thereof. In another preferred embodiment, said GS ligand is MS or a salt, an isomer, a pro-drug, a metabolite or a structural analog thereof. In a further preferred embodiment, said GS ligand is a hydrazine or a salt, an isomer, a pro-drug, a metabolite or a structural analog thereof. In an additional preferred embodiment, said GS ligand is a bisphosphonate or a salt, an isomer, a pro-drug, a metabolite or a structural analog thereof. Optionally, said GS ligand or salt, isomer, pro-drug, metabolite or structural analog thereof, can be used in combination with an other drug intended to prevent/treat hyperammon(emi)a or resulting in hyperammon(em)ia, or an other drug intended to prevent/treat conditions and disorders related to deficiency or excess of glutamate or resulting in deficiency or excess of glutamate, or an other drug intended to prevent/treat conditions and disorders related to deficiency or excess of glutamine or resulting in deficiency or excess of glutamine. Thus, said conditions and disorders are preferably selected from those disclosed in Tables 2 (in particular those marked in “italic”) and 3. In a preferred embodiment, without to be limited thereto, said conditions and disorders are selected from the group consisting of Platelet disorders, Cardiac arrhythmias, Reiter's syndrome, Lupus erythematosus and associated conditions, Rheumatoid arthritis and associated conditions, Bone and joint injuries, Osteoporosis, Schizophrenia and other psychotic disorders, Penile and scrotal disorders, Sexual function and fertility disorders, Blood brain barrier defect and Nervous system disorders.

The GS ligand can be used in combination with an other drug. In particular, these drugs include: i) drugs intended to prevent/treat hyperammon(em)ia and/or hyperammon(em)ia symptoms e.g. flumazenil, zinc, sodium benzoate, sodium phenylacetate and arginine hydrochloride, ii) drugs resulting in hyperammon(em)ia e.g. anti-cancer therapies (5-fluorouracil, asparaginase, . . . ) and antiepileptics (sodium valproate, primidone, . . . ), iii) drugs altering glutamate receptor(s) function(s) e.g. ampakines, glutamate release inhibitors (lamotrigine, riluzole, . . . ), glutamate receptor antagonists (D-AP5, ketamine, memantine, MK-801, . . . ) and glycine, iv) drugs intended to prevent/treat disorders where GS protein/activity is altered or relatively ineffective e.g. anti-epileptic treatments (benzodiazepines, carbamazepine, ethosuximide, hydantoins, gabapentin, lamotrigin, phenobarbital, primidone, valproate, . . . ) and antibiotics (aminoglycosides, cephalosporins, chloramphenicol, macrolides, penicillins, quinolones, rifampicine, sulfonamides, tetracyclines, trimethoprim-sulfamethoxazole, . . . ) in certain forms of epilepsy and critical sepsis, respectively, v) drugs intended to combat neoplasms and/or inflammatory diseases, e.g. anti-proliferative (5-fluorouracil, asparaginase, . . . ), and vi) anti-inflammatory drugs or drugs preventing from the occurrence of inflammation, in particular glucocorticoids.

Preferably, said “other GS regulating ligands” are used at “low doses” i.e. doses resulting in concentrations which allows to activate and/or inhibit i.e. regulate the activity of GS in the target cells/tissues/organs/organism, without any strict inhibition, thus usually no secondary safety and tolerability issues. In a most preferred embodiment, said “other GS regulating ligands” are used at doses resulting in an increase of the activity of GS in the target cells/tissues/organs/organism.

In particular, the present invention concerns the use of GF, salts thereof, isomers thereof, pro-drugs thereof, metabolites thereof and structural analogs thereof, as a medicament, preferably at “low doses” i.e. doses resulting in regulation of GS activity in the target cells/tissues/organs/organism, in the manufacture of a pharmaceutical composition for preventing and/or treating conditions and disorders which involve or can be influenced by GS activity, directly or indirectly, in particular those related to absolute or relative deficiency or excess of glutamate, and those related to absolute or relative deficiency or excess of glutamine, with the exception of the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain. In a preferred embodiment, said metabolite or analog of GF is selected from the group consisting of the compounds disclosed in Tables 9 and 13, preferably Table 9. Preferably, the compound is selected from the group consisting of GF, 4-methylphosphinico-2-oxo-butanoic acid, 3-methylphosphinicopropionic acid, 4-methylphosphinico-2-hydroxybutanoic acid, 4-methylphosphinicobutanoic acid, 2-methylphosphinicoacetic acid, 2-acetamido-4-methylbutanoic acid, gamma-hydroxy phosphinothricin, gamma-methyl phosphinothricin, gamma-acetoxy phosphinothricin, alpha-methyl phosphinothricin, alpha-ethyl phosphinothricin, cyclohexane phosphinothricin, cyclopentane phosphinothricin, tetrahydrofuran phosphinothricin, s-phosphonomethyl homocysteine sulfoxide, s-phosphonomethyl homocysteine sulfone, 2-amino-4-[(phosphonomethyl)hydroxyphosphinyl-1]butanoic acid, 2-amino-4-phosphono butanoic acid, 4-amino-4-phosphono butanoic acid, 2-amino-2-methyl-4-phosphono butanoic acid, 4-amino-4-(hydroxymethylphosphinyl)-4-methyl butanoic acid, 2-methoxycarbonyl-4-phosphono butamoic acid, methyl 4-amino-4-phosphono butanate, 4-amino-4-(hydroxymethylphosphinyl)butanoic acid, 2-amino-4-(hydroxymethylphosphinyl)butanoic acid, phosphinothricin, s-phosphonomethyl homocysteine, 4-(phosphonoacetyl)-L-alpha-aminobutyrate, threo-4-hydroxy-D-glutamic acid, erythro-4-fluoro-D,L-glutamic acid, 4-amino-4-(hydroxy-methyl-phosphinoyl)-butyric acid, 2-methoxycarbonyl-4-phosphono butanoic acid, methyl 4-amino-4-phosphono butanoate and 2-amido-4-phosphono butanoic acid, and salts and isomers thereof. Optionally, GF, salts thereof, isomers thereof, pro-drugs thereof, metabolites thereof and structural analogs thereof is used in combination with an other etiologic and/or symptomatic treatment i.e. a drug selected from the group consisting of drugs intended to prevent and/or treat conditions and disorders which involve or can be influenced by GS activity, in particular the conditions and disorders related to absolute or relative excess of ammonia, those related to absolute or relative deficiency or excess of glutamate, and those related to absolute or relative deficiency or excess of glutamine. In particular, these include: i) drugs intended to prevent/treat hyperammon(em)ia and/or hyperammon(em)ia symptoms e.g. flumazenil, zinc, sodium benzoate, sodium phenylacetate and arginine hydrochloride, ii) drugs resulting in hyperammon(em)ia e.g. anti-cancer therapies (5-fluorouracil, asparaginase, . . . ) and antiepileptics (sodium valproate, primidone, . . . ), iii) drugs altering glutamate receptor(s) function(s) e.g. ampakines, glutamate release inhibitors (lamotrigine, riluzole, . . . ), glutamate receptor antagonists (D-AP5, ketamine, memantine, MK-801, . . . ) and glycine, iv) drugs intended to prevent/treat disorders where GS protein/activity is altered or relatively ineffective e.g. anti-epileptic treatments (benzodiazepines, carbamazepine, ethosuximide, hydantoins, gabapentin, lamotrigin, phenobarbital, primidone, valproate, . . . ) and antibiotics (aminoglycosides, cephalosporins, chloramphenicol, macrolides, penicillins, quinolones, rifampicine, sulfonamides, tetracyclines, trimethoprim-sulfamethoxazole, . . . ) in certain forms of epilepsy and critical sepsis, respectively, v) drugs intended to combat neoplasms and/or inflammatory diseases, e.g. anti-proliferative (5-fluorouracil, asparaginase, . . . ), and vi) anti-inflammatory drugs or drugs preventing from the occurrence of inflammation, in particular glucocorticoids.

The present invention also concerns a method for the prevention and/or the treatment of any condition or disorder selected from those disclosed in Tables 2-4, with the exception of the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain, comprising administering an efficient amount of GF or a salt thereof, an isomer thereof, a pro-drug thereof, a metabolite thereof or a structural analog thereof, thereby preventing and/or treating said condition or disorder. Preferably, said conditions and disorders are selected from those disclosed in Tables 24, preferably Tables 2-3, with the exception of the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain. In a particular embodiment, said conditions and disorders are selected from the group consisting of i) Platelet disorders, ii) Cardiac disorders, particularly Cardiac arrhythmia, Cardiomyopathies, and myocarditis, iii) Adrenal gland disorders, particularly cortical hyperfunctions and hypofunctions (incl iatrogenic), and Hypothalamus and pituitary gland disorders, iv) Gastrointestinal inflammatory conditions, particularly Inflammatory bowel disease, Gastrointestinal motility and defaecation conditions e.g. Irritable bowel syndrome, Dyspeptic signs and symptoms e.g. Dyspepsia and Flatulence, bloating and distension, Gastrointestinal ulceration and perforation, and Malabsorption conditions, v) General disorders and administration site conditions such as Asthenic conditions, Inflammations, Mucosal findings abnormal, discomfort, and Tissue disorders, more particularly Trophic disorders e.g. Atrophy and Denervation atrophy, vi) Allergic conditions, more particularly Allergic bronchitis and Asthma, Autoimmune disorders and Immune disorders vii) Injury, poisoning and procedural complications such as Administration site reactions, Chemical injury and poisoning, Injuries by physical agents, and other Procedural and device related injuries and complications e.g. adverse effects of corticoids, viii) Metabolism and nutrition disorders such as Appetite and general nutritional disorders, Cushing's syndrome, Hypercatabolism and Hypercorticoidism, ix) Bone disorders, particularly Osteoporosis, Connective tissue disorders, particularly Lupus erythematosus, Joint disorders, and Muscle disorders ix) Central nervous system inflammations, Demyelinating disorders, particularly Multiple sclerosis, Memory loss, Movement disorders (incl Parkinsonism), Nervous system neoplasms benign, particularly Gliomas benign, Coma states, Cognitive deterioration and Confusion postoperative, Anaesthetic complication neurological, Motor neurone diseases e.g. Amyotrophic lateral sclerosis, Neuromuscular disorders and Neuromuscular junction dysfunction, Peripheral neuropathies, Seizures (incl subtypes), Sleep disturbances, Spinal nerve root disorders and Structural brain disorders. x) Adjustment disorders, Anxiety disorders and symptoms, more particularly Obsessive compulsive disorder and Stress disorders, Changes in physical activity, Cognitive and attention disorders and disturbances, Communication disorders and disturbances, Depressed mood disorders and disturbances, Dissociative disorders, Disturbances in thinking and perception, Eating disorders and disturbances, Impulse control disorders, Manic and bipolar mood disorders and disturbances, Mood disorders and disturbances, more particularly Affect alterations, Emotional and mood disturbances and Mood disorders due to a general medical condition, Personality disorders and disturbances in behaviour, Mental disorders due to a general medical condition e.g. corticoids, Schizophrenia and other psychotic disorders, Sleep disorders and disturbances, Somatoform and factitious disorders, and Suicidal and self-injurious behaviours, xi) Menopause and related conditions xii) Bronchial conditions e.g. Allergic bronchitis and Bronchitis chronic and Bronchospasm e.g. Asthma, Respiratory tract inflammatory and immunologic conditions, Parenchymal lung disorders and Pulmonary oedemas, Pulmonary vascular disorders, Respiratory disorders, more particularly Conditions associated with abnormal gas exchange, Cough, xiii) Skin and subcutaneous tissue disorders signs and symptoms, xiv) Vascular disorders such as Arteriosclerosis and vascular insufficiency, Circulatory collapse and shock, Embolism and thrombosis, Blood brain barrier defect, and Ear and Ocular vascular disorders, Vascular haemorrhagic disorders, Vascular hypertensive disorders, Vascular inflammations, and Venous varices.

In particular, the present invention concerns the use of MS, salts thereof, isomers thereof, pro-drugs thereof, metabolites thereof and structural analogs thereof, preferably at “low dose” i.e. doses resulting in regulation of GS activity in the target cells/tissues/organs/organism, in the manufacture of medicaments for preventing and/or treating all types of conditions and disorders which involve or can be influenced by GS activity, directly or indirectly, in particular those related to absolute or relative deficiency or excess of glutamate, and those related to absolute or relative deficiency or excess of glutamine, with the exception of the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain. The present invention also concerns a method for the prevention and/or the treatment of any condition or disorder selected from those disclosed in Tables 24, with the exception of the conditions and disorders related to cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain, comprising administering an efficient amount of MS or a salt thereof, an isomer thereof, a pro-drug thereof, a metabolite thereof or a structural analog thereof, preferably at a “low dose” i.e. doses resulting in regulation of GS activity in the target cells/tissues/organs/organism, thereby preventing or treating said condition or disorder. Preferably, said conditions and disorders are selected from those disclosed in Tables 24, preferably Tables 2 (in particular the conditions and disorders in “italic”) and 3, with the exception of the conditions and disorders related cerebral ischemia, hyperammonemia (marked in “double-underlined” in Table 2), bacterial, viral and fungal infectious disorders (antimicrobial effect), neoplasm (cytotoxic effect), neurogenerative diseases (Alzheimer disease, Huntington's and other polyglutamine disorders) and pain. In a particular embodiment, said conditions or disorders are selected from the group consisting of i) Platelet disorders, ii) Cardiac disorders, particularly Cardiac arrhythmia, Cardiomyopathies, and myocarditis, iii) Adrenal gland disorders, particularly cortical hyperfunctions and hypofunctions (incl iatrogenic), and Hypothalamus and pituitary gland disorders, iv) Gastrointestinal inflammatory conditions, particularly Inflammatory bowel disease, Gastrointestinal motility and defaecation conditions e.g. Irritable bowel syndrome, Dyspeptic signs and symptoms e.g. Dyspepsia and Flatulence, bloating and distension, Gastrointestinal ulceration and perforation, and Malabsorption conditions, v) General disorders and administration site conditions such as Asthenic conditions, Inflammations, Mucosal findings abnormal, discomfort, and Tissue disorders, more particularly Trophic disorders e.g. Atrophy and Denervation atrophy, vi) Allergic conditions, more particularly Allergic bronchitis and Asthma, Autoimmune disorders and Immune disorders vii) Injury, poisoning and procedural complications such as Administration site reactions, Chemical injury and poisoning, Injuries by physical agents, and other Procedural and device related injuries and complications e.g. adverse effects of corticoids, viii) Metabolism and nutrition disorders such as Appetite and general nutritional disorders, Cushing's syndrome, Hypercatabolism and Hypercorticoidism, ix) Bone disorders, particularly Osteoporosis, Connective tissue disorders, particularly Lupus erythematosus, Joint disorders, and Muscle disorders ix) Central nervous system inflammations, Demyelinating disorders, particularly Multiple sclerosis, Memory loss, Movement disorders (incl Parkinsonism), Nervous system neoplasms benign, particularly Gliomas benign, Coma states, Cognitive deterioration and Confusion postoperative, Anaesthetic complication neurological, Motor neurone diseases e.g. Amyotrophic lateral sclerosis, Neuromuscular disorders and Neuromuscular junction dysfunction, Peripheral neuropathies, Seizures (incl subtypes), Sleep disturbances, Spinal nerve root disorders and Structural brain disorders. x) Adjustment disorders, Anxiety disorders and symptoms, more particularly Obsessive compulsive disorder and Stress disorders, Changes in physical activity, Cognitive and attention disorders and disturbances, Communication disorders and disturbances, Depressed mood disorders and disturbances, Dissociative disorders, Disturbances in thinking and perception, Eating disorders and disturbances, Impulse control disorders, Manic and bipolar mood disorders and disturbances, Mood disorders and disturbances, more particularly Affect alterations, Emotional and mood disturbances and Mood disorders due to a general medical condition, Personality disorders and disturbances in behaviour, Mental disorders due to a general medical condition e.g. corticoids, Schizophrenia and other psychotic disorders, Sleep disorders and disturbances, Somatoform and factitious disorders, and Suicidal and self-injurious behaviours, xi) Menopause and related conditions xii) Bronchial conditions e.g. Allergic bronchitis and Bronchitis chronic and Bronchospasm e.g. Asthma, Respiratory tract inflammatory and immunologic conditions, Parenchymal lung disorders and Pulmonary oedemas, Pulmonary vascular disorders, Respiratory disorders, more particularly Conditions associated with abnormal gas exchange, Cough, xiii) Skin and subcutaneous tissue disorders signs and symptoms, xiv) Vascular disorders such as Arteriosclerosis and vascular insufficiency, Circulatory collapse and shock, Embolism and thrombosis, Blood brain barrier defect, and Ear and Ocular vascular disorders, Vascular haemorrhagic disorders, Vascular hypertensive disorders, Vascular inflammations, and Venous varices. In a preferred embodiment, said metabolites and analogs of MS are selected from the group consisting of the compounds disclosed in Tables 10 and 13, preferably Table 10.

Optionally, the present invention concerns the use of hydrazines, salts thereof, isomers thereof, pro-drugs thereof, metabolites thereof and structural analogs thereof, preferably at “low doses” i.e. doses resulting in regulation of GS activity in the target cells/tissues/organs/organism, in the manufacture of medicaments for preventing and/or treating all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, with the exception of the conditions and disorders disclosed in Table 6. The present invention also concerns a method for the prevention and/or the treatment of any condition or disorder selected from those disclosed in Tables 1-4, preferably those disclosed in Tables 1-3, with the exception of the conditions and disorders disclosed in Table 6, comprising administering an efficient amount of a hydrazine or a salt thereof, an isomer thereof, a pro-drug thereof, a metabolite thereof or a structural analog thereof, preferably at “low doses” i.e. doses resulting in regulation of GS activity in the target cells/tissues/organs/organism, thereby preventing or treating said condition or disorder. Preferably, said conditions and disorders are selected from those disclosed in Tables 1-4, preferably Tables 2 (in particular the conditions and disorders in “italic”) and 3, with the exception of the conditions and disorders disclosed in Table 6. In a particular embodiment, said conditions or disorders are selected from the group consisting of hyperammon(em)ia, i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders, and i) Platelet disorders, ii) Endocrine disorders, particularly cortical hyperfunctions and hypofunctions (incl iatrogenic), iii) Gastrointestinal inflammatory conditions, Gastrointestinal motility and defaecation conditions, Dyspeptic signs and symptoms, and Malabsorption conditions, iv) General disorders and administration site conditions such as Mucosal findings abnormal, and Tissue disorders, more particularly Trophic disorders e.g. Atrophy and Denervation atrophy, v) Injury, poisoning and procedural complications such as Administration site reactions, Chemical injury and poisoning, Injuries by physical agents, and other Procedural and device related injuries and complications, vi) Metabolism and nutrition disorders such as Appetite and general nutritional disorders, Cushing's syndrome, Hypercatabolism and Hypercorticoidism, vii) Bone disorders, particularly Osteoporosis, Connective tissue disorders, and Muscle disorders, viii) Nervous system disorders, particularly Demyelinating disorders, encephalopathies, Mental impairment disorders, Nervous system neoplasms benign, particularly Gliomas benign, Cognitive deterioration and Confusion postoperative, Anaesthetic complication neurological, Motor neurone diseases e.g. Amyotrophic lateral sclerosis, Neuromuscular disorders and Neuromuscular junction dysfunction, Seizures, Sleep disturbances and Structural brain disorders, ix) Adjustment disorders, Anxiety disorders and symptoms, Dementia and amnestic conditions, Dissociative disorders, Eating disorders and disturbances, Impulse control disorders, Mental disorders due to a general medical condition e.g. corticoids, Schizophrenia and other psychotic disorders, Sleep disorders and disturbances, Somatoform and factitious disorders, and Suicidal and self-injurious behaviours, x) Renal and urinary disorders xi) Bronchospasm e.g. Asthma, Pulmonary oedemas, Conditions associated with abnormal gas exchange, and Cough, xiii) Vascular disorders such as Blood brain barrier defect.

In a preferred embodiment, said metabolites and analogs of hydrazine are selected from the group consisting of the compounds disclosed in Table 11. In a preferred embodiment, said conditions and disorders are related to or result in hyperammon(em)ia or result in hyperammon(em)ia.

In particular, the present invention concerns the use of bisphosphonates, salts thereof, isomers thereof, pro-drugs thereof, metabolites thereof and structural analogs thereof (by extention Mg²⁺ analogs like Sr²⁺), preferably at “low doses” i.e. doses resulting in regulation of GS activity in the target cells/tissues/organs/organism, in the manufacture of medicaments for preventing and/or treating all types of conditions and disorders which involve or can be influenced by GS activity, in particular those related to absolute or relative excess of ammonia, those related to absolute or relative deficiency or excess of glutamate, and those related to absolute or relative deficiency or excess of glutamine, with the exception of the conditions and disorders disclosed in Table 7. The present invention also concerns a method for the prevention and/or the treatment of any condition or disorder selected from those disclosed in Tables 1-4, with the exception of the conditions and disorders disclosed in Table 7, comprising administering an efficient amount of a bisphosphonate or a salt thereof, an isomer thereof, a pro-drug thereof, a metabolite thereof or a structural analog thereof, preferably at “low doses” i.e. doses resulting in regulation of GS activity in the target cells/tissues/organs/organism, thereby preventing or treating said condition or disorder. Preferably, said conditions and disorders are selected from those disclosed in Tables 1-4, preferably Tables 2 (in particular the conditions and disorders in “italic”) and 3, with the exception of those disclosed in Table 7. In a particular embodiment, said conditions and disorders are selected from the group consisting of i) Platelet disorders, ii) Cardiac disorders, iii) Ear disorders, iv) Endocrine disorders, particularly cortical hypofunctions, v) Eye disorders, vi) Gastrointestinal disorders, in particular Gastrointestinal inflammatory conditions, Gastrointestinal motility and defaecation conditions, Dyspeptic signs and symptoms, Gastrointestinal ulceration and perforation, and Malabsorption conditions, vii) General disorders and administration site conditions such as Inflammations, Mucosal findings abnormal, and Tissue disorders, more particularly Trophic disorders, viii) Immune disorders, in particular Autoimmune disorders and Immune disorders, ix) Injury, poisoning and procedural complications such as Administration site reactions, Chemical injury and poisoning, Injuries by physical agents, and other Procedural and device related injuries and complications, x) Metabolism and nutrition disorders such as Hypercatabolism, xi) Connective tissue disorders, particularly Lupus erythematosus, xii) Nervous system disorders, xiii) Psychiatric disorders, xiv) Renal and urinary disorders, xv) Respiratory, thoracic and mediastinal disorders, xvi) Vascular disorders such as Blood brain barrier defect, Vascular hypertensive disorders, Vascular inflammations, and Venous varices. In a preferred embodiment, said conditions and disorders are related to or result in hyperammon(em)ia or result in hyperammon(em)ia and are preferably selected from the group consisting of) i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders.

In another additional embodiment, a GS ligand selected from the group consisting of GF, MS, hydrazines and bisphosphonates, and salts, isomers, pro-drugs, metabolites and structural analogs thereof, is used to activate and/or inhibit i.e. regulate GS activity, intended for the regulation of all types of functions which involve GS activity or can be influenced by GS activity in non-vertebrate organisms with eukaryotic cells such as invertebrate animals (arthropods, protozoa, shellfish, worms, . . . ), algae, fungi (yeasts, molds, . . . ) or plants, but also prokaryotes e.g. algae or bacteria, aiming to facilitate their growth or protect them from stresses and disorders. In a most preferred embodiment, GS ligand is used at “low doses” resulting in activation and/or inhibition of GS activity in the target cells/tissues/organs/organisms. Therefore, the invention concerns the use of a GS ligand selected from the group consisting of GF, MS, hydrazines and bisphosphonates, and salts, isomers, pro-drugs, metabolites and structural analogs thereof, for the preparation of a composition aiming to facilitate the growth of non-vertebrate organisms with eukaryotic cells such as invertebrate animals (arthropods, protozoa, shellfish, worms, . . . ), algae, fungi (yeasts, molds, . . . ) or plants, or prokaryotes e.g. algae or bacteria, or to protect them from stresses and disorders, wherein said GS ligand is used at a “low dose” i.e. a dose resulting in regulation of GS activity without any strict inhibition, provided that if said GS ligand is GF, MS or a bisphosphonate, plants are excluded.

Furthermore, the present invention also concerns methods for screening, identifying and/or developing a new biological active compound for the treatment of all types conditions and disorders which involve or can be influenced by GS activity, comprising a competitive assay based on the activity of the native GS with tianeptine or NF. Accordingly, the present invention concerns methods for screening, identifying and/or developing a medicament for prevention and/or treatment of asthma, cough, depression (major depression with or without melancholia, dysthymic disorders, depressed bipolar disorder, . . . ), irritable bowel syndrome, mnemo-cognitive disorders, nonulcer dyspepsia, pain, psychoneurotic disorders (neurotic or reactive states of depression, anxiodepressive states with somatic complaints such as digestive problems, anxiodepressive states observed in alcoholic detoxification, . . . ), stress, Vascular inflammations and venous varices, comprising essentially the screening and the identification of compounds regulating GS activity.

The present invention also concerns methods for monitoring treatments with a drug selected from the group consisting of tianeptine, NF, GF, hydrazines, bisphosphonates (by extension strontium), salts, isomers, pro-drugs, metabolites and structural analogs thereof comprising determining biological markers related GS enzyme like GS activity, GS expression, glutamate, glutamine and NH₄. By monitoring is intended in particular determining the treatment efficiency or the appropriate dose of GS ligand. The present invention also concerns the use of biological markers related to the GS enzyme like GS activity, GS expression, glutamate, glutamine and NH₄ to monitor treatments with a drug selected from the group consisting of NF, tianeptine, GF, hydrazines, bisphosphonates, strontium, salts, isomers, pro-drugs, metabolites and structural analogs thereof.

Finally, the present invention concerns methods for screening, identifying and/or developing treatments regulating the GS activity in organisms with eukaryotic cells other than plants e.g. algae, (vertebrate and invertebrate) animals including humans, and fungi (yeasts, molds, . . . ), and prokaryotes e.g. algae or bacteria, comprising an assay with GF, MS, an hydrazine e.g. isoniazid or iproniazid, or a bisphosphonate e.g. pamidronate or risedronate (or Sr²⁺), or salts thereof, isomers thereof, pro-drugs thereof, metabolites thereof or structural analogs thereof.

The present invention also concerns the use of a GS ligand, preferably selected from the group consisting of tianeptine, NF, GF and MS as well as hydrazines, bisphosphonates and metal ions, isomers thereof, pro-drugs thereof, metabolites thereof and structural analogs thereof, for preparing a medicament for increasing the efficiency or/and preventing or decreasing the side effect of (gluco)corticoid treatment. The present invention further concerns a method for increasing the efficiency or/and preventing or decreasing the side effect of (gluco)corticoid treatment in a subject comprising administering an efficient amount of a GS ligand like tianeptine GF and MS as well as of NF, hydrazines, bisphosphonates and metal ions, isomers thereof. In particular, the GS ligand is used at low or very low doses. In particular, GS ligand has a synergic effect with glucocorticoid, allowing the dose of glucocorticoid used in combination with a GS ligand to be decreased with the same therapeutic effect and less side effects.

### LEGEND TO THE FIGURES

FIG. 1: Analytic HPLC (FIG. 1A) and mass spectrometry (FIG. 1B) profiles of biotinylated tianeptine.

FIG. 2: Western blotting on BALB/c mouse hippocampus membrane proteins. Membrane proteins with peroxidase conjugated streptavidin in absence (lane 1) or presence (lane 2) of n-octyl glucoside. Membrane proteins with biotinylated tianeptine and peroxidase conjugated streptavidin in absence (lane 3) or presence of n-octyl glucoside (lane 4).

FIG. 3: 2D gel electrophoresis and western blotting on BALB/c mouse hippocampus membrane proteins. The gel in FIG. 3A is stained with colloidal blue (Coomassie R 250). The arrows point the two consecutive spots at 45 kDa, which are recognised by biotinylated tianeptine in western blot as it is shown in FIG. 3B.

FIG. 4: In silico study of the interaction between tianeptine and bovine brain GS. The model shows the tianeptine molecule (ball and stick) in its binding site (amino acids at a distance equal or less than 5 Å of the center of the tianeptine molecule are shown as sticks). Glu A327 linked by a hydrogen bond to the NH of tianeptine is shown in red. The arginines A344 and A355 linked by a salt bridge to the carboxyl group of tianeptine are shown in dark blue. The two electropositive arginines A339 and A359 counterbalancing the electronegative SO₂ group of tianeptine are shown in blue. The other amino acids, forming the combining site (Glu A129, Glu A131, Tyr A179, His A210, Val A213, Asn A264, His A269, His A271, Pro A346 and Val A347) are coloured in yellow. Comparing the active site residues in the glufosinate (phosphinotricin)-glutamine synthetase complex (Gill and Eisenberg, 2001) with those in the tianeptine-glutamine synthetase complex, 10 out of the 12 amino acids are similar. The charge asymmetry of the binding site is conserved with the positive charges neutralizing the carboxyl group and the SO₂ group of tianeptine, and Glu A327 is blocked with a hydrogen bond of the NH group in the tianeptine cycle. Tianeptine thus recognizes the glutamine synthetase binding site in its closed form. This blocks the glutamate entrance to the catalytic site.

FIG. 5: Effects of glufosinate (FIG. 5A), iproniazid (FIG. 5B), L-methionine sulfoximine (FIG. 5C) and tianeptine (FIG. 5D) on sheep brain GS subunit (monomer) activity. GS activity was measured as the amount gamma-glutamyl hydroxamate produced (absorbance at 535 nm) in presence of 50 mM L-glutamine.

FIG. 6: Inhibition of sheep brain GS activity (absorbance related to gamma-glutamylhydroxamate formation) by MS (5 mM) and tianeptine (28 μM) in presence of various concentrations of L-glutamine (0.75-12.5 mM).

FIG. 7: Effects of glufosinate (FIG. 7A), iproniazid (FIG. 7B), L-methionine sulfoximine (FIG. 7C) and tianeptine (FIG. 7D) on sheep brain GS activity. Activity of GS was measured as the amount of gamma-glutamyl hydroxamate produced (absorbance at 535 nm) in presence of 50 mM L-glutamine.

FIG. 8: Thin layer chromatography with different concentrations of tianeptine in presence of GS before (A) and after (B) 24 hours dialysis against reaction buffer (see material and methods). Lane 3: reaction buffer with 20 μM ADP; Lane 4: reaction buffer with 20 μM ADP, 5 nM tianeptine and 0.06 units GS; Lane 5: reaction buffer with 20 μM ADP, 1 nM tianeptine and 0.06 units GS; Lane 6: reaction buffer with 20 μM ADP, 0.1 nM tianeptine and 0.06 units GS. Lanes 1, 2 and 7 both in A and B are controls and have not been dialysed. Lane 1: 20 μM ADP in distilled water; Lane 2: 5 nM tianeptine in presence (A) or absence (B) of reaction buffer; Lane 7: 0.06 units GS in distilled water.

FIG. 9: Inhibition of sheep brain GS activity by tianeptine (1-5 nM) and its recovery after a 24 h dialysis against the reaction buffer supplemented with 20 μM ADP.

FIG. 10: Effects of L-glutamate (0.0016-10 mM) on the inhibition of sheep brain GS activity by 5 μM tianeptine in presence of 12.5 mM L-glutamine. The effect of tianeptine on GS activity is dependent on the concentration of L-glutamate.

FIG. 11: Effects of tianeptine (100 nM) on extracellular free amine induced by glutamate (1(1), 10(2), 100(3) and 500(4) μM) after 1 h incubation of C6 cells in serum free HBSS. The effects of tianeptine alone (0.1(1), 1(2), 10(3), 100(4) nM), NMDA alone (1(1), 10(2), 100(3) and 500(4) μM), and NMDA (1(1), 10(2), 100(3) and 500(4) M) plus tianeptine (100 nM) are shown for comparison. The effect of tianeptine on production of free amine is dependent on the dose of L-glutamate.

FIG. 12: Effects of pamidronate on Wistar rat brain GS activity in absence (FIG. 12A) or presence (FIG. 12B) of 2β-mercaptoethanol (25 mM). Activity of GS was measured as the amount of gamma-glutamyl hydroxamate produced (absorbance at 535 nm) in presence of 50 mM L-glutamine.

FIG. 13: Effects of risedronate on Wistar rat brain GS activity in absence (FIG. 13A) or presence (FIG. 13B) of 2β-mercaptoethanol (25 mM). Activity of GS was measured as the amount of gamma-glutamyl hydroxamate produced (absorbance at 535 nm) in presence of 50 mM L-glutamine.

FIG. 14: Brain GS activity (FIG. 14A) and expression (FIG. 14B) 24 h after a single intraperitoneal administration of different doses of MS or tianeptine (Tia) (0.078, 0.312, 1.25, 5, 20 mg/kg) in BALB/c mice. GS activity was measured as the amount of gamma-glutamyl hydroxamate produced (absorbance at 535 nm) in presence of 50 mM L-glutamine. GS expression was assessed by western blotting and estimated using densitometry (AU/px²).

FIG. 15: Brain GS activity (A) and expression (B) after a 7 days repeated administration of glufosinate (GF) (0.1, 3, 15 mg/kg), L-methionine sulfoximine (MS) (0.5, 15, 45 mg/kg) or tianeptine (Tia) (0.5, 15, 45 mg/kg) in BALB/c mice. All the treatments were administered once a day intraperitoneally. The animals were decapitated 1 h after the last administration on day 7. GS expression was determined using ELISA (absorbance at 405 nm).

FIG. 16: GS activity in the cortex (FIG. 16A), hippocampus (FIG. 16B) and thalamus/hypothalamus (FIG. 16C) of ICR mice 24 h after a single intraperitoneal administration of glufosinate (GF) or tianeptine (Tia). Activity of GS was measured as the amount of gamma-glutamyl hydroxamate produced (absorbance at 535 nm) in presence of 50 mM L-glutamine.

FIG. 17: Effects of tianeptine on Wistar rat brain GS activity in presence of ammonium dioxy persulfate (APS) (1.5%). Activity of GS was measured as the amount of gamma-glutamyl hydroxamate produced (absorbance at 535 nm) in presence of 50 mM L-glutamine.

FIG. 18: Effects of L-methionine sulfoximine (MS) and tianeptine (Tia) on extracellular NH₄⁺ induced by NH₄Cl 10 mM (NH₄) after 1 h in C6 cells in serum free HBSS. The amount of NH₄⁺ was determined according to Nessler's method.

FIG. 19: Effects of L-methionine sulfoximine (MS) and tianeptine (Tia) on extracellular NH4⁺ induced by glutamate 50 mM (Glu) and NH4Cl 10 mM (NH₄) after 1 h incubation of C6 cells in serum free HBSS. The amount of NH₄⁺ was determined according to Nessler's method.

FIG. 20: Effects of L-methionine sulfoximine (MS) and tianeptine (Tia) on extracellular free amine induced by glutamate 50 mM (Glu) after 1 h incubation of C6 cells in serum free HBSS. Quantitative Kaiser's method was used to determine the free amine concentration.

FIG. 21: Effects of L-methionine sulfoximine (MS) and tianeptine (Tia) on extracellular free amine induced by NH4Cl 10 mM (NH4) after 1 h incubation of C6 cells in serum free HBSS. Quantitative Kaiser's method was used to determine the free amine concentration.

FIG. 22: Effects of L-methionine sulfoximine (MS) and tianeptine (Tia) on extracellular free amine induced by glutamate 50 mM (Glu) and NH4Cl 10 mM (NH4) after 1 h incubation of C6 cells in serum free HBSS. Quantitative Kaiser's method was used to determine the free amine concentration.

FIG. 23: Effects of L-methionine sulfoximine (MS) and tianeptine (Tia) on extracellular free amine (absorbance at 570 nm) after 1 h and 24 h incubation in C6 cells in serum free HBSS. MS alone interacts with the Kaiser's method (from 6.9·10⁻⁷ M).

FIG. 24: Effects of glutamine (2 mM) on extracellular ammonium ion (A) and free amine (B) concentrations induced by NH₄Cl 10 mM (NH4) in presence L-methionine sulfoximine 5 mM (MS) or tianeptine 100 nM (Tia) after 1 h incubation in C6 cells in serum free HBSS. The blunting effects of MS and tianeptine on the NH4Cl-induced increase of ammonium were not reversed by glutamine.

FIG. 25: Effects of L-methionine sulfoximine (MS) and tianeptine (Tia) on total apoptosis (early apoptosis and necrosis) induced by 10 mM NH4Cl (NH4) after 72 h in C6 cells cultured in DMEM.

FIG. 26: Effects of L-methionine sulfoximine (MS) and tianeptine (Tia) on total apoptosis (early apoptosis and necrosis) induced by 50 mM glutamate (Glu) after 24 h in C6 cells cultured in DMEM.

FIG. 27: Effects of L-methionine sulfoximine (MS) and tianeptine (Tia) on total apoptosis (early apoptosis and necrosis) induced by 50 mM glutamate (Glu) plus NH4Cl 10 mM (NH4) after 24 h in C6 cells cultured in DMEM.

FIG. 28: Effect of a repeated administration of glufosinate (GF) (0.1, 3 and 15 mg/kg), L-methionine sulfoximine (MS) (0.5, 15 and 45 mg/kg) or tianeptine (Tia) (0.5, 15 and 45 mg/kg) compared to vehicle (PCB) on resting time (%) in an open field task (10 min) in Wistar rats administered with 35 mg/kg corticosterone. All the treatments were administered once a day during 15 days. GF, MS and Tia were administered intraperitoneally. Corticosterone was administered subcutaneously. Rats administered with both corticosterone's vehicle and treatment's vehicle were tested during the same experiment for comparison (Control).

FIG. 29: Effect of a repeated administration of glufosinate (GF) (0.1, 3 and 15 mg/kg), L-methionine sulfoximine (MS) (0.5, 15 and 45 mg/kg) or tianeptine (Tia) (0.5, 15 and 45 mg/kg) compared to vehicle (PCB) on immobility (sec) in a forced swimming test (5 min) in Wistar rats administered with 35 mg/kg corticosterone. All the treatments were administered once a day during 19 days. GF, MS and Tia were administered intraperitoneally. Corticosterone was administered subcutaneously. Rats administered with both corticosterone's vehicle and treatment's vehicle were tested during the same experiment for comparison (Control).

FIG. 30: Effect of a 21 days repeated administration of glufosinate (GF) (0.1, 3 and 15 mg/kg), L-methionine sulfoximine (MS) (0.5, 15 and 45 mg/kg) or tianeptine (Tia) (0.5, 15 and 45 mg/kg) compared to vehicle (PCB) on the decrease of surface of CA3 pyramidal neurons induced by a daily administration of 35 mg/kg corticosterone in Wistar rats. All the treatments were administered once a day during 21 days. The animals were decapitated 1 h after the last administration on day 21. GF, MS and Tia were administered intraperitoneally. Corticosterone was administered subcutaneously. Rats administered with both corticosterone's vehicle and treatment's vehicle were tested during the same experiment for comparison (Control).

FIG. 31: Effect of a repeated administration of glufosinate (GF) (0.1, 3 and 15 mg/kg), L-methionine sulfoximine (MS) (0.5, 15 and 45 mg/kg) or tianeptine (Tia) (0.5, 15 and 45 mg/kg) compared to vehicle (PCB) on whole brain GS activity (FIG. 31A) and expression (FIG. 31B) in Wistar Rats administered with 35 mg/kg corticosterone. All the treatments were administered once a day during 21 days. The animals were decapitated 1 h after the last administration on day 21. GF, MS and Tia were administered intraperitoneally. Corticosterone was administered subcutaneously. GS expression was determined using ELISA (absorbance at 405 nm). Rats administered with both corticosterone's vehicle and treatment's vehicle were tested during the same experiment for comparison (Control).

FIG. 32: Effect of a repeated administration of glufosinate (GF) (0.1, 3 and 15 mg/kg), L-methionine sulfoximine (MS) (0.5, 15 and 45 mg/kg) or tianeptine (Tia) (0.5, 15 and 45 mg/kg) compared to vehicle (PCB) on liver (FIG. 32A) and lung (FIG. 32B) GS activity in Wistar Rats administered with 35 mg/kg corticosterone. All the treatments were administered once a day during 21 days. The animals were decapitated 1 h after the last administration on day 21. GF, MS and Tia were administered intraperitoneally. Corticosterone was administered subcutaneously. Rats administered with both corticosterone's vehicle and treatment's vehicle were tested during the same experiment for comparison (Control).

FIG. 33: Effect of a repeated administration of glufosinate (GF) (0.1, 3 and 15 mg/kg), L-methionine sulfoximine (MS) (0.5, 15 and 45 mg/kg) or tianeptine (Tia) (0.5, 15 and 45 mg/kg) compared to vehicle (PCB) on brain glucocorticoid receptor (GR) expression (FIG. 33A) and adrenal weight (FIG. 33B) in Wistar rats administered with 35 mg/kg corticosterone. All the treatments were administered once a day during 21 days. The animals were decapitated 1 h after the last administration on day 21. GF, MS and Tia were administered intraperitoneally. Corticosterone was administered subcutaneously. GR expression was determined using ELISA (absorbance at 405 nm). Rats administered with both corticosterone's vehicle and treatment's vehicle were tested during the same experiment for comparison (Control).

FIG. 34: Protein diffusion in agarose gel. The interaction between NF and GS is shown in agarose gel (0.1%). Fifty μl of NF, dissolved in CH₃OH, was deposit in central well (C). Purified pig GS at dose of 3 mg/ml and 50 μl/well was applied to well 2. Well 1 and 3 are negative controls filed with 50 μl of TBS or FCS (3 mg/ml) respectively. Arrows show the precipitated NF/GS complex both in protein as detected by Commassie blue (A) and in phase contrast photography (B).

FIG. 35: Spot immuno-blotting experiment. Precipitated NF/GS complex was cut out from agarose gel in FIG. 34. The complex was heated to melt the agarose and centrifuged. Both supernatant (1) and pellet (2) were applied on a PVDF membrane. The spots in “A” was revealed with polyclonal rabbit anti-GS antibodies whereas the spots in “B” was subjected to polyclonal anti-GR antibodies.

FIG. 36: Western blot analyses on complex NF/purified pig GS with fixed NF concentration. Increasing concentrations of purified pig GS (from lane 1-8 protein amounts of 0.0023 to 3 mg/ml, respectively) were mixed with fixed concentration of NF (1.2e⁻⁵ M). Precipitated protein were separated and transferred to nitrocellulose membranes. The transferred proteins were revealed by polyclonal rabbit anti-GS antibodies. In (A) electrophoresis was performed in native conditions, in absence of 2β-mercaptoethanol. In (B) electrophoresis was performed in denaturating conditions, in presence of 2β-mercaptoethanol.

FIG. 37: Western blot analyses on complex NF/rat brain homogenate with fixed concentration of NF. Increasing protein concentrations from rat brain homogenate (from lane 1-8 protein amounts of 0.036 to 0.182 mg/ml, respectively) were mixed with fixed concentration of NF (1.2e⁻⁵ M). Precipitated protein were separated and transferred to nitrocellulose membranes. The transferred proteins were revealed by polyclonal rabbit anti-GS antibodies. In (A) electrophoresis was performed in native conditions, in absence of 2β-mercaptoethanol. In (B) electrophoresis was performed in denaturating conditions, in presence of 2β-mercaptoethanol.

FIG. 38: Western blot analyses on complex NF/purified pig GS with fixed protein concentration. Increasing NF concentrations (from lane 3-18 different concentrations of NF from 6.3e⁻¹⁹ to 4.8e⁻⁷ M, respectively) were mixed with a fixed concentration of purified pig GS (3 mg/ml). Precipitated proteins were separated and transferred to nitrocellulose membranes. The transferred proteins were revealed by polyclonal rabbit anti-GS antibodies. The electrophoresis was performed in denaturating conditions, in presence of 2β-mercaptoethanol. Lane 1 is control protein (FCS) and lane 2 is NF in absence of protein.

FIG. 39: Effects of NF on Wistar rat brain GS activity. Serial dilutions of NF (from 2.11e⁻¹⁷ to 7.87e⁻¹² M) were incubated with rat brain homogenates in absence of 2β-ME. Activity of GS was measured as the amount of gamma-glutamyl hydroxamate produced (absorbance at 535 nm) in presence of 50 mM L-glutamine.

FIG. 40: Effects of GF and NF on BALB/c mouse brain GS activity. Mice received a single i.p. injection of GF (1 mg/kg), NF (10 and 100 mg/kg) or vehicle (PCB) 24 h before being sacrificed. GS activity was assessed in cortex (A), hippocampus (B) and thalamus/hypothalamus (C) respectively.

FIG. 41: Effects of NF on Wistar rat brain GS activity. Rats received a single i.p. injection of NF (1 and 10 mg/kg) or vehicle (PCB) 1 h before being sacrificed. GS activity was assessed in cortex (A), hippocampus (B) and thalamus/hypothalamus (C) respectively.

FIG. 42: Effects of chronic administration of GF and Tianeptine (Tia) on Wistar rat brain cortex GS activity. Rats received a daily i.p. injection of GF (0.1 mg/kg), Tia (10 mg/kg) or Vehicle (PCB) together with methylprednisolone (5 mg/kg) for 7 weeks. GS activity was assessed in cortex (A), hippocampus (B) and thalamus/hypothalamus (C). Rats were sacrificed 1 h after the last administrations.

FIG. 43: Effects of chronic administration of GF and Tianeptine (Tia) on Wistar rat brain cortex GS activity. Rats received a daily i.p. injection of GF (0.1 mg/kg), Tia (10 mg/kg) or Vehicle (PCB) together with methylprednisolone (5 mg/kg) for 7 weeks. GS activity was assessed in cortex (A), hippocampus (B) and thalamus/hypothalamus (C). Rats were sacrificed 24 h after the last administrations.

FIG. 44: Effects of chronic administration of GF and NF on Wistar rat adrenal glands' weight. Rats received a daily i.p. injection of GF (0.1, 0.01 and 0.001 mg/kg), NF (1 and 10 mg/kg) or Vehicle (PCB) together with methylprednisolone (5 mg/kg) for 7 weeks.

FIG. 45: Effects of GF and Tianeptine (Tia) on PTZ induced seizure in Wistar rats. Rats received an i.p. injection of GF (0.5, 5 and 25 mg/kg), Tia (1, 10 and 50 mg/kg) or Vehicle (PCB) 1 h before PTZ administration (50 mg/kg i.p.). Seizure was scored according to a standard scoring method over the 20 min following PTZ administration. Rats were sacrificed 1 h after PTZ administration.

FIG. 46: Effects of GF and Tianeptine (Tia) on brain GS activity in Wistar rats administered with PTZ. Rats received an i.p. injection of GF (0.5, 5 and 25 mg/kg), Tia (1, 10 and 50 mg/kg) or Vehicle (PCB) 1 h before PTZ administration (50 mg/kg i.p.). GS activity was assessed in cortex (A), hippocampus (B) and thalamus/hypothalamus (C). Rats were sacrificed 1 h after PTZ administration.

FIG. 47: Effects of Li₂CO₃ on Wistar rat brain GS activity. Serial dilutions of Li₂CO₃ (from 1e⁻² to 2.11e⁻²² M) were incubated with rat brain homogenates in absence of 2β-ME. Activity of GS was measured as the amount of gamma-glutamyl hydroxamate produced (absorbance at 535 nm) in presence of 50 mM L-glutamine.

FIG. 48: Effects of Sr²⁺ on Wistar rat brain GS activity. Serial dilutions of Sr²⁺ (from 1e⁻¹⁰ to 6.27e⁻¹⁷ M) were incubated with rat brain homogenates in absence of 2β-ME. Activity of GS was measured as the amount of gamma-glutamyl hydroxamate produced (absorbance at 535 nm) in presence of 50 mM L-glutamine.

### DETAILED DESCRIPTION OF THE INVENTION

The Inventors have surprisingly discovered that tianeptine, an atypical antidepressant with neuroprotective properties, is a specific ligand of the enzyme glutamine synthetase (GS; EC 6.3.1.2). To investigate the effects of tianeptine on GS activity, an usual method used to investigate GS activity (Meister 1985) had to be amended. In fact, tianeptine lost its activity in presence of the reducing agent 2β-mercaptoethanol (β-ME) due to its interaction with SO₂. Thus, the effects of tianeptine on GS activity were first tested in absence of β-ME i.e. on the whole GS enzyme instead on GS subunits (see below); β-ME was replaced by dithiothreitol (DTT) in the classical experiments investigating the effects on GS subunits (monomers) i.e. in presence of a reducing agent. Transferase activity was tested rather synthetase activity because of its high sensitivity and lower background activity due to the interaction of glutamate with ferric (II) solution. Surprisingly, depending on the concentration/dose and experimental or (patho)physiological condition, tianeptine in non-reducing conditions could activate or inhibit GS activity in animals and bacteria like observed previously, only in plants, with GF and MS, and derivatives thereof (Estigneeva et al., 2003). These regulatory effects of tianeptine on GS function can likely underlie its already proposed clinical applications (see above) contrary to its unclear effects on 5-HT uptake that are usually proposed. They can also be reconciled with the numerous other previous speculations about its action mechanism and pharmacological profile, including its observed hormetic properties (Ceyhan et al., 2005; McEwen et al., 1993 and 2004; Plaisant et al., 2003; Reagan et al., 2004). This includes the emerging pharmacological profile of tianeptine which suggests that tianeptine may serve to balance glutamatergic neurotransmission i.e. combat pathological stimulation only, whilst maintaining the level of physiological activation (U.S. Pat. No. 6,599,896 B1; McEwen et al., 2004; Plaisant et al., 2003; Reagan et al., 2004). So, the Inventors have found a solution for one of the classical paradoxes against the monoamine theory of depression: “If blocking serotonin reuptake is important in the treatment of depression, then why is tianeptine, which enhances serotonin reuptake, an effective antidepressant? ”, a fair explanation for the neuroprotective effects of tianeptine, a plausible mechanism for its effects on asthma which are usually considered “provocative and worth exploring” when proposed to be related to effects on serotonin, etc.

In vertebrate animals including humans, as well as in other (all) organisms with eukaryotic cells such as algae, invertebrate animals (arthropods, protozoa, shellfish, worms, . . . ), fungi (yeasts, molds, . . . ) or plants, but also prokaryotes e.g. algae or bacteria, L-glutamine serves as the most important carrier of nitrogen and one of the major build stones in protein synthesis. Acting as a respiratory fuel, it provides also energy. These explain why glutamine homeostasis is carefully balanced. The equilibrium is due to a precise control of glutamine consumption and production, both within cells, tissues, organs, . . . . Transport of glutamine into and out of cells plays an important role in the control of this process. However, ultimately, the balance between glutamine anabolism and catabolism, which relies largely on the activity of two enzymes, glutamine synthetase and glutaminase, is crucial.

Glutamine catabolism is initiated through its deamidation to form glutamate. This can proceed via a number of cytosolic transamidase enzymes that use the γ-amido nitrogen of glutamine in a variety of metabolic syntheses (Zalkin and Smith, 1998). The rate at which these reactions utilize glutamine depends upon the metabolic demand for the reaction products and is, therefore, not appropriate for control of glutamine homeostasis. On the contrary, the mitochondrial enzyme glutaminase catalyzes the hydrolysis of the γ-amido group of glutamine to form glutamate and ammonia. Ammonia can be used to form carbamoyl phosphate or can diffuse from the mitochondria and the cell itself. Glutamate can be further deaminated by glutamate dehydrogenase (GDH) to form α-ketoglutarate and thus enter the citric acid cycle. Thus, the increase of glutamine catabolism through glutaminase, under physiological conditions, does not lead to production of excessive amounts of specific metabolites.

In contrast to the many enzymes that utilize glutamine as a substrate, only one enzyme, GS, is responsible for de novo synthesis of glutamine. This ligase catalyses, under normal conditions, the formation of glutamine from glutamate and ammonia.

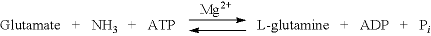

The glutamine synthetases of animal origin resemble each other with respect to amino acid composition, subunit structure and molecular weight (Meister 1985). Most of animal cells make a version with eight subunits, each of which has an active site for production of glutamine. Briefly, when performing its reaction, the active site of GS binds to glutamate and ammonia, and to an ATP molecule that powers the reaction. In vitro, GS converts around 90% of glutamate to glutamine when it is incubated with equal concentrations of L-glutamate, ammonium ion and ATP, in presence of Mg²⁺, at pH 7.0 and 37° C. GS activity depends on the concentrations of its substrates and products, of divalent cations like Mg²⁺ and Mn²⁺, of ADP/ATP, . . . . Its mechanism has been described as ternary, either order or random ter, i.e. the interaction of its substrates at different concentrations and cellular conditions can lead to production of various metabolites. This is schematized below:

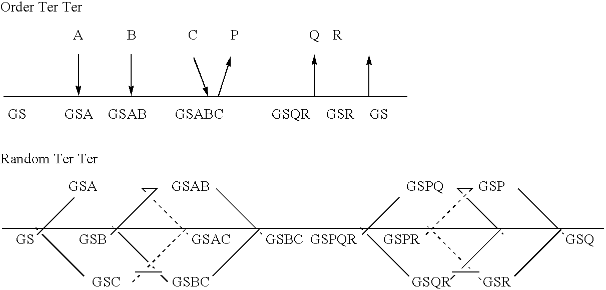

where substrates are A, B, C, and products are P, Q, R.

Partial reactions catalyzed by GS include the γ-glutamyl transfer reaction, the arsenolysis of glutamine, the synthesis of γ-glutamyl hydroxamates and γ-carboxyl-activated glutamate derivatives, . . . (Meister, 1985). All these reactions can theoretically take place e.g. the latter is activated in the absence of ammonia, where GS catalyses the conversion of glutamate to 5-oxoproline. Glutamate attaches to the enzyme only when both ATP and a divalent cation (usually Mg⁺) are present. So the reactions catalyzed by GS are clearly not straightforward. The schema below illustrates the mechanism of action of GS with reversal and partial reactions as well as the products of the metabolization of L-methionine-S-sulfoximine (MS; L-MSO) (Meister, 1985).

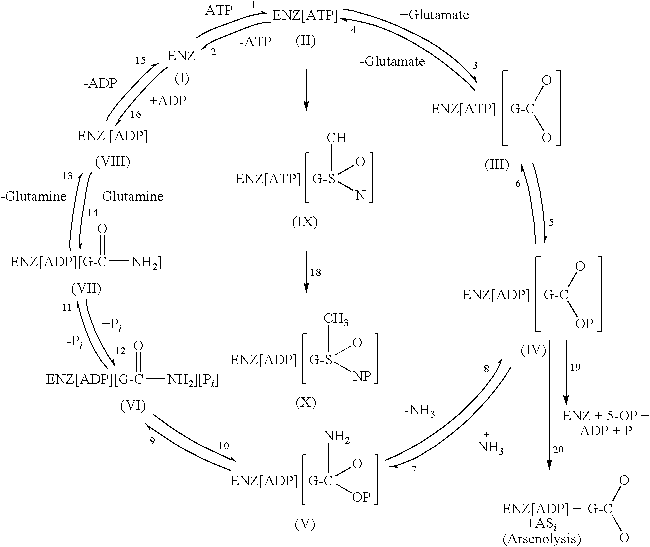

Scheme of the mechanism of action of glutamine synthetase: G, ⁻OOCCH(NH₃⁺)(CH₂)₂. Glutamine synthesis: 1, 3, 5, 7, 9, 11, 13, 15. Reversal of synthesis: 16, 14, 12, 10, 8, 6, 4, 2. Partial reactions: γ-Glutamyl transfer: 16, 14, 12, 10, 8, 7 (+NH₂OH), 9, 11, 13. Arsenolysis of glutamine: 16, 14, 12 (+Asᵢ), 10, 8, 20. Phosphorylation of L-methionine-S-sulfoximine: 1, 17, 18. Cyclization: formation of 5-oxoproline (5-OP): 1, 3, 5, 19. Phosphorylation of glutamate analog (cycloglutamate): 1, 3, 5. Acyl phosphate (β-glutamyl ˜P, carbamyl ˜P, acetyl ˜P)+ADP [symbol] ATP: 6, 4, 2 (adapted from Meister, 1985).

GS active sites can also bind to other compounds, which may partially block the action of the enzyme e.g. to amino acids like alanine, glycine, histidine, serine, tryptophane, . . . but also adenosine monophosphate, carbamyl phosphate, cytidine triphosphate, glucosamine 6-phosphate, . . . . As the concentrations of these “effectors” rise, more and more GS sites are occupied, eventually shutting down the whole enzyme. On the contrary, certain amino acids or xenobiotic compounds such as glufosinate (GF; phosphinothrycin (PPT)) and L-methioninesulfoximine (MS; L-MSO) and derivatives thereof, which are usually considered as GS inhibitors, at low concentrations/doses in plants were observed by a Russian group to behave as GS activators (Pushkin et al., 1974 and 1975; Evstigneeva et al., 2003). So, a large body of evidence indicates that GS effectors can cause subunit but also whole enzyme conformational changes, which result in GS activation and/or inhibition (Eisenberg et al., 2000).

Expression of GS in mammalian is regulated by transcriptional and post-transcriptional mechanisms. In particular increased transcription occurs due to glucocorticoid action and regulation of protein stability is linked to glutamine concentration. Glucocorticoid administration as well as various stresses have been observed to elevate GS mRNA level through glucocorticoid receptor-dependent mechanisms (Abcouwer et al., 1995 and 1996; Ardawi et al., 1990; Ardawi and Majzoub, 1991; Chandrasekhar et al., 1999; Lukaszewicz et al., 1997). However, not always there is a correlation between the expression of GS at both mRNA and protein levels. The ability of glutamine to promote GS protein turnover via a post-transcriptional mechanism, in which the rate of GS protein degradation is altered, is one of the bases of this irregularity. Through such mechanisms, glutamine production is adapted to the glutamine demand in a tissue-specific fashion (Arad et al., 1976; Arad and Kulka, 1978; Milman et al., 1975; Feng et al., 1990; Lacoste et al., 1982; Patel et al., 1986). In first approach, the specific activity of animal GS seems to be less affected by post-translational modifications contrary to bacterial GS. In fact, in animals, GS nitrification is very likely, but no one has found adenylation nor phosphorylation as observed in bacteria (Eisenberg et al., 2000; Kosenko et al., 2003).

Xenobiotics have been demonstrated to influence GS expression too. For instance MS (100 mg/kg) and dexamethasone (0.5 mg/kg) were reported individually to augment rat lung GS protein levels 4-fold and 2-fold, respectively, and their combination 12-fold (Labow et al., 1998).

So depending on cell type, tissue, organ, species, (patho)physiological condition or treatment, GS mRNA, GS protein level and GS activity are obviously variable. Because both GS substrates i.e. ammonia and L-glutamate are usually not limiting, the rate of L-glutamine formation is usually highly dependent upon the activity of GS, thus on cofactors and effectors concentrations.

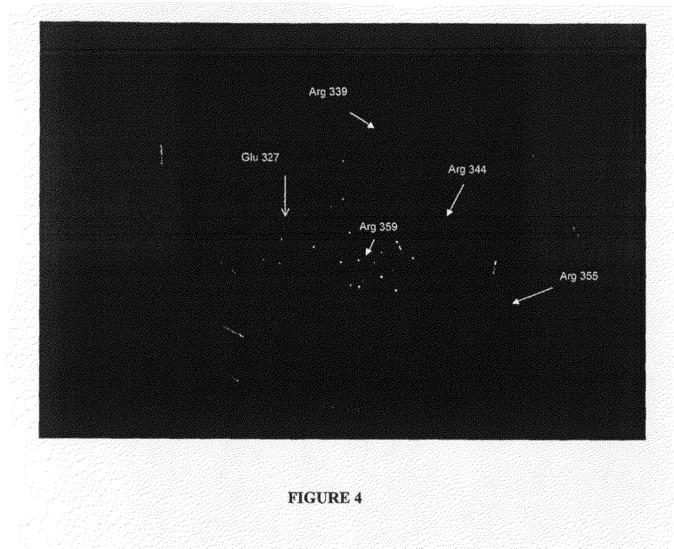

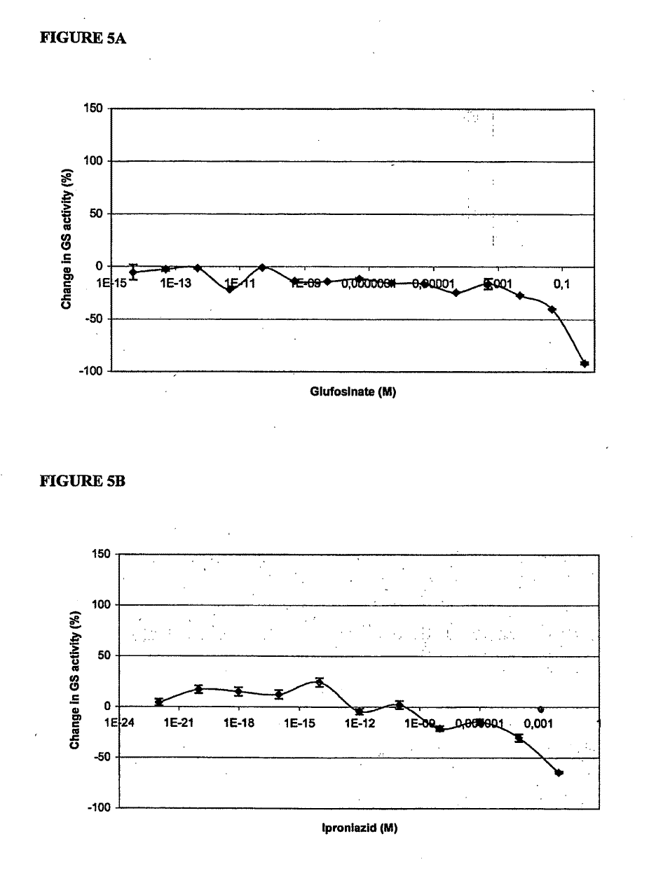

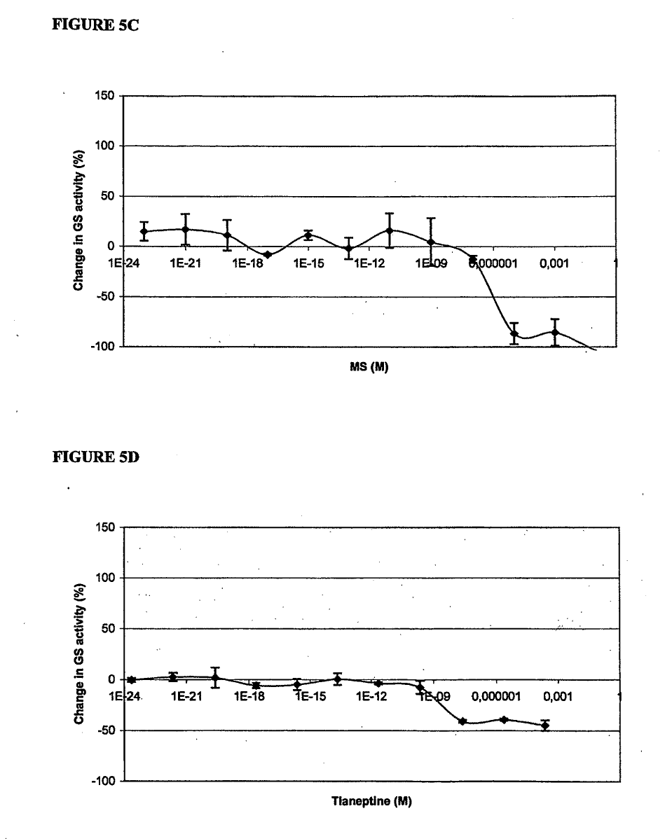

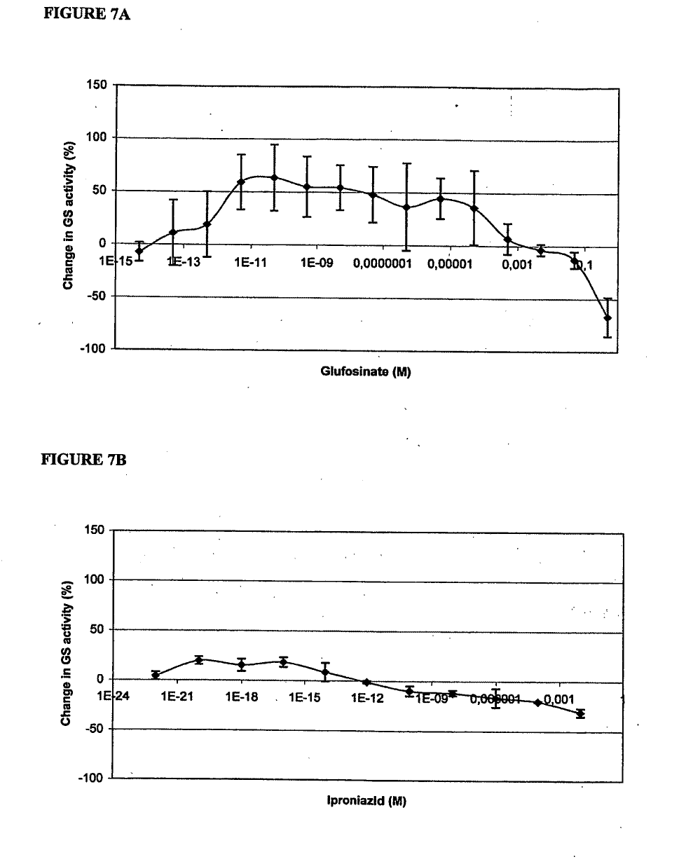

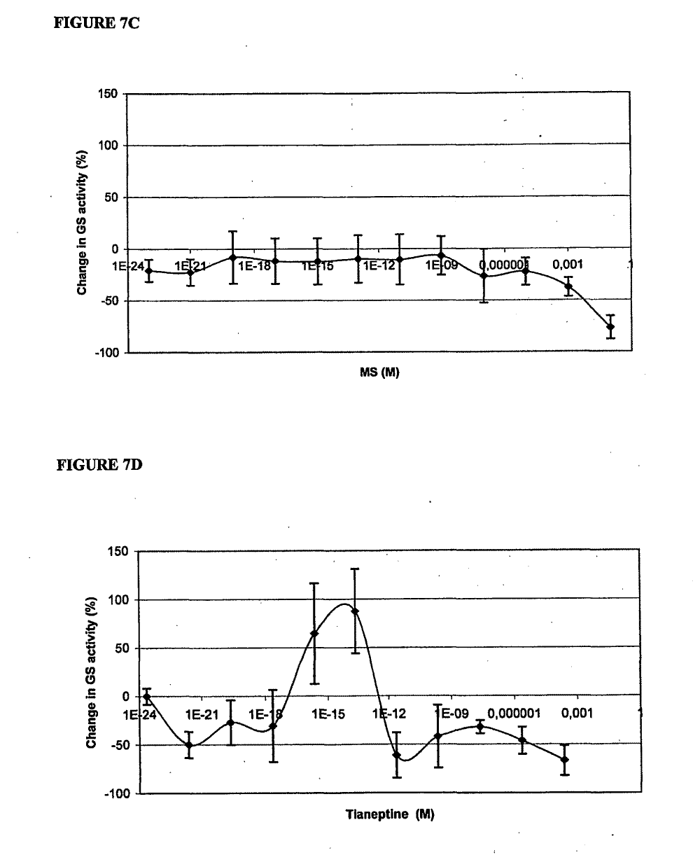

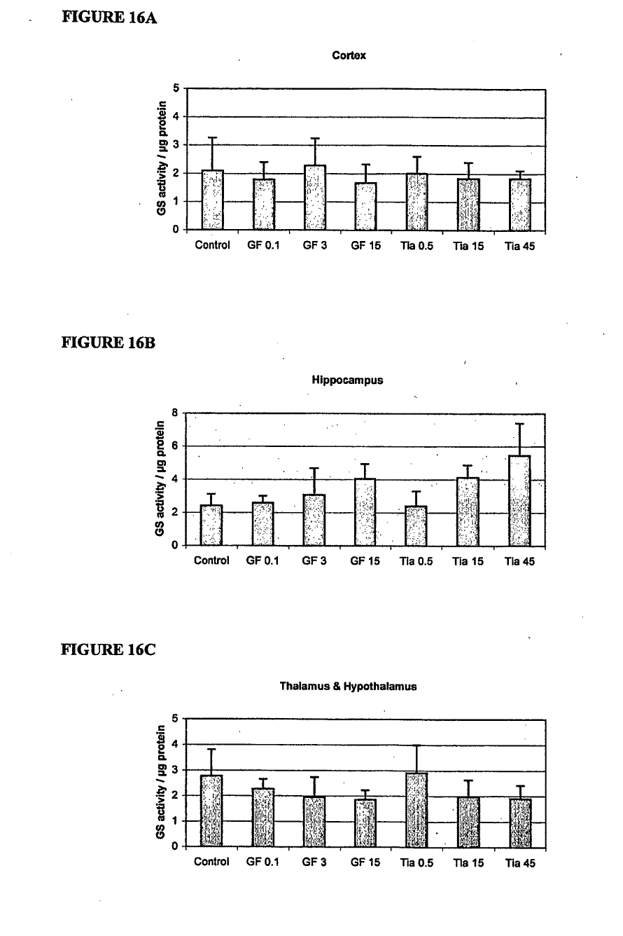

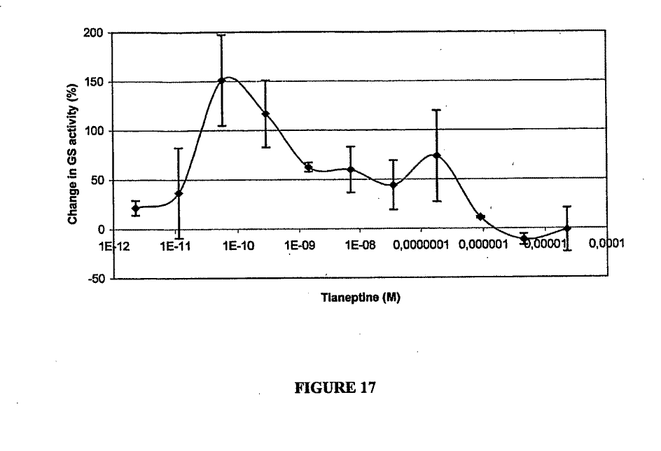

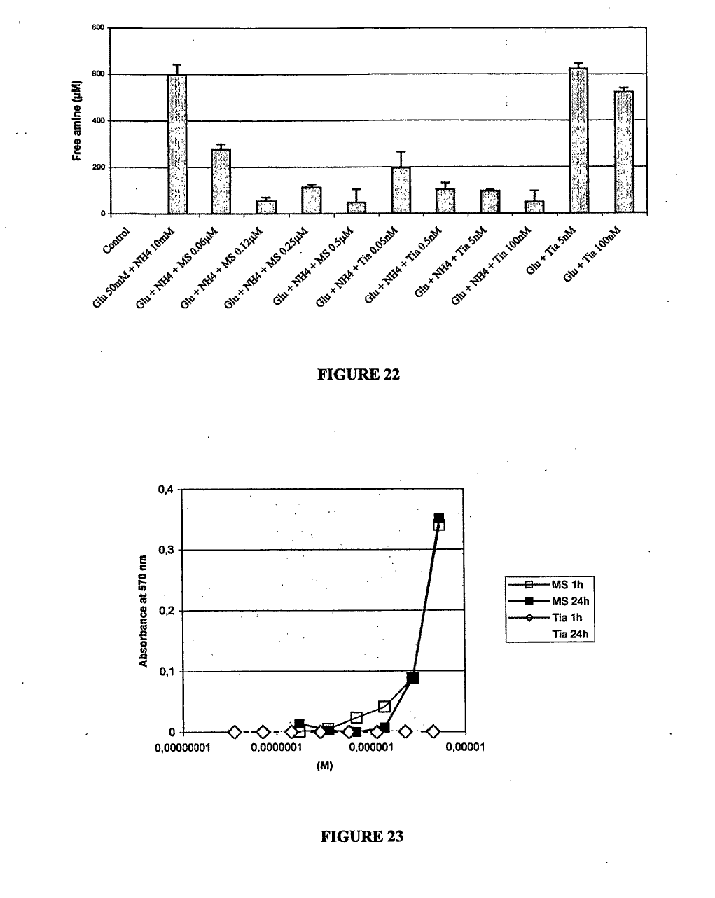

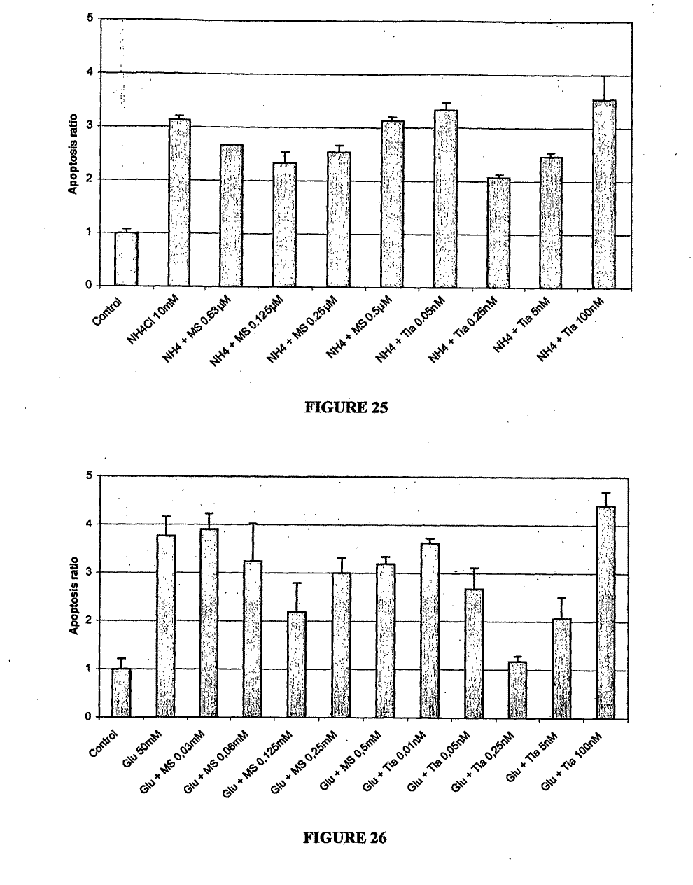

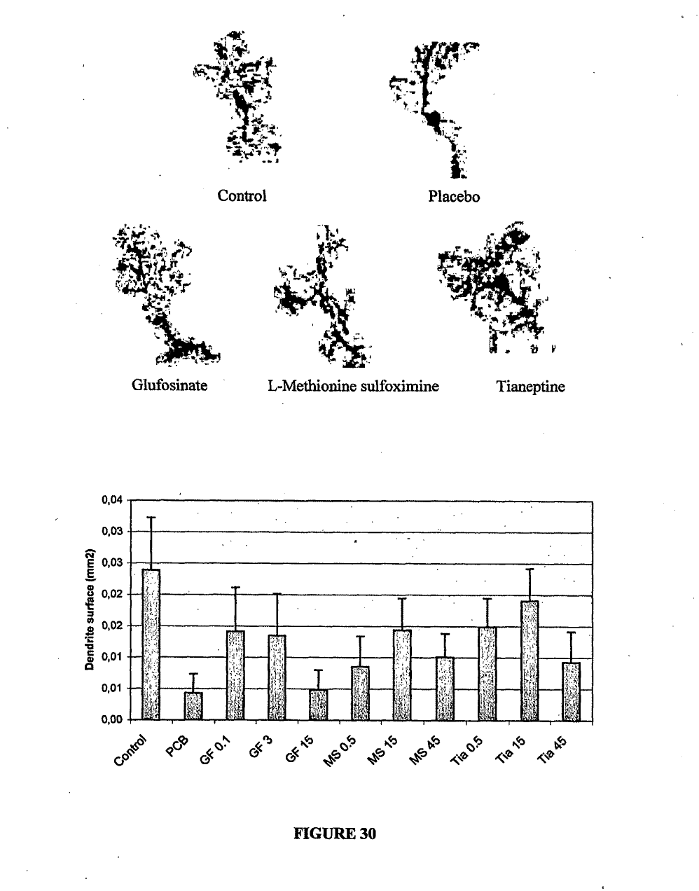

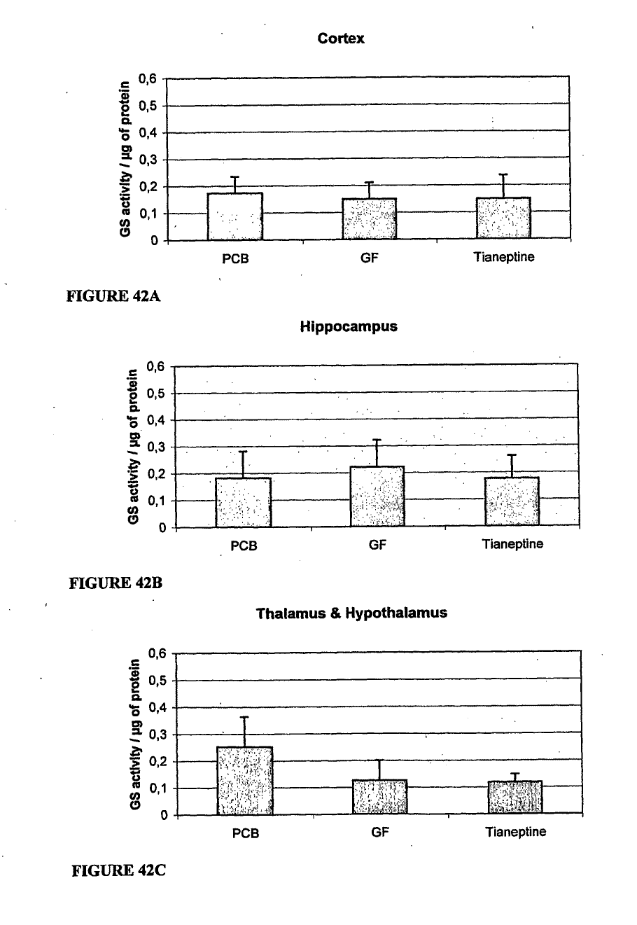

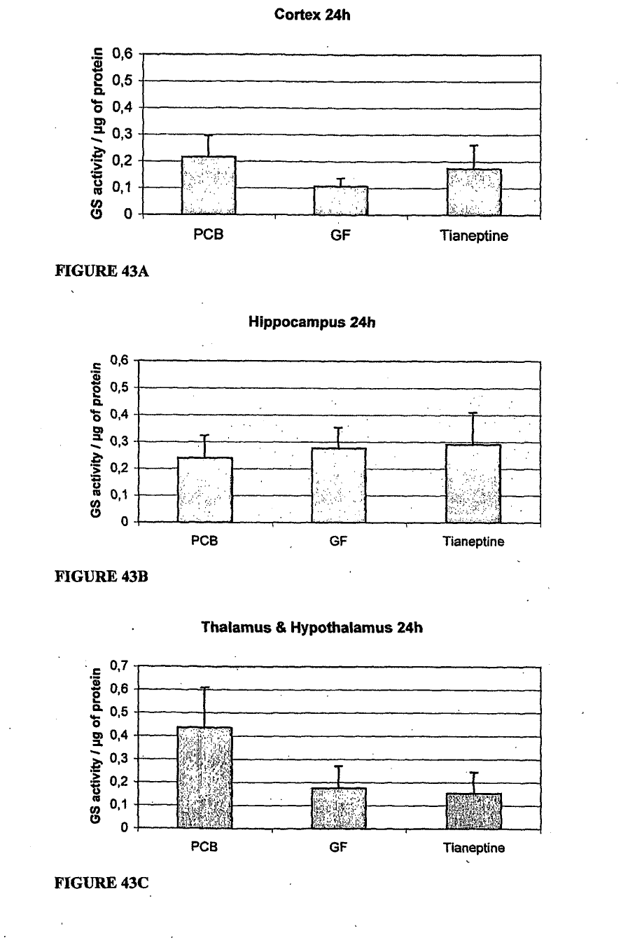

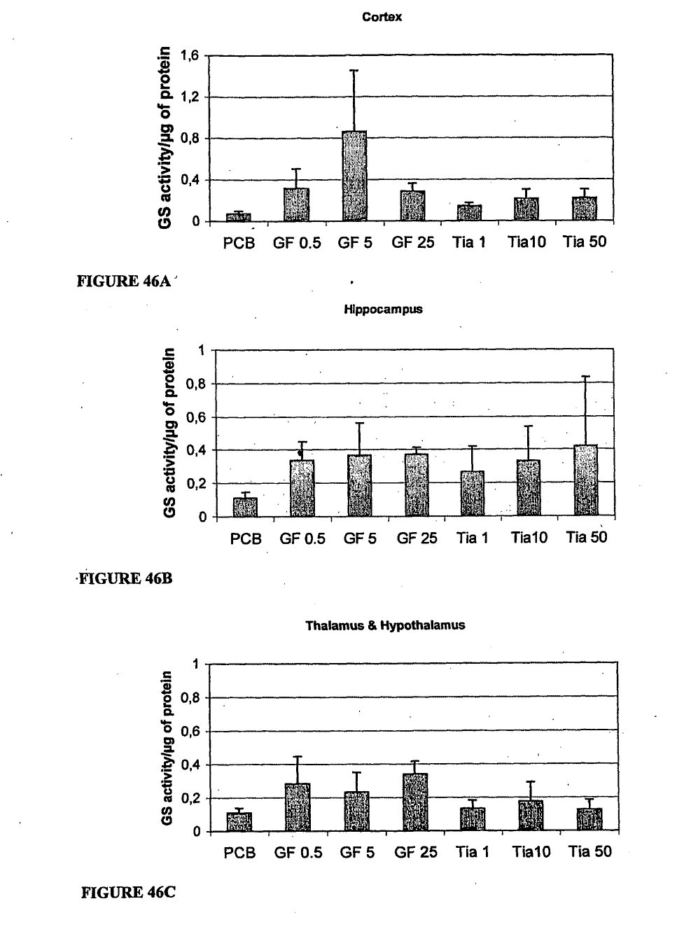

The Inventors have discovered that tianeptine is a selective ligand of the GS subunit catalytic site (FIG. 4) and when it binds consequently the catalysing activity of the subunit is impeded (FIGS. 4 and 5D). However, while “high concentrations” of tianeptine, i.e. concentrations which occupy (more or less) all the subunits of the enzyme, result “obligatorily” in inhibition of GS activity (FIGS. 5D, 6 and 7D), “lower concentrations”, i.e. concentrations where only one (or few) GS subunit(s), are occupied can result in an increase or a decrease of GS activity, or can be pharmacologically tolerated, depending on the experimental and/or (patho)physiological condition (FIGS. 7D, 14A and 15A). Considering that tianeptine-induced GS activation can be observed in vitro on purified GS whole enzyme (FIG. 7D; GS activity assessed in absence of reducing agent), but not on isolated subunits (FIG. 5D; GS activity assessed in presence of reducing agent), it may be speculated that tianeptine, in (impeding) binding to one (or few) GS subunit(s) cause conformational changes that result in the activation of the other non occupied subunits; since tianeptine seems to bind GS only in its closed form (FIG. 4), its regulatory activity might depend on the number of sites already occupied by the natural substrates, cofactors and effectors. Tianeptine can also protect GS from oxidative stress (FIG. 17) and raise GS protein expression in vivo (FIGS. 14B, 15B and 31B). So, while “high concentrations” of tianeptine result “obligatorily” in an inhibition of GS activity—these concentrations are toxic in eukaryotes and prokaryotes, and might serve as cytolytics e.g. to combat neoplasms in animals, or growth in algae, fungi, plants or bacteria—, “low concentrations” of tianeptine have no effect or result in an increase or a decrease of GS activity, depending on the experimental and/or (patho)physiological condition. So tianeptine can be proposed to regulate all types of functions which involve or can be influenced by GS activity, directly or indirectly, therefore be of major therapeutic interest in preventing and/or treating conditions and disorders related to (relative or absolute excess of) ammonia, (relative or absolute deficiency or excess of) glutamate, and/or (absolute or relative deficiency or excess of) glutamine. In fact, tianeptine as well as GF and/or MS at “low concentrations/doses”) were observed, in vitro, to prevent cellular apoptosis in conditions of elevated ammonia and/or elevated glutamate concentrations (FIGS. 25-27) and, in vivo, to counteract hippocampal structural changes associated with chronic glucorticoid administration (FIG. 30) and pentylenetetrazole-induced seizures (FIG. 45; Ceyhan et al., 2005) without safety and tolerability issues, and these effects could be explained by their regulatory influence on GS (FIGS. 16, 18-22, 31, 42-44 and 46). Tianeptine and GF, at the same “low doses” in Wistar rats, could be used to increase and decrease GS activity in the hippocampus in the chronic glucorticoid administration (FIG. 42-43) and pentylenetetrazole-induced seizures (FIG. 46; Ceyhan et al., 2005) models, respectively; these latter models are demanding an increase of GS activity/response and comprising an altered GS activity, respectively. Thus, at the “low doses” used no strict, systematic GS inhibition was observed.

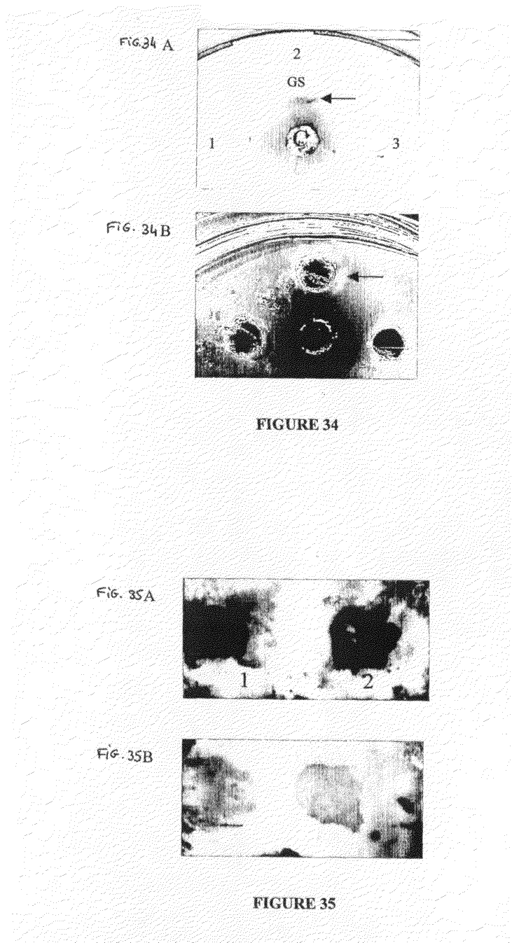

In addition, the Inventors identified the target of naftazone (NF). NF is a synthetic molecule metabolized in humans by reduction and glucuronidation which leads to the formation of 11-hydroxy, 2-naphtyl)semicarbazide-1 b-Oglucopyranosiduronic acid (Fig. XB) (Herber et al., 1995). It used for the treatment of venous insufficiency and varices (Klein-Soyer et al., 1995). NF has been observed to have protective effects against lysosomial disruption and lipid peroxydation in different models of inflammation (Agha and Gad, 1995). It has been shown also to inhibit ex vivo and in vitro platelet aggregation induced by ADP and thrombin (Durand et al., 1996). Finally, it was recently observed to decrease glutamate levels in the cerebro spinal fluid (Mattei et al., 1999). Naftazone and its glucuronide derivative reduced respectively spontaneous and evoked glutamate release. So, naftazone and related beta-naphtoquinone have been claimed to be used for the prevention and/or treatment of glutamate cytotoxicity (US 20020115617 A1). The Inventors have demonstrated for the first time that naftzone is a regulatory ligand of the GS subunit catalytic site (FIG. 34-38) and when it binds consequently the catalysing activity and 4D enzyme structure are modified (FIGS. 34-41; NF induces multimeric formation).

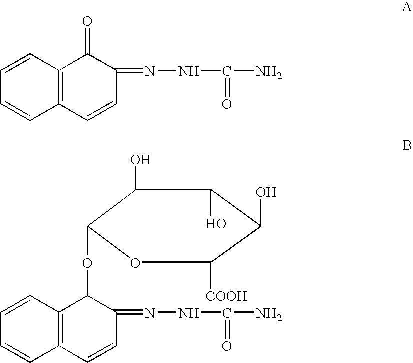

Chemical structure of naftazone (1-oxo, 2 naphtyl semicarbazone) (A) and its metabolite 1-(1-hydroxy, 2-naphtyl) semicarbazide1 b-O-glucopyranosiduronic acid (B).

Furthermore, resulting from the use of tests on the native GS enzyme i.e. in absence of reducing agent (due to their former experience/discovery with tianeptine), the Inventors have discovered that GS ligands which are usually considered as GS inhibitors in animals like GF and MS, as well as hydrazines, bisphosphonates (by extention Mg²⁺ analogs like Sr²⁺), can affect GS activity more or less as tianeptine does i.e. exhibit regulatory properties on native GS activity.

Certain hydrazines were already known to bind GS (Rueppel et al., 1972). The Inventors have demonstrated for the first time that it is also the case for the well established antituberculous agent isoniazid and its derivative the typical monoamine oxidase inhibitor (MAOI) antidepressant iproniazid. Certain bisphosphonates like [(5-chloro-pyridin-2-ylamino)-phosphono-methyl]-phosphinic acid were already known to bind GS (likely on Mg²⁺ binding site) and to exhibit an herbicidal activity. The Inventors have shown for the first time that bisphosphonates which are usually used for the treatment of osteoporosis and osteolytic disease like pamidronate and risedronate bind GS as well and behave as regulatory GS ligands. Strontium (Sr) is a member of the alkaline earth elements with an intermediate position between calcium and barium. It is present at small physiological amounts in the organism, in particular in bone, and can be considered as a Mg analog. As ranelate salt, it has just obtained the authorization of marketing in Europe in the indication of treatment of post-menopausal osteoporosis. The Inventors have shown for the first time that Sr can behave as a regulatory GS ligand.

All these “other GS ligands” present a GS regulating activity in animals when they are used at concentrations/doses lower as compared to the concentrations/doses which inhibit GS activity systematically (FIGS. 7, 12-17, 31-32; GS activity assessed in absence of reducing agent): in outline i) at “low concentrations/doses” where only one (or few) subunit(s) of the enzyme are occupied, they result in an increase or a decrease of GS activity, or can be pharmacologically tolerated depending on the experimental or (patho)physiological condition, in particular on the concentrations of substrates, cofactors and effectors, and treatment modalities, while ii) at “high concentrations/doses” where (more or less) all the subunits of the enzyme are occupied, they behave strictly as GS inhibitors. These effects on GS function may underlie, at least in part, the already proposed clinical applications of GF, MS, NF, hydrazines (see Table 6) and bisphosphonates (by extention Mg²⁺ analogs like Sr²⁺; see Table 7). So there is compelling evidence for the use of GS regulating ligands like GF and MS but also of NF, hydrazines and bisphosphonates (by extention Mg²⁺ analogs like Sr²⁺) and isomers thereof, pro-drugs thereof, metabolites thereof and structural analogs thereof, either alone or combined, for treating conditions and disorders demonstrated or proposed to be sensitive to tianeptine, even though their selectivity for GS, their interaction with GS or their pharmacokinetics differ to some extent; for instance MS is a non-selective, irreversible GS ligand (Eisenberg et al., 2000; Rao and Meister, 1972; Meister and Tate, 1976) while GF is a selective, irreversible GS ligand, and tianeptine is a selective, reversible GS ligand (FIGS. 4, 8-9). There are convergent arguments for the use of GS ligands like GF and MS as well as hydrazines and bisphosphonates, isomers thereof, pro-drugs thereof, metabolites thereof and structural analogs thereof, in obtaining regulators of GS activity intended for regulating all types of functions which involve GS activity or can be influenced by GS activity, directly or indirectly, and/or preventing and/or treating all types of conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, either isolated or combined. Conversely there are also reasonable arguments to propose the use of tianeptine or NF, isomers thereof, pro-drugs thereof metabolites thereof and structural analogs thereof, for preventing and/or treating all conditions and disorders demonstrated or proposed to be sensitive to GS ligands like GF MS, hydrazines and bisphosphonates, or isomers thereof, pro-drugs thereof, metabolites thereof or structural analogs thereof.

Preferably, “GS regulating ligand(s)” encompasses compounds able to bind GS (subunit) in its catalytic site, and more particularly in the glutamate binding site. Preferably, these compounds are able to compete with a compound selected from the group consisting of glutamate, GF, MS, and tianeptine for the catalytic site of GS. In a preferred embodiment, without to be limited thereto, this compound present an inhibition constant (Ki) equal or less than 10⁻⁴ M.

Preferred examples of GS ligand(s) are selected from the group consisting of tianeptine, GF, MS, NF, hydrazines, bisphosphonates (by extention Mg²⁺ analogs like Sr²⁺) and glutamate analogs, and isomers thereof, pro-drugs thereof, metabolites thereof and structural analogs thereof (Tables 8-13, 15). More preferably, GS ligand(s) are selected from the group consisting of tianeptine, GF and MS, and isomers thereof, pro-drugs thereof, metabolites thereof and structural analogs thereof (Tables 8-10).

Examples of tianeptine isomers, pro-drugs, metabolites and structural analogs include, without to be limited thereto, the compounds disclosed in Table 8.

Examples of NF metabolites and structural analogs include, without to be limited thereto, the compounds disclosed in Table 15.

Examples of GF isomers, pro-drugs, metabolites and (structural) analogs include, without to be limited thereto, the compounds disclosed in Tables 9 and 13, preferably in Table 9. Preferably, GF isomers, pro-drugs, metabolites and structural analogs are selected from the group consisting of glufosinate, gamma-hydroxy phosphinothricin, gamma methyl phosphinothricin, gamma-acetoxy phosphinothricin, alpha-methyl phosphinothricin, alpha-ethyl phosphinothricin, cyclohexane phosphinothricin, cyclopentane phosphinothricin, tetrahydrofuran phosphinothricin, S-phosphonomethyl homocysteine, s-phosphonomethyl homocysteine sulfoxide, s-phosphonomethyl homocystein sulfone, 4-(phosphonoacetyl)-L-alpha-aminobutyrate, threo-4-hydroxy-D-glutamic acid, erythron-4-fluoro-D,L-glutamic acid, 2-amino-4-[(phosphonomethyl)hydroxyphosphinyl]butanoic acid, 2-amino-4-phosphono butanoic acid, 2-amino-2-methyl-4-phosphono butanoic acid, 4-amino-4-phosphono butanoic acid, 4-amino-4-(hydroxymethylphosphinyl)butanoic acid, 4-amino-4-(hydroxymethylphosphinyl)-4-methyl butanoic acid, 4-amino-4-phosphono butanamide, 2-amoido-4-phosphono butanoic acid, 2-methyoxycarbonyl-4-phosphono butanoic acid methyl 4-amino-4-phosphono butanate and 2-amido-4-phosphono butanoic acid, and salts thereof and isomers thereof.

Examples of MS isomers, pro-drugs, metabolites and (structural) analogs include, without to be limited thereto, the compounds disclosed in Tables 10 and 13, preferably in Table 10.

Preferably, “hydrazines” encompasses compounds able to bind GS in its catalytic site, and more particularly in the ammonium binding site. Preferably, this compound is able to compete with a compound selected from the group consisting of iproniazid and isoniazid for the catalytic site of GS. Preferred examples of hydrazines binding to GS are selected from the group of iproniazid and isoniazid, and isomers thereof, pro-drugs thereof, metabolites thereof and structural analogs thereof (Table 11). Preferably, the hydrazine is selected from the group which comprises isoniazid, iproniazid, pyridoxal isonicotinoyl hydrazone, pyridoxal benzoyl hydrazone, salicylaldehyde isonicotinoyl hydrazone, salicyaldehyde benzoyl hydrazone, 2-hydroxy-1-naphthaldehyde isonicotinoyl hydrazone, 2-hydroxy-1-naphthaldehyde benzoyl hydrazone, isonicotinic acid N′-isopropyl-hydrazide, 4-chloro-N(2-morpholinoethyl)benzamide, 5-methyl-isoxazole-3-carboxylic acid N′-(3-methyl-benzyl)-hydrazide, phenethyl-hydrazine (phenelzine), DL-Serine 2-(2,3,4-trihydroxybenzyl)hydrazide (benserazide), butylmalonic acidmono-(1,2-diphenylhydrazide) (bumadizone), 5-(3,3-dimethyl-1-triazenyl)-1H-imidazole-4-carboxamide (dacarbazine), 1,4-Dihydrazino-5-azaphthalazine (dihydralazine), 1-hydrazinophthalazine (hydralazine), {1-[2-(4-Chloro-phenoxy)-ethoxy]-ethyl}-hydrazine (iproclozid), 6-Methyl-[1,2]oxazinane-3-carboxylic acid N′-cyclohexyl-hydrazide (isocarboxazid), pyridine-4-carboxylic 2-[2-(benzylcarbamoyl)ethyl]hydrazide (nialamide), 5-nitro-2-furaldehyde p-hydroxybenzoylhydrazone (nifuroxazide), cyclohexa-2,4-dienecarboxylic acid hydrazide (phenicarbazide), N-(3,4-dihydro-phthalazin-6-yl)-N′-(1,2-dihydro-pyridin-4-yl)-hydrazine (picodralazine) and N-isopropyl[(methyl2hydrazino)methyl]-p-toluamide (procarbazine), and salts thereof and isomers thereof.

Preferably, “bisphosphonates” encompasses compounds able to bind GS in its catalytic site, and more particularly in the Mn²⁺ binding sites. Preferably, this compound is able to compete with a compound selected from the group consisting of Mn²⁺, Mg²⁺, Sr²⁺, pamidronate, risedronate, and [(5-Chloro-pyridin-2-ylamino)-phosphono-methyl]-phosphinic acid for the catalytic site of GS. Preferred examples of bisphosphonates binding to GS are selected from the group consisting of pamidronate, risedronate and [(5-Chloro-pyridin-2-ylamino)-phosphono-methyl]-phosphinic acid, and isomers thereof, pro-drugs thereof, metabolites thereof and structural analogs thereof (Table 12). Preferably, bisphosphonates are selected from the group consisting of 1-phosphono-2-pyridin-2-ylamino)-ethyl]-phosphonic acid, 6-(2-Amino-ethyl)-pyridin-3-Ol-phosphonic acid, [3,4-Dichloro-phenyl)-phosphono-methyl]-phosphonic acid, 2-amino-4-[(phosphonomethyl)hydroxyphosphinyl]butanoic acid, [(5-Chloro-pyridin-2-ylamino)-phosphono-methyl]-phosphonic acid, alendronate, clodronate, etidronate, ibandronate, pamidronate, risedronate and zoledronate, and salts thereof and isomers thereof.

Examples of glutamate analogs include, without to be limited thereto, the compounds disclosed in Table 13.

“Prodrug” of a compound refers to a chemical entity which, after administration to a subject, is converted in the compound and which displays similar therapeutic activity and/or biological effect to the compound.

“Metabolite” of a compound refers to a chemical entity which is obtained following the administration of the compound to a subject and which displays similar therapeutic activity and/or biological effect to the compound.

“Structural analog” of a compound refers to a chemical entity which comprises chemical changes which do not affect substantially the therapeutic properties and/or biological activity of the compound.

In the context of the present invention, when a combination of a GS regulating ligand and an other active ingredient is considered, the composition containing the GS regulating ligand according to the present invention with the other active ingredient can be as a combined preparation for simultaneous, separate or sequential use in the same therapy. It will be appreciated that the compounds of the combination may be administered simultaneously, either in the same or different pharmaceutical formulations or sequentially. If there is sequential administration, the delay in administering the second active ingredient should not be such as to lose the benefit of the efficacious effect of the combination of the active ingredients.

Whenever within this whole specification “treatment of a condition or disorder” or the like is mentioned with reference to a GS regulating ligand, there is meant:

a) a method for treating a condition or disorder, said method comprising administering a GS regulating ligand to a subject in need of such treatment;
b) the use of a GS regulating ligand for the treatment of a condition or a disorder;
c) the use of a GS regulating ligand for the manufacture of a pharmaceutical preparation for the treatment of a condition or a disorder; and/or
d) a pharmaceutical preparation comprising a dose of a GS regulating ligand that is appropriate for the treatment of a condition or a disorder.

By “treatment” one means in the present application a treatment aiming to prevent or to cure a condition or a disorder. By “cure/treat a condition or a disorder” one means that the treatment alleviates, reduces or abolishes its symptom(s) and/or cause(s) (etiology).

By “low concentrations/doses” one means in the present application that the GS ligand allows to activate and/or inhibit the activity of GS depending on the experimental or (patho)physiological condition in the target cells/tissues/organs/organism without any strict, systematic inhibition. By “high concentrations/doses” is intended that the GS ligand behaves at that doses strictly as a GS inhibitor in the target cells/tissues/organs/organism. By “regulate” is preferably intended that the GS activity can be increased or decreased, when appropriate.

To increase GS activity in certain cells, tissues, organs, one would preferably start with a very low concentration/dose and increase it progressively based on the monitoring of biochemical markers (GS activity, glutamate, glutamine, NH₄, . . . ) and/or clinical symptoms. To decrease GS activity in certain cells, tissues, organs, one will preferably start with a higher concentration/dose, below the maximum tolerated concentration/dose and adapt it i.e. decrease or increase it progressively based on the monitoring of biochemical markers (GS activity, glutamate, glutamine, NH₄, . . . ) and/or clinical symptoms. One skilled in the art can readily determine an effective very low concentration/dose and a “dose below the maximum tolerated concentration/dose”, by taking into account factors such as the size, weight, age and sex of the subject, patho(physiological condition and route of administration. Generally, estimate of an oral “very ow” daily intake is about 100, 200, 300, 400, 500, 600, 700, 800, 900, or 1000 times less than the “dose below the maximum tolerated” daily intake. For instance, for GF and MS, estimated oral “very low” and “below the maximum tolerated” daily intakes are 0.0002 mg/kg bw and 0.02 mg/kg bw, respectively.

### Functions, Conditions and Disorders Related to Ammonia

Ammonia is a molecule with many physiological roles. It serves as an important substrate for several enzymatic reactions, and is a normal product of degradation of proteins and other nitrogen compounds.

In mammals, liver usually has the responsibility to detoxify systemic (plasma) ammonia through its incorporation into urea that will be eliminated in urine. Although normally from protein digestion large quantities of ammonia enter the portal vein, arterial ammonia is maintained at a low concentration (50-100 μM range).

Number of conditions and disorders are associated with hyperammonemia i.e. a systemic increase of ammonia concentration. In some of these conditions and disorders, hyperammonemia is the primary defect; in others, it is secondary. Liver failure (due to cirrhosis, hepatitis, intoxication, . . . ), inborn errors of metabolism (urea cycle disorders, transport defects of intermediates in the urea cycle, organic acidurias, . . . ), porto-systemic shunts as well as “non-hepatic” critical conditions/disorders, alone or combined, can result in a systemic ammonia concentration increase (see Tables 1 and 2 (the “double-underlined” conditions and disorders”), and in particular, i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders (Jones and Weissenborn, 1997; Hawkes et al., 2001; Felipo and Butterworth, 2002b; Leonard and Morris, 2002). High concentrations of ammonia can be also more localized due to local increased protein degradation and/or deficiency of local detoxification/elimination/use e.g. local critical stress due to anoxia, cancer, degeneration, infection, inflammation, ischaemia, surgery, trauma, . . . . Potential factors and mechanisms which can underlie these conditions and disorders are disclosed in Table 4, without to be limited thereto.

Increased ammonia concentration has particularly deleterious effects in the central nervous system. Depending upon the species, the age, and the magnitude and duration of exposure, ammonia may result in a spectrum of neuropsychiatric symptoms including asterixis (flapping tremor), irritability, lethargy, mental retardation, somnolence, disorientation, seizure and coma, and in neuronal cell damage or loss; brain edema sufficient to cause increased intracranial pressure and death by brain herniation is a classic complication of severe acute hyperammonemia (Jones and Weissenborn, 1997; Hawkes et al., 2001; Felipo and Butterworth, 2002b; Leonard and Morris, 2002). Clinical signs associated with localized ammonia increases depend on the cell/tissue/organ function(s), and duration, intensity and nature of the local condition/disorder.

The molecular mechanisms of ammonia toxicity with regard to CNS function are still incompletely understood (Jones and Weissenborn, 1997; Hawkes et al., 2001; Felipo and Butterworth, 2002a,b; Kosenko et al., 2003). One hypothesis stresses the interference of ammonia with glutamate/glutamine metabolism, suggesting that toxic effects may be mediated by subsequent ammonia metabolism rather than by ammonia itself (Hawkins et al., 1993; Zwingmann et al., 1998). Since brain lacks an effective urea cycle, cerebral ammonia removal will rely mainly on glutamine synthesis (Cooper et al., 1985; Cooper and Plum, 1987). Glutamine concentration in the cerebrospinal fluid correlates reasonably with the clinical grade of hepatic encephalopathy (Hourani et al., 1971). High brain glutamine concentrations are commonly found in patients with hepatic encephalopathy as well as in experimentally induced hyperammonemia (Kanamori et al., 1997; De Graaf et al., 1991; Ross et al., 1996; Vogels et al., 1997). Glutamine synthetase is highly concentrated in astrocytes (Norenberg and Martinez-Hernandez, 1979) and a considerable body of evidence implicates astrocytes in the pathogenesis of hepatic encephalopathy (Norenberg, 1981 and 1986; Norenberg et al., 1987). GS ligands known as GS inhibitors have been reported to alleviate acute deleterious effects of high ammonia concentration on astrocytes. In glial cell cultures, MS was observed to prevent the cell swelling seen with exposure to 5 mM ammonium salt (Norenberg and Bender, 1994; Isaacks et al., 1999). In vivo, MS was observed to prevent the increase in cortical glutamine concentration, water content and intracranial pressure as well as the swelling of astrocyte perivascular processes, that are seen in rats when plasma ammonia concentration is increased to 500-600 μM (preferably administered at a dose below 2.5-10 mg/kg; US 2003/0144357 A1; Takahashi et al., 1991; Takahashi et al., 1992; Willard-Mack et al., 1996). However, the astrocytic glutamine is partly released into the blood (Norenberg and Martinez-Hernandez, 1979), which means a net reaction to remove excess ammonia from the brain. A rise in brain GS activity may improve the metabolic balance between astrocytes and neurons in hyperammoniemic situations; in particular it may alleviate excessive glutamatergic activation (Tsacopoulos et al., 1997; Butterworth, 1993; Norenberg et al., 1992; Kosenko et al., 2003). GS is not working at its maximum rate in hyperammoniemic situations (Kosenko et al., 2003). So it is now most often considered useful to increase GS activity in hyperammoniemic situations, at least in conditions of chronic hyperammonemia (Kosenko et al., 2003).

While measures to decrease the quantities of ammonia which enter the circulation from protein digestion in the gastrointestinal tract are most often a corner-stone in the treatment of hyperammoniemic syndromes (for the general principles relevant to the management of hepatic and non-hepatic hyperammonemia see: Jones and Weissenborn, 1997; Hawkes et al., 2001; Felipo and Butterworth, 2002b), altering peripheral GS activity may additionally improve systemic ammonia detoxification/removal. Since skeletal muscle is an important organ involved in systemic ammonia removal, a rise in muscular GS activity may be particularly useful. For instance, zinc supplementation has been proposed in the prevention of hepatic encephalopathy by activating glutamine synthetase; an increased uptake of ammonia by leg skeletal muscle and an increased release of glutamine from leg skeletal muscle were observed after oral supplementation with zinc sulfate in patients with decompensated liver cirrhosis (Yoshida et al., 2001). Activating GS activity may as well improve local ammonia detoxification in case of local increase of ammonia (see also below the importance of local de novo synthesis of glutamine).

Accordingly, the present invention concerns the use of tianeptine, salts thereof, isomers thereof, pro-drugs thereof, metabolites thereof and structural analogs thereof, in the manufacture of medicaments intended for prevention and/or treatment of conditions and disorders related to or resulting in a local excess of ammonia or a systemic excess of ammonia i.e. hyperammon(em)ia.

The present invention also concerns a method of treatment of hyperammon(em)ia in a subject comprising administering to said subject an efficient amount of tianeptine or a salt thereof, an isomer thereof, a pro-drug thereof, a metabolite thereof or a structural analog thereof. Preferably, said systemic ammonia concentration increases are related to a severe liver disease (due to cirrhosis, hepatitis, intoxication, . . . ), an inborn error of metabolism (urea cycle disorder, transport defect of intermediate in the urea cycle, organic aciduria, . . . ), a porto-systemic shunt, and/or a “non hepatic” (critical) disorder (see Tables 1 and 2 (the “double-underlined” conditions and disorders). Said conditions and disorders related to or resulting in a local excess of ammonia or a systemic excess of ammonia are selected from the group consisting of those disclosed in Tables 1 and 2 (the double-underlined conditions and disorders) hyperammon(em)ia, and in particular, i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders. Preferably, said localized ammonia increases are related to a local critical stress due to anoxia, cancer, degeneration, infection, inflammation, ischaemia, surgery, trauma, . . . . Potential factors and mechanisms which can underlie these conditions and disorders are disclosed in Table 4, without to be limited thereto.

For instance, tianeptine metabolites and structural analogs are described in FR 2 104 728 and FR 2 635 461 (the disclosure thereof being incorporated herein by reference) or in Sanchez-Mateo et al., 2003. In particular, tianeptine analogs can have the following formula:

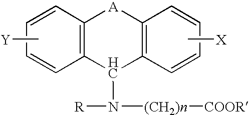

in which A represents a bridge selected from the group consisting of (CH₂)ₘ—, —CH═CH—, —CH₂)ₚ—O—, —(CH₂)ₚ—S—, —CH₂)ₚ—SO₂—, —(CH₂)ₚ—NR₁—, and —SO₂—NR₂—; m is an integer 1, 2, or 3; p is 1 or 2; R1 represents a hydrogen atom or a lower alkyl radical containing 1 to 5 carbon atoms, and R2 represents a lower alkyl radical containing 1 to 5 carbon atoms, X and Y may be the same or different and each represents a hydrogen atom or a halogen atom (e.g., F, Cl, Br); R and R′ may be the same or different and each represents a hydrogen atom or a lower alkyl radical containing 1 to 5 carbon atoms; and n is an integer from 1 to 12. Preferably, A is —SO₂—NR₂—.

In a preferred embodiment, tianeptine metabolites or structural analogs are selected from the compounds disclosed in Table 8.

Accordingly, the present invention concerns also the use of tianeptine, salts thereof, isomers thereof, pro-drugs thereof, metabolites thereof and structural analogs thereof, in the manufacture of medicaments to be combined with other (etiologic and/or symptomatic) drugs or measures intended i) to prevent/treat hyperammonemia and/or hyperammonemia symptoms, in particular benzodiazopine receptor antagonists (flumazenil, . . . ), zinc supplementation and measures to minimize absorption of nitrogenous substances e.g. dietary restriction, evacuation of bowel, lactulose (or a related sugar) and/or broad spectrum antibiotics (for example, neomycin) in liver failure; or sodium benzoate and sodium phenylacetate in conjunction with a low protein diet and a specific amino acid supplementation in carbamylphosphate synthetase, ornithine transcarbamylase or arginosuccinate synthetase deficiency; or sodium benzoate, sodium phenylacetate, arginine hydrochloride and/or haemodialysis, after solid-organ transplantation; or ii) resulting in hyperammonemia, in particular certain anaesthetic agents (halothane, enflurane, . . . ), anti-cancer therapies (5-fluorouracil, asparaginase, . . . ) and antiepileptics (sodium valproate, primidone, . . . ) (Jones and Weissenborn, 1997; Hawkes et al., 2001; Felipo and Butterworth, 2002b; Leonard and Morris, 2002). The present invention also concerns a pharmaceutical composition comprising tianeptine or a salt thereof, an isomer thereof, a pro-drug thereof, a metabolite thereof or a structural analog thereof, and at least one other drug intended to prevent/treat hyperammon(em)ia, in particular flumazenil and zinc. Alternatively, the present invention concerns a pharmaceutical composition comprising tianeptine or a salt, an isomer, a pro-drug, a metabolite or a structural analog thereof, and at least one other drug resulting in hyperammonemia e.g. anti-cancer therapies (5-fluorouracil, asparaginase, . . . ) or antiepileptics (sodium valproate, primidone, . . . ).

More particularly, the present invention concerns the use of NF, salts thereof, isomers thereof, pro-drugs thereof, metabolites thereof and structural analogs thereof, for the preparation of medicaments for prevention and/or treatment of hyperammon(em)ia. The present invention also concerns a method of treatment of hyperammon(em)ia in a subject comprising administering to said subject an efficient amount of NF or a salt thereof, an isomer thereof, a pro-drug thereof, a metabolite thereof or an analog thereof. Preferably, said hyperammonemia is related to a severe liver disease (due to cirrhosis, hepatitis, intoxication, . . . ), an inborn error of metabolism (urea cycle disorder, transport defect of intermediate in the urea cycle, organic aciduria, . . . ), a porto-systemic shunt and/or a “non hepatic” (critical) disorder (see Table 1 and 2 (the “double-underlined” conditions and disorders, with the exception of those disclosed in Table 14. Said conditions and disorders related to or resulting in a local excess of ammonia or a systemic excess of ammonia are selected from the group consisting of those disclosed in Tables 1 and 2 (the double-underlined conditions and disorders) hyperammon(em)ia, and in particular, i) Hepatobiliary disorders such as Hepatic and hepatobiliary disorders, particularly hepatic fibrosis and cirrhosis, hepatic metabolic disorders e.g. alpha-1 anti-trypsin deficiency, Haemochromatosis, Hepatic siderosis and Hepato-lenticular degeneration, Hepatic vascular disorders e.g. Budd-Chiari syndrome, Portal vein occlusion, Portal vein phlebitis and Portal vein stenosis, and other Hepatocellular damage and hepatitis e.g. Alcoholic liver disease, Chronic hepatitis, Hepatic necrosis, Hepatitis alcoholic, Hepatitis chronic active, Hepatitis chronic persistent, Hepatitis fulminant, Hepatitis toxic, Peliosis hepatitis and Reye's syndrome, ii) Metabolic and nutritional disorders congenital, particularly Urea cycle enzyme disorders and Transport defects of intermediates in the urea cycle, Organic acidurias and Lipid metabolism disorders, iii) Other metabolic causes, particularly (Distal) Renal tubular acidosis and Hyperinsulinaemic hypoglycaemia, iv) Porto-systemic shunts, v) Blood and lymphatic system disorders, particularly Neoplasms, Leukaemias, Transplantation therapy and Intensive chemotherapy, vi) Infections and infestations, and vii) Renal and urinary disorders. Preferably, said localized ammonia increase is related to a local critical stress e.g. anoxia, cancer, degeneration, infection, inflammation, ischaemia, surgery, trauma, . . . . Potential factors and mechanisms which can underlie these conditions and disorders are disclosed in Table 4, without to be limited thereto. In a particular embodiment, NF or salts thereof, isomers thereof, pro-drugs thereof, metabolites thereof or structural analogs thereof, can be combined with other measures/drugs intended i) to prevent/treat hyperammonemia and/or hyperammonemia symptoms, in particular benzodiazepine receptor antagonists (flumazenil, . . . ), zinc supplementation and/or measures to minimize absorption of nitrogenous substances i.e. dietary restriction, evacuation of bowel, lactulose (or a related sugar) and/or broad spectrum antibiotics (for example, neomycin) in liver failure; or sodium benzoate and sodium phenylacetate in conjunction with a low protein diet and a specific amino acid supplementation in carbamylphosphate synthetase, ornithine transcarbamylase or arginosuccinate synthetase deficiency; or sodium benzoate, sodium phenylacetate, arginine hydrochloride and/or haemodialysis after solid-organ transplantation; or ii) resulting in hyperammonemia e.g. anaesthetic agents (halothane, enflurane, . . . ), anti-cancer therapies (5-fluorouracil, asparaginase, . . . ) or antiepileptics (sodium valproate, primidone, . . . ) (Jones and Weissenborn, 1997; Hawkes et al., 2001; Felipo and Butterworth, 2002b; Leonard and Morris, 2002). Therefore, the present invention concerns a pharmaceutical composition comprising NF or a salt thereof, an isomer thereof, a pro-drug thereof, a metabolite thereof or a structural analog thereof, and a drug intended to prevent/treat hyperammonemia and/or hyperammonemia symptoms, for instance flumazenil, zinc, sodium benzoate, sodium phenylacetate and arginine hydrochloride, or resulting in hyperammonemia e.g. anti-cancer therapies (5-fluorouracil, asparaginase, . . . ) or antiepileptics (sodium valproate, primidone, . . . ). In a preferred embodiment, the efficient dose is a “low dose” i.e. a dose resulting in concentrations of GF which allows to regulate GS activity in the target cells/tissues/organs/organism. In a preferred embodiment, NF metabolites or (structural) analogs are selected from the group consisting of the compounds in Tables 15.

The present invention also concerns a method of treatment of hyperammon(em)ia comprising administering an effective amount of an “other GS regulating ligand” selected from the group consisting of hydrazines and bisphosphonates, and salts thereof, isomers thereof, pro-drugs thereof, metabolites thereof and structural analogs thereof. Preferably, the therapeutical amount of this compound is a “low dose” i.e. a dose resulting in concentrations of ligand which allows to regulate GS activity in the target cells/tissues/organs/organism. The present invention also concerns the use of GS ligands like hydrazines and bisphosphonates and salts thereof, isomers thereof, pro-drugs thereof, metabolites thereof and structural analogs thereof, for the preparation of medicaments for prevention and/or treatment of hyperammon(em)ia. Furthermore, the present invention contemplates a pharmaceutical composition comprising at least one GS ligand selected from the group of hydrazines and bisphosphonates, at a “low dose”, wherein said dose result in concentrations of ligand which allows to regulate GS activity in the target cells/tissues/organs/organism. In a particular embodiment, said GS ligand like hydrazines and bisphosphonates, and salts thereof, isomers thereof, pro-drugs thereof, metabolites thereof and structural analogs thereof, administered at a “low dose”, can be associated with other measures/drugs intended i) to prevent/treat hyperammonemia and/or hyperammonemia symptoms, in particular benzodiazepine receptor antagonists (flumazenil, . . . ), zinc supplementation and/or measures to minimize absorption of nitrogenous substances i.e. dietary restriction, evacuation of bowel, lactulose (or a related sugar) and/or broad spectrum antibiotics (for example, neomycin) in liver failure; or sodium benzoate and sodium phenylacetate in conjunction with a low protein diet and a specific amino acid supplementation in carbamylphosphate synthetase, ornithine transcarbamylase or arginosuccinate synthetase deficiency; or sodium benzoate, sodium phenylacetate arginine hydrochloride and/or haemodialysis after solid-organ transplantation; or ii) resulting in hyperammonemia e.g. anaesthetic agents (halothane, enflurane, . . . ), anti-cancer therapies (5-fluorouracil, asparaginase, . . . ) or antiepileptics (sodium valproate, primidone, . . . ) (Jones and Weissenborn, 1997; Hawkes et al., 2001; Felipo and Butterworth 2002b; Leonard and Morris, 2002). Therefore, the present invention concerns a pharmaceutical composition comprising a GS ligand like an hydrazine or a bisphosphonate, at a “low dose”, and a drug intended to prevent/treat hyperammon(em)ia for instance flumazenil, zinc, sodium benzoate, sodium phenylacetate or arginine hydrochloride, or resulting in hyperammonemia e.g. anti-cancer therapies (5-fluorouracil, asparaginase, . . . ) or antiepileptics (sodium valproate, primidone, . . . ).

In a particular embodiment, the GS regulating ligand can be selected from the group of hydrazines and salts thereof, isomers thereof, pro-drugs thereof, metabolites thereof and structural analogs thereof, preferably those disclosed in Table 11.

In another particular embodiment, the GS regulating ligand can be selected from the group of bisphosphonates (by extention Mg²⁺ analogs like Sr²⁺), and salts thereof, isomers thereof, pro-drugs thereof, metabolites thereof and structural analogs thereof, preferably selected from those disclosed in Table 12.

In a particular embodiment, the GS regulating ligand can be an analog of L-glutamate or glutamate or a salt thereof, an isomer thereof, a prodrug thereof, a metabolite thereof or structural analog thereof, preferably selected from those disclosed in Table 13.

### Functions, Conditions and Disorders Related to Glutamate

The amino acid L-glutamate plays an important role both in neuronal and non-neuronal tissues as a fundamental metabolite/substrate of the tricarboxylic acid cycle and amino acid metabolism (Hertz, 1979; Hertz et al., 1999; Hertz and Zielke, 2004). It is also accepted as a specific excitatory amino acid in the nervous tissues, where it is the major excitatory neurotransmitter at synaptic junctions (Orrego and Villanueva, 1993), and as an extracellular autocrine and/or paracrine signal mediator in peripheral nervous and non-nervous tissues (Hinoi et al., 2004).

In the nervous tissues, glutamate acts as an excitatory neurotransmitter via stimulation of a complex multiprotein receptor system composed of several different glutamate receptor subtypes, both ligand gated ion channel receptors (N-methyl-D-aspartate (NMDA), α-amino-3-hydroxy-5-methyl-4 isoxazole propionic acid (AMPA) and kainate (Ka) receptors) and G-protein coupled (metabotropic) receptors. This complex beside mediating excitatory neurotransmission plays a key role in synaptogenesis and formation of neuronal circuitry. Compelling evidence indicates that it plays a crucial role in how initial alterations in the activity of CNS are transformed into more persistent and sometimes permanent modifications of the neuronal activity and excitability. These mechanisms are thought, in particular, to underlie learning and memory. On the contrary, excessive activation of glutamate receptors is believed to participate in neurodegeneration following a wide range of neurological insults including hypoglycemia, ischaemia, (epileptic) seizures or trauma. Chronic neurodegenerative disorders such as Alzheimer's disease, amyotrophic lateral sclerosis, Huntington's chorea or AIDS encephalopathy are alike assumed to involve neuronal cell death related to excitotoxic properties of glutamate.

In order to use the common amino acid L-glutamate as a neurotransmitter, it has been necessary to construct a discriminating mechanism that greatly restricts the availability of glutamate. The most far-reaching innovations have been: i) to exclude the nervous tissues (brain) from access to glutamate present in the systemic circulation (blood-brain barrier), thereby making it autonomous in the production and disposal of glutamate; ii) to surround glutamatergic synapses with glial cells and endow these cells with much more efficient glutamate uptake carriers than the neurons themselves, so that most released glutamate is rapidly inactivated by uptake in glial cells; iii) to provide glial cells but not neurons the key enzyme (glutamine synthetase) that is involved in the return of accumulated glutamate to neurons by amidation to glutamine, which has no transmitter activity, and can be safely released to the extracellular space, returned to neurons and deaminated to glutamate (glutamine-glutamate cycle) (Sibson et al., 1997; Zwingmann et al., 1998 and 2002)—in neurons, glutamate serves of course also as a respiratory fuel, gluconeogenic precursor, carrier of nitrogen, precursor of γ-aminobutyric acid (GABA) (Paulsen et al., 1988), etc. (see below)—iv) to restrict to glial cells two key enzymes (pyruvate carboxylase and cytosolic malic enzyme) that are involved, respectively, in de novo synthesis (from glucose) of the carbon skeleton of glutamate, and in the return of the carbon skeleton of excess glutamate to the metabolic pathway for glucose oxidation. As a consequence of these innovations, neurons constantly require new carbon skeletons from glial cells. When these supplies are withdrawn, neurons are unable to generate amino acid transmitters and their rate of oxidative metabolism is impaired. Thus, given the commensalism that exists between neurons and glia, brain function has to be viewed not just as a series of interactions between neurons, but also as a series of interactions between neurons and their collaborating glial cells (Kvamme 1998; Hertz et al., 1999; Hertz and Zielke, 2004).

Clearance of synaptic glutamate by glial cells is required for a normal nervous function (“signal-to-noise ratio”). An increase in excitatory synapses meets an elevation of GS activity in glial cells, which in turn participates in an effective tissue function. This was suggested in different models for instance in established retinal tissue paradigmes (Gorovits et al., 1997; Shaked et al., 2002); in primary culture of retinal tissue i) glutamate serves as a neurotransmitter in photoreceptors, bipolar cells and ganglion cells (Massey and Miller, 1990); however ii) insult leads to accumulation of relatively high levels of glutamate in the extracellular fluid (Louzada et al., 1992; Neal et al., 1994); iii) administration of glutamate leads to neuronal cell death (David et al., 1988; Zeevalk et al., 1989; Zeevalk and Nicklas, 1990); and iv) glutamate receptor antagonists protect against neuronal degeneration (Mosinger et al., 1991). Hormonal induction of the endogenous GS gene in retinal glial cells correlates with a decline in neuronal degeneration (Gorovits et al., 1997), whereas inhibition of GS activity by MS can lead to increased cell death (Gorovits et al., 1997; Shaked et al., 2002); finally, a supply of purified GS enzyme causes a dose-dependent decline in the extent of cell death (Gorovits et al., 1997). So, from the pharmacological and therapeutic point of view, it is appropriate to combat pathological stimulation whilst maintaining a physiological activation.

The Inventors have discovered that tianeptine, depending on the concentration/dose and experimental or (patho)physiological condition, can activate or inhibit GS activity in vitro as well as in vivo (FIGS. 5-7, 14-16, 31-32). At “low concentrations/doses”, it behaves as a GS regulator. Tianeptine was also observed, in vitro, to prevent cellular apoptosis in conditions of elevated ammonia and/or elevated glutamate concentrations (FIGS. 25-27) and, in vivo, to counteract hippocampal CA3 structural changes associated with chronic glucorticoid administration (FIG. 30), to prevent PTZ induced seizure (FIG. 45), and these effects could be explained by their influence on GS (FIGS. 16, 18-22, 31, 42-44 and 46)). Tianeptine could also protect GS from oxidative stress (FIG. 17) and raise GS protein expression in vivo (FIG. 14B, 15B and 31B). These properties of tianeptine can likely participate in its neuroprotective effects (U.S. Pat. No. 6,599,896), its effects on psychoneurotic disorders (FR 2 104 728), stress (FR 2 635 461), pain (FR 2 104 728; U.S. Pat. No. 3,758,528) and seizure (Ceyhan et al., 2005), but also on asthma, cough (FR 2 104 728), nonulcer dyspepsia (U.S. Pat. No. 6,683,072) and irritable bowel syndrome (U.S. Pat. No. 6,683,072), in regulating the clearance of glutamate (and/or the neosynthesis of glutamine/glutamate; see below).

Regulating the clearance of extracellular glutamate (and neosynthesis of glutamine/glutamate) can result in an original pharmacological profile. In particular, all glutamate receptor subtypes may be less activated in situations of high glutamate concentration. Accordingly, the present invention extends the use of tianeptine, salts thereof, isomers thereof, pro-drugs thereof, metabolites thereof and structural analogs thereof, in the manufacture of medicaments to be used in the prevention and/or treatment of all conditions and disorders related to an extracellular excess of glutamate, whatever its cause(s) and the activated glutamate receptor subtype(s), with the exception of the already claimed or proposed clinical applications of tianeptine. In fact, all the already claimed or proposed clinical applications of tianeptine, which are disclosed in Table 5, in particular asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of acquired immune deficiency syndrome (AIDS), . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke, are likely, at least in part, associated and/or related to (absolute or relative) excess of glutamate. Thus, the additional target conditions and disorders complete these already claimed conditions and disorders from the nervous system disorders, psychiatric disorders, gastrointestinal disorders, and respiratory, thoracic and mediastinal disorders SOCs. They comprise in particular conditions and disorders from other “peripheral” SOCs (see Table 2). The present invention also concerns a method of prevention and/or treatment of all these additional conditions and disorders related to excess of glutamate, with the exception of the conditions and disorders disclosed in Table 5, comprising administering an effective amount of tianeptine or of salt thereof, an isomer thereof, pro-drug thereof, a metabolite thereof or a structural analog thereof. Preferably, tianeptine, salts thereof, isomers thereof, pro-drugs thereof, metabolites thereof and structural analogs thereof, are used at “low” doses i.e. doses resulting in concentrations of tianeptine which can regulate GS activity in the target cells/tissues/organs/organism. Tianeptine or salts thereof, isomers thereof, pro-drugs thereof, metabolites thereof or structural analogs thereof, may be in particular combined with treatments preventing from excitotoxic properties of glutamate through other mechanisms e.g. with glutamate release inhibitors (lamotrigine, riluzole, . . . )or glutamate receptor antagonists (D-AP5, ketamine, memantine, MK-801, . . . ). Therefore, the present invention also concerns a pharmaceutical composition comprising tianeptine or a salt, an isomer, a pro-drug, a metabolite or a structural analog thereof, and at least one other drug intended to prevent from excitotoxic properties of glutamate through other mechanisms e.g. glutamate release inhibitors (lamotrigine, riluzole, . . . ) or glutamate receptor antagonists (D-APS, ketamine, memantine, MK-801, . . . ). Contemplated conditions and disorders to be treated by tianeptine, or salts thereof, isomers thereof, pro-drugs thereof, metabolites thereof or structural analogs thereof are preferably selected from the group consisting of i) Gastrointestinal inflammatory conditions, Gastrointestinal ulceration and perforation, and Malabsorption conditions, ii) General disorders and administration site conditions such as Mucosal findings abnormal and Tissue disorders, particularly Trophic disorders e.g. Atrophy and Denervation atrophy, iii) Autoimmune disorders and Immune disorders, iv) Infections and infestations, v) Injury, poisoning and procedural complications such as Administration site reactions, Chemical injury, Injuries by physical agents, and other Procedural and device related injuries and complications, vi) Metabolism and nutrition disorders, particularly Catabolic state and Hypercatabolism, vii) Bone disorders, particularly Osteoporosis, Connective tissue disorders, particularly Lupus erythematosus, Joint disorders, Muscle disorders, and Tendon, ligament and cartilage disorders, xiii) Neoplasms benign, malignant and unspecified, particularly Gliomas benign xix) Respiratory tract inflammatory and immunologic conditions, and Bronchitis chronic xx) Arteriosclerosis, Circulatory collapse and shock, Vascular injuries and Venous varices. Accordingly, the present invention extends the use of NF, salts thereof, isomers thereof, pro-drugs thereof, metabolites thereof and structural analogs thereof, in the manufacture of medicaments to be used in the prevention and/or treatment of all conditions and disorders related to an extracellular excess of glutamate, whatever its cause(s) and the activated glutamate receptor subtype(s), with the exception of the already claimed or proposed clinical applications of NF. In fact, all the already claimed or proposed clinical applications of tianeptine, which are disclosed in Table 14. The present invention also concerns a method of prevention and/or treatment of all these additional conditions and disorders related to excess of glutamate, with the exception of the conditions and disorders disclosed in Table 14, comprising administering an effective amount of NF or of salt thereof, an isomer thereof, pro-drug thereof, a metabolite thereof or a structural analog thereof. Preferably, NF, salts thereof, isomers thereof, pro-drugs thereof, metabolites thereof and structural analogs thereof, are used at “low” doses i.e. doses resulting in concentrations of NF which can regulate GS activity in the target cells/tissues/organs/organism. NF or salts thereof, isomers thereof, pro-drugs thereof, metabolites thereof or structural analogs thereof, may be in particular combined with treatments preventing from excitotoxic properties of glutamate through other mechanisms e.g. with glutamate release inhibitors (lamotrigine, riluzole, . . . ) or glutamate receptor antagonists (D-AP5, ketamine, memantine, MK-801, . . . ). Therefore, the present invention also concerns a pharmaceutical composition comprising NF or a salt, an isomer, a pro-drug, a metabolite or a structural analog thereof, and at least one other drug intended to prevent from excitotoxic properties of glutamate through other mechanisms e.g. glutamate release inhibitors (lamotrigine, riluzole, . . . ) or glutamate receptor antagonists (D-AP5, ketamine, memantine, MK-801, . . . ). Preferably, said conditions and disorders are selected from the group consisting of i) Adrenal gland disorders, particularly cortical hyperfunctions and hypofunctions (incl iatrogenic), ii) Gastrointestinal inflammatory conditions, particularly Inflammatory bowel disease, Gastrointestinal motility and defaecation conditions e.g. Irritable bowel syndrome, Dyspeptic signs and symptoms e.g. Dyspepsia and Flatulence, bloating and distension, Gastrointestinal ulceration and perforation, and Malabsorption conditions, iii) Allergic conditions, more particularly Allergic bronchitis and Asthma, Autoimmune disorders and Immune disorders, iv) Infections and infestations, v) Metabolism and nutrition disorders such as Appetite and general nutritional disorders, Cushing's syndrome, Hypercatabolism and Hypercorticoidism, vi) Bone disorders, particularly Osteoporosis, Connective tissue disorders, particularly Lupus erytheimatosus, Joint disorders, Muscle disorders, and Tendon, ligament and cartilage disorders, vii) Adjustment disorders, Anxiety disorders and symptoms, more particularly Obsessive compulsive disorder and Stress disorders, Cognitive and attention disorders and disturbances, Depressed mood disorders and disturbances, Manic and bipolar mood disorders and disturbances, Mood disorders and disturbances, more particularly Affect alterations, Emotional and mood disturbances and Mood disorders due to a general medical condition, Mental disorders due to a general medical condition, xiii) Neoplasms benign, malignant and unspecified, particularly Gliomas benign viii) Bronchial conditions e.g. Allergic bronchitis and Bronchitis chronic and Bronchospasm e.g. Asthma, Cough. In a preferred embodiment, NF or a salt thereof, an isomer thereof, a pro-drug thereof, a metabolite thereof or a (structural) analog thereof is selected from Table 15.

Additionally, the present invention concerns a method of treatment of conditions and disorders related to an (absolute or relative) glutamate imbalance (deficiency or excess) in a subject comprising administering an effective amount of a GS ligand selected from the group consisting of GF, MS, NF, hydrazines and bisphosphonates, and salts thereof, isomers thereof, pro-drugs thereof, metabolites thereof and structural analogs thereof. Preferably, the therapeutical amount of this compound is a “low dose” i.e. a dose resulting in concentrations of ligand which regulate GS activity in the target cells/tissues/organs/organism. The present invention also concerns the use of GS ligands selected from the group consisting of GF, MS, hydrazines and bisphosphonates, and salts thereof, isomers thereof, pro-drugs thereof, metabolites thereof and structural analogs thereof, for the preparation of medicaments for prevention and/or treatment of conditions and disorders related to (absolute or relative) deficiency or excess of glutamate, with the exception of: if said GS ligand is GF or MS, the conditions and disorders related to cerebral ischemia, hyperammonemia, bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is a hydrazine, the conditions and disorders disclosed in Table 6; and, if said GS ligand is a bisphosphonate, the conditions and disorders disclosed in Table 7. Preferably, the conditions and disorders to be treated are selected from those disclosed in Tables 2 and 3. Furthermore, the present invention contemplates a pharmaceutical composition comprising at least one GS ligand selected from the group consisting of GF, MS, hydrazines and bisphosphonates, at a “low dose”, wherein said “low dose” result in concentrations which regulate the GS activity, and at least one other drug intended to prevent from excitotoxic properties of glutamate through other mechanisms e.g. glutamate release inhibitors (lamotrigine, riluzole, . . . ) or glutamate receptor antagonists (D-AP5, ketamine, memantine, MK-801 . . . ). In a preferred embodiment, said GS ligand is GF or a salt thereof, an isomer thereof, a pro-drug thereof, a metabolite thereof or a (structural) analog thereof, preferably a compound selected from Tables 9 and 13, more preferably Table 9. In an other embodiment, said GS ligand is MS or a salt thereof, an isomer thereof, a pro-drug thereof, a metabolite thereof or a (structural) analog thereof, preferably a compound selected from Tables 10 and 13, more preferably Table 10. In a preferred embodiment, said hydrazine, salt thereof, isomer thereof, pro-drug thereof, metabolite thereof or structural analog thereof is selected from Table 11. Preferably it is isoniazid or iproniazid, or a salt thereof, an isomer thereof, a pro-drug thereof, a metabolites thereof or a structural analogs thereof. In a preferred embodiment, said bisphosphonate, or salt thereof, isomer thereof, pro-drug thereof, metabolite thereof or structural analog thereof is selected from Table 12. Preferably it is pamidronate, risedronate or [(5-Chloro-pyridin-2-ylamino)-phosphono-methyl]-phosphinic acid, or a salt thereof, an isomer thereof, a pro-drug thereof, a metabolite thereof or a structural analogs thereof.

Regulating glutamate clearance of extracellular glutamate (and neosynthesis of glutamine/glutamate) may also participate to reduce/treat the hyperglutamatergic consequences of NMDA receptor dysfunctions implicated in pathophysiologic processes of neuropsychiatric illnesses such as schizophrenia (Anand et al., 2000). Lamotrigine, a drug inhibiting glutamate release, was observed to reduce the neuropsychiatric effects of ketamine, a NMDA receptor antagonist, in humans. Lamotrigine significantly decreased ketamine-induced perceptual abnormalities, positive and negative symptoms, and learning and memory impairment. On the contrary, it increased the immediate mood-elevating effects of ketamine, which might be not the case with drugs regulating both glutamate release and clearance of extracellular glutamate, in particular those regulating GS activity.

Accordingly, the present invention extends the use of tianeptine, salts thereof, isomers thereof, pro-drugs thereof, metabolites thereof and structural analogs thereof, in the manufacture of medicaments intended to reduce the hyperglutamatergic consequences of glutamate receptor dysfunctions, in particular of NMDA receptor dysfunctions implicated in the pathophysiologic processes of neuropsychiatric illnesses such as schizophrenia, with the exception of the conditions and disorders disclosed in Table 5. Tianeptine, salts thereof, isomers thereof, pro-drugs thereof, metabolites thereof and structural analogs thereof might be combined with treatments altering glutamate receptor(s) function(s), in particular with NMDA glutamate receptor antagonists (D-AP5, ketamine, memantine, MK-801, . . . ), with the aim to optimize their therapeutic effects in particular to reduce their hyperglutamatergic consequences mediated by non-NMDA glutamate receptors (adverse effects). Therefore, the present invention contemplates a pharmaceutical composition comprising such combinations. Similarly, instead of tianeptine, GS ligands selected from the group consisting of GF, MS, NF, hydrazines and bisphosphonates, and salts thereof, isomers thereof, pro-drugs thereof, metabolites thereof and structural analogs thereof, can be used at “low doses” i.e. doses resulting in concentrations which regulate GS activity (directly or indirectly). All these GS ligands can be combined with other treatments preventing (in particular through other mechanisms) from these hyperglutamatergic consequences of glutamate receptor dysfunctions e.g. ampakines, glutamate release inhibitors (lamotrigine, riluzole, . . . ), glycine, . . . . Therefore, the present invention contemplates a pharmaceutical composition comprising such combinations.

Regulating the clearance of extracellular glutamate (and neosynthesis of glutamine/glutamate) by regulating GS activity may be also particularly useful in disorders with extracellular glutamate excess where GS protein/activity is altered. This was reported for instance for hyperammonemia (Kosenko et al., 2003; see above), temporal epilepsy (Eid et al., 2004) and sepsis (Tumani et al., 2000). A deficiency in glutamine synthetase in astrocytes has been proposed to be the molecular basis for extracellular glutamate accumulation and seizure generation in mesial temporal lobe epilepsy, a drug resistant type of epilepsy (Eid et al., 2004); in more than 40% of cases, this disorder cannot be controlled by medication, and anteromedial temporal lobectomy with hippocampectomy is needed for seizure control in certain of these patients. Tianeptine and GF . . . demonstrated to be effective in PTZ-induced seizure (FIG. 45; Ceyhan et al., 2005). Apoptosis of dentate granular cells in the hippocampal formation during bacterial meningitis has been proposed to be due to the inability of hippocampal glutamine synthetase to metabolize excess amounts of glutamate (Tumani et al., 2000).

Accordingly, the present invention extends the use of tianeptine, salts thereof, isomers thereof, pro-drugs thereof, metabolites thereof and structural analogs thereof, in the manufacture of medicaments to be used for preventing and/or treating conditions and disorders with high extracellular glutamate where GS protein/activity is altered or insufficiently or too effective, e.g. in hyperammonemia or critical sepsis, with the exception of the conditions and disorders disclosed in Table 5. Tianeptine, salts thereof, isomers thereof, pro-drugs thereof, metabolites thereof and structural analogs thereof, may be in particular combined with etiologic and symptomatic treatments in these indications e.g. symptomatic ammonia lowering measures/treatments (see above)or antibiotics (aminoglycosides, cephalosporins, chloramphenicol, macrolides, penicillins, quinolones, rifampicine, sulfonamides, tetracyclines, trimethoprim-sulfamethoxazole, . . . ). Similarly, instead of tianeptine, GS ligands selected from the group consisting of GF, MS, NF, hydrazines and bisphosphonates, and salts thereof, isomers thereof, pro-drugs thereof, metabolites thereof and structural analogs thereof, also can be used in the manufacture of medicaments intended for preventing and/or treating conditions and disorders with high extracellular glutamate where GS protein/activity is altered or insufficiently or too effective, preferably at “low doses” i.e. doses resulting in concentrations which regulate GS activity (directly or indirectly), with the exception of: if said GS ligand is GF or MS, the conditions and disorders related to cerebral ischemia, hyperammonemia, bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is NF the conditions and disorders disclosed in Table 14; if said GS ligand is a hydrazine, the conditions and disorders disclosed in Table 6; and, if said GS ligand is a bisphosphonate, the conditions and disorders disclosed in Table 7. Thus, the present invention concerns a pharmaceutical composition comprising a drug selected from the group consisting of tianeptine, GF, MS, NF, hydrazines and bisphosphonates, and salts thereof, isomers thereof, pro-drugs thereof, metabolites thereof and structural analogs thereof, and etiologic and symptomatic treatments in these indications e.g. symptomatic ammonia lowering treatments (see above) or anti-epileptic treatments (benzodiazepines, carbamazepine, ethosuximide, hydantoins, gabapentin, lamotrigin, phenobarbital, primidone, valproate, . . . ).

Endogenous glutamate production can be stimulated or triggered by a variety of inflammatory mediators, including arachidonate metabolites and reactive oxygen species (Lipton and Gendelman, 1995; Harder et al., 1998; Farooqui et al., 1995). There is also a lot of evidence supporting glutamate-induced injury is mediated by nitric oxide and other free radicals, including superoxide anions. NMDA-induced injury in primary cortical cultures (Dawson et al., 1991) as well as in cerebellar (Garthwaite et al., 1986) or hippocampal slices (Izumi et al., 1992) have been reported to be mediated by excessive NO synthesis. Glutamate neurotoxicity is susceptible to modulation by intervention at several levels, which include blockade of the ionotropic receptors, inhibition of NO synthesis and inhibition of NO-induced activation of poly(ADP-ribose) polymerase (PARP). The major role of glutamate in determining neurological disorders is characterized by an increasing damage of cell components, including mitochondria, leading to cell death, and reactive oxygen species are generated in death process. Glucocorticoids as well as various stresses increase GS expression (Abcouwer et al., 1995 and 1996; Ardawi 1990; Ardawi and Majzoub 1991; Chandrasekhar et al., 1999; Lukaszewicz et al., 1997). Cytokines are likely involved in the mechanism(s) of action of tianeptine e.g. tianeptine and its enantiomers S 16190 and S 16191 have neuroprotective effects against deleterious effects of certain cytokines in cortex and white matter (Plaisant et al., 2003). One of these cytokines, IL-1β, which is normally present in the hippocampus, inhibits neuronal plasticity and is believed to be a causative agent for depression (Dantzer et al., 1998 and 1999; Rothwell, 1997). Tianeptine has been shown to antagonise the behavioural effects of lipopolysaccharide (LPS) as well as of chronic administration of IL-1β (Castanon et al., 2001), and to inhibit fos expression induced by LPS in the rat paraventricular nucleus (Castanon et al., 2003).

Accordingly, the present invention extends the use of tianeptine, salts thereof, isomers thereof, pro-drugs thereof, metabolites thereof and structural analogs thereof, in the manufacture of medicaments intended for prevention and/or treatment of all conditions and disorders with high extracellular glutamate combined with, related to or resulting in an inflammatory process, with the exception of the conditions and disorders disclosed in Table 5. All the already claimed or proposed (central and peripheral) clinical applications of tianeptine likely or possibly, at least in part, are related to or result in an inflammatory process. Thus, additional target conditions and disorders complete these already claimed conditions and disorders. They comprise in particular conditions and disorders from “peripheral” SOCs (see below and Table 2). Tianeptine, salts thereof, isomers thereof, pro-drugs thereof, metabolites thereof and structural analogs thereof, may be in particular combined with anti-inflammatory drugs or drugs preventing from the occurrence of inflammation. Tianeptine may allow to decrease the doses of glucocorticoids in conditions and disorders with high extracellular glutamate combined with, related to or resulting in an inflammatory process—this was for instance reported in asthma (Lechin et al., 1998)- and/or prevent adverse effects of glucocorticoids at high doses e.g. impairment of antioxidant status (Orzechowski et al., 2000). Similarly, instead of tianeptine, GS ligands selected from NF, GF, MS, hydrazines and bisphosphonates, and salts thereof, isomers thereof, pro-drugs thereof, metabolites thereof and structural analogs thereof, can also be used, preferably at “low doses” i.e. doses resulting in concentrations which regulate GS activity in the target cells/tissues/organs/organism (directly or indirectly). Accordingly, the present invention extends the use of a GS ligand selected from GF, MS, NF, hydrazines and bisphosphonates, and salts thereof, isomers thereof, pro-drugs thereof, metabolites thereof and structural analogs thereof, in the manufacture of a medicament intended for prevention and/or treatment of all conditions and disorders with extracellular excess of glutamate combined with, related to or resulting in an inflammatory process, with the exception of: if said GS ligand is GF or MS, the conditions and disorders related to cerebral ischemia, hyperammonemia, bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is NF the conditions and disorders disclosed in Table 14; if said GS ligand is a hydrazine, the conditions and disorders disclosed in Table 6; and, if said GS ligand is a bisphosphonate, the conditions and disorders disclosed in Table 7. Thus, the present invention concerns a pharmaceutical composition comprising a drug selected from the group consisting of tianeptine, GF, MS, NF, hydrazines and bisphosphonates, and salts thereof, isomers thereof, pro-drugs thereof, metabolites thereof and structural analogs thereof, and anti-inflammatory drugs or drugs preventing from the occurrence of inflammation. Preferably, the anti-inflammatory drug is a (gluco)corticoid drug.

Finally, neuronal death results from multifactorial processes that are inter-related and vary depending on the (patho)physiological condition. A number of robust and important experimental evidence conflicts with the notion that endogenous glutamate excitotoxicity is the sole contributor to neuronal death. For instance, very high extracellular levels of glutamate, far above those measured in models of neurological disorders, must be reached to initiate neuronal death. Thus, drugs like tianeptine that are able to regulate GS activity depending on the pathophysiological condition can be protective by increasing the resistance of cells e.g. neurons and astrocytes to deleterious mechanisms that are not necessary directly or mainly related to glutamatergic transmission (Obrenovitch et al., 2000; see below).

In addition to its “excitatory amino acid neurotransmitter” role in the nervous tissues, evidence is emerging for a role of glutamate as an extracellular signal mediator in a myriad of peripheral nervous and non-nervous locations (Skerry and Genever, 2001; Hinoi et al., 2004). Recent reports give support to the expression of glutamate signaling molecules in adrenal gland (Watanabe et al., 1994; Yoneda & Ogita 1986; Kristensen, 1993; Hinoi et al., 2002b,c), bone (osteocyte, osteoblast, and osteoclast) (Mason et al., 1997; Chenu et al., 1998; Hinoi et al., 2002a and 2003), (cerebral) endothelium (Krizbai et al., 1998), autonomic and sensory ganglions (Shigemoto et al., 1992; Watanabe et al., 1994), heart (Gill et al., 1998), liver (Storto et al., 2000b), gastrointestinal tract (Sninsky et al., 1994; Shannon & Sawyer 1989), kidney (Leung et al., 2002, Deng et al., 2002), lung (Said et al., 1996), lymphocytes (Lombardi et al., 2001), macrophages (Dickman et al., 2004), pancreas (Inagaki et al., 1995; Gonoi et al., 1994; Hayashi et al., 2003; Tong et al., 2002), pineal gland (Govitrapong et al., 1986; Sato et al., 1993; Yamada et al., 1998; Yatsushiro et al., 2000), pituitary gland (Kiyama et al., 1993; Hinoi and Yoneda, 2001), platelets (Franconi et al., 1996), skin (Genever et al., 1999a), male lower urogenital tract and testis (Storto et al., 2001; Nagata et al., 1999; Gonzalez-Cadavid et al., 2000), thymus (Storto et al., 2000a), . . . . Glutamate behaves as a neuro-, auto- and/or paracrine transmitter in these locations; of course it is also an important gustatory stimulus that is believed to signal dietary protein (Chaudhari et al., 2000).

In these peripheral locations, glutamate acts via the same complex multiprotein receptor system as in the CNS i.e. ligand gated ion channels and/or G-protein coupled (metabotropic) receptors. The expression—at least 16 complementary deoxyribonucleic acids encoding for the glutamate receptors have been identified and may be modified further by alternate splicing or ribonucleic acid editing (Gasic and Hollmann, 1992; Hollmann and Heinemann, 1994)—and distribution of these signaling molecules is specific of each species, each location, each condition, . . . . For instance in rat heart, GluR 2/3, GluR 5/6/7, Ka 2, and NMDAR 1 subunits are expressed within nerve terminals, ganglia, conducting fibers, nerve bundles and some to myocardiocytes (particularly in the atrium), each with a distinct pattern of distribution (Gill et al., 1998; Leung et al., 2002). Glutamate receptors are also expressed in the aorta, pulmonary artery, . . . . This sophisticated “anatomo-functional” organization—in fact, the receptor complexes of different subunit combinations exhibit varied pharmacological and electrophysiological properties, including ionic channel selectivity to Na⁺, K⁺ and Ca²⁺ (Gasic and Hollmann, 1992; Hollmann and Heinemann, 1994)—suggests that glutamate receptors play a specific role in cardiac electrophysiology and pathology; secondarily, glutamate receptors might be involved in cardiac dysfunctions associated with intoxication with excitatory amino acids present in food or food additives such as domoic acid or monosodium L-glutamate. In the (rat) kidney, the NMDAR1 subunit is present in cortex and medulla, and its expression increases with development; of the NMDAR2 subunits, only the NMDAR2C is present (Leung et al., 2002). NMDA receptors have been observed to exert a tonic vasodilatory influence (Deng et al., 2002), this effect being likely related to an effect on the proximal tubule; at this level, GS activity is a key factor that limits the release of ammonia, generated by renal cells, into the urine and/or renal vein (Ferrier et al., 1999).

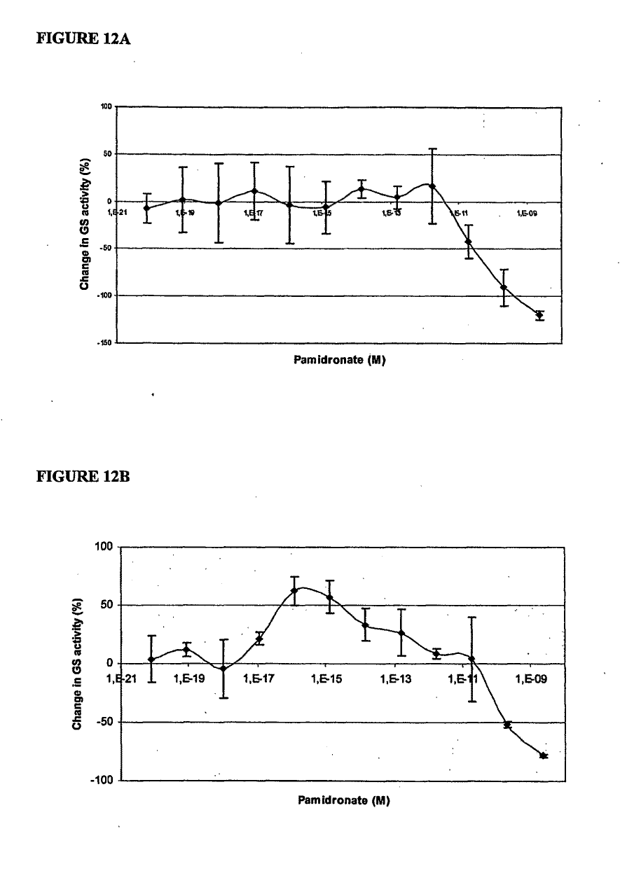

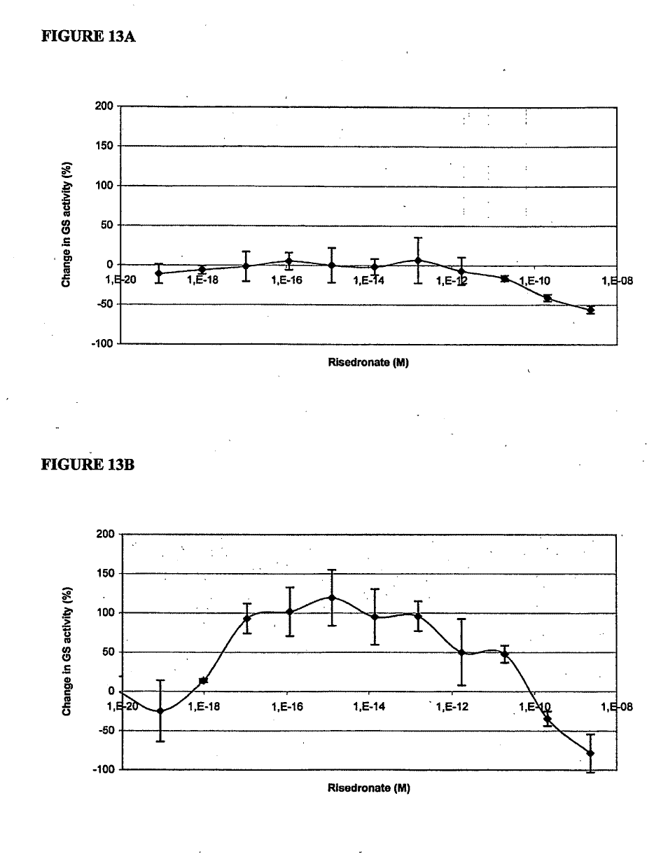

This complex system confers a finely tuned control over a wide range of physiological functions, conditions and disorders. For instance in bone, where GS expression in osteoblasts can be induced by glucocorticoids (Olkku et al., 2004), glutamate plays a crucial role in influencing proliferation, differentiation, activity as well as apoptosis of both osteoblasts and osteoclasts. An innervation of bone by glutamate-containing nerves has been demonstrated (Chenu, 2002). There is compelling evidence for an osteoblastic source of glutamate. Osteoblasts express all the components of neuronal presynaptic machinery required for glutamate release (Bhangu et al., 2001). Glutamate exocytosis from osteoblasts has been demonstrated, prevention of which inhibits cell survival and differentiation (Hinoi et al., 2002b; Genever and Skerry, 2001). There are also several arguments suggesting osteoblasts use glutamate as a signalling molecule to perceive, record and respond to mechanical stimulation (Spencer and Genever, 2003; Spencer et al., 2004). Osteoporosis has been proposed to result from an age-related failure in these adaptive osteogenic responses (Lanyon and Skerry, 2001; Frost, 2002). NMDA receptors are expressed on osteoblasts and osteoclasts (Patton et al., 1998; Chenu et al., 1998; Peet et al., 1999; Hitchcock et al., 2003). NMDA receptor antagonists can inhibit bone formation as well as bone resorption (Peet et al., 1999; Birch et al., 1997); these effects are likely mostly to be mediated through impaired cell differentiation (Peet et al., 1999; Hinoi et al., 2003; Merle et al., 2003). As such, manipulation of these signalling pathways offers real therapeutic potential for the treatment of bone disorders characterised by inappropriate changes in bone formation and/or bone resorption. Glutamate might participate to prime the skeleton to avoid inappropriate changes in bone formation/resorption ratio, in particular those due to biomechanical factors, using mechanisms similar or identical to neuronal long-term potentiation (Spencer et al., 2004). In that respect, the discovery that bisphosphonates such as pamidronate and risedronate, like [(5-Chloro-pyridin-2-ylamino)-phosphono-methyl]-phosphinic acid, and Sr²⁺ alter GS activity (probably through docking into GS Mn²⁺ binding sites) fortifies the therapeutic potential of this approach for the treatment of bone disorders (FIGS. 12-13) (Kafarski et al., 2000). Indeed bisphosphonates are largely employed as therapeutic agents for treatment of bone disorders, in particular of osteolytic bone metastases, osteoporosis associated with postmenopause and long-term corticosteroid therapy, and Paget's disease of bone (see Table 7); and their modes of action remain unclear.

Mechanisms similar or identical to neuronal long-term potentiation likely play an important role in peripheral tissues/organs. For instance, they participate in the prolonged alteration in responsiveness of cerebral endothelial cells after exposure to high glutamate concentrations e.g in ischemic conditions (Krizbai et al., 1998). Exposure of cerebral endothelial cells to glutamate increases the phosphorylation of calcium/calmodulin dependent protein kinase II (Krizbai et al., 1998). In its phosphorylated form, CAM-PKII loses its Ca²⁺ dependency and retains its activity (Bronstein et al., 1993). Thus, vascular glutamate signalling is believed to play an important role in the breakdown of the blood brain barrier in cerebral injuries e.g. in ischemic conditions; this effect is mediated by nitric oxide through NMDA receptors (Fergus and Lee, 1997; Meng et al., 1995).

Over-activation of peripheral glutamate receptors may be also a pathogenic mechanism of injury and inflammation in peripheral locations; conversely, endogenous glutamate production may also be stimulated or triggered by a variety of inflammatory mediators in peripheral locations. For instance, excessive activation of NMDA receptors may be involved in the pathogenesis of some inadequately understood forms of “neurogenic” pulmonary edema like occurring at high altitude as well as of inflammatory injury of asthmatic airways (Said et al., 1996). NMDA receptor activation in perfused, ventilated rat lung triggers acute injury marked by increased pressures needed to ventilate and perfuse the lung, and by high-permeability edema (Said et al., 1996). This injury can be prevented by competitive NMDA receptor antagonists and by NMDA channel-blockers, and is reduced in the presence of Mg²⁺ (Said et al., 1996). As with NMDA-induced injury in CNS, this lung injury requires L-arginine, is associated with increased production of NO, and is attenuated by NO synthase inhibitors (Dawson et al., 1993; Said et al., 1996); inhibitors of NMDA receptors also greatly attenuate oxidant lung injury caused by paraquat or xanthine oxidase, supporting the role for endogenous activation of NMDA receptors in mediating certain forms of lung injury (Said et al., 2000). The neuropeptide vasoactive intestinal peptide and inhibitors of poly(ADP-ribose) polymerase (PARP) can also prevent this injury, but without inhibiting NO synthesis, both acting by inhibiting the toxic action of NO (Said et al., 1996). All these findings provide a molecular-biological basis for the excitotoxic actions of glutamate in (rat) lungs and airways. NMDAR 1 subunits were detected in peripheral, midlung and mainstem lung regions, as well as in the trachea and the alveolar macrophages (Dickman et al., 2004). NMDAR 2C subunits were detected in peripheral and mid-lung (Dickman et al., 2004). NMDAR 2D are the dominant subunits in the peripheral gas-exchange zone of lung and in alveolar macrophages (Dickman et al., 2004).

Peripheral tissues are protected from excess of extracellular glutamate through different mechanisms: barriers which protect from “circumfluent” glutamate, amino acid metabolism (“recycling”), in which glutamine synthetase plays an important role in catalyzing the formation of glutamine from glutamate and ammonia, and glutamate metabolism through the tricarboxylic acid cycle. Clearance of glutamate (and ammonia) is required for the normal function of peripheral tissues and an increase or a regulation of GS activity may therefore be prone to protect peripheral tissues from high glutamate (plus ammonia) concentrations (Haussinger et al., 1985); it provides also glutamine which plays an important role to maintain normal cell and tissue function (see below).

Tianeptine can behave as a GS regulator at “low concentrations/doses”. Such an regulation of the release/recycling of glutamate (as well as of the synthesis of glutamine; see below) can likely participate in its already reported peripheral properties e.g. on asthma, cough (FR 2 104 728), irritable bowel syndrome (U.S. Pat. No. 6,683,072), nonulcer dyspepsia (U.S. Pat. No. 6,683,072), pain (FR 2 104 728; U.S. Pat. No. 3,758,528) and stress (FR 2 635 461). For instance, the very impressive clinical effects of tianeptine on asthma i.e. “dramatic and sudden decrease of clinical rating” and “increase in pulmonary function” (Lechin et al., 1998) which were considered “provocative and worth exploring” by most of the scientific community because they were suggested to be related to effects on serotonin, can be reasonably explained by the effects of tianeptine on GS (see above). Glucocorticoids, which are the most effective long-term control medicine currently available in asthma and which increase GS activity and expression in lungs (Labow et al., 1998), could be even omitted in young asthmatic patients treated with tianeptine (Lechin et al., 1998); supplemental glutamine might participate in these protective effects in preventing from oxidative stress (see below).

The clinical effects of tianeptine on irritable bowel syndrome can be alike reasonably explained by a regulation of GS activity. In fact glutamate is likely to play a role as an excitatory neurotransmitter in the enteric nervous system (ENS) (Kirchgessner, 2001). For instance, in the guinea-pig, glutamate immunoreactivity has been detected in subsets of submucosal and myenteric neurons in the ileum. At this level, glutamate is selectively concentrated in terminal axonal vesicles and can be released after application of an appropriate stimulus (Wiley et al., 1991; Liu et al., 1997a; Sinsky and Donnerer, 1998; Reis et al., 2000). Enteric neurons are endowed both with the neuronal transporter excitatory amino acid carrier 1 (EAAC1) and the vesicular glutamate transporter 2 (VGLUT2) (Liu et al., 1997a; Tong et al., 2001). Functional studies are consistent with a role of glutamate in the modulation of motor and secretory functions in the gut (Wiley et al., 1991; Sinsky and Donnerer, 1998; Cosentino et al., 1995; Rhoads et al., 1995). Glutamate may in particular participate in the modulation of the enteric cholinergic function—glutamate receptors are abundantly expressed on enteric cholinergic neurons (Liu et al., 1997a)—, since activation of NMDA receptors enhances acetylcholine release from myenteric neurons in the ileum and colon (Wiley et al., 1991; Cosentino et al., 1995); this latter effect is likely responsible for glutamate-induced contractions of the guinea-pig ileum (Wiley et al., 1991; Sinsky and Donnerer, 1998). So there are structural and functional bases for a glutamatergic modulation of enteric neurons in the ENS (Giaroni et al., 2003). At this level, glutamate receptors of the NMDA type may participate to the facilitatory modulation of excitatory cholinergic pathways, which retain a primary role in the control of motility. Secondarily, glutamate might also have a role in the modulation of sensory pathways, as already suggested in the guinea-pig ileum and rat colon (Kirchgessner, 2001; McRoberts et al., 2001). Hypothetically, in the complex neuronal circuitries, which constitute the ENS, glutamate might also participate to activity-dependent plasticity (Giaroni et al., 1999). Secondarily, glutamate receptors might be involved in alterations of the gastrointestinal functions consequent to an alimentary intoxication, as observed after ingestion of food contaminated with domoic acid, a ionotropic non-NMDA glutamate receptor agonist (Teitelbaum et al., 1990). Finally, we cannot exclude that this system may represent an underlying support for glutamate mediated excitotoxicity in the ENS. Indeed, in the guinea-pig, prolonged stimulation of enteric ganglia by glutamate was observed to cause necrosis and apoptosis in ileal enteric neurons, which seemed primarily mediated via NMDA receptors (Kirchgessner et al., 1997). So excitotoxicity induced by glutamate may be a critical factor in the pathogenesis of some diseases of the gastrointestinal tract, as it is the case for some neurological disorders in the CNS (Obrenovitch and Urenjak, 1997).

The effects of tianeptine on GS can also plausibly explain the clinical effects of tianeptine on pain. Indeed glutamate is expressed in the sensory pathways e.g. in the trigeminal and dorsal root ganglions (Watanabe et al., 1994) in sensory nerve terminals (Ault and Hildebrand, 1993; Carlton et al., 1995). Administration of glutamate into a joint produces a hyperalgesic response, while glutamate receptor antagonists can provide protection against its development (Sluka et al., 1994; McNearney et al., 2000). GS inhibitors were observed to alleviate acute or chronic pain (WO 2004/0284448 A). The concentration of glutamate in the synovial fluid of humans with active arthritis, in particular in Reiter's, systemic lupus erythematosus and infectious arthropathies, is increased, which suggest in addition that glutamate mediated events may contribute to the pathogenesis of arthritic conditions (McNearney et al., 2000). Etc.

Finally, although it is clear that further investigations are required to clarify the role(s) played by glutamate (receptors) and GS in the different peripheral cells/tissues/organs under normal conditions and different types of conditions and disorders, tianeptine, due to its discovered mechanism of action on GS and already demonstrated therapeutic properties, should offer real therapeutic potential in a wide range of additional peripheral disorders associated with an (absolute or relative) deficiency or excess of glutamate.

Tianeptine may be first useful in varied conditions/disorders associated with (altered) cell differentiation. For instance in bone, tianeptine indications might not only involve bone disorders characterised by inappropriate and excessive changes in bone formation and/or bone resorption (see above) but also haematopoietic disorders. In particular, bone marrow megakaryocytes, which primary function is the production and release of functional platelets, express NMDA subunits (NMDAR1, NMDAR2A and NMDAR2D). Cell lines as well as primary megakaryocytes incubated with a NMDA glutamate receptor antagonist fail to produce proplatelet structures, and lack the cytoplasmic characteristics and organelles essential for the production of functioning platelets (Genever et al., 1999b; Hitchcock et al., 2003).

Tianeptine may be also useful in disorders associated with mucous membrane (barrier) injuries or disruptions e.g. for the treatment of skin diseases and enhancement of wound healing (epidermal renewal). Glutamate plays an important role as a signal of cutaneous barrier homeostasis and epidermal hyperplasia induced by barrier disruption (Genever et al., 1999a; Fuziwara et al., 2003). In resting skin epidermis, immunoreactive NMDA-, AMPA- and metabotropic-type glutamate receptors are colocalized with the specific glutamate transporter EAAC1 in basal layer keratinocytes, and GLT-1 (glutamate transporter type 1), a related transporter, is expressed suprabasally. In full-thickness skin wounds, marked modifications in the distribution of N-methyl-D-aspartate receptors and EAAC1 are observed during re-epithelialization. Topical application of L-glutamic acid, L-aspartic acid (non-specific glutamate receptor agonists) and N-methyl-D-aspartate (NMDA type receptor agonist) delays the barrier recovery rate after barrier disruption with tape stripping. On the other hand, topical application of D-glutamic acid (a non-specific antagonist of glutamate receptor), MK 801 or D-AP5 (NMDA-type receptor specific antagonists) accelerates the barrier repair. Topical application of MK-801 also promotes the healing of epidermal hyperplasia induced by acetone treatment under low environmental humidity. Immediately after barrier disruption on skin organ culture, secretion of glutamic acid from skin is significantly increased.

Tianeptine may be as well useful in immunological disorders. The Inventors already mentioned (see above) the increased glutamate in the synovial fluid of patients with active arthritis, in particular in Reiter's and systemic lupus erythematosus (McNearney et al., 2000). In addition, functional glutamate receptors are present in different thymic cell compartments, and might have a role in the intrathymic lympho-stromal relationships regulating thymocyte differentiation (Storto et al., 2000a). How these receptors are activated is matter of speculation. Glutamate receptors may respond to glutamate, which may either enter the thymus from the bloodstream (where it is present in micromolar concentrations) or may be released from stromal cells or thymocytes. Glutamate receptors are also found on peripheral blood lymphocytes and elevated extracellular glutamate concentrations may inhibit peripheral lymphocyte functions (Kostanyan et al., 1997; Droge et al., 1988; Lombardi et al., 2001); an inverse correlation between extracellular glutamate levels and T lymphocyte proliferation has been observed (Droge et al., 1988). The relevance of this observation could be substantial, because of the evidence that excitatory amino acid neurotransmission is related to many pathological conditions characterized by impaired immune functions (AIDS, Alzheimer disease, hepatic encephalopathy, chronic epilepsy, multiple sclerosis, . . . ), and that high plasma glutamate concentrations (commonly observed in patients with AIDS and neoplastic diseases) inhibit lymphocyte responses to mitogens and macrophage cysteine release into the extracellular space (Droge et al., 1988; Eck et al., 1989). We can presume that either elevated plasma glutamate concentrations or large glutamate release in tissular extracellular spaces e.g. in nervous tissues (Olney, 1990; Lipton & Rosenberg, 1994) may impair lymphocyte functions and have important secondary immunological consequences.

Tianeptine may be in addition useful in conditions and disorders associated with, or resulting in abnormal regulation of endocrine tissues. Glutamate receptors are believed to participate in the regulation of hormone secretion in varied endocrine tissues e.g. in pancreas, adrenal glands, pineal gland or pituitary gland. Pancreatic islets express both NMDA and non-NMDA ionotropic glutamate receptors. Activation of AMPA receptors can potentiate insulin and glucagon secretion by modulating the intrinsic properties of α- and β-cells (Bertrand et al., 1993; Gonoi et al., 1994; Inagaki et al., 1995; Liu et al., 1997b; Weaver et al., 1996 and 1998). The endocrine pancreas can be influenced by glutamate via activation of metabotropic glutamate receptors (Tong et al., 2002). Etc. NMDA receptor channels are also likely constitutively expressed in rat adrenal medulla, and adrenal medulla secretion may be regulated by glutamate (Watanabe et al., 1994; Yoneda and Ogita 1986; Kristensen, 1993; Hinoi et al., 2002c). In the pineal gland, pinealocytes accumulate L-glutamate in microvesicles and secrete it through an exocytic mechanism. The secreted glutamate binds to constitutively expressed metabotropic glutamate receptors and inhibit norepinephrine-stimulated melatonin synthesis in neighboring pinealocytes. GluR1, a functional isoform of the AMPA glutamate receptor, might participate in a signaling cascade that enhances and expands a cholinergic and glutamatergic signal throughout the pineal gland (Yatsushiro et al., 2000). These pineal systems are believed to function as a negative regulatory mechanism for melatonin synthesis (Govitrapong et al., 1986; Yamada et al., 1998; Yatsushiro et al., 2000). Finally, in the pituitary gland, GluR6:7 subunits of kainate receptors are likely constitutively expressed in some anterior pituitary cells, and pituicytes in the neural lobe thus may be probably regulated by glutamate (Hinoi and Yoneda, 2001; Kiyama et al., 1993). Etc.

Tianeptine may be even useful in certain sexual disorders. In fact, there is evidence for the expression of glutamate receptors in testis (immunocytochemical analysis of bioptic samples from human testis shows a high expression of mGlu5 receptors inside the seminiferous tubuli, whereas mGlu1a immunoreactivity is restricted to intertubular spaces). In mature human sperm, mGlu5 receptors are present and active (Storto et al., 1999 and 2001; Nagata et al., 1999). Some sexual responses, in particular penile erection, are controlled by neural circuits in the brain and spine that are stimulated by the binding of excitatory amino acids to postsynaptic NMDA receptors. In penile, urinary bladder and ventral prostate tissues from adult male rats, and homologous surgical tissues from human male patients, all the essential NMDA receptors subunits are present. These tissues bind NMDA receptor ligands and NMDA receptor antagonists relax strips of these tissues (Gonzalez-Cadavid et al., 2000).

Therefore, the present invention extends the use of tianeptine, salts thereof, isomers thereof, pro-drugs thereof, metabolites thereof and structural analogs thereof, in the preparation of medicaments to be used for prevention and/or treatment of all conditions and disorders which are related to (absolute or relative) deficiency or excess of glutamate, in particular selected from those disclosed in Tables 2 and 3, whatever their cause and their pathophysiology, with the exception of the already proposed clinical applications of tianeptine (disclosed in Table 5). The present invention also concerns methods of treatment for these additional conditions and disorders which are related to or result in (absolute or relative) deficiency or excess of glutamate, comprising administering an efficient amount of tianeptine or a salt thereof, an isomer thereof, a pro-drug thereof, a metabolite thereof or a structural analog thereof. Preferably, said conditions and disorders are selected from those disclosed in Tables 2 and 3, excluding those disclosed in Table 5. In fact, all the already proposed clinical applications of tianeptine, including asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of AIDS, . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke, are likely related to or result in, at least in part, (absolute or relative) deficiency or excess of glutamate. Thus the additional target conditions and disorders for tianeptine complete these already claimed conditions and disorders. They comprise in particular the above mentioned conditions and disorders from “peripheral” SOCs. Considering the large and complex distribution of the different glutamate receptor subtypes, the importance and variety of their already known physiological and pathophysiological roles, and the quasi ubiquitous distribution and critical role of GS in cellular metabolism, a myriad of central and peripheral disorders can be speculated to be related to or result in (absolute or relative) deficiency or excess of glutamate and to benefit from a treatment with tianeptine. Considering that it is almost impossible to clearly distinguish the conditions and disorders which can be the targets of tianeptine due to its effects on glutamate from those due to its effects on de novo synthesis of glutamine, all the potential indications have been gathered in the Tables 2 and 3 which present a list of conditions and disorders which (can) benefit from medications activating, modulating/regulating or inhibiting GS activity. These conditions and disorders are reported according to the MedDRA classification (MedDRA Version 7.1). For most system organ classes (SOC), we have indicated the included high level group terms (HLGT) of particular interest; for certain HLGT, we have indicated the included high level terms (HLT) of particular interest; for certain HLT, we have indicated the included preferred terms (PT) of particular interest. Potential indications, for which we have emphasized that they are or can be related to (absolute or relative) deficiency or excess of glutamate, are marked in “italic”. All the already claimed or proposed clinical applications of tianeptine are “bold-underlined”. Potential factors and mechanisms which can underlie these conditions and disorders are disclosed in Table 4, without to be limited thereto.

Similarly, instead of tianeptine, GS ligands selected from the group consisting of GF, MS, NF, hydrazines and bisphosphonates, and salts thereof, isomers thereof, pro-drugs thereof, metabolites thereof and structural analogs thereof, can also be used, at “low doses” i.e. doses resulting in concentrations which regulate GS activity (directly or indirectly) in the target cells/tissues/organs/organism, to prevent and/or treat these conditions and disorders. In particular, GS ligands selected from the group consisting of GF, MS, NF, hydrazines and bisphosphonates, and salts thereof, isomers thereof, pro-drugs thereof, metabolites thereof and structural analogs thereof, can be used in the preparation of medicaments intended for prevention and/or treatment of conditions and disorders which are related to or result in (absolute or relative) deficiency or excess of glutamate, in particular selected from those disclosed in Tables 2 and 3, whatever their cause and their pathophysiology, with the exception of: if said GS ligand is GF or MS, the conditions and disorders related to cerebral ischemia, hyperammonemia, bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is NF the conditions and disorders disclosed in Table 14; if said GS ligand is a hydrazine, the conditions and disorders disclosed in Table 6; and, if said GS ligand is a bisphosphonate, the conditions and disorders disclosed in Table 7.

GS ligands selected from the group consisting of tianeptine, GF, MS, NF, hydrazines and bisphosphonates, and salts thereof, isomers thereof, pro-drugs thereof, metabolites thereof and structural analogs thereof, may be in particular combined with treatments preventing from excitotoxic properties of glutamate through other mechanisms e.g. with drugs altering glutamate receptor(s) function(s), in particular with NMDA glutamate receptor antagonists (D-AP5, ketamine, memantine, MK-801, . . . ), and glucocorticoids (to decrease the doses of glucocorticoids and/or prevent adverse effects of glucocorticoids. The present invention contemplates also a pharmaceutical composition comprising such combination and its use for the preparation of a medicament or in a method of treatment.

### Functions, Conditions and Disorders Related to Glutamine

In animals, glutamine is the most abundant free amino acid. It has the largest free pool and one of the highest fluxes over organs of all amino acids (Young and Ajami, 2001). Glutamine serves as a fuel source that is more or less important depending on the cell type and (patho)physiological condition; for instance it is crucial in the gastrointestinal tract and immune system (Wilmore, 2001; Newsholme, 2001). It is involved in essential cellular syntheses. It provides nitrogen for the synthesis of purine and pyrimidine nucleotides, which are necessary for the synthesis of new DNA and RNA. It is involved in the synthesis of agents like glutamate, glutathione, gamma aminobutyric acid, glucose but also arginine, citrulline, tryptophane, . . . . It participates in homeostatic functions e.g. in maintaining normal acid-base balance by providing the ammonia that is necessary to counterbalance acidic compounds and/or in carrying potentially toxic ammonia to the kidneys for excretion. During metabolic acidosis, the kidneys can siphon off large amounts of glutamine. It is involved in the production of the body's most important antioxidants, glutathione (Welbourne and Dass, 1982; Cao et al., 1998; Hong et al., 1992; Manhart et al., 2001) and the amino acid taurine (Boelens et al., 2003). Glutathione is present at high concentrations in most mammalian cells and has many protective and metabolic functions. It is quantitatively the most important endogenous scavenger with the ability to hinder from oxidative injury caused by oxygen-derived free radicals and peroxides (Beutler, 1989). Glutathione protects cell membranes by reducing peroxides. By maintaining the thiol groups of many proteins in the reduced form, it ensures their normal function, thereby influencing the activity of several enzymes (Meister, 1991). Glutathione also prevents activation of transcription factor nuclear factor-kB, a stimulator involved in the synthesis of certain cytokines and adhesion molecules (Roth et al., 2002), and thus inhibits inflammatory responses. Etc. All this illustrates the critical role of glutamine in vivo (for review see Labow et al., 2001; Hertz and Zielke, 2004; Melis et al., 2004).

Animals have evolved normally effective mechanisms to control glutamine homeostasis. These mechanisms are usually effective in maintaining a constant plasma glutamine level, even when faced with variable dietary intake and changing glutamine demand. Significant decreases in plasma glutamine concentration are only observed in states of critical illness e.g. cancer with intense chemotherapy and/or radiotherapy, endotoxemia, sepsis, starvation, surgery, trauma, implicating that glutamine may become a conditionally essential amino acid in patients with catabolic disease(s) (Houdijk et al., 1998; Labow et al., 1998; Planas et al., 1993; Eaton, 2003; Miller, 1999; Neu et al., 2002). In catabolic states, which are characterized by a loss tissue/body mass related to the disease itself, to its treatment and/or to physical inactivity (Cuthbertson et al., 1979), the consumption of glutamine exceeds glutamine synthesis and supply from proteolysis, resulting in depletion of the glutamine stores; large amounts of glutamine are released from muscle (Karinch et al., 2001) while glutamine consumption in certain cells and tissues e.g. in the immune system increases (Newsholme, 2001). The goal of nutritional support in catabolic patients is to prevent loss of lean body mass as far as possible. Besides preventing loss of lean body mass, nutritional supplementation with high-doses of glutamine influence the response of the patient to metabolic stress, in particular by protection of body cells from oxidative stress, enhancement of the immune system and enforcement of the gut barrier function (Melis et al., 2004).

In certain cells e.g. in astrocytes, all the glutamine has to be synthesized de novo from glutamate and ammonia by GS (see above). In others e.g. for certain functions in the gastrointestinal tract, de novo synthesized glutamine is more effective as compared to exogenous glutamine (see above; Weiss et al., 1999; Le Bacquer et al., 2001; Li et al., 2004). Consequently, GS activity is or may become conditionally limiting in certain cells, tissues or organs. For instance, GS activity in glial cells is critical for the moment-to-moment sustenance of astrocytic but also neuronal function in the nervous tissue (Barnett et al., 2000). In certain pathologic states, although glutamine plasma level is normal, glutamine deprivation can occur in specific cells/tissues, due to either an increase of local demand and/or insufficient local synthesis of glutamine. It might be the case for the dendritic atrophy of CA3 pyramidal neurons induced by glucocorticoids against which we observed that tianeptine but also other GS regulating ligands could be protective (FIG. 30).

GS activity and expression are susceptible to inhibition by oxidative stress (Kosenko et al., 2003). During most critical cellular, tissular, organ, multi-organ as well as whole body disorders/failures, a state of oxidative stress occurs due to an imbalance between increased production of reactive oxygen species and natural antioxidant depletion. This can trigger death in certain cells in case of glutamine deprivation (Lee et al., 1998; Shohami et al., 1999; Eaton, 2003). In certain catabolic states, glutamine supplementation can restore decreased glutathione concentrations and, to some extent, prevent ongoing oxidative stress with the subsequent risk of tissue damage (Robinson et al., 1992; Flaring et al., 2003). Glutamine also regulates the production of taurine. One function of taurine is to trap chlorinated oxidants by producing the nontoxic, long-lived, taurine chloroamine, and therefore protect the cell from self-destruction (Marcinkiewicz et al., 1995). As taurine is exceptionally abundant in the cytosol of inflammatory cells, especially in neutrophils, taurine travels with the migrating neutrophils to the damaged tissue to combat free radicals (Koyama et al., 1992). Glutamine supplementation was also observed to prevent extracellular water retention in catabolic patients; and this effect could be also exercised through taurine, because taurine can serve as an osmoregulator (Scheltinga et al., 1991). Glutamine has been also shown to increase survival of distal pulmonary epithelial cells during hyperoxia; while high oxygen concentrations which are used in the treatment of acute respiratory distress syndrome and hyaline membrane disease are known to damage alveolar epithelial cells through the release of free oxygen radicals (McGrath-Morrow and Stahl, 2002).

So, various agents and mechanisms regulate GS expression and activity in a tissue-specific fashion. For instance, an increase of glutamine export by skeletal muscles and lungs is associated with increased glutamine consumption by other organs such as the gut, liver and immune system, in response to various stresses; unlike skeletal muscle, which contains substantial quantities of free glutamine, the lung primarily contributes glutamine synthesized de novo from glutamate and ammonia (Ardawi, 1990; Labow et al., 1998 and 1999). Glucocorticoid hormones, which are the primary mediator of GS expression during stress, act on the lung and skeletal muscle rather fast. This hormonal regulation allows GS mRNA levels to increase during catabolic states. However, the ultimate level of GS enzyme expression is further governed by a post-transcriptional mechanism regulating GS protein stability. In a unique form of product feedback, GS protein turnover is increased by glutamine. This mechanism appears to provide a way to index the production of glutamine to its intracellular concentration and, therefore, to its systemic demand (Labow et al., 2001). Both GS mRNA and GS protein expression increase markedly in atrophic muscle after denervation (Falduto et al., 1992) like after chronic exposure to exogenous glucocorticoids (Hickson et al., 1996). In addition to produce significant muscle atrophy, both of these stimuli induce glutamine efflux and deplete muscle glutamine stores (Babij et al., 1986; Hundal et al., 1990).

The Inventors already (see above) mentioned that functional glutamate receptors are present in different thymic cell compartments and might have a role in the intrathymic lympho-stromal relationships regulating thymocyte differentiation (Storto et al., 2000a). Glutamate receptors were also found on peripheral blood lymphocytes where elevated extracellular glutamate concentrations inhibit peripheral lymphocyte function (Droge et al., 1988; Kostanyan et al., 1997; Lombardi et al., 2001). In addition, glutamine has been demonstrated to play important roles in the immune response. It provides nitrogen for the synthesis of purine and pyrimidine nucleotides, which are necessary for the synthesis of new DNA and RNA during proliferation of lymphocytes, and for mRNA synthesis and DNA repair in macrophages (Newsholme, 2001; Curi et al., 1999). It is an important source of energy for the white blood cells. Freshly isolated lymphocytes, macrophages and neutrophils utilize glutamine at a rate similar to or greater than glucose (Ardawi and Newsholme, 1983). Neutrophils increase their phagocytic activity and rate of production of superoxide when glutamine is offered, in a dose-dependent manner (Furukawa et al., 2000). Furthermore, glutamine was shown to improve the function of neutrophils by reducing the adrenaline-induced inhibition of superoxide production (Garcia et al., 1999), and improving their mitochondrial functionality, ATP generation and protection from apoptosis (Pithon-Curi et al., 2003); the latter effect may be mediated by glutathione. In patients undergoing surgery, parenteral supplementation with glutamine increase HLA-DR expression on monocytes postoperatively (Spittler et al., 2001). Boelens et al. confirmed this result in trauma patients which was accompanied by a decrease in infectious morbidity (Houdijk et al., 1998; Houdijk et al., 1999; Boelens et al., 2002). Glutamine, therefore, is believed to be crucial for the functionality of the immune system.

The Inventors already mentioned that glutamine is utilized as a major fuel and nucleotide substrate by intestinal mucosal cells and the gut-associated immune system (Scheppach et al., 1994; McCauley et al., 1998; Wiren et al., 1998), and therefore could prevent intestinal atrophy in certain conditions or disorders (Ziegler et al., 2000 and 2003). The barrier function of the gastrointestinal tract can be impaired following stresses due to injury or surgery, as well as inflammation (Bjarnason et al., 1995; Israeli et al., 2004). This loss of barrier function may play a role in the translocation of bacteria and endotoxins across the gut wall, subsequently resulting in sepsis, prolonged systemic inflammatory response or, eventually, multiple organ failure (Deitch, 1992; Mainous et al., 1994). In an animal experiment, Potsic et al. studied the effect of dietary and endogenously produced glutamine on gut integrity in artificially fed baby rats (Potsic et al., 2002). Glutamine supplementation turned out to improve gut wall integrity. Methionine sulfoximine was given as a glutamine synthetase inhibitor and subsequently gut wall integrity was decreased.

So there is compelling evidence indicating that de novo synthesis of glutamine by glutamine synthetase plays an important role to maintain for normal cell, tissue and organ functions.

On the contrary, other works have draw attention to the impact of excess of de novo glutamine synthesis in certain conditions and disorders e.g. in hepatic encephalopathy where increased accumulation of glutamine was observed to aggravate anaerobic glycolysis and energetic failure (Albrecht, 2003; Zwingmann et al., 2003). Excess of glutamine induces mitochondrial permeability transition (MPT) in astrocytes (Rao et al., 2003); astrocytic mitochondria are more vulnerable to the effects of glutamine in induction of MPT compared to neuronal mitochondria e.g. to manganese induced MPT (Rao et al., 2004). The mechanism(s) by which glutamine selectively induces MPT in astrocytes is not completely understood. However, increased production of free radicals and associated oxidative stress are generally considered major factors in MPT induction (Castilho et al., 1995; Halestrap et al., 1997). Similarly, treatment of various cells with antioxidants showed attenuation of MPT, including in astrocytes exposed to laser irradiation (Jou et al., 2002) and in rat hepatocytes treated with ethanol (Higuchi et al., 2001). Glutamine has been shown to cause the production of free radicals in astrocytes in a concentration/dose-dependent manner (Jayakumar et al., 2004). So it is likely that glutamine-induced MPT is mediated by oxidative stress. MPT induction by excess of glutamine in astrocytes may be due to increased free radical production by hydrolysis of glutamine, and high ammonia concentrations in mitochondria resulting from glutamine hydrolysis may be responsible for the effects of glutamine; treatment of astrocytes with the mitochondrial glutaminase inhibitor, 6-diazo-5-oxo-L-norleucine, completely blocked free radical formation and MPT (Murthy et al., 2001; Jayakumar et al., 2004; Norenberg et al., 2004). Finally, by interfering with mitochondrial functions, excess of de novo synthesis of glutamine may participate to impairment of energy metabolism in susceptible cells in other disorders e.g. inflammatory, immunological or ischaemic disorders.

Accordingly, the present invention concerns the use of tianeptine, salts thereof, isomers thereof, pro-drugs thereof, metabolites thereof and structural analogs thereof, in the preparation of medicaments to be used for prevention and/or treatment of conditions and disorders which are related to (absolute or relative) deficiency or excess of glutamine and/or sensitive to glutamine deprivation or glutamine supplementation, whatever their cause and their pathophysiology, with the exception of the already proposed clinical applications of tianeptine, which are disclosed in Table 5. All the already proposed clinical applications of tianeptine, including asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of AIDS, . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke, might be related to a (absolute or relative) deficiency or excess of glutamine. Thus, the additional target conditions and disorders for tianeptine complete these already claimed conditions and disorders. They comprise in particular the above-mentioned conditions and disorders from “peripheral” SOCs. Considering the critical role of glutamine in vivo and the quasi ubiquitous distribution of GS, a myriad of central and peripheral disorders can be speculated to be related to an (absolute or relative) deficiency or excess of glutamine and to benefit from a treatment with tianeptine. Since it is quasi impossible to distinguish the conditions and disorders which can benefit from the effects of tianeptine on de novo synthesis of glutamine from those which can benefit from its effects on glutamate metabolism, all these potential indications have been gathered in the Tables 2 and 3. These tables present a list of conditions and disorders which can benefit from drugs prone to regulate GS activity. As above-mentioned, hyperammon(em)ia can result in an excess of glutamine. Therefore the conditions and disorders which (can) result, alone or combined, in a localized or a systemic ammonia increase are also claimed (see below; Table 1 and 4). All these conditions and disorders are reported according to the MedDRA classification (MedDRA Version 7.1). For most system organ classes (SOC), we have indicated the included high level group terms (HLGT) of particular interest; for certain HLGT, we have indicated the included high level terms (HLT) of particular interest; for certain HLT, we have indicated the included preferred terms (PT) of particular interest. All the already claimed or proposed clinical applications of tianeptine are “bold-underlined”. Potential factors and mechanisms which can underlie these conditions and disorders are disclosed in Table 4, without to be limited thereto.

Similarly, instead of tianeptine, GS ligands selected from the group consisting of GF, MS, NF, hydrazines and bisphosphonates, and salts thereof, isomers thereof, pro-drugs thereof, metabolites thereof and structural analogs thereof, can also be used in the preparation of a medicament intended for prevention and/or treatment of conditions and disorders which are related to (absolute or relative) deficiency or excess of glutamine and/or sensitive to glutamine deprivation or glutamine supplementation, whatever their cause and their pathophysiology, at “low doses” i.e. doses resulting in concentrations which can activate or inhibit i.e. regulate GS activity in the target cells/tissues/organs/organism (directly or indirectly), with the exception of: if said GS ligand is GF or MS, the conditions and disorders related to cerebral ischemia, hyperammonemia, bacterial, viral and fungal infectious disorders (antimicrobial effect), neoplasm (cytotoxic effect), neurogenerative diseases (Alzheimer disease, Huntington's and other polyglutamine disorders) and pain; if said GS ligand is NF the conditions and disorders disclosed in Table 14; if said GS ligand is a hydrazine, the conditions and disorders disclosed in Table 6; and, if said GS ligand is a bisphosphonate, the conditions and disorders disclosed in Table 7. For these ligands, all the already proposed clinical applications of tianeptine i.e. asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of AIDS, . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke, which might be related to a (absolute or relative) deficiency or excess of glutamine, are claimed.

Thus “low doses” of a GS ligand selected from the group consisting of tianeptine, GF, MS, NF, hydrazines and bisphosphonates, and salts thereof, isomers thereof, pro-drugs thereof, metabolites thereof and structural analogs thereof, may serve to support/regulate GS (homeostatic) activity/role in preventing and/or treating conditions and disorders related to critical cellular stresses, in particular in conditions of hyperammon(em)ia and/or excess of glutamate, or in catabolic states. Potential factors and mechanisms which can underlie these conditions and disorders are disclosed in Table 4, without to be limited thereto.

Preferably, a “low dose” of a GS ligand selected from the group consisting of tianeptine, GF, MS, NF, hydrazines and bisphosphonates, and salts thereof, isomers thereof, pro-drugs thereof, metabolites thereof and structural analogs thereof, will be combined with other etiologic and/or symptomatic measures e.g. glutamine nutritional supplementation in catabolic states including AIDS, cancer, heart failure, infection, inflammation, ischaemia, trauma, . . . , in intensive care unit patients, subjects engaged in intense exercise, . . . , to help in preserving the immune and gut barrier functions, and accelerate their rate of recovery, or measures to control nitrogenous supply in certain conditions and disorders associated with excess of glutamine. Therefore, the present invention concerns a pharmaceutical composition comprising a GS ligand selected from the group consisting of tianeptine, GF, MS, NF, hydrazines and bisphosphonates, and salts thereof, isomers thereof, pro-drugs thereof, metabolites thereof and structural analogs thereof, and other etiologic and/or symptomatic drugs. In particular, GS ligands selected from the group consisting of tianeptine, GF, MS, NF, hydrazines and bisphosphonates, and salts thereof, isomers thereof, pro-drugs thereof, metabolites thereof and structural analogs thereof, may be associated with glucocorticoids to improve their efficacy and/or prevent their adverse effects.

Inhibition of glutamine synthesis has the potential to reduce cell growth and to inhibit glutathione synthesis in tumors; if so, such inhibition should increase oxidative stress and hinder the ability of tumor cells to resist the effects of both chemical and radiation therapy.

Accordingly, the present invention concerns the use of GS ligands selected from the group consisting of tianeptine, GF, MS, NF, hydrazines and bisphosphonates, and salts thereof, isomers thereof, pro-drugs thereof, metabolites thereof and structural analogs thereof, for the preparation of cytolytics medicaments, for instance to combat neoplasms or certain inflammatory diseases. GS ligands selected from the group consisting of tianeptine, GF, MS, NF, hydrazines and bisphosphonates, and salts thereof, isomers thereof, pro-drugs thereof, metabolites thereof and structural analogs thereof, are administered to obtain a target-specific drug delivery i.e. concentrations which “saturate” the subunits of the enzyme in the targeted cells/tissues/organs (administration with a targeted carrier, local administration, . . . ), and/or combined with anti-proliferative (5-fluorouracil, asparaginase, . . . ) and/or anti-inflammatory therapies. The therapeutical amount result in concentrations of compound which can regulate GS activity in the rest of the organism i.e. normal cells/tissues/organs.

Others Organisms with Eukaryotic Cells and Prokaryotes

Others (all) organisms with eukaryotic cells such as invertebrate animals (arthropods, protozoa, shellfish, worms, . . . ), algae, fungi (yeasts, molds, . . . ) or plants, and also prokaryotes e.g. algae or bacteria, express GS (iso)enzyme(s). In these (lower) organisms GS proteic structure(s), regulation(s) and (patho)physiological role(s) differ to some extent compared to those observed in vertebrate animals.

For instance in plants, GS occurs in two major octameric forms, one in the cytosol (GS1) and one in the chloroplast (GS2), with a highly sophisticated pattern of distribution and regulation (Lea, 1997; Miflin and Habash, 2002; Ishiyama et al., 2004). GS1 may be either homomeric or heteromeric; molecular biological studies have identified a number of genes encoding GS1 subunits from various plant species. The isoenzymes of GS1 show organ- and cell-specific patterns of expression and are developmentally regulated. Edwards et al. using promoter analysis of the GS3A gene of pea, suggested that cytosolic GS is preferentially expressed in the vascular tissue of leaves (Edwards et al., 1990). Many subsequent studies have confirmed the importance of the location of GS1 in the phloem and related vascular tissues (Tobin and Yamaya, 2001). GS1 is also localized in roots, and in a number of specialist tissues and organs involved in the generation and transport of reduced nitrogen. For instance, a nodule-specific GS1 isoenzyme is formed during the onset of nitrogen fixation (Lara et al., 1983) and one of the maize GS1 genes is preferentially highly expressed in the pedicels of developing kernels (Rastogi et al., 1998). GS1 diversity leads to very sophisticated changes in the nature of GS as the plant and its individual organs pass through different development stages. For instance, the GS1 subunit composition of sugar beet changes with respect to nitrogen nutrition and organ ontogeny (Brechlina et al., 2000). Changes in subunit composition in Phaseolus vulgaris leaves are due to the differential expression of the various GS1 genes during development and ageing (Cock et al., 1991). On the contrary, the plastidic form of GS (GS2), which is widely distributed in the chloroplast, is generally regarded as universal; GS2 is also present in plastids in roots and other non-green tissues, this distribution differing between species and with respect to plastid subtypes (Tobin and Yamaya, 2001). Finally in plants, the majority of primary nitrogen enters through the roots as nitrate, and nitrate reductase and nitrite reductase sequentially reduce the nitrogen to ammonium. GS1 is the major form of GS in roots, and so the ammonium taken up from the soil is directly converted to glutamine by its reaction; the role of GS2 in leaves is to reassimilate the NH₃ generated by photorespiration. The amide group from glutamine can be then transferred to glutamate by the action of the glutamate synthase (GOGAT). So ammonium is assimilated into glutamine and glutamate through a consecutive reaction of GS and glutamate synthase (GOGAT). This so-called GS/GOGAT cycle is of crucial importance for growth and development in plants; in fact, glutamine and glutamate are the donors for the biosynthesis of major nitrogen-containing compounds, including amino acids, nucleotides, chlorophylls, polyamines and alkaloids (Miflin and Lea, 1980; Lam et al., 1996; Lea et al., 1997; Miflin and Habash, 2002).

The nature of the metabolism occurring via GS depends on the environment of the plant, which may act directly or through the metabolic status of the plant and its different tissues, and varies over the course of the day (Stitt et al., 2002). The reactions and the forms of GS involved differ according to the plant organ under consideration. Within an organ, the role of GS and the metabolism in progress differ according to the tissue, cell or subcellular compartment. Within any location, the metabolism differs according to the developmental stage of that part of the plant. In this regard, it is important to realize that developmental stages such as vegetative and reproductive growth are not linear but overlapping. Thus, the nature of GS and its regulation has to be approached by taking into account the multidimensional nature of nitrogen metabolism and appreciating the large differences that occur in glutamine metabolism between various locations in the matrix. The plant has evolved mechanisms to enable it to cope with this complexity and which enable it to survive in competition with other plants in its environment. In seed plants, this must place the greatest importance on the success of the seed, because mechanisms that do not support effective reproduction will not have been maintained during evolution.

Because engineering of nitrogen assimilation is very important agriculturally, improvement of nitrogen assimilation by molecular approach has been attempted for several years. Recent reports have shown that overproduction of GS can lead to a better performance of nitrogen utilization through the promoted recycling of ammonia released during photorespiration but can not enhance net nitrogen assimilation. GS transgenic plants showed improved growth under nitrogen-limiting conditions only when they were grown initially under nitrogen-sufficient conditions (Migge et al., 2000; Fuentes et al., 2001). On the contrary, overexpressing GS activity in roots can lead to a decrease in plant biomass production due to a lower nitrate uptake accompanied by a redistribution to the shoots of the newly absorbed nitrogen which can not be reduced due to the lack of nitrate reductase activity in this organ (Limami et al., 1999). Nitrogen assimilation requires not only inorganic nitrogen but also the carbon skeleton 2-oxoglutarate (2-OG) that is produced through sequential reactions from photoassimilated carbohydrates (Gallardo et al., 1999; Migge et al., 2000; Fuentes et al., 2001; Oliveira et al., 2002; Limami et al., 2002).

While MS and GF, and derivatives thereof exhibit herbicidal activity at “high concentrations” (this effect is accounted for by impairment of nitrogen metabolism, resulting from inhibition of GS; excess ammonium and glutamine deficiency act in concert to cause plant death) (Lea and Ridley, 1989; Sadunishvili et al., 1996), lower concentrations of MS and GF, and derivatives thereof have been reported recently to have an opposite effect; glutamine synthetase was observed to be activated with a concomitant stimulation of plant growth and productivity (Evstigneeva et al., 2003). Accordingly, the present invention concerns also the use of tianeptine or NF, salts thereof, isomers thereof, pro-drugs thereof, metabolites thereof and structural analogs thereof, in the manufacture of treatments intended for the regulation or inhibition of all types of functions which involve GS activity or can be influenced by GS activity, directly or indirectly, with the aim to facilitate plant growth or protect plants from certain stresses and disorders, or combat plant growth, respectively. Therefore, in a particular embodiment, the present invention concerns the use of tianeptine or NF, salts thereof, isomers thereof, pro-drugs thereof, metabolites thereof and structural analogs thereof, at “low doses”, to facilitate plant growth or protect plants from certain stresses and disorders. In an alternative embodiment, the present invention concerns the use of tianeptine or NF, salts thereof, isomers thereof, pro-drugs thereof, metabolites thereof and structural analogs thereof, at “high doses”, as herbicides.

In particular, tianeptine and NF, or salts thereof, isomers thereof, pro-drugs thereof, metabolites thereof or structural analogs thereof, may be associated crop tolerant to glufosinate i.e. expressing the gene coding for phosphinothricin tolerance (bar), which encodes the enzyme phosphinothricin acetyltransferase (PAT) (PAT is derived from Streptomyces hygroscopicus and Streptomyces viridochromogenes; EP 275957 was filed in 1987 by Aventis CropScience (former AgrEvo), with the aim to facilitate their growth, protect them from certain stresses and disorders, or combat their growth.

At low rates, GF was previously observed to improve the yield of crop plants which are resistant to glutamine synthetase inhibitors (U.S. Pat. No. 5,739,082). However, the mechanism underlying this latter effect is still unknown.

According to recent reports (United Nations Programme) worldwide nitrogen pollution is one of the main threats to human survival and environment. Sixty percent of nitrogen resulting from human activities is inorganic crop fertiliser, the global use of which has increased 10-fold in the last half century (UNEP, 1999). Plants typically take up less than half of the N fertiliser applied with the majority lost to the atmosphere or dissolved in surface of groundwater (Vitousek et al., 1997). Nitrogen is a rate-limiting element in plant growth. Although the ability of plants to take up nitrate or reduce nitrate uptake is not limited, it is the ability to incorporate nitrogen into proteins which appear to be limited (Lam et al., 1995). Nitrogenous fertiliser accounts for 40% of costs associated with crops (Sheldrick et al., 1987). Increasing the efficiency of nitrogen use would be cost effective and would minimise problems of ground water contamination by excess nitrate application (Sheldrick et al., 1987). Conversely in starving condition, when plants suffer from low concentration of nitrogen or light (in photosynthetic plants), activation of GS would facilitate assimilation of NH₄ and change plants homeostasis, which would help them better resist the stress (Glevarec et al., 2004).

Accordingly, the present invention concerns also the use of tianeptine or NF, or salts thereof, isomers thereof, prodrugs thereof, metabolites thereof or structural analogs thereof, in the manufacture of treatments intended to reduce inorganic crop fertilizer requirement. Tianeptine or NF, salts thereof, isomers thereof, pro-drugs thereof, metabolites thereof, and structural analogs thereof might be in particular associated with crop with a low N requirement (Foyer and Ferrario, 1994).

Conversely, high doses of tianeptine or NF can be used as a herbicide, since acute inhibition of GS by tianeptine or NF at normal growing conditions decreases the production of glutamine and increases the accumulation of ammonia resulting in death of plant.

Depending on the type of cell, tissue, organ, organism, species, physiological or pathophysiological condition, . . . , GS activity and de novo synthesis of glutamine can be more or less limiting. For instance, GS has been proposed to play a crucial role in the synthesis of a poly-L-glutamate-glutamine cell wall component which is found exclusively in pathogenic mycobacteria. Treatment of Mycobacterium tuberculosis with MS or with antisense oligodeoxyribonucleotides specific to Mycobacterium tuberculosis GS mRNA is associated with an inhibition of the formation of this poly-L-glutamate-glutamine cell wall structure. Paralleling this effect, these agents inhibit bacterial growth, indicating that GS plays an important role in these bacteria homeostasis. MS can block the growth in broth cultures of pathogenic mycobacteria, including M. tuberculosis, M. bovis, M. avium, but has no effect on non-pathogenic mycobacteria as well as non-mycobacterial microorganisms at the doses used. It blocks the growth of M. tuberculosis and M. avium within human mononuclear phagocytes, the primary host cells of these pathogens, and at the concentrations which are effective are completely non-toxic to the mammalian cells, likely reflecting a 100-fold greater sensitivity to MS of bacterial GS than of mammalian GS. Finally MS protects guinea pigs challenged by aerosol with a highly virulent strain of M. tuberculosis from i) death, ii) disease, as manifested by protection against weight loss, and iii) growth and dissemination of M. tuberculosis in animal organs, as manifested by decreased CFU in the lungs and spleen. MS acts synergistically with the major antituberculosis drug isoniazid (INH), reducing CFU in guinea pig organs by ˜1.5 log units below the level attained with INH alone. In comparison, GF was less selective and had only a very minor inhibitory effect on mycobacterium growth (U.S. Pat. No. 6,013,660), so as it has never been proposed to be used as a drug.

Accordingly, the present invention concerns the use of tianeptine or NF, salts thereof, isomers thereof, pro-drugs thereof, metabolites thereof and structural analogs thereof, in the preparation of medicaments for the treatment of pathogenic mycobacterium growth. The present invention also concerns a method of treatment of pathogenic mycobacterium growth in a subject comprising administering to said subject an efficient amount of tianeptine or a salt thereof, an isomer thereof, a pro-drug thereof, a metabolite thereof or a structural analog thereof. Preferably said pathogenic mycobacterium is selected from the group consisting of M. tuberculosis, M. bovis, M. avium, atypical mycobacteria (M. avium Complex, M. chelonae, M. fortuitum, M. kansasii, M. marinum, M. scrofulaceum, M. ulcerans), M. leprae more preferably the group consisting of M. tuberculosis, M. bovis, and M. avium. More preferably, said pathogenic mycobacterium is M. tuberculosis. More particularly, said treatment of pathogenic mycobacterium growth can be used as treatment of leprosy, paratuberculosis or tuberculosis.

More than 50 years after its discovery, isoniazid remains one of the primary drug for the chemotherapy of tuberculosis. Although several hypotheses have been proposed to explain its efficacy, its mechanism of action remains unclear. Its isopropyl derivative, iproniazid, is a well-established antidepressant. Developed in 1951 with isoniazid, it was found to have mood-elevating effects in patients with tuberculosis. In 1952, Zeller and Barsky found that iproniazid, in contrast to isoniazid, was capable of inhibiting the enzyme monoamine oxydase (MAO). Since then, iproniazid has been used for the treatment of depressed patients. MAO inhibitors had an important impact on the development of modern biological activity.

Based on the above-mentioned observations related to MS and tianeptine, the Inventors have tested isoniazid and iproniazid on GS activity and have found that both these drugs are GS ligands.

Accordingly, the present invention in particular concerns a method of treatment of pathogenic mycobacterium growth in a subject comprising administering to said subject an efficient amount of tianeptine or NF or a salt thereof, an isomer thereof, a pro-drug thereof, a metabolite thereof or a structural analog thereof, preferably associated with an other antituberculous agents e.g. isoniazid, ethambutol, pyrazinamide, rifampicin or streptomycin. Preferably said pathogenic mycobacterium is selected from the group consisting of M. tuberculosis, M. bovis and M. avium. More preferably, said pathogenic mycobacterium is M. tuberculosis. The present invention also concerns a pharmaceutical composition comprising such combinations.

On the contrary, activation of the GS is a feasible therapeutic strategy to protect bacteria from stress or facilitate their growth.

Finally, the present invention concerns the use of tianeptine, salts thereof, isomers thereof, pro-drugs thereof, metabolites thereof and structural analogs thereof, in obtaining activators, inhibitors and/or modulators of GS activity intended for the activation, inhibition and/or modulation of all types of functions which involve GS activity or can be influenced by GS activity, directly or indirectly, in non-vertebrate organisms with eukaryotic cells such as invertebrate animals (arthropods, protozoa, shellfish, worms, . . . ), algae, fungi (yeasts, molds, . . . ) or plants, and also prokaryotes e.g. in algae or bacteria, aiming to facilitate their growth or protect them from stresses and disorders, or, inversely, aiming to combat their growth. The present invention concerns methods to facilitate the growth, protect from stresses and disorders, or, inversely, combat growth, of non-vertebrate organisms with eukaryotic cells such as invertebrate animals (arthropods, protozoa, shellfish, worms, . . . ), algae, fungi (yeasts, molds, . . . ) or plants, and also of prokaryotes e.g. algae or bacteria, comprising the administration an efficient amount of tianeptine or a salt thereof, an isomer thereof, a pro-drug thereof, a metabolite thereof or a structural analog thereof.

### Pharmaceutical Form

The pharmaceutical composition according to the present invention can be presented in pharmaceutical forms that are suitable for administration by the oral, parenteral (intramuscular, intravenous, intratissular, intratumoral, subcutaneous, . . . ), per- or trans-cutaneous, nasal, rectal, perlingual, ocular or respiratory route, especially injectable preparations, aerosols, eye or nose drops, sublingual tablets, glossettes, soft gelatin capsules, hard gelatin capsules, lozenges, suppositories, creams, ointments, dermal gels, dermal patches, etc., those forms allowing the immediate release or delayed and controlled release, and/or targeted distribution of the active ingredient.

As basic salts, there may be used, without any limitation, sodium hydroxide, potassium hydroxide, calcium hydroxide or aluminium hydroxide, alkali metal or alkaline earth metal carbonates, or organic bases such as triethylamine, benzylamine, diethanolamine, tert-butylamine, dicyclohexylamine and arginine. As acid salts, there may be used without any limitation, hydrochloric acid, sulphuric acid, phosphoric acid, tartaric acid, malic acid, maleic acid, fumaric acid, oxalic acid, methanesulphonic acid, ethanesulphonic acid, camphoric acid and citric acid. A preferred salt of tianeptine is the sodium salt.

The dosage of the pharmaceutical composition according to the present invention varies principally according to the nature of the therapeutic indication. For instance, tianeptine, which p.o. dosage ranges from 12.5 mg to 300 mg per dose in its usual indications in human, could be effective at lower doses (<12.5 mg) in certain new indications, and GF within estimate of acceptable daily intake (<0.02 mg/kg) in certain indications.

### Screening Methods

In an other aspect of the present invention, the discovery of the target of tianeptine and NF, and that tianeptine but also GS ligands such as GF, MS, hydrazines and bisphosphonates can behave as activators or regulators of GS activity i.e. can activate, inhibit or be tolerated depending on the dose and experimental or (patho)physiological condition, allows to design methods for screening, identifying and/or developing new drugs for prevention and/or treatment of all conditions and disorders previously proposed as clinical applications of tianeptine i.e. those disclosed in Table 5 and in particular asthma, cough, depression (major depression with or without melancholia, dysthymic disorders, depressed bipolar disorder, . . . ), irritable bowel syndrome, mnemo-cognitive disorders, neurodegenerative diseases (such as cerebral hypoxia, cerebral ischaemia, cerebral traumatism, cerebral ageing, Alzheimer's disease, multiple sclerosis, amyotrophic lateral sclerosis, demyelinating pathologies, encephalopathies, myalgic encephalomyelitis, chronic fatigue syndrome, post-viral fatigue syndrome, the state of fatigue and depression following a bacterial or viral infection, the dementia syndrome of AIDS, . . . ), nonulcer dyspepsia, pain, psychoneurotic disorders (neurotic or reactive states of depression, anxiodepressive states with somatic complaints such as digestive problems, anxiodepressive states observed in alcoholic detoxification, . . . ), seizure, stress and stroke; and those disclosed in Table 14, and in particular Vascular disorders from Arteriosclerosis, stenosis, vascular insufficiency and necrosis, Embolism and thrombosis, Vascular disorders NEC, Vascular haemorrhagic disorders, Vascular inflammations and Venous varices groups, and Nervous system disorders from the Central nervous system vascular disorders, Encephalopathies, Mental impairment disorders, Movement disorders (incl Parkinsonism), Neurological disorders of the eye, Neuromuscular disorders and Seizures (incl subtypes) groups such as acute and chronic neurodegenerative diseases, Alzheimer's, Huntington's, Parkinson's diseases, multiple sclerosis, amyotrophic lateral sclerosis, spinal muscular atrophy, retinopathy, and traumatic brain injury, drug-induced neurotoxicity, pain, hormonal balance, blood pressure, thermoregulation, respiration, learning, pattern recognition, memory, and disorders subsequent to hypoxia or hypoglycaemia.

Preferred therapies according to the invention involve regulation, activation, inhibition and/or modulation of GS, in particular of astrocytic GS. The invention is generally useful for identifying and developing drugs intended to treat patients affected by conditions and disorders which involve or can be influenced by GS activity, directly or indirectly, in particular those related to absolute or relative excess of ammonia, those related to absolute or relative deficiency or excess of glutamate, and those related to absolute or relative deficiency or excess of glutamine, either isolated or associated, for instance those mentioned in Tables 1-4.

Preferred inhibitors of enzymatic conversion of glutamate to glutamine by GS are: drugs binding at the GS catalytic site such as tianeptine or NF or ligands like GF, MS and glutamate analogs, drugs which compete with amine at the amine binding site of the enzyme such as hydrazines like iproniazid and isoniazid, and drugs docking into GS Mn²⁺ binding sites such as bisphosphonates like pamidronate, risedronate and [(5-Chloro-pyridin-2-ylamino)-phosphono-methyl]-phosphinic acid.

The present invention concerns a method for screening, identifying and/or developing a new drug active in at least one of the conditions disclosed in Tables 5 and 14, wherein the method comprises the screening and the identification of compounds activating, inhibiting and/or modulating/regulating GS activity, in particular the activity of the native GS.

In a first embodiment, the method comprises:

a) contacting in vitro a candidate compound with GS or a fragment thereof; and,
b) determining the ability of said candidate compound to bind said glutamine synthetase or a fragment thereof.

Binding to said glutamine synthetase or a fragment thereof provides an indication of the compound's ability to modulate the activity of said glutamine synthetase.

In a second embodiment, the method comprises:

a) contacting in vitro a candidate compound with GS in conditions allowing GS activity; and,
b) determining the ability of said candidate compound to activate, inhibit and/or modulate the activity of said GS.

In a third embodiment, the method comprises:

a) adding to a cell expressing GS a candidate compound; and,
b) determining the ability of said candidate compound to activate, inhibit and/or modulate the activity of said GS.

Optionally, said screening method is performed in vivo, ex vivo or in vitro.

Preferably, said cell is an astrocyte. More preferably, said astrocyte is incubated with the candidate compound in absence of glutamine. The contacting step is performed for a period of time and under culture conditions adequate to expose the cell to glutamate and the candidate compound. The ability to activate and/or inhibit the GS activity can be performed by determining the cellular survival in the culture or the increase of glutamine in the extracellular medium. Optionally, said astrocytes can be in culture in the presence of neurones.

In a preferred embodiment of the screening methods according to the present invention, said GS fragment comprises or consists of the catalytic site, the amine binding site and/or the GS Mn²⁺ binding site. By fragment is intended for instance a peptide from 5 to 100, preferably 10 to 50 amino acids. More preferably, said fragment comprises or consists of more or less one part of the sequence ITGTNAEVMPAQWEFQIGPCEGIR (SEQ ID No 1).

In a preferred embodiment of the screening methods according to the present invention, said GS activity is the conversion of glutamate to glutamine, or of glutamine to glutamate.

In a fourth embodiment, the method uses at least one functional antibody directed against said GS. A functional antibody refers to an antibody, a fragment thereof or a derivative thereof, which specifically binds GS and is able to modify its activity. In a preferred embodiment of the screening methods according to the present invention, said GS fragment comprises or consists of the catalytic site or the amine binding site. By fragment is intended for instance a peptide from 5 to 100, preferably 10 to 50 amino acids. More preferably, said fragment comprises or consists of more or less one part of the sequence of SEQ ID No 1. This method of screening comprises contacting a candidate compound with a functional antibody and a target molecule, wherein the target molecule comprises an epitope of GS which is recognized by said functional antibody, and determining whether said candidate compound inhibits the binding of said functional antibody to said target molecule. Preferably, inhibition of the binding of said functional antibody to said target molecule is determined by inhibition ELISA. This method is detailed in the PCT patent application n^{o} WO2005040820.

In a fifth embodiment, the method comprises a step of molecular modeling using the 3D structure of GS (Gill and Eisenberg, 2001). The molecular modeling allows the selection and/or the design of drug which binds GS, more particularly in its catalytic site, in its amine binding site or in its Mn²⁺ binding site(s).

In a further embodiment, the method comprises determining the effect of said candidate compound on one or several exhibited symptoms in a subject. Said subject can be an animal or a human model for a condition or a disorder. Said subject can also be a patient.

In an additional embodiment, the method combines several above-mentioned methods. For instance, the method can comprise several steps selecting from the group consisting of a step of molecular modeling, a step of in vitro binding assay, a step of in vitro functional assay, a step of toxicity evaluation, and a step of in vivo functional assay.

The present invention also concerns a method for screening, identifying and/or developing a new biologically active compound (drug) comprising a competition assay on GS with a candidate compound and tianeptine. The new biologically active compounds (drugs) are those that show a capacity to compete with tianeptine or NF for the binding to GS. Competition assays are well-known by the man skilled in the art. For instance, said method comprises the following steps:
a) contacting in vitro, GS or a fragment thereof with a candidate compound;
b) adding tianeptine or NF; and,
c) determining the ability of said candidate compound to bind GS or a fragment thereof.

Preferably, said GS fragment comprises the catalytic site. More preferably, said fragment comprises or consists of the sequence SEQ ID No 1. Preferably, said tianeptine is labeled (e.g. by fluorescence or radioactivity). The ability of said candidate compound to bind GS or a fragment thereof is determined by the measure of the released tianeptine. This method can comprise additional steps such as determining the ability of the selected compound to activate, inhibit and/or modulate GS (in vitro and/or in vivo), determining the toxicity of the selected compound and determining the biological activity of the selected compound on a animal model, a healthy subject or a patient.

The present invention also concerns a method for screening, identifying and/or developing a new biologically active drug comprising a competition assay on GS with a candidate compound and a hydrazine like iproniazid or isoniazid. The new biologically active drugs are those that show a capacity to compete with a hydrazine for the binding to GS. Competitive assays are well-known by the man skilled in the art. For instance, said method comprises the following steps:
a) contacting in vitro GS or a fragment thereof with a candidate compound;
b) adding a hydrazine; and
c) determining the ability of said candidate compound to bind GS or a fragment thereof.

Preferably, said GS fragment comprises the amine binding site. Preferably, said hydrazine is labeled, (i.e., by fluorescence or radioactivity). The ability of said candidate compound to bind GS or a fragment thereof is determined by the measure of the released hydrazine.

The present invention also concerns a method for screening, identifying and/or developing a new biologically active drug comprising a competition assay on GS with a candidate compound and a bisphosphonate like pamidronate or risedronate. The new biologically active drugs are those that show a capacity to compete with a bisphosphonate for the binding to GS. Competitive assays are well-known by the man skilled in the art. For instance, said method comprises the following steps:
a) contacting in vitro GS or a fragment thereof with a candidate compound;
b) adding a bisphosphonate; and
c) determining the ability of said candidate compound to bind GS or a fragment thereof.

Preferably, said GS fragment comprises a Mn²⁺ binding site. Preferably, said bisphosphonate is labeled (e.g. by fluorescence or radioactivity). The ability of said candidate compound to bind GS or a fragment thereof is determined by the measure of the released bisphosphonate.

The methods include binding assays and/or functional assays, and may be performed in vitro, in cell systems, in vertebrates, etc. The above screening assays may be performed in any suitable device, such as dishes, flasks, plates, tubes, etc. Typically, the assay is performed in multi-wells plates. Several test compounds can be assayed in parallel. Furthermore, the test compound may be of various origin, nature and composition. It may be any organic or inorganic substance, such as a lipid, peptide, polypeptide, nucleic acid, small molecule, etc., in isolated or in mixture with other substances. The compounds may be all or part of a combinatorial library of products, for instance.

Further aspects and advantages of the present invention will be disclosed in the following experimental section, which should be regarded as illustrative and not limiting the scope of the present invention.

### EXAMPLES

### Material and Methods

Most of chemical products including glufosinate, iproniazid, isoniazid, L-methionine sulfoximine, pamidronate and tianeptine were provided by Sigma-Aldrich. Mice and rats were purchased from Charles River Laboratories and Harlan.

### 1 Screening of Tianeptine's Target Protein

### 1.1 Biotinylation of Tianeptine

Tianeptine was chemically coupled to a long chain EZ-Linked-biotin-PEO-amine (PEO: polyethylene oxide) according to the manufacturer recommendations Pierce). Biotin conjugated tianeptine was separated from unconjugated ones by using a phase inverse C18 column and eluted with acetonitrile (0-100% gradient). The purity of the biotinylated tianeptine was checked using mass spectrometry.

### 1.2 Isolation and Identification of Tianeptine's Target Protein

### 1.2.1 Membrane Preparation

Based on literature and on a preliminary immuno-histological study in mice administered intraperitonealy (i.p.) with biotinylated tianeptine, we choose to work on hippocampus membrane proteins. Hippocampi from 10 BALB/c mice were isolated, minced and left for 1 h in 300 mM Tris HCl solution buffer (TBS) on ice. Thereafter, the preparation was homogenised and centrifuged at 10400 g for 20 min at 4° C. The supernatant was centrifuged at 141400 g for 1 h at 4° C. The latter was repeated twice. Finally, the resultant pellet was diluted in 500 μl of TBS and stored at −20° C. until use.

### 1.2.2 SDS-PAGE and Western Blotting

Mice hippocampus membrane proteins (twenty μg per well) were separated on a 12% sodium dodecyle sulfate polyacrylamide electrophoresis (SDS-PAGE) gel in presence of 2β-mercaptoethanol. This was performed in absence and in presence of 100 mM n-octyl glucoside. The separated proteins were thereafter transferred to a nitrocellulose membrane (pore size 0.20 μm). Non-specific binding sites were blocked with 5% low fat skimmed milk in TBS buffer supplemented with 0.1% tween 20. The transferred proteins were allowed to react with biotinylated tianeptine (100 mM) for 2 h at room temperature. After one quick wash with TBS 0.1% tween 20, horseradish peroxidase conjugated streptavidin was subjected to the transferred proteins for 2 h at room temperature. Finally, the bound tianeptine, after several washes, was revealed using an electro chemiluminescence procedure (ECL).

### 1.2.3 2D Gel Electrophoresis and Western Blotting

Mice hippocampus membrane proteins were first focalized on a pH strip (range 3-10) over night at room temperature according to the standard procedure. The strip was then placed over a 12% acrylamide/bisacrylamide gel in presence of PDEA (2-(2PyridinylDithiol) Ethane Amine) and separated proteins were transferred to a nitrocellulose membrane (pore size 0.20 μm). Non-specific binding sites were blocked with 5% low fat skimmed milk in TBS buffer supplemented with 0.1% tween 20. Transferred proteins were allowed to react with biotinylated tianeptine (100 mM) for 2 h at room temperature. After one quick wash with TBS 0.1% tween 20, horseradish peroxidase conjugated streptavidin was subjected to the transferred proteins for 2 h at room temperature. Finally, after several wash, the bound tianeptine was revealed using ECL.

### 1.2.4 Mass Spectrometry Analysis—Sequence Determination

The spots issued from 2D gel electrophoresis/western blotting were digested and analysed for identifying the protein involved. The peptide sequences obtained form the latter were compared with sequences from protein banks.

### 1.3 In Silico Study of the Interaction Between Tianeptine and GS

Starting from the glufosinate (phosphinotricin)-glutamine synthetase complex structure (PDB 1FPY) (Gill and Eisenberg, 2001), a glutamine synthetase monomer was created in which glufosinate and Mn²⁺ were deleted. The tianeptine molecule was constructed in silico and minimalized using the AMPAC/MOPAC module of Accelerys (San Diego, Calif.). It was manually docked in the glutamine binding site of glutamine synthetase and the complex minimalized by conjugated gradient minimalization of the DISCOVER module fixing the atoms of the glutamine synthetase molecule. To explore the position of tianeptine in the binding site, a molecular dynamic simulation was performed at 300° K for 100 psec after an equilibration phase of 10 psec. A second complex was obtained with a lower potential energy than the minimalized one. After minimalization of this complex by conjugated gradient, a further approach was used to take into account the possible charge effects on the complex. The complex was charged at a pH of 7.4 and subjected to a new round of molecular dynamics using a distance dependent dielectric constant. Three conformations with the lowest potential energy were chosen for further minimalization by conjugated gradient using a distance dependent dielectric constant.

### 2 Characterization of GS Ligands Pharmacological Effects

### 2.1 Study of the Effects of GS Ligands In Vitro

### 2.1.1 Effects of GF, Iproniazid, MS and Tianeptine on Purified Sheep Brain GS Activity

The effects of glufosinate (GF), iproniazid, L-methionine sulfoximine (MS) and tianeptine on purified sheep brain GS activity were assessed according to Meister's method (Meister 1985). Briefly the assay mixture (final volume 0.5 ml) contained 0.1 M imidazole-HCl buffer (pH 7.2), 10 mM sodium arsenate, 20 mM MnCl₂, 125 mM hydroxylamine sodium, different concentrations of L-glutamine (0.1-50 mM), 20 μM ADP (sample buffer) and purified sheep brain GS. To study the effects on GS subunits (monomers), 25 mM 2β-mercaptoethanol (β-ME) was added to the assay mixture, with the exception of the experiments with tianeptine. In fact, tianeptine lost its activity in presence of β-ME due to its interaction with SO₂; thus β-ME had to be replaced by DTT (dithiothreitol) (25 mM). After 15 min incubation at 37° C., 0.5 ml of ferric chloride reagent (ferric [α]chloride 0.37 M with trichloroacetic acid 0.2 M) was added to stop the reaction. The formation of gamma-glutamylhydroxamate was assessed by reading the absorbance of the resultant solution at 535 nm against reagent blanks (the same sample buffer without either hydroxylamine, L-glutamine or ADP).

L-Glutamine+NH₂OH->L-γ-glutamyl-hydroxamate+NH₃
(in presence of sodium arsenate, Mn²° and ADP)
   2.1.2 Nature of the Interaction Between Tianeptine and GS Purified from Sheep Brain

### 2.1.2.1 Effect of a Over-Night Dialysis on the Binding/Effect of Tianeptine on Purified Sheep Brain GS

Two different concentrations of tianeptine (1 and 5 nM) were allowed to react with 3.4 units of GS purified from sheep brain. The reaction samples were prepared as follow: 200 μl of sample buffer (see above 2.1.1), 50 μl of tianeptine solution and 10 μl (3.4 units) of GS with ADP (20 μM). Half of the samples were immediately incubated for 15 min at 37° C. Before stopping the reaction with ferric chloride solution, a few μl of each sample were taken to be subjected to thin layer chromatography with a migration buffer composed of 9:1 DCM (dichloromethane)/methanol V/V. GS activity was assessed on the rest as mentioned above for purified sheep brain GS. The second half of the samples was removed to microtubes, which were dialysed against the sample buffer over night at 4° C. Before stopping the reaction, the day after, a few μl of each sample were taken to be subjected to thin layer chromatography with a migration buffer composed of 9:1 DCM (dichloromethane)/methanol V/V, and GS activity was assessed on the rest. Effect of L-glutamate on the effect of tianeptine on GS Activity

The effects of increasing concentrations of L-glutamate (0.016-10 mM) on the inhibitory effect of 5 μM tianeptine were assessed on GS purified from sheep brain. GS activity was assessed as mentioned above in 2.1.1. Higher concentration than 10 mM of L-glutamate (100 and 500 mM) increased background activity so no specific activity could be measured. The effect of tianeptine (100 nM) on extracellular free amine induced by glutamate (1, 10, 100, 500 μM) after 1 h incubation in C6 cells in serum free HBSS was also assessed. The effect of tianeptine (100 nM) in presence of N-methyl-D-aspartate (NMDA) (1, 10, 100, 500 μM) on extracellular free amine after 1 h incubation in C6 cells in serum free HBSS were tested for comparison. These experiments were performed according to the method mentioned below in 3.2.1.

### 2.1.3 Nature of the Interaction Between NF and GS

### 2.1.3.1. Interaction of NF and Purified Pig Brain GS in an Agarose Gel

Five ml of agarose 0.1% were let to polymerise in a Petri dish. Five wells with equal volume and distance from each other, 1 central and 4 peripherals, were made on it using a 300 μl tip. The central well was filed with 50 μl of NF solution (1 mg/kg). Fifty microlitre of purified pig brain GS (3 mg/ml) was added to one peripheral well. In two other wells either 50 μl of TBS or FCS was added. The gel was humidified and left at 37° C. for 48 h.

### 2.1.3.2 Interaction of NF and GS Using a Precipitation Method

Working buffer contained 0.1 M imidazole-HCl buffer (pH 7.2), L-glutamine (50 mM), 20 μM ADP, 20 mM MnCl₂, 125 mM hydroxylamine sodium and 10 mM sodium arsenate was mixed either with fixed (1.2 E⁻⁵ M) or variable concentrations (6.3e⁻¹⁹ to 4.8e⁻⁷ M) of NF dissolved in methanol. The final concentration of methanol per assay tube was 10% V/V. Fixed (3 mg/ml) or variable (0.023 to 3 mg/ml) concentrations of either purified brain pig GS or rat brain homogenates were added to each assay tube. The tubes were incubated a room temperature for 5 min. Methanol (10%) and FCS were used as negative controls. The tubes were thereafter centrifuged at 700 g. Supernatant was discarded and the pellets were suspended in 100 μl working buffer and incubated for 15 min at 37° C. The reaction was finally stopped and absorbance was measured as described above.

### 2.1.3.3 Spot Blot of the Proteins Precipitated by NF

Twenty μl of agarose gel with precipitated NF/protein complexes issued from diffusion study was applied to methanol activated PVDF membrane. Non-specific binding sites were blocked by 5% low fat skimmed milk in TBS buffer, supplemented with 0.1% tween 20. Transferred proteins were allowed to react with either polyclonal rabbit anti-GS (1/3000) or anti-GR (1/3000) antibodies for 30 min at room temperature. After one quick wash with TBS, 0.1% tween 20, horseradish peroxidase conjugated goat anti-rabbit antibody was subjected (1/5000) to the transferred proteins for 30 min at room temperature. After several wash the bound antibody was revealed using ECL.

### 2.1.3.4 SDS-PAGE and Western Blotting of the Protein Precipitated by NF

Twenty μl per well of precipitated NF/protein complex were separated on 8% SDS-PAGE gel in absence or presence of 2β-mercaptoethanol. The proteins separated in presence or the absence of SDS were thereafter transferred to a nitrocellulose membrane (20 μm). Non-specific binding sites were blocked by 5% low fat skimmed milk in TBS buffer, supplemented with 0.1% tween 20. Transferred proteins were thereafter allowed to react with either polyclonal rabbit anti-GS (1/3000) or anti-GR (1/3000) antibodies for 30 min at room temperature. After one quick wash with TBS, 0.1% tween 20, horseradish peroxidase conjugated goat anti-rabbit antibody was subjected (1/5000) to the transferred proteins for 30 min at room temperature. After several wash the bound antibody was revealed using ECL.

### 2.1.3.5 Effects of NF on GS Activity in Rat Brain Homogenates

Wistar rat brain homogenates were incubated with naftazone (7.87e⁻¹² to 2.11e⁻¹⁷ M) in absence of 2-βME. GS activity was assessed as mentioned above for purified sheep brain GS.

### 2.1.4 Effects of Pamidronate and Risedronate on GS Activity in Rat Brain Homogenates

Wistar rat brain homogenates were incubated with pamidronate and risedronate (2.27·10⁻⁹ to 7.96·10⁻²¹ or 8.75·10⁻²⁰ M) in presence or absence of 2-βME. GS activity was assessed as mentioned above for purified sheep brain GS.

### 2.1.5 Effects of Lithium and Strontium on GS Activity in Rat Brain Homogenates

Wistar rat brain homogenates were incubated with lithium (1e⁻² to 2.11e⁻²² M) and strontium (10⁻¹⁰ to 6.27·10⁻¹⁷) in absence of 2-βME. GS activity was assessed as mentioned above for purified sheep brain GS.

### 2.2 Study of the Effects of GS Ligands on GS Activity and Expression In Vivo

### 2.2.1 Effects of GF, MS and Tianeptine on Brain GS Activity and Expression in Mice

The effects of GF, MS and/or tianeptine on whole brain GS activity and expression were assessed i) 24 h after a single intraperitoneal (i.p.) administration in ICR mice [MS (0.078, 0.312, 1.25, 5, 20 mg/kg); tianeptine (0.078, 0.3125, 1.25, 5, 20 mg/kg); GS protein expression determined using western blot], and ii) 1 h after the last administration of a repeated (for 7 days) daily i.p. administration in BALB/c mice [GF (0.1, 3, 15 mg/kg); MS (0.5, 15, 45 mg/kg); tianeptine (0.5, 15, 45 mg/kg); GS protein expression determined by ELISA].

### 2.2.1.1 Determination of GS Activity

The brain's right hemisphere from each mouse was deep frosted in liquid nitrogen immediately after euthanasia and kept at −80° C. until use. On the day of assay, membranes were prepared in a chilled extraction buffer composed of HEPES (5 mM), sucrose (320 mM) and EDTA (1 mM). The pH was set to 7.4 with TRIS (100 mM). Ten μl of each brain mixture was incubated for 15 min at 37° C. in presence of 250 μl of sample buffer (see above 2.1.1). The enzymatic reaction was stopped using 250 μl of ferric chloride reagent. The absorbance was measured at 535 nm after centrifugation at 10000 g for 10 min, and the protein concentration determined using a BCA (bicinchoninic acid) kit (read at 562 nm; Pierce). GS activity results were expressed as absorbance at 535 nm per μg of protein.

### 2.2.1.2 Determination of GS Protein Expression by Western Blot

The brain's left hemisphere of each mouse was homogenised at 4° C. in a buffer consisting of 50 mM Tris, 350 mM glucose and 5 mM MgCl₂ (pH=7). The homogenate was centrifuged at 10000 g for 20 min at 4° C. The supernatant was centrifuged for 3×1 h at 20000 g at 4° C. The resulting pellet was suspended in TBS (pH=7) and the quantity of protein was determined using a BCA kit. Twenty μg per well of protein from each sample were applied to a 12% acryl/bisacrylamid gel in presence of SDS and 2β-mercaptoethanol. Separated proteins were thereafter transferred to a 0.2 μm PVDF membrane. After saturation with 5% low fat milk in TBS (pH=7) supplemented with 0.1% tween 20, membranes were incubated with goat monoclonal anti-GS antibodies (1/5000; AbCam) over night at 4° C. After several washes, the membranes were subjected to anti-goat antibodies conjugated to peroxidase (1/10000) for 1 h. Membranes were extensively washed and the complex of GS/antibody was revealed using an ECL method. Revealed western blots were analysed by densitometry (MacBas software). The results were expressed in absorbance unit per pixel² and μg of protein.

### 2.2.3 Determination of GS Protein Expression by ELISA

Brain homogenate (prepared as above mentioned for GS activity) were tested for GS expression. After centrifugation at 10500 g for 20 min at 4° C., the supernatant was collected. Its protein concentration was determined using a BCA kit. Fifty μl of different dilutions (0.2 to 0.025) of each sample in coating buffer (sodium carbonate buffer; pH 9.6) were incubated on microtitre plates (Falcon, USA) over a night at 4° C. After a single wash with TBS tween 0.1% (washing buffer), the plates were saturated with TBS tween 0.1% supplemented with 3% low fat graded dried milk (Biorad) for 1 h at 37° C. Anti-GS antibodies raised in goat (1/1000 dilution) were allowed to react for 1 h at 37° C. After 3 washes with washing buffer, the plates were incubated with peroxidase conjugated anti-goat antibodies for 1 h at 37° C., and washed 3 times with washing buffer and 4 times with just TBS. The bound antibodies were revealed with TMB (trimethyl benzidine) and H₂O₂ as substrates. The reaction was stopped after incubating the microtitre plates for 15 min at 37° C. with HCl 1 M. The absorbance was read at 405 nm on a microtitre plate reader. The results were expressed in absorbance per μg of protein.

### 2.2.2 Effects of GF and Tianeptine on GS Activity in Cortex, Hippocampus and Thalamus/Hypothalamus in ICR Mice

The effects of GF (0.1, 3 and 15 mg/kg), tianeptine (0.5, 15, 45 mg/kg) and placebo on GS activity in cortex, hippocampus and thalamus/hypothalamus were assessed in ICR mice 24 h after a single i.p. administration. Cortex, hippocampi and thalamus/hypothalamus were carefully isolated immediately after euthanasia (as mentioned above for hippocampi). Membranes were prepared and the activity of GS determined in these different structures (as mentioned above for the whole brain).

### 2.2.3 Effects of GF and NF on GS Activity in Cortex, Hippocampus and Thalamus/Hypothalamus in BALB/c Mice

The effects of GF (0.1 mg/kg), NF (10 and 100 mg/kg) and placebo on GS activity in cortex, hippocampus and thalamus/hypothalamus were assessed in BALB/c mice 24 h after a single i.p. administration. Cortex, hippocampi and thalamus/hypothalamus were carefully isolated immediately after euthanasia. Membranes were prepared and the activity of GS determined in these different structures (as mentioned above for the whole brain).

### 2.2.4 Effects of NF on GS Activity in Cortex, Hippocampus and Thalamus/Hypothalamus in Wistar Rats

The effects of NF (1 and 10 mg/kg) and placebo on GS activity in cortex, hippocampus and thalamus/hypothalamus were assessed in Wistar rats 24 h after a single i.p. administration. Cortex, hippocampi and thalamus/hypothalamus were carefully isolated immediately after euthanasia. Membranes were prepared and the activity of GS determined in these different structures (as mentioned above for the whole brain).

### 3 Evaluation of the Therapeutic Potential of GS Ligands

### 3.1 Effect of Tianeptine on Rat Brain GS in Condition of Oxidative Stress

Rat brain membrane preparation was pre-incubated for 2 min in sample buffer (see above 2.1.1) in presence of L-glutamine (50 mM) and ammonium dioxy persulfate (APS) (1.5%). Different concentrations of tianeptine (1.1·10⁻⁴ to 2.3·10⁻¹² M) were added. GS activity was assessed as mentioned above for purified sheep brain GS.

3.2 Effects of MS and Tianeptine on C6 Cells in Condition of Excess of Ammonia and/or Glutamate

### 3.2.1 Effects on Extracellular Ammonium Ion and Free Amine Concentrations

C6 glioblastoma cells were sealed to 6 wells culture plates at 10⁵ cells per well in presence of DMEM (Dulbecco's modified Egle's medium) and 10% fetal calf serum (FCS), and incubated 24 h at 37° C. with 5% CO₂ before the start of the assay. Thereafter, after discarding DMEM medium, they were washed 3 times with HBSS (Hank's Balanced salt solution), distributed at 10⁵ cells per well into 6 wells culture plates in FCS free HBSS, and incubated for 1 h at 37° C. and 5% CO₂ with either glutamate or glutamine and/or NH₄Cl, with or without MS or tianeptine. Media free of cells after 1 h were collected centrifuged and kept in −20° C. until analysis.

Determination of ammonium ion concentration: the ammonium ion concentration was determined using Nessler's quantitative method (Zhloba et al., 1968). Supernatants collected from treated cell cultures were mixed with NaOH, KI and HgCl. The amount of ammonium ion was reflected by the amount of NH₂Hg₂I₃ formed. The samples were centrifuged and the supernatants were carefully removed. The pellets were collected, washed three times with mili Q water and left to dry at 100° C. overnight before weighting them on a high precision balance.

Determination of free amine concentration: free amine concentrations were determined using Kaiser's quantitative ninhydrin technique (Sarin et al., 1981). The culture mediums were mixed with 3 different solutions i.e. phenol/ethanol, KSCN/pyridine and ninhidrin/ethanol and heated at 100° C. during 7 min. These mixtures were read at 570 nm. The amount of free amine for each sample was calculated according to the following formula:

$$Free amines ( mol \text{/} l ) = \frac{\begin{matrix}( \begin{matrix}absorbance of sample - \\ absorbance of blank\end{matrix} ) \times \\ dilution \times {10}^{6}\end{matrix}}{\begin{matrix}Extinction coefficient \times \\ sample volume in ml\end{matrix}}$$

where extinction coefficient is 1.5×10⁴ in m⁻¹ cm⁻¹

### 3.2.2 Effects on Apoptosis

C6 glioblastoma cells were sealed to 6 wells culture plates at 10⁵ cells per well in presence of DMEM (Dulbecco's modified Egle's medium) and 10% fetal calf serum (FCS), and incubated 24 h at 37° C. with 5% CO₂ before addition of glutamate and/or NH₄Cl, with or without MS or tianeptine. The plates were incubated either for 24 h or 72 h at 37° C. and 5% CO₂. Apoptosis was evaluated using a fluocytometric technique. Briefly, medium from each well was first collected. The cells were washed twice with HBSS. After a brief incubation with child trypsin/EDTA (1 min), they were collected and centrifuged at 250 g for 10 min. They were then incubated with FITC conjugated annexin V for 15 min in complete obscurity. Just before counting the cells, propidium iodide was added to each sample. The (total) apoptosis was the sum of early and late apoptosis (FACScalibur 4 colors, BD Bioscience).

### 3.3 Effects of GF, MS and Tianeptine in Conditions of High Glucocorticoid Exposure

### 3.3.1 Experiment Using Corticosterone

Wistar rats were administered during 21 days once a day with corticosterone (35 mg/kg) s.c. plus GF (0.1, 3 and 15 mg/kg), MS (0.5, 15 and 45 mg/kg), tianeptine (0.5, 15 and 45 mg/kg) or vehicle (PCB) i.p., or with both corticosterone's vehicle and treatment's vehicle (Control).

### 3.3.1.1 Behavioral Explorations

Rat's global activity was explored through an open field task after 15 days of administration of corticosterone plus GF, MS, tianeptine or vehicle (PCB), or of both corticosterone's vehicle and treatment's vehicle (Control). One hour before the experiment, the animal was administered its treatment and placed into the experimental room. At the time of the experiment, the animal was gently placed in the center of the arena (78 cm×78 cm×49 cm) and left free to explore it (10 min session). Its behavior was recorded with a numeric video camera. The video records were analyzed offline using the SMART automated tracking program (Panlab). Resting time was the sum of the time spend immobile; immobility was defined as no movement with a speed exceeding 2.5 cm/sec.

A forced swimming task was also done after 19 days of administration of corticosterone plus GF, MS, tianeptine or vehicle (PCB), or of both corticosterone's vehicle and treatment's vehicle (Control). One hour before the experiment, the animal was administered its treatment and placed into the experimental room. At the time of experiment, it was gently placed in an individual cylinder (50 cm×20 cm in diameter) filled with water (25° C.) up to 30 cm. A rat was judged to be immobile when it floated and made only movements necessary to keep the head above the water. At the end of swimming session, the rat was removed from the cylinder, dried with towels, placed in a cage for 15 min rest and recovery, and then returned to its home cage.

### 3.3.1.2 Golgi Procedure to Assess the Surface of CA3 Pyramidal Neurons

One hour after the last drug(s)/vehicle(s) administration on day 21, each rat was sacrificed and its brain was collected. The right hemisphere was fixed in 4% formaldehyde and the left hemisphere was frozen in liquid nitrogen. The latter was kept at −80° C. until use (see below 3.3.3). The right hemisphere fixed in formaldehyde was cut with a vibratome (100 μm slices) in a bath of 3% potassium dichromate and processed according to a modified version of the single-section Golgi impregnation procedure of Gabbott & Somogyi (Gabbott & Somogyi, 1984). The slices were incubated over a night in 3% potassium dichromate in distilled water, then rinsed in distilled water and mounted on plain slides. A coverslip was glued over the sections at the four corners. The slide assemblies were incubated in darkness over night in a 5% silver nitrate water solution. Two days later the slide assemblies were dismounted, and the tissue sections were rinsed in distilled water and mounted according to the standard procedures.

Golgi-impregnated neurons according to Magarinos and McEwen (Magarinos and McEwen, 1995) had to possess the following characteristics to be included in the analysis: a) location within the CA3c subregion of the dorsal hippocampus; b) dark and consistent impregnation throughout the extent of all the dendrites; c) relative isolation from neighboring impregnated cells, which could interfere with the analysis; and d) cell bodies in the middle third of the tissue section, to avoid analysis of impregnated neurons that extended well into other sections. For each brain 6-10 pyramidal cells at 400× magnification were digitalized and traced. Each digitalized traced pyramidal neuron was extracted from the raw image and its surface area was measured using the Scion Image software. The slides were coded before analysis and code was broken only after the analysis was completed.

### 3.3.1.3 Determination of GS Activity in Whole Brain, Liver and Lungs

Brain's left hemisphere, liver and lungs were collected immediately after euthanasia and frosted in liquid nitrogen. These organs were kept at −80° C. until use. On the day of the assay, they were homogenised in sample buffer HEPES (5 mM) with (EDTA) (1 mM). The pH was set to 7.4 with TRIS (100 mM). GS activity was assessed as mentioned above for purified sheep brain GS (see 2.1.1).

### 3.3.1.4 Determination of the Expression of GS and Glucocorticoid Receptor by ELISA

Brain's left hemisphere homogenates from rats (prepared as above mentioned in 2.1.1) were tested for expression of GS and/or glucocorticoids receptors. After centrifugation at 10500 g for 20 min at 4° C., the supernatant was collected and its protein concentration was determined using a BCA kit. Fifty μl of different dilutions (0.2 to 0.025) of each sample in coating buffer (sodium carbonate buffer, pH 9.6) were incubated on microtitre plates (Falcon, USA) over a night at 4° C. After a single wash with TBS tween 0.1% (washing buffer), the plates were saturated with TBS tween 0.1% supplemented with 3% low fat graded dried milk (Biorad) for 1 h at 37° C. Either anti-GS antibodies raised in goat or goat anti-rabbit anti-glucocorticoids receptor antibodies (1/1000 dilution) were allowed to react for 1 h at 37° C. After 3 washes with washing buffer, the plates were either incubated with peroxidase conjugated anti-goat or goat anti rabbit antibodies for 1 h at 37° C., and washed 3 times with washing buffer and 4 times with just TBS. The bound antibodies were revealed with TMB (trimethyl benzidine) and H₂O₂ as substrates. The reaction was stopped after an incubation of 15 min at 37° C. with HCl 1 M. The absorbance was read at 405 nm on a microtitre plate reader. The result was expressed in absorbance per μg of protein.

### 3.3.2 Experiments Using Methylprednisolone

3.3.2.1 Effects of GF and Tianeptine on GS Activity in the Cortex, Hippocampus and Thalamus/Hypothalamus of Wistar Rats Administered Chronically with Methylprednisolone

Wistar rats were administered during 42 days once a day with methyprednisolone (5 mg/kg i.p.) plus GF (1 mg/kg), tianeptine (10 mg/kg) or vehicle (PCB) (i.p.). Twenty-four hours after the last administrations, they were euthanatized. Brain's left hemisphere were collected and immediately frosted in liquid nitrogen. These organs were kept at −80° C. until use. On the day of the assay, they were homogenised in sample buffer HEPES (5 mM) with (EDTA) (1 mM). The pH was set to 7.4 with TRIS (100 mM). GS activity was assessed as mentioned above for purified sheep brain GS (see 2.1.1).

3.3.2.2 Effect of GF and Tianeptine on Adrenal Weight of Wistar Rats Administered with Methylprednisolone

Wistar rats were administered during 21 days once a day with methyprednisolone (5 mg/kg i.p.) plus GF (0.001, 0.01, 0.1 mg/kg), NF (1 and 10 mg/kg) or vehicle (PCB; i.p). Twenty-four hours after the last administrations, they were euthanatized. A complete necropsy was performed and main organs weighted.

### 3.4 Effects of GF and Tianeptine in a Model of PTZ-Induced Seizures

Wistar rats were administered GF (0.5, 5 and 25 mg/kg i.p.), tianeptine (1, 10 and 50 mg/kg i.p.) or vehicle (PCB) 1 hour before pentylenetetrazole (PTZ; 50 mg/kg i.p.) administration.

### 3.4.1 Monitoring of PTZ Induced-Seizures

Immediately after PTZ injection, the seizure activity was monitored for 20 min. Resulting seizures were classified using the procedure of Lamberty and Klitgaard: Stage 0 (no response), Stage 1 (ear and facial twitching), Stage 2 (convulsive waves through the body), Stage 3 (myoclonic jerks, rearing), Stage 4 (turn over onto side position) and Stage 5 (turn over onto back position, with generalized tonic-clonic seizures).

### 3.4.2 Determination of GS Activity in Cortex, Hippocampus and Thalamus/Hypothalamus of PTZ Treated Wistar Rats.

One hour after a single i.p. administration of PTZ, the rats were euthanatized. Their cortex, hippocampi and thalamus/hypothalamus were carefully isolated (as mentioned above for hippocampi). Membranes were prepared and the activity of GS determined in these different structures (as mentioned above for the whole brain).

### Main Results

### 1 Screening of Tianeptine's Target Protein

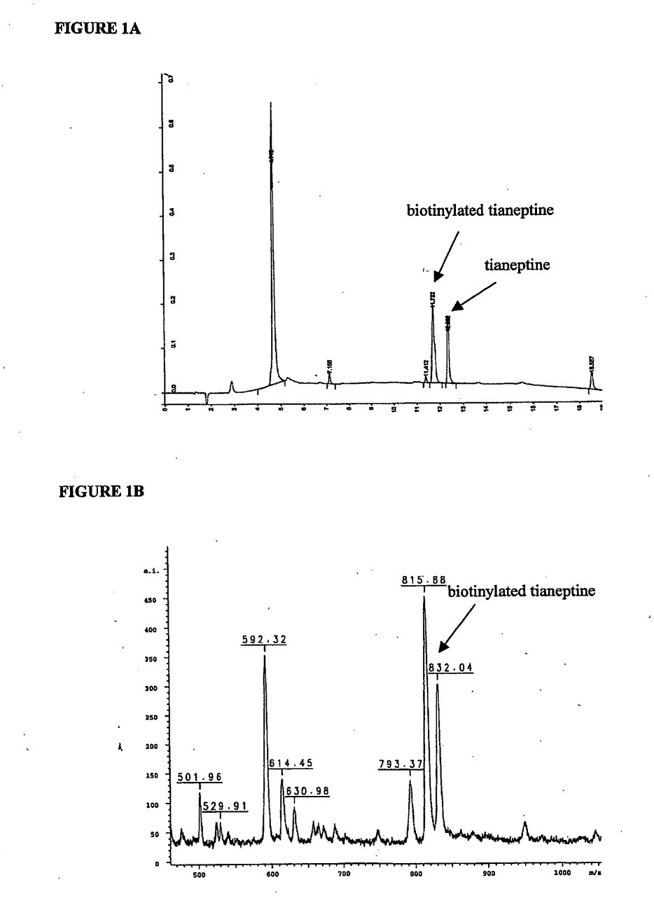

### 1.1 Biotinylation of Tianeptine (FIG. 1)

The biotinylation of tianeptine required a 3 days reaction period with regular addition of N-(3-Dimethylaminopropyl)-N′-ethylcarbodiimide (EDC). Biotin conjugated tianeptine was separated from unconjugated ones by using a phase inverse C18 column; conjugated tianeptine was eluted at 60% acetonitrile (retention time 11.7 min) (FIG. 1A). The yield of biotinylation was estimated 76%. The mass of biotinylated tianeptine was checked by mass spectrometry (FIG. 1B).

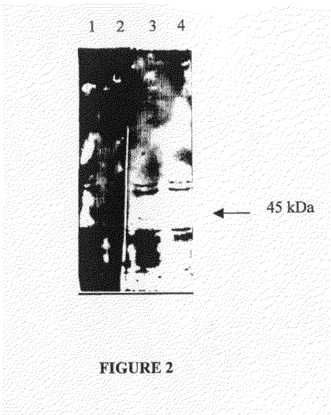

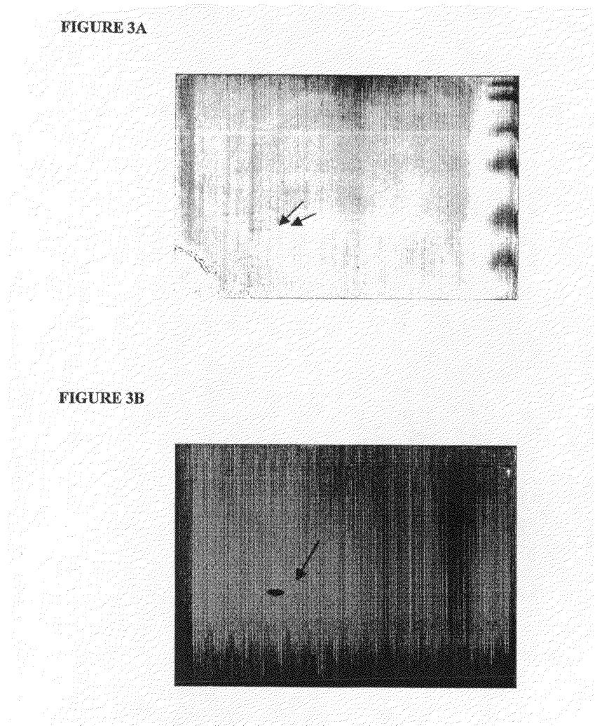

### 1.2 Isolation and Identification of Tianeptine's Target Protein (FIGS. 2-3)

Western blot on mouse hippocampus membrane proteins in absence of n-octyl glucoside revealed a bond at 45 kDa (FIG. 2). In the presence of n-octyl glucoside the bond vanished (FIG. 2), which indicated this 45 kDa protein revealed by peroxydase conjugated streptavidin was probably a cytoplasmic rather than membrane anchored protein.

The same membrane preparation with 2D gel electrophoresis followed by western blotting revealed two neighboring spots at 42.146 kDa with an isoelectric point at pH 6.5 (FIGS. 3 A and B). Mass spectrometry confirmed the former and the sequence obtained from the digested spots matched (with scores 1.897×10⁶ and 6,6×10⁵ respectively for mouse glutamine synthetase. The interaction between biotinylated tianeptine and GS, based on the latter analyses, appeared to be at the enzyme's catalytic site with peptide sequence ITGTNAEVMPAQWEFQIGPCEGIR (for both spots).

### 1.3 In Silico Study of the Interaction Between Tianeptine and Bovine Brain GS (FIG. 4)

In silico study of the interaction between tianeptine and bovine brain GS was almost identical with the one between glufosinate and the same GS (Gill and Eisenberg, 2001). The three conformations with the lowest potential energy, which were chosen for the final minimalization by conjugated gradient using a distance dependent dielectric constant, resulted in the same complex after minimalization.

### 2 Characterization of GS Ligands Pharmacological Effects

### 2.1 In Vitro Effects of GS Ligands

### 2.1.1 Effects of GF, Iproniazid, MS and Tianeptine on Purified Sheep Brain GS Activity (FIGS. 5, 6, 7, 9)

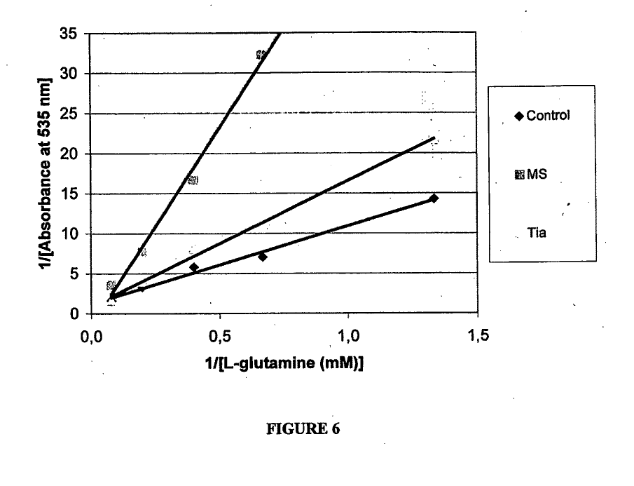

In presence of 2β-ME, GF, iproniazid, MS and tianeptine could inhibit GS activity at “high” concentrations (FIG. 5-7). GF, MS and tianeptine are competitive with L-glutamine (FIG. 6; Meister 1985; Gill and Eisenberg, 2001), while iproniazid competes with hydroxylamine on the NH₄ site (Rueppel et al., 1972).

In absence of 2βME, GF and tianeptine could inhibit GS activity at “high” concentrations but also activate GS activity at “low concentrations” (10⁻⁴ to 10⁻¹¹ and 10⁻¹³ to 10⁻¹⁶M, respectively) (FIG. 7).

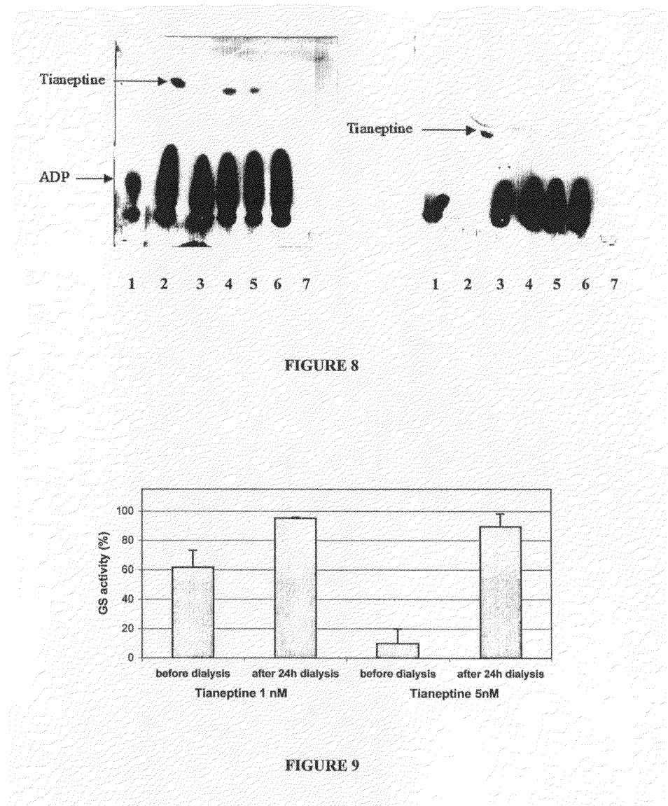

### 2.1.2 Effects of Dialysis on the Effect of Tianeptine on Purified Sheep Brain GS Activity (FIGS. 8-9)

The interaction between tianeptine and GS was reversible as it is illustrated in FIGS. 8 and 9 where decreased GS activity due to 1 and 5 nM tianeptine is reversed after over night dialysis against the reaction buffer. GF and MS are known to bind GS irreversibly (Meister 1985).

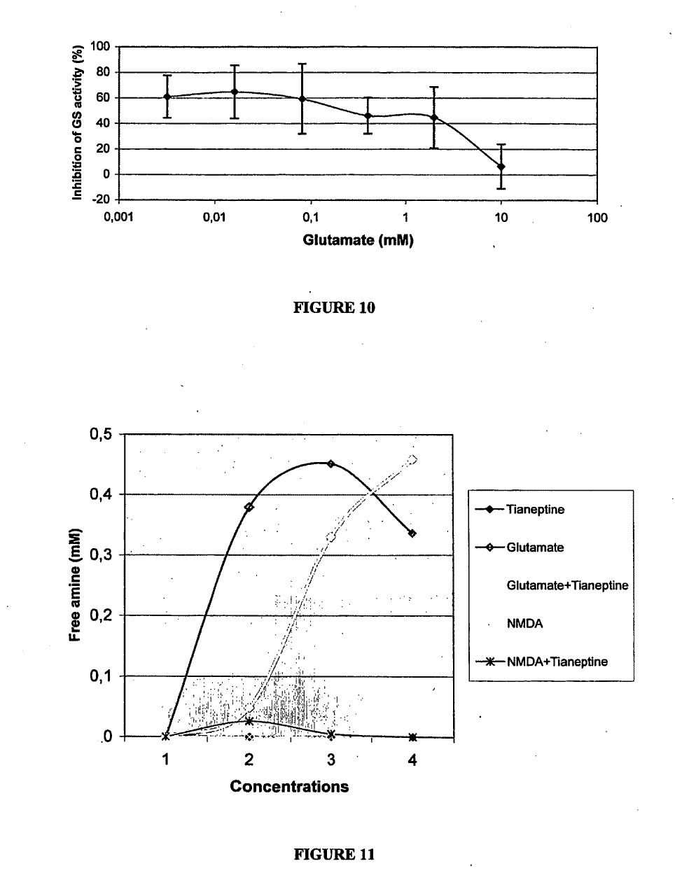

### 2.1.3 Effects of L-Glutamate on the Effect of Tianeptine (FIGS. 10-11)

The effect of increasing concentration of L-glutamate (0.0016-10 mM) were assessed on purified sheep brain GS in presence of 5 μM tianeptine (FIG. 10). The inhibitory effect of tianeptine on GS activity decreased as the concentration of L-glutamate increased. Higher concentration than 10 mM of L-glutamate (100 and 500 mM) increased background activity so no specific activity could be measured.

The inhibitory effect of tianeptine on extracellular free amine induced by glutamate after 1 h incubation in C6 cells in serum free HBSS decreased also as the concentration of L-glutamate increased (FIG. 11). At the highest concentration (500 μM), tianeptine (100 nM) was even observed to increase extracellular free amine induced by glutamate. Tianeptine (100 nM) and/or N-methyl-D-aspartate (NMDA) (1, 10, 100, 500 μM) had no effect on extracellular free amine after 1 h incubation in C6 cells in serum free HBSS.

### 2.1.4 Interaction Between NF and GS (FIGS. 34-38)

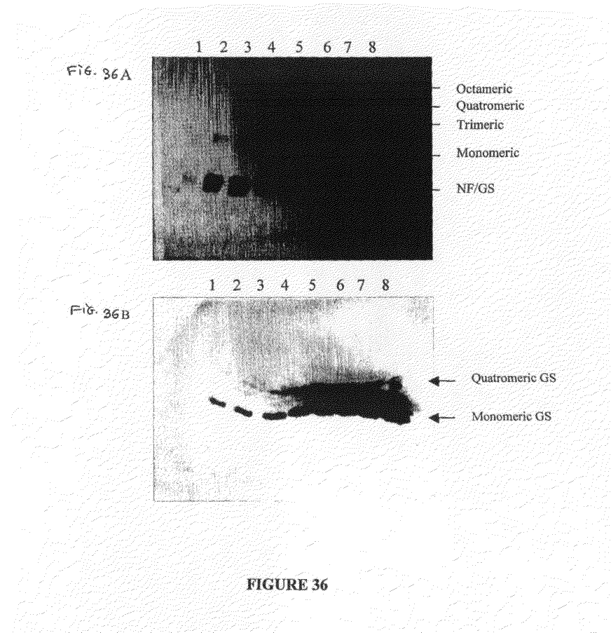

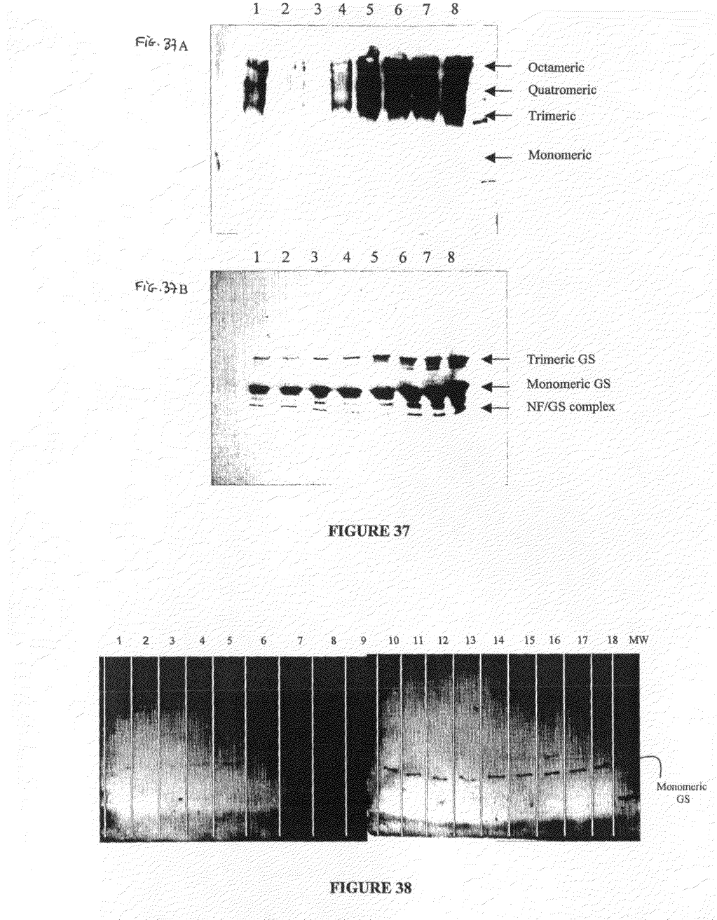

In agarose (0.1%) gel, the interaction between NF and purified pig GS revealed a clear precipitated protein. The precipitation was assessed using Commassie blue (FIG. 34. Dotblot analyses revealed specifically glutamine synthetase (FIG. 35). This was highlighted using hydrophile/hydrophobe phase extraction methods. Negative controls were methanol treated FCS in presence and absence of naftazone. No protein was extracted from FCS. Extracted proteins both from purified pig GS and whole rat brain homogenates subjected to gel electophoresis western blot revealed specifically GS protein. In presence of 2-βME only monomeric GS was detected while in absence of 2-βME multimeric GS was detected (FIG. 36-37). In another experiment different concentration of NF precipitated dose dependently GS protein (FIG. 38). Subjecting precipitated proteins both from purified pig GS and whole rat brain homogenates to rabbit anti-GR antibodies did not reveal any protein. Masspectrometric analyses from a corresponding bound isolated on a 8% bis/akrylamide gel in native conditions, from rat brain homogenate showed 30% presence of glutamine synthetase and 70% G-actin.

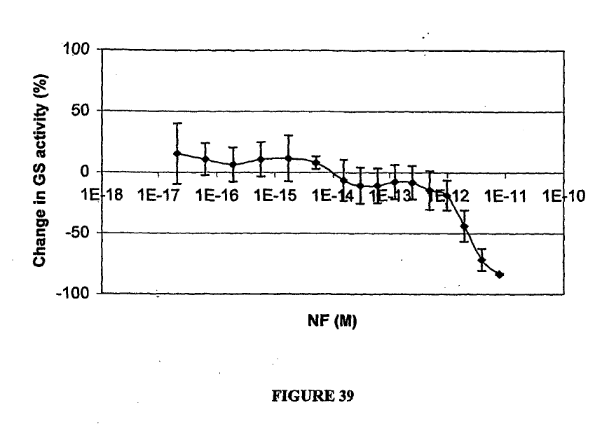

### 2.1.5 Effects of NF on Rat Brain GS Activity (FIG. 39)

In absence of 2β-ME, NF could inhibit GS activity at “high” concentration range 10⁻¹² to 10⁻¹⁴ M

### 2.1.6 Effects of Pamidronate and Risedronate on Rat Brain GS Activity (FIG. 12-13)

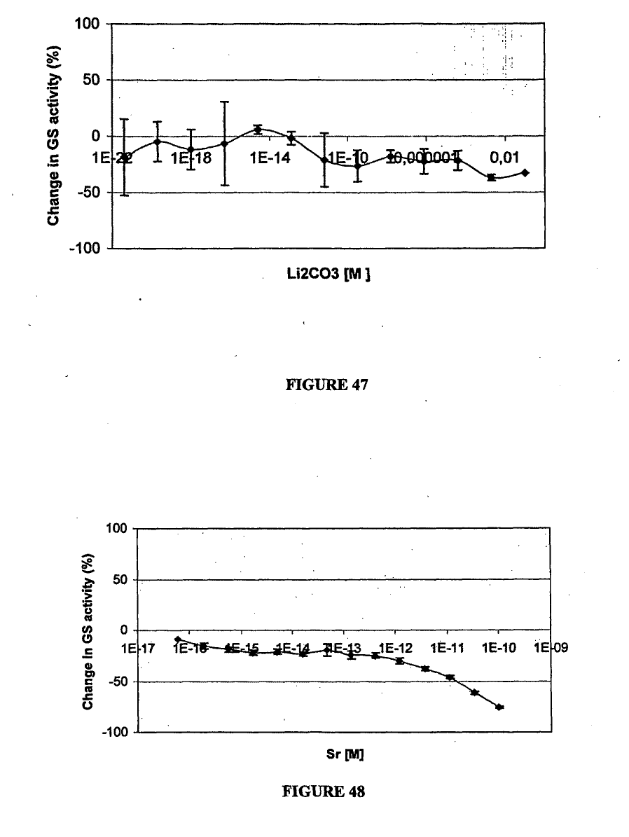

In presence of 2β-ME, pamidronate and risedronate could inhibit GS activity at “high” concentrations (FIGS. 12A and 13A). In absence of 2β-ME, pamidronate and risedronate could inhibit GS activity at “high” concentrations but also activate GS activity at “low concentrations” (10⁻¹² to 10⁻¹⁷ M) (FIGS. 12B and 13B) Effects of lithium and strontium on rat brain GS activity FIG. 47-48)

In absence of 2β-ME, lithium and strontium could inhibit GS activity from 10⁻¹² M with maximum 40% decrease at 0.01 M and 75%, decrease at 10⁻¹⁰ M, respectively.

### 2.2 Study of the Effects of GS Ligands on GS Activity and Expression In Vivo

### 2.2.1 Effects of GS Ligands on Whole Brain GS Activity and Expression

2.2.1. Effects of MS and Tianeptine on Whole Brain GS Activity and Expression 24 h after a Single i.p. Administration in BALB/c Mice (FIG. 14)

The effects of different doses of MS (0.078, 0.312, 1.25, 5, 20 mg/kg) and tianeptine (0.078, 0.312, 1.25, 5, 20 mg/kg) were assessed. Although there was no direct correlation between GS activity and GS expression, the effects of MS and tianeptine on both GS activity and GS expression were dose-dependent. MS and tianeptine could increase GS activity, the highest activity being observed at 0.312 mg/kg for MS and 1.25 mg/kg for tianeptine. The differences between doses as regard to GS expression were less pronounced for both molecules.

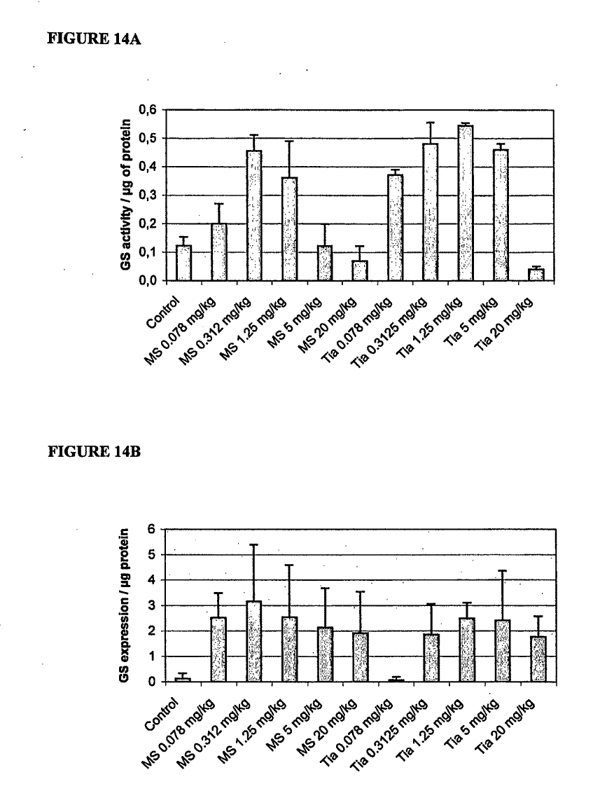

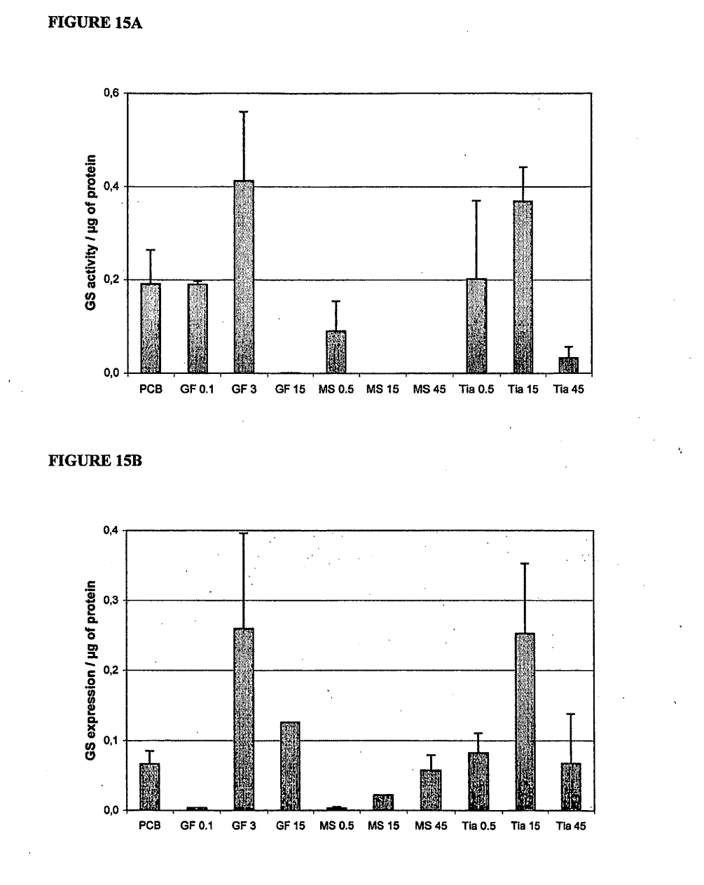

2.2.1.2 Effects of GF, MS and Tianeptine on Whole Brain GS Activity and Expression After a 7 Days Repeated i.p. Administration in BALB/c Mice (FIG. 15)

The effects of different doses of GF (0.1, 3, 15 mg/kg), MS (0.5, 15, 45 mg/kg) and tianeptine (0.5, 15, 45 mg/kg) were assessed. Although there were no direct correlation between GS activity and GS expression, the effects of GF, MS and tianeptine were dose-dependent on both GS activity and GS expression. GF and tianeptine could increase GS activity at the 3 mg/kg and 15 mg/kg doses, respectively. At these doses GF and tianeptine increased also GS expression in the brain. At higher doses, GF and tianeptine decreased GS activity, as this was also the case for the three doses of MS, which decreased GS activity proportionally the dose.

2.2.2 Effects of GF and Tianeptine on Cortex, Hippocampus and Thalamus/Hypothalamus GS Activity after a Single Administration in ICR Mice FIG. 16)

The effects of different doses of GF (0.1, 3 and 15 mg/kg) and tianeptine (0.5, 15, 45 mg/kg) were assessed. Twenty four hours after administration, GF and tianeptine had no significant effect on GS activity in the cortex, while they dose-dependently increased GS activity in the hippocampus and decreased GS activity in the thalamus/hypothalamus.

### 2.2.3 Effects of GF on Cortex, Hippocampus and Thalamus/Hypothalamus GS Activity

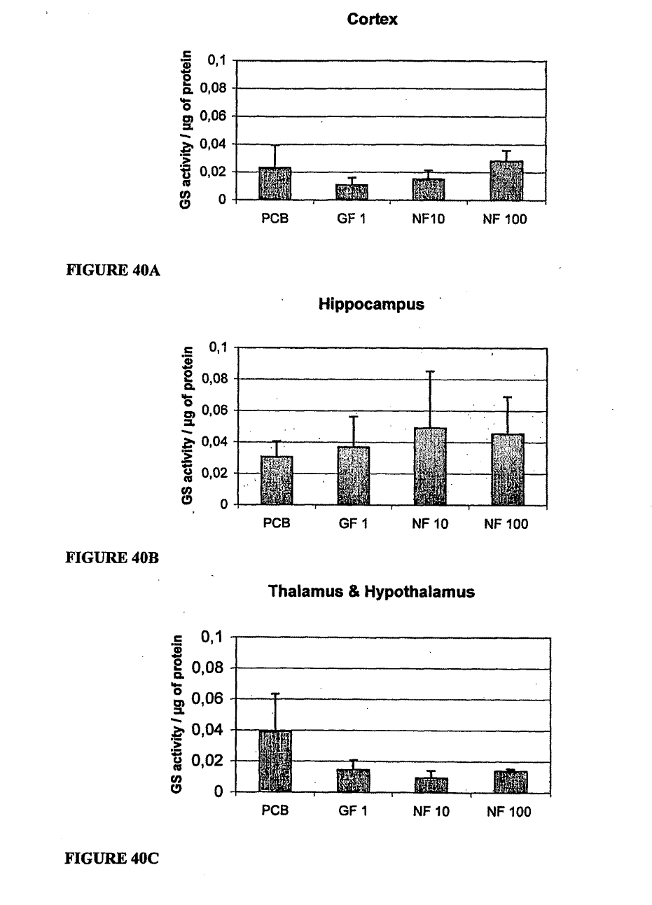

### 2.2.3.1 After a Single Administration in Balb/C Mice FIG. 40)

The effects of two doses of NF (10 and 100 mg/kg) were assessed versus vehicle and GF (1 mg/kg). Twenty four hours after administration, GS activity was decreased in the NF (not dose-dependent effect) and GF groups in the thalamus/hypothalamus. A trend to an increase of GS activity was observed in the hippocampus of the NF groups.

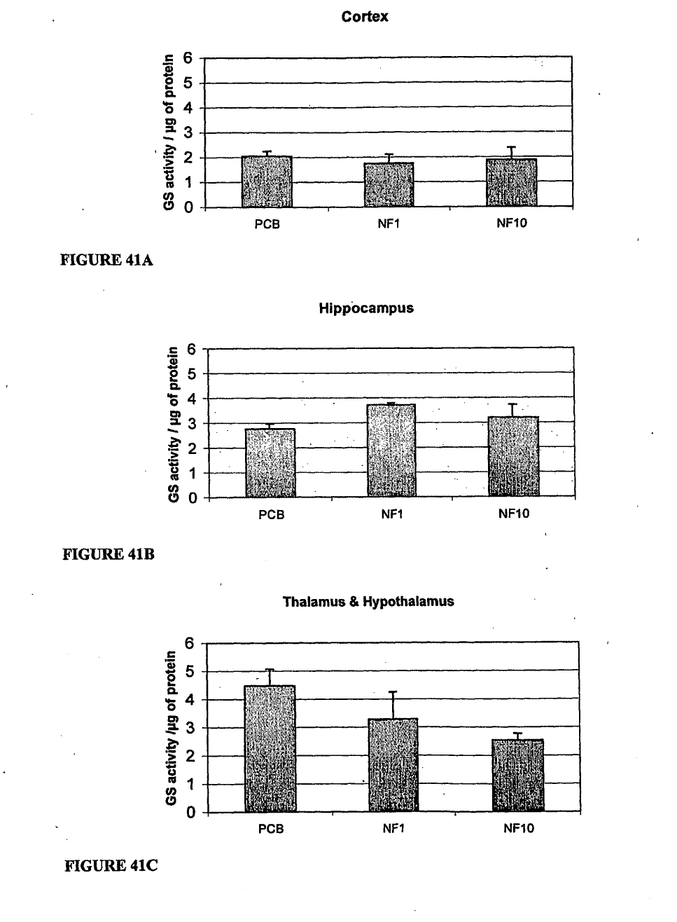

### 2.2.4 After a Single Administration in Wistar Rats (FIG. 41)

The effects of two doses of NF (1 and 10 mg/kg) were assessed. Twenty four hours after administration, NF has dose-dependently decreased GS activity in the thalamus/hypothalamus and increased GS activity in the hippocampus (maximum effect at 1 mg/kg). No significant treatment-effect was observed in the cortex.

### 3 Evaluation of the Therapeutic Potential of GS Ligands

### 3.1 Effect of Tianeptine on Rat Brain GS in Condition of Oxidative Stress (FIG. 17)

GS activity in presence of APS (1.5%) decreased about 35%. Tianeptine at concentrations between 10⁻⁶ and 10⁻¹¹ M protected GS activity from APS. It was even able to increase GS activity from concentrations 10⁻⁸ to 10⁻¹⁰ M.

3.2 Effects of MS and Tianeptine on C6 Cells in Condition of Excess of Ammonia and/or Glutamate

### 3.2.1 Effects on Extracellular Ammonium Ion and Free Amine (FIGS. 11, 18-24)

Different concentrations of MS (0.06, 0.12, 0.25 and 0.5 μM) and tianeptine (0.05, 0.5, 5 and 100 nM) were tested in cultures of C6 glioblastoma cells in serum free HBSS supplemented with either glutamate (50 mM) and/or NH₄Cl (10 mM).

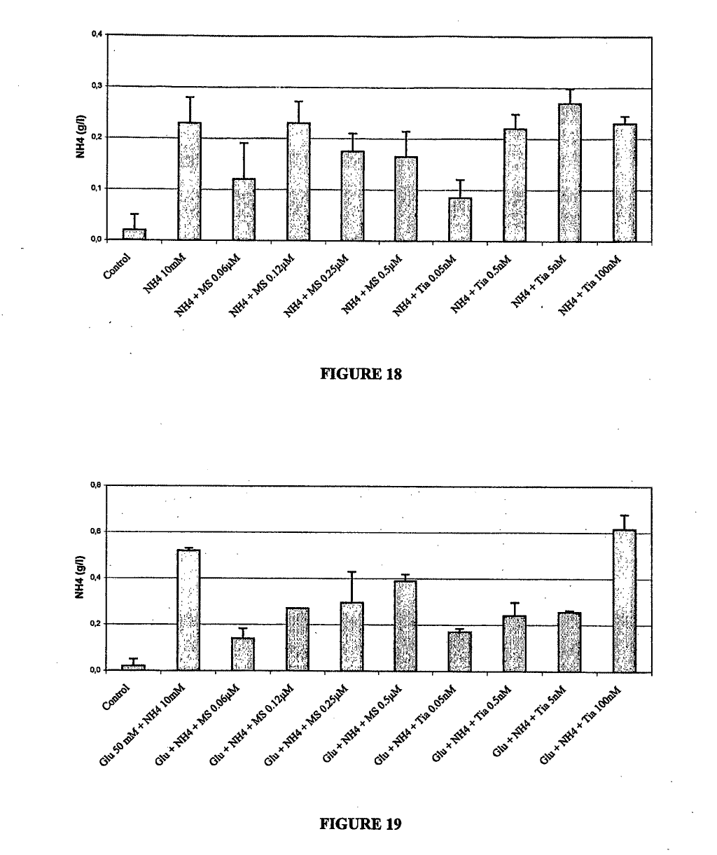

No extracellular NH₄⁺ could be detected after addition of L-glutamate (50 mM). On the contrary, addition of NH₄Cl (10 mM) clearly increased the extracellular concentration of NH₄⁺; and MS and tianeptine at low concentrations (0.06 nM and 0.05 nM, respectively) could blunt this increase (FIG. 18). The latter was emphasized when culture medium was supplemented both with L-glutamate (50 mM) and NH4Cl (10 mM) (FIG. 19).

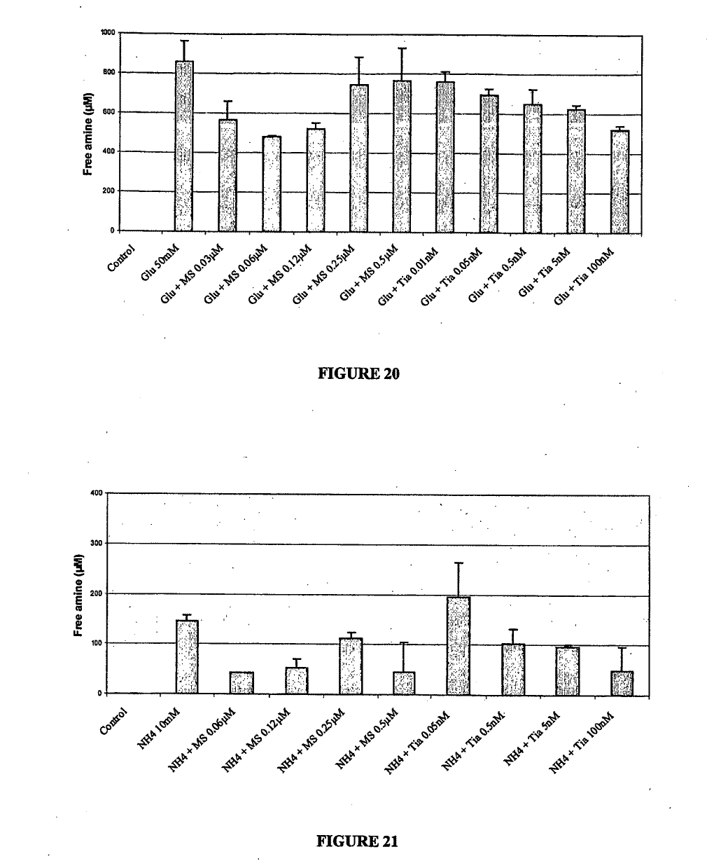

The production of free amine increased in cultures of C6 glioblastoma cells in serum free HBSS supplemented with glutamate (FIG. 11, 20). In comparison, the extracellular concentrations of free amine in cultures supplemented with NH₄Cl (10 mM) were only slightly increased; and those in the cultures supplemented both with glutamate (50 mM) and NH₄Cl (10 mM) were intermediate. MS and tianeptine could blunt these increases (FIG. 20-22). The nature of the dose-effect relationship appeared to be condition-dependent. MS alone was observed to interact with the Kaiser's method (FIG. 23). Thus, it was difficult to conclude about the “biphasic” dose-effect relationship which was observed with MS. For tianeptine, the blunting effect was clearly proportional to the dose.

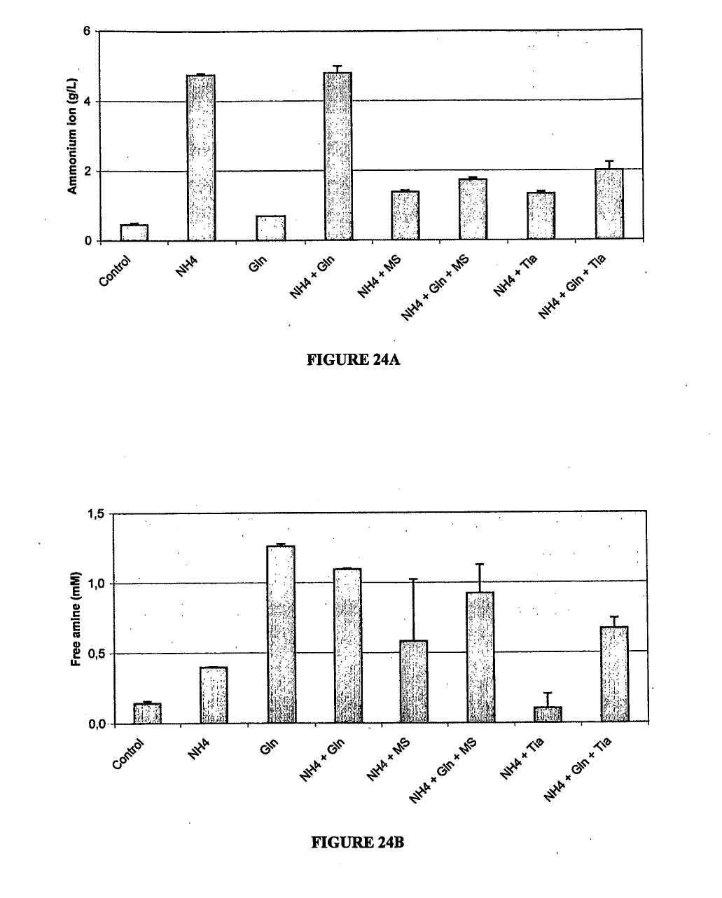

Glutamine (2 mM) did not alter the effects MS (5 mM) and tianeptine (100 nM) on extracellular ammonium ion and free amine concentrations induced by NH₄Cl (10 mM) after 1 h in C6 cells in serum free HBSS (FIG. 24).

### 3.2.2 Effect of MS and Tianeptine on Apoptosis in C6 Cells (FIGS. 25-27)

Total apoptosis (early apoptosis and necrosis) induced by hyperammonia (NH₄Cl 10 mM) was assessed after 72 h in C6 glioblastoma cells cultured in DMEM supplemented with 10% fetal calf serum (FCS). Both MS and tianeptine decreased total apoptosis at low concentrations (0.125 μM and 0.25 nM respectively) (FIG. 25).

Total apoptosis induced by L-glutamate (Glu 50 mM) was assessed after 24 h in C6 glioblastoma cells cultured in DMEM supplemented with 10% FCS. Both MS and tianeptine decreased total apoptosis at low concentrations (0.125 μM and 0.25 nM respectively) (FIG. 26).

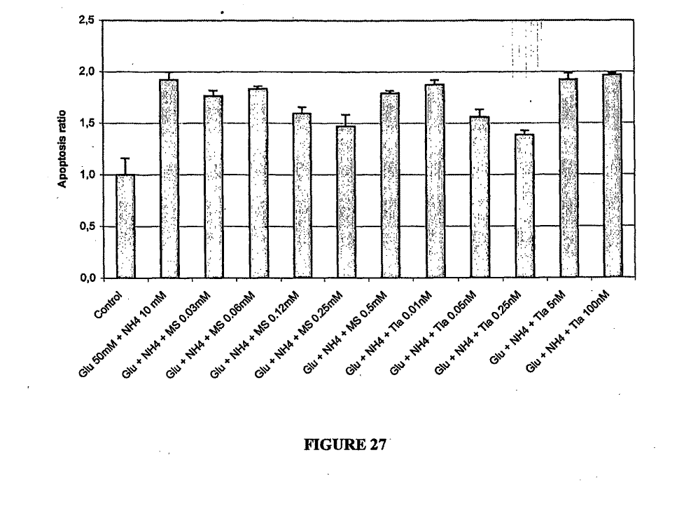

Total apoptosis induced by the association hyperammonia (NH4Cl 10 mM) plus glutamate (Glu 50 mM) was assessed after 24 h in C6 glioblastoma cells cultured in DMEM supplemented with 10% FCS. Both MS and tianeptine decreased total apoptosis at low concentrations (0.25 μM and 0.25 nM respectively) (FIG. 27).

### 3.3 Effects of GF, MS and Tianeptine in Conditions of High Glucocorticoid Exposure

3.3.1 Experiments with Corticosterone

Wistar rats were administered during 21 days once a day with corticosterone (35 mg/kg) s.c. plus GF (0.1, 3 and 15 mg/kg), MS (0.5, 15 and 45 mg/kg), tianeptine (0.5, 15 and 45 mg/kg) or vehicle (PCB) i.p., or with both corticosterone's vehicle (s.c.) and treatment's vehicle (i.p.) (Control).

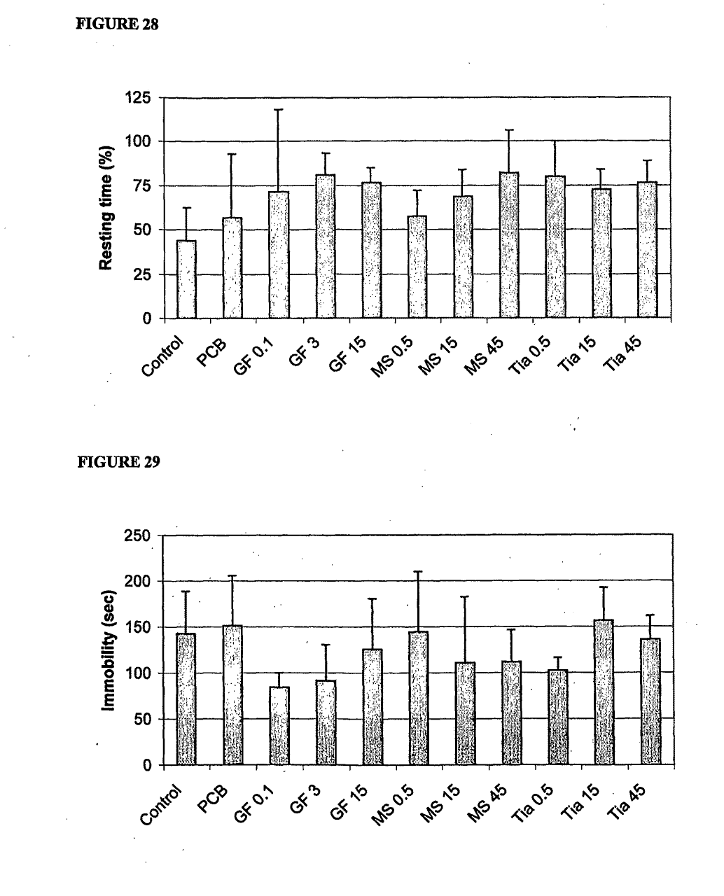

### 3.3.1.1 Behavioral Explorations (FIGS. 28-29)

Behavior was assessed in an open field task at day 15 (FIG. 28) and in a forced swimming task at day 19 (FIG. 29).

Resting time in the open field task was observed to be higher in the rats administered with corticosterone plus GF, MS or tianeptine compared to the rats administered with corticosterone and vehicle (PCB) and those administered with both corticosterone's vehicle and treatment's vehicle (Control), whose resting time remained comparable. This increase in resting time was only proportional to the dose for MS.

Immobility in the forced swimming test was observed to be comparable or lower in the rats administered with corticosterone plus GF, MS or tianeptine compared to the rats administered with corticosterone and vehicle (PCB) and the rats administered with both corticosterone's vehicle and treatment's vehicle (Control), whose duration of immobility remained comparable. A clear decrease in immobility was observed in the animals administered with 0.1 mg/kg of GF and 0.5 mg/kg of tianeptine.

### 3.3.1.2 Morphology of CA3 Pyramidal Neurons (FIG. 30)

Rats administered during 21 days with corticosterone plus GF, MS or tianeptine showed morphological changes of pyramidal neurons in the hippocampus CA3 region compared to the rats administered with corticosterone and vehicle (PCB) and those administered with both corticosterone's vehicle and treatment's vehicle (Control) (FIG. 30). GF at the doses 0.1 and 3 mg/kg could prevent in part the deleterious effects of corticosterone on CA3 pyramidal neurons. For MS and tianeptine, the best protective effects were observed at the 15 mg/kg dose, and 0.5 and 15 mg/kg doses, respectively (FIG. 30). Only GF at the 15 mg/kg dose failed to blunt the effects of corticosterone.

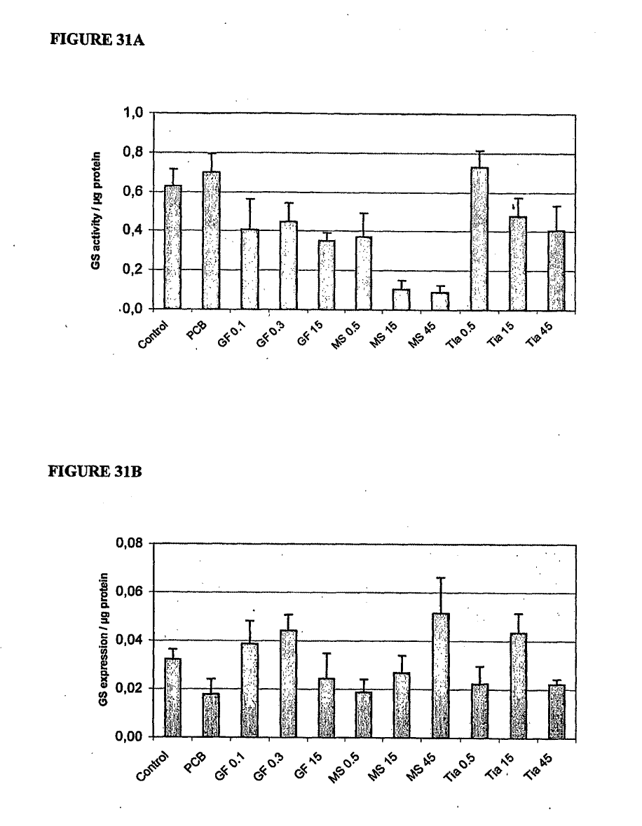

### 3.3.1.3 GS Activity and Expression in the Whole Brain (FIG. 31)

A dose-dependent decrease in brain GS activity was observed in the rats administered with corticosterone plus GF, MS or tianeptine compared to the rats administered with corticosterone and vehicle (PCB) and those administered with both corticosterone's vehicle and treatment's vehicle (Control); brain GS activities in the PCB and control groups were comparable (FIG. 31A). GS activity in the rats treated with 0.5 mg/kg of tianeptine remained in the same range as observed in the rats from the PCB and Control groups. The lowest GS activities were observed with MS at the and 45 mg/kg doses.

Brain GS expression was observed to be higher in the rats treated with corticosterone plus GF at the doses of 0.1 and 3 mg/kg, MS at the dose of 45 mg/kg or tianeptine at the dose of 15 mg/kg compared to the rats administered with corticosterone and vehicle (PCB) or, to a lesser extent, those administered with both corticosterone's vehicle and treatment's vehicle (Control) (FIG. 31B). Brain GS expression in the rats administered with corticosterone and vehicle (PCB) was clearly lower compared to this in the rats administered with both corticosterone's vehicle and treatment's vehicle (Control).

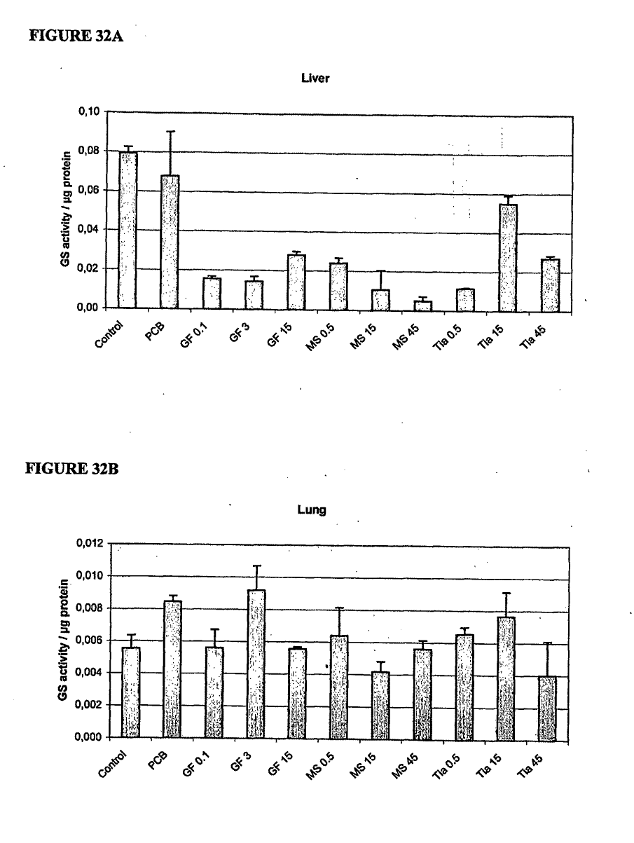

### 3.3.1.4 Determination of GS Activity in Liver and Lungs (FIG. 32)

A clear decrease in liver GS activity was observed in the rats administered with corticosterone plus GF, MS or tianeptine compared to the rats administered with corticosterone and vehicle (PCB) and those administered with both corticosterone's vehicle and treatment's vehicle (Control); liver GS activities in the PCB and Control groups were comparable (FIG. 32A). Only GS activity in the rats treated with 0.5 mg/kg tianeptine remained in the same range as observed in the rats from the PCB and Control groups.

Lung GS activity in the rats administered with corticosterone and vehicle (PCB) was increased compared to the activity in the rats administered with both corticosterone's vehicle and treatment's vehicle (Control). A decrease in lung GS activity was observed in most rats administered with corticosterone plus GF, MS or tianeptine compared to the rats administered with corticosterone and vehicle (PCB) (FIG. 32B). Only GS activity in the rats treated with 3 mg/kg GF and 15 mg/kg tianeptine remained in the same range as observed in the rats from the PCB group.

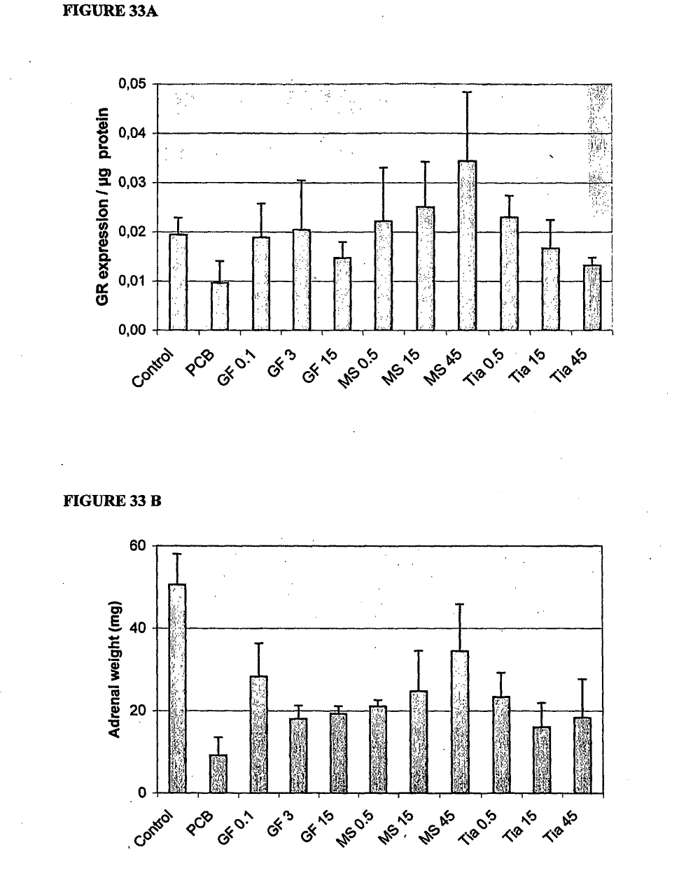

### 3.3.1.5 Glucocorticoid Receptor (GR) Expression in the Whole Brain and Adrenal Weight (FIG. 33)

Brain GR expression was observed to be higher in the rats treated with corticosterone plus GF at the doses of 0.1 and 3 mg/kg, MS at the doses of 0.5, 15 and 45 mg/kg or tianeptine at the dose of 0.5 mg/kg compared to the rats administered with corticosterone and vehicle (PCB) (FIG. 33A). This increase in GR expression appeared to be proportional to the dose for MS and inversely proportional to the dose for tianeptine.

Repeated administration of corticosterone decreased the adrenal weight. Adrenal weight was observed to be higher in the rats treated with corticosterone plus GF, MS or tianeptine compared to the rats administered with corticosterone and vehicle (PCB) (FIG. 33B); however, it remained lower compared to the rats administered with corticosterone's vehicle and treatment's vehicle (Control). Notably the dose-effect patterns were similar to those observed for brain GR expression (FIG. 33A).

### 3.3.2 Experiments Using Methylprednisolone

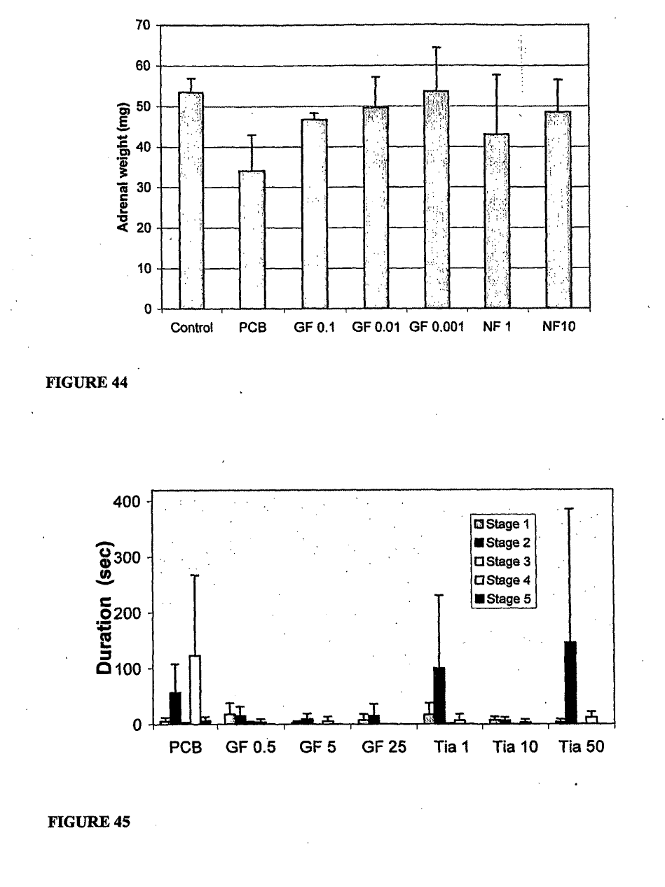

3.3.2.1 Effects of GF and Tianeptine on GS Activity in the Cortex, Hippocampus and Thalamus/Hypothalamus of Wistar Rats Administered with Methylprednisolone (FIGS. 42-43)
3.3.2.2 The Effects of a Chronic Treatment with GF (1 Mg/Kg) or Tianeptine (10 Mg/Kg) were Assessed in Rats Administered with Methyprednisolone (5 Mg/Kg i.p.). One Hour and 24 h Hours after the Last Administration, GF and Tianeptine Decreased GS Activity in the Thalamus/Hypothalamus, while a Trend to an Decrease and Increase of GS Activity was Observed in the Cortex and Hippocampus, Respectively. Effects of GF and NF on Adrenal Weight in Wistar Rats Administered with Methylprednisolone (FIG. 44).

The effects of a chronic treatment with GF (0.001, 0.01, 0.1 mg/kg) or naftazone (1 and 10 mg/kg) were assessed in rats administered with methyprednisolone (5 mg/kg i.p.) during 21 days. Adrenal weight was observed to be higher in the rats treated with methylprednisolone plus GF (non dose-dependent effect) or NF (dose-dependent effect) compared to the rats administered with methylprednisolone and vehicle (PCB). At the lower doses of GF (0.001 mg/kg), methylprednisolone-induced adrenal atrophy appeared even to be fully prevented. Repeated administration of methylprednisolone decreased adrenal weight.

### 3.4 Effects of GF and Tianeptine in a Model of PTZ-Induced Seizures

### 3.4.3 Effects of GF and Tianeptine on PTZ-Induced Seizures (FIG. 45)

GF and tianeptine decreased seizure occurrence after PTZ administration. This effect was not dose-dependent for GF and U-shaped (maximum effect at 10 mg/kg) for tianeptine.

### 3.4.4 Effects of GF and Tianeptine on GS Activity in the Cortex, Hippocampus and Thalamus/Hypothalamus of PTZ-Administered Wistar Rats (FIG. 46)

One hour after PTZ administration, GS activity was low in the cortex, hippocampus and thalamus. GF and tianeptine increased GS activity in the cortex, hippocamu and thalamus/hypothalamus. These treatment effects were not proportional to the dose.

TABLE 1
Conditions and disorders which (can) result in a systemic ammonia concentration
increase (hyperammonemia), reported according to the MedDRA classification
(MedDRA Version 7.1). For certain system organ classes (SOC), the included high
level group terms (HLGT) of particular interest are indicated; for certain HLGT, the
included high level terms (HLT) of particular interest are indicated; for certain HLT,
the included preferred terms (PT) of particular interest are indicated; for certain PT,
the included low level terms (LLT) of particular interest are indicated. Certain
conditions and disorders could not be reported according to the MedDRA
classification and are reported according their usual denomination used in the literature.

```
Hepatobiliary disorders (SOC)
  In particular
    Hepatic and hepatobiliary disorders (HLGT)
      In particular
        Cholestasis and jaundice (HLT)
          In particular Hepatitis cholestatic (PT)
        Hepatic and hepatobiliary disorders NEC (HLT)
          In particular Cerebrohepatorenal syndrome (PT), Complications of
          transplanted liver (PT), Hepatic infection (PT), Hepatic lesion (PT),
          Hepatic trauma (PT), Liver and pancreas transplant rejection (PT), Liver
          disorder (PT), Liver transplant rejection (PT), Polycystic liver disease (PT)
        Hepatic enzymes and function abnormalities (HLT)
        Hepatic failure and associated disorders (HLT)
          In particular
            Hepatic failure (PT)
              In particular Acute hepatic failure (LLT), Acute liver failure
              (LLT), Chronic hepatic failure (LLT), End stage liver disease
              (LLT), Failure liver (LLT), Fulminant hepatic failure (LLT),
              Hepatic failure (LLT), Hepatic insufficiency (LLT), Hepatobiliary
              insufficiency (LLT), Subacute hepatic failure (LLT)
            Hepatorenal failure (PT)
            Hepatorenal syndrome (PT)
        Hepatic fibrosis and cirrhosis (HLT)
          Biliary cirrhosis (PT), Biliary cirrhosis primary (PT), Biliary fibrosis (PT),
          Cardiac cirrhosis (PT), Cirrhosis alcoholic (PT), Congenital hepatic
          fibrosis (PT), Cryptogenic cirrhosis (PT), Hepatic cirrhosis (PT), Hepatic
          fibrosis (PT), Lupoid hepatic cirrhosis (PT)
        Hepatic infections (excl viral) (HLT)
        Hepatic metabolic disorders (HLT)
          In particular Alpha-1 anti-trypsin deficiency (PT), Haemochromatosis
          (PT), Hepatic siderosis (PT), Hepato-lenticular degeneration (PT),
          Hereditary haemochromatosis (PT)
        Hepatic vascular disorders (HLT)
          In particular Budd-Chiari syndrome (PT), Portal vein occlusion (PT),
          Portal vein phlebitis (PT), Portal vein stenosis (PT)
        Hepatic viral infections (HLT)
          In particular Hepatitis B (PT), Hepatitis C (PT), Hepatitis D (PT),
          Hepatitis non-A nonB non-C (PT)
        Hepatocellular damage and hepatitis NEC (HLT)
          In particular Alcoholic liver disease (PT), Chronic hepatitis (PT), Hepatic
          necrosis (PT), Hepatitis alcoholic (PT), Hepatitis chronic active (PT),
          Hepatitis chronic persistent (PT), Hepatitis fulminant (PT), Hepatitis toxic
          (PT), Peliosis hepatitis (PT), Reye's syndrome (PT)
    Hepatobiliary neoplasms (HLGT)
Metabolic and nutritional disorders congenital (HLGT)
    Urea cycle enzyme disorders
      In particular Arginase deficiency (PT) (argininaemia), Argininosuccinate
      synthetase deficiency (PT) (Citrullinaemia), Argininosuccinate lyase deficiency
      (PT) (Argininosuccinic aciduria), Carbamoyl phosphate synthetase deficiency
      (PT), N-acetylglutamate synthetase deficiency, Ornithine transcarbamoylase
      deficiency (PT)
    Transport defects of intermediates in the urea cycle
      In particular Lysinuric protein intolerance (PT), Hyperammonaemia-
      hyperornithinaemia-homocitrullinuria syndrome
    Organic acidurias
      Methylmalonic aciduria (PT) and other organic aciduria, Propionic aciduria
    Lipid metabolism disorders
      Medium-chain acetyl-coenzyme A dehydrogenase deficiency (PT); (Congenital)
      carnitine deficiency (PT), more particularly because of mutation of the gene
      encoding carnitine transporter; Long chain fatty acid oxidation defects and other
      related disorders
Other inborn errors
    Pyruvate carboxylase deficiency; Ornithine aminotransferase deficiency
Other metabolic causes
  (Distal) Renal tubular acidosis (PT); Hyperinsulinaemic hypoglycaemia
Porto-systemic shunts
  In particular due to Hepatobiliary disorders (SOC) (see above); Congenital absence of the
  portal vein; Surgical
Blood and lymphatic system disorders (SOC)
  In particular
    Haematopoietic neoplasms (excl leukaemias and lymphomas) (HLGT)
    Leukaemias (HLGT)
      In particular Leukaemias acute myeloid (HLT); Leukemia chronic myeloid (HLT)
    Plasma cell neoplasms (HLGT), in particular multiple myelomas (HLT)
    Allogenic bone marrow transplantation therapy (PT)
    Allogenic peripheral blood progenitor cell transplantation
    Drugs related hyperammonaemia
      In particular Intensive chemotherapy, more particularly with 5-fluorouracil and
      asparaginase; Anaesthetic agents, more particularly halothane and enflurane;
      Sodium valproate; Primidone
Infections and infestations (SOC)
  In particular with Herpes simplex in newborn (systemic infection); Urea splitting
  organisms; Urease positive bacteria
Renal and urinary disorders (SOC)
  In particular Subureteric injection for vesicoureteric reflux; Urinary diversion, more
  particularly with ureterosigmoidostomy and ileal conduit; Urinary tract infections, more
  particularly with urease positive bacteria
Other causes
  Muscular disorder e.g. rhabdomyolysis due to carnitine palmitoyltransferase deficiency;
  Parenteral nutrition, in particular arginine deficient total parenteral nutrition; Solid organ
  transplantation; Transient hyperammonaemia of the newborn; Upper gastrointestinal
  bleeding; Critical systemic illnesses and injuries; Idiopathic; Etc.
Abbreviations:
HLGT: high level group term;
HIT: high level term;
LLT: low level term;
NEC: not elsewhere classified;
PT: preferred term;
SOC: system organ class.
```

TABLE 2

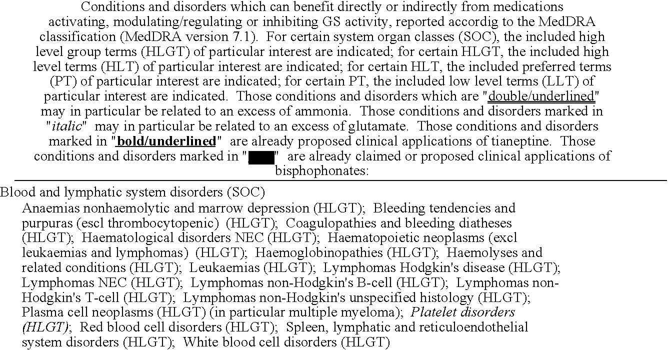

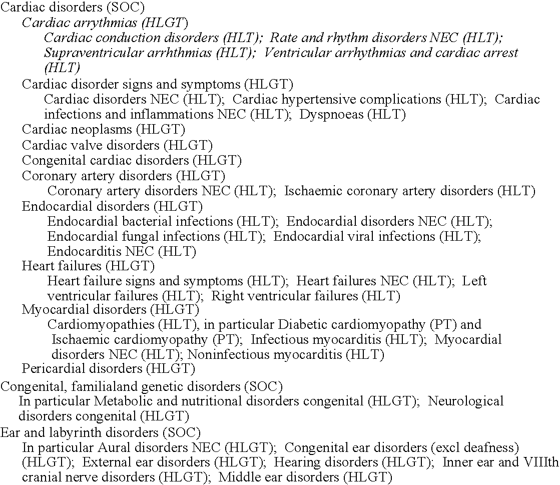

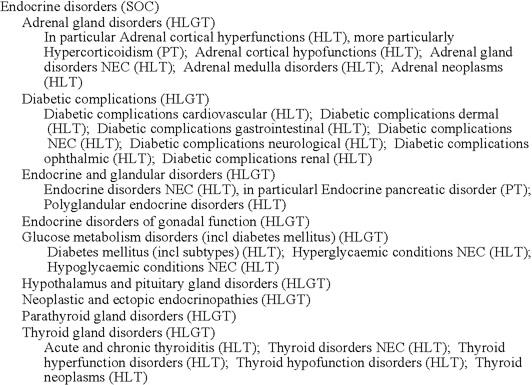

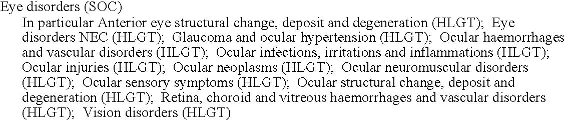

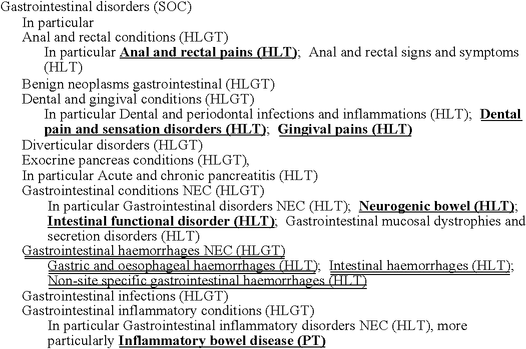

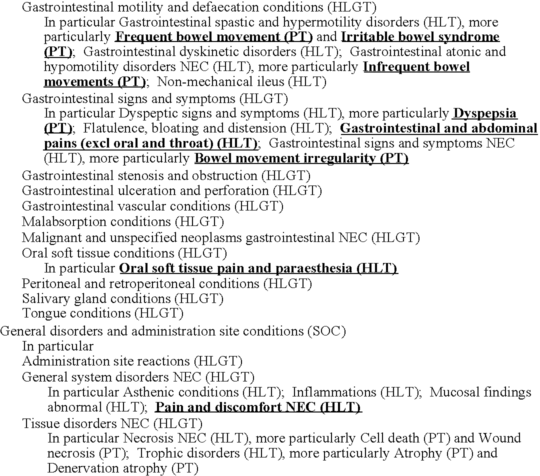

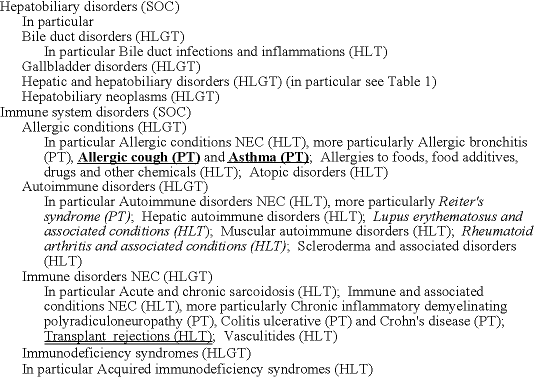

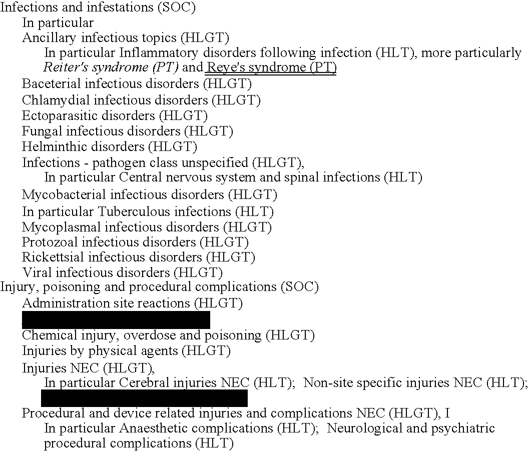

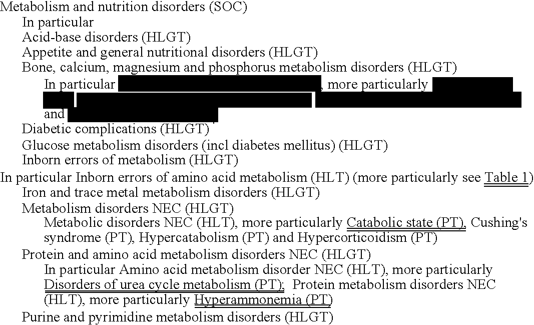

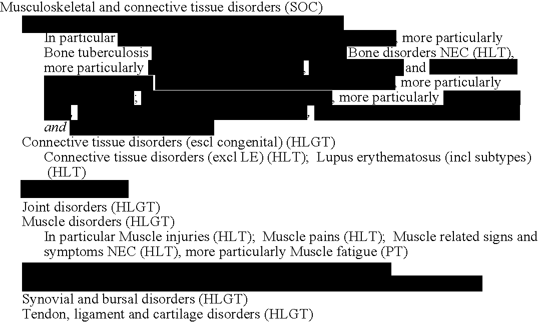

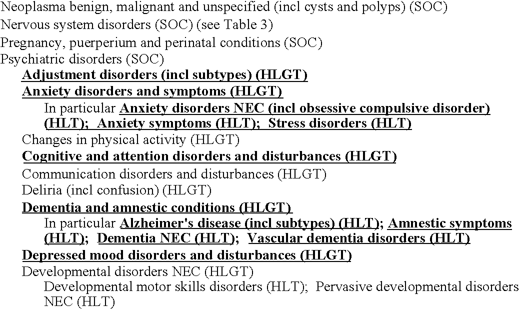

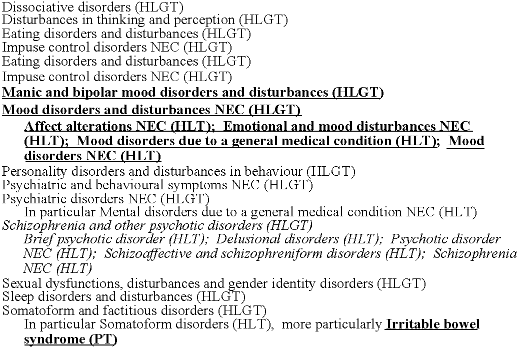

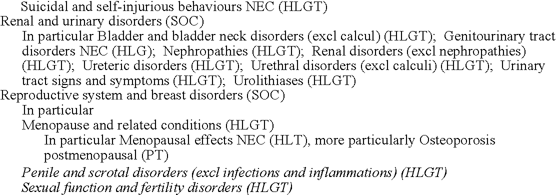

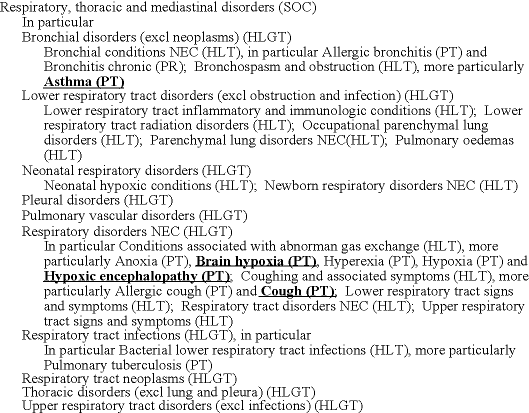

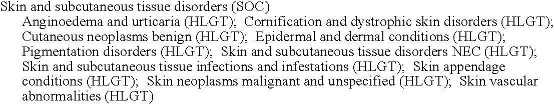

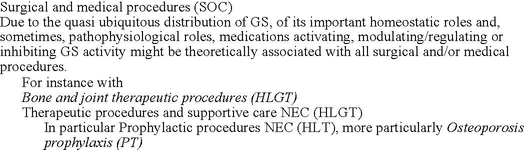

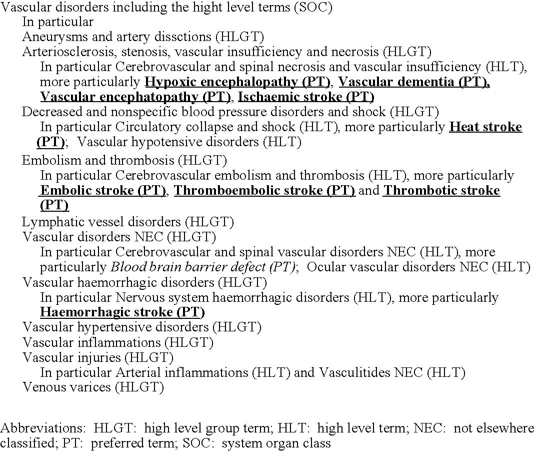

TABLE 3
Conditions and disorders of the nervous system which (can) benefit directly
or indirectly from medications activating, modulating/regulating or inhibiting
GS activity (reported according to the MedDRA classification; MedDRA
Version 7.1). For certain system organ classes (SOC), the included high
level group terms (HLGT) of particular interest are indicated; for certain HLGT,
the included high level terms (HLT) of particular interest are indicated; for certain
HLT, the included preferred terms (PT) of particular interest are indicated; for
certain PT, the included low level terms (LLT) of particular interest are indicated.
All these conditions and disorders are, more or less, related to or result in absolute
or relative deficiency or excess of glutamate. Those conditions and disorders marked
in “double/underlined“ are related to or result in an excess of ammonia Those
conditions and disorders marked in “bold/underlined“ are already proposed
clinical applications of tianeptine.

```
Nervous system disorders (SOC)
  Central nervous system infections and inflammations (HLGT)
    Central nervous system abscesses (HLT); Central nervous system inflammatory
    disorders NEC (HLT); Encephalitis NEC (HLT); Encephalitis nonviral infectious
    (HLT); Encephalitis of viral origin (HLT); Meningeal bacterial infections (HLT);
    Meningeal fungal infections (HLT); Meningeal viral infections (HLT); Meningitis
    NEC (HLT); Myelitis (incl infective) (HLT); Nervous system infections NEC
    (HLT)
  Central nervous system vascular disorders (HLGT)
      Central nervous system aneurysms (HLT);
      Central nervous system haemorrhages and cerebrovascular accidents (HLT)
        In particular Embolic stroke (PT), Haemorrhagic stroke (PT),
        Ischaemic stroke (PT), Thromboembolic stroke (PT) and Thrombotic
        stroke (PT)
      Central nervous system vascular disorders NEC (HLT)
        In particular Blood brain barrier defect (PT)
      Cerebrovascular venous and sinus thrombosis (HLT)
      Transient cerebrovascular events (HLT)
      Traumatic central nervous system haemorrhages (HLT)
  Congenital and peripartum neurological conditions (HLGT)
  Cranial nerve disorders (excl neoplasms) (HLGT)
  Demyelinating disorders (HLGT)
    In particular Multiple sclerosis acute and progressive (HLT)
  Encephalopathies (HLGT)
      Encephalopathies NEC (HLT), in particular AIDS encephalopathy (PT),
      Anoxic encephalopathy (PT), Encephalopathy allergic (PT), Encephalopathy
      neonatal (PT), Hypertensive encephalopathy (PT), Hypoxic
      encephalopathy (PT)
      Encephalopathies toxic and metabolic (HLT), in particular Hepatic
      encephalopathy (PT), Hypoglycaemic encephalopathy (PT), Reye's syndrome
      (PT) and Toxic induced encephalopathy (PT)
  Headaches (HLGT)
    Headaches NEC (HLT); Migraine headaches (HLT)
  Increased intracranial pressure and hydrocephalus (HLGT)
    Hydrocephalic conditions (HLT); Increased intracranial pressure disorders (HLT)
  Mental impairment disorders (HLGT)
    Alzheimer's disease (incl subtypes) (HLT); Dementia (excl Alzheimer's type)
    (HLT); Developmental disorders cognitive (HLT); Memory loss (excl
    dementia) (HLT); Mental impairment (excl dementia and memory loss)
    (HLT); Mental retardations (HLT)
  Movement disorders (incl Parkinsonism) (HLGT)
    Choreiform movements (HLT); Dyskinesias and movement disorders NEC (HLT);
    Dystonias (HLT); Paralysis and paresis (excl congenital and cranial nerve) (HLT);
    Parkinson's disease and parkinsonism (HLT); Tremor (excl congenital) (HLT);
    Nervous system neoplasms benign (HLGT); Gliomas benign (HLT); Meningiomas
    benign (HLT); Nervous system neoplasms benign NEC (HLT); Neuromas (HLT)
  Nervous system neoplasms malignant and unspecified NEC (HLGT)
    Central nervous system neoplasms malignant NEC (HLT); Glial tumours
    malignant (HLT); Meningiomas malignant (HLT); Nervous system neoplasms
    malignant NEC (HLT); Nervous system neoplasms unspecified malignancy NEC
    (HLT); Pineal parenchymal neoplasms (HLT)
  Neurological disorders NEC (HLGT)
    Abnormal reflexes (HLT); Cerebellar coordination and balance disturbances
    (HLT); Coma states (HLT), in particular Coma hepatic (PT); Cortical dysfunction
    NEC (HLT), in particular Cognitive deterioration (PT) and Confusion
    postoperative (PT); Disturbances in consciousness NEC (HLT); Nervous system
    disorders NEC (HLT), in particular Anaesthetic complication neurological (PT),
    Central nervous system lesion (PT) and Neurodegenerative disorder (PT);
    Neurological signs and symptoms NEC (HLT); Paraesthesias and dysaesthesias
    (HLT); Pupillary signs (HLT); Sensory abnormalities NEC (HLT); Speech and
    language abnormalities (HLT); Vertigos NEC (HLT)
  Neurological disorders of the eye (HLGT)
    Neurologic visual problems NEC (HLT); Ocular signs and symptoms NEC (HLT)
  Neuromuscular disorders (HLGT)
    Autonomic nervous system disorders (HLT), in particular Neurogenic bowel (PT)
    and Stress incontinence (PT); Motor neurone diseases (HLT), in particular
    Amyotrophic lateral sclerosis (PT); Muscle tone abnormal (HLT);
    Neuromuscular disorders NEC (HLT); Neuromuscular junction dysfunction (HLT)
  Peripheral neuropathies (HLGT)
    Acute polyneuropathies (HLT), in particular Guillain-Barre syndrome (PT);
    Chronic polyneuropathies (HLT); Inherited neuropathies (HLT);
    Mononeuropathies (HLT); Peripheral neuropathies NEC (HLT)
  Seizures (incl subtypes) (HLGT)
    Absence seizures (HLT); Generalised tonic-clonic seizures (HLT); Partial
    complex seizures (HLT), in particular Temporal lobe epilepsy (PT); Partial
    simple seizures NEC (HLT); Seizures and seizure disorders NEC (HLT)
  Sleep disturbances (incl subtypes) (HLGT)
    Abnormal sleep-related events (HLT); Disturbances in initiating and maintaining
    sleep (HLT); Disturbances in sleep phase rhythm (HLT); Narcolepsy and
    hypersomnia (HLT); Sleep apnoeas (HLT); Sleep disturbances NEC (HLT)
  Spinal cord and nerve root disorders (HLGT)
    Cervical spinal cord and nerve root disorders (HLT); Lumbar spinal cord and nerve
    root disorders (HLT); Spinal cord and nerve root disorders NEC (HLT); Spinal
    cord and nerve root disorders traumatic (HLT)
  Structural brain disorders (HLGT)
    Structural brain disorders (HLT), in particular Brain contusion (PT) and Brain
    damage (PT)
Abbreviations:
HLGT: high level group term;
HLT: high level term;
NEC: not elsewhere classified;
PT: preferred term;
SOC: system organ class.
```

TABLE 4
Factors and mechanisms which underlie conditions and
disorders which can be prevented or treated, alone or in
combination, with medications activating, modulating/regulating
or inhibiting GS activity. Certain of these factors and
mechanisms are reported according to the MedDRA
classification (MedDRA Version 7.1).

Age related factors (HLT) e.g. elderly (PT), menopause (PT),
post menopause (PT)
Allergic factors
Biomechanical factors
Catabolic states
Chemical factors e.g. acids, ammonia, bases, ions
(bivalent metal ions, . . . )
Degenerative processes
Dietary factors e.g. alcohol, monosodium L-glutamate, protein deficiency,
starvation
Drugs e.g. chemotherapies, corticosteroids, sodium valproate
Environmental issues (HLT)
Exercise e.g. extremely taxing exercice
Haemorrhagia
Hyperoxia
Hypoxia
Immune causes e.g. immunodeficiency, hypersensitivity, transplantation
Inflammatory processes e.g. connective tissue diseases, inflammatory
bowel diseases, myositis
Infections e.g. due to bacteria, fungi, protozoaires, viri, worms, . . . ,
or endotoxemia
Ischaemia
Metabolic stresses e.g. abnormal acid-base balance, hyperammonemia,
hypercorticism, hypoglycemia/hyperglycemia, siderosis
Neoplasm (benign/malignant)
Oxidative stresses e.g. free oxygen radicals, chlorinated oxidants
Physical stresses e.g. electricity, heat/cold, pressure,
radiations/radiotherapy
Surgery
Toxic factors e.g. antiseptic agents, detergents, paraquat
Trauma
. . .

TABLE 5
Conditions and disorders already claimed or proposed as clinical applications of
tianeptine (reported according to the MedDRA classification; MedDRA Version 7.1).

Anal and rectal pains (HLT); Dental pain and sensation disorders (HLT); Gingival pains (HLT);
Neurogenic bowel (HLT); Intestinal functional disorder (HLT); Inflammatory bowel disease (PT);
Frequent bowel movements (PT); Irritable bowel syndrome (PT); Infrequent bowel movements
(PT); Dyspepsia (PT); Gastrointestinal and abdominal pains (excl oral and throat) (HLT); Bowel
movement irregularity (PT); Oral soft tissue pain and paraesthesia (HLT); Pain and discomfort
NEC (HLT); Allergic cough (PT); Asthma (PT); Adjustment disorders (incl subtypes) (HLGT);
Anxiety disorders and symptoms (HLGT); Anxiety disorders NEC (incl obsessive compulsive
disorder) (HLT); Anxiety symptoms (HLT); Stress disorders (HLT); Cognitive and attention
disorders and disturbances (HLGT); Dementia and amnestic conditions (HLGT); Alzheimer's
disease (incl subtypes) (HLT); Amnestic symptoms (HLT); Dementia NEC (HLT); Vascular
dementia disorders (HLT); Depressed mood disorders and disturbances (HLGT); Manic and
bipolar mood disorders and disturbances (HLGT); Mood disorders and disturbances NEC (HLGT);
Affect alterations NEC (HLT); Emotional and mood disturbances NEC (HLT); Mood disorders due
to a general medical condition (HLT); Mood disorders NEC (HLT); Brain hypoxia (PT); Cough
(PT); Hypoxic encephalopathy (PT); Vascular dementia (PT); Vascular encephalopathy (PT);
Ischaemic stroke (PT); Heat stroke (PT); Embolic stroke (PT); Thromboembolic stroke (PT);
Thrombotic stroke (PT); Haemorrhagic stroke (PT); Central nervous system haemorrhages and
cerebrovascular accidents (HLT); Traumatic central nervous system haemorrhages (HLT);
Demyelinating disorders (HLGT); Multiple sclerosis acute and progressive (HLT); AIDS
encephalopathy (PT); Anoxic encephalopathy (PT); Hypertensive encephalopathy (PT); Mental
impairment disorders (HLGT); Dementia (excl Alzheimer's type) (HLT); Developmental disorders
cognitive (HLT); Memory loss (excl dementia) (HLT); Mental impairment (excl dementia and
memory loss) (HLT); Mental retardations (HLT); Neurodegenerative disorder (PT); Amyotrophic
lateral sclerosis (PT); Seizures (incl subtypes) (HLGT)
Abbreviations:
HLGT: high level group term;
HLT: high level term;
NEC: not elsewhere classified;
PT: preferred term.

TABLE 6
Conditions and disorders already proposed as clinical applications of hydrazines
(reported according to the MedDRA classification; MedDRA Version 7.1).

If said hydrazine is benserazide: Parkinson's disease and parkinsonism (HLT) (known
inhibitory properties on the aromatic L-aminoacid decarboxylase)
If said hydrazine is bumadizone: conditions and disorders sensitive to analgesic-antipyretic and
anti-inflammatory agents
If said hydrazine is dacarbazine: neoplasms (known antineoplastic properties)
If said hydrazine is dihydralazine: Heart failure (HLGT), Pre-eclampsia (PT), Vascular
hypertensive disorders (HLGT) and conditions and disorders sensitive to vasodilators
If said hydrazine is hydralazine: Heart failure (HLGT), Pre-eclampsia (PT), Vascular
hypertensive disorders (HLGT) and conditions and disorders sensitive to vasodilators
If said hydrazine is iproclozide: Mood alterations with depressive symptoms (HLGT) and
Depressive disorders (HLT) (known inhibitory properties on the monoamine oxidase), and
Ischaemic coronary artery disorders (HLT)
If said hydrazine is iproniazid: Mood alterations with depressive symptoms (HLGT) and
Depressive disorders (HLT) (known inhibitory properties on the monoamine oxidase), and
Vascular hypertensive disorders (HLGT)
If said hydrazine is isocarboxazide: Mood alterations with depressive symptoms (HLGT) and
Depressive disorders (HLT) (known inhibitory properties on the monoamine oxidase),
Ischaemic coronary artery disorders (HLT), Vascular hypertensive disorders (HLGT) and
conditions and disorders sensitive to vasodilators
If said hydrazine is isoniazid: Mycobacterial infectious disorders (HLGT) and Crohn's disease (PT)
If said hydrazine is nialamide: Mood alterations with depressive symptoms (HLGT) and
Depressive disorders (HLT) (known inhibitory properties on the monoamine oxidase),
Ischaemic coronary artery disorders (HLT), Vascular hypertensive disorders (HLGT) and
conditions and disorders sensitive to vasodilators
If said hydrazine is nifuroxazide: Gastrointestinal infections (HLGT)
If said hydrazine is phenicarbazide: Mood alterations with depressive symptoms (HLGT),
Depressive disorders (HLT), Migraine headaches (HLT) and Crohn's disease (PT)
If said hydrazine is picadralazine: vascular hypertensive disorders (HLGT), and conditions and
disorders sensitive to vasodilators
If said hydrazine is procarbazine: neoplasms (antineoplastic properties)

Abbreviations:
HLGT: high level group term;
HLT: high level term;
PT: preferred term.

TABLE 7
Conditions and disorders already proposed as clinical applications of bisphosphonates
(reported according to the MedDRA classification; MedDRA Version 7.1).

Bone and joint injuries (HLGT); Post-traumatic osteoporosis (PT); Osteoporosis (PT);
Osteoporosis circumscripta cranii (PT); Osteoporosis postmenopausal (PT); Senile osteoporosis
(PT); Bone disorders (excl congenital and fractures) (HLGT); Bone and joint infections (excl
arthritis) (HLT); Bone disorders NEC (HLT); Bone erosion (PT); Osteolysis (PT); Post-traumatic
osteoporosis (PT); Bone related signs and symptoms (HLT); Bone pain (PT); Metabolic bone
disorders (HLT); Fractures (HLGT); Musculoskeletal and connective tissue neoplasms
(benign/malignant) (HLGT); Bone cancer metastatic (PT); Metastases to bone (PT); Gaucher's
disease (PT); Protozoal infectious disorders (HLGT); Vascular calcification (PT)
Abbreviations:
HLGT: high level group term;
HLT: high level term;
NEC: not elsewhere classified;
PT: preferred term.

TABLE 8
Tianeptine, metabolites and analogs.

| | |
|---|---|
| 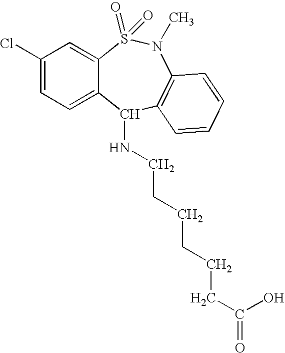 | Tianeptine |
| 7-(8-Chloro-11-methyl-10,10-dioxo-10,11- | |
| dihydro-5H-10λ⁶-thia-11-aza-dibenzo | |
| [a,d]cyclohepten-5-ylamino)-heptanoic acid | |
| 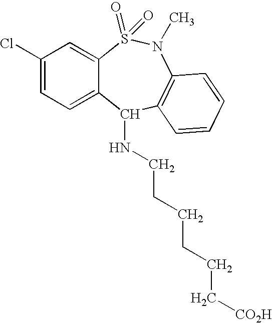 | Metabolite |
| 5-(8-Chloro-11-methyl-10,10-dioxo-10,11- | |
| dihydro-5H-10λ⁶-thia-11-aza-dibenzo | |
| [a,d]cyclohepten-5-ylamino)-pentanoic acid | |
| 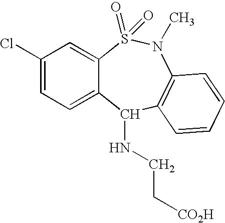 | Metabolite |
| 3-(8-Chloro-11-methyl-10,10-dioxo-10,11- | |
| dihydro-5H-10λ⁶-thia-11-aza-dibenzo | |
| [a,d]cyclohepten-5-ylamino)-propionic acid | |
| 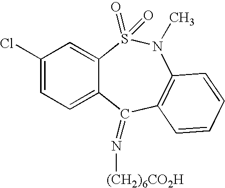 | Analog |
| 7-(8-Chloro-11-methyl-10,10-dioxo-10,11- | |
| dihydro-10λ⁶-thia-11-aza-dibenzo | |
| [a,d]cyclohepten-5-ylideneamino)-heptanoic acid | |
| 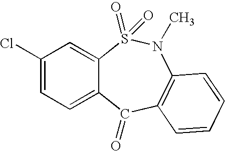 | Analog |
| 8-Chloro-11-methyl-10,10-dioxo-10,11- | |
| dihydro-10λ⁶-thia-11-aza- | |
| dibenzo[a,d]cyclohepten-5-one | |
| 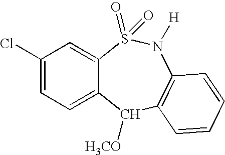 | Analog |
| 8-Chloro-5-methoxy-5,11-dihydro-10-thia-11-aza- | |
| dibenzo[a,d]cycloheptene 10,10-dioxide | |
| 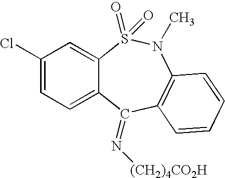 | Analog |
| 5-(8-Chloro-11-methyl-10,10-dioxo-10,11- | |
| dihydro-10λ⁶-thia-11-aza-dibenzo | |
| [a,d]cyclohepten-5-ylideneamino)-pentanoic acid | |
| 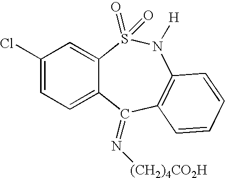 | Analog |
| 5-(8-Chloro-10,10-dioxo-10,11-dihydro-10λ⁶- | |
| thia-11-aza-dibenzo[a,d]cyclohepten-5- | |
| ylideneamino)-pentanoic acid | |
| 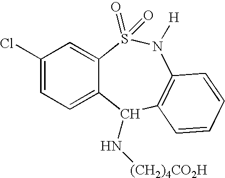 | Analog |
| 5-(8-Chloro-10,10-dioxo-10,11-dihydro-5H-10λ⁶- | |
| thia-11-aza-dibenzo[a,d]cyclohepten-5-ylamino)- | |
| pentanoic acid; compound with methane | |
| 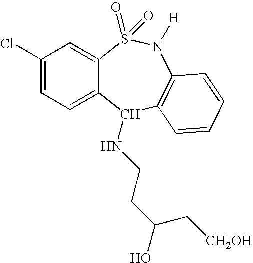 | Analog |
| 5-(8-Chloro-10,10-dioxo-10,11-dihydro-5H-10λ⁶- | |
| thia-11-aza-dibenzo[a,d]cyclohepten-5-ylamino)- | |
| pentane-1,3-diol | |
| 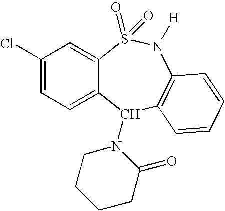 | Analog |
| 1-(8-Chloro-10,10-dioxo-10,11-dihydro-5H-10λ⁶- | |
| thia-11-aza-dibenzo[a,d]cyclohepten-5-yl)-piperidin- | |
| 2-one | |

TABLE 9
GF, metabolites and analogs

| | |
|---|---|
| 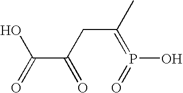 | Metabolite Ruhland et al., 2002 |
| 4-Methylphosphinico-2-oxo-butanoic acid | |
| 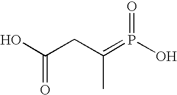 | Metabolite Ruhland et al., 2002 |
| 3-Methylphosphinicopropionic acid | |
| 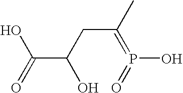 | Metabolite Ruhland et al., 2002 |
| 4-Methylphosphinico-2-hydroxybutanoic | |
| acid | |
| 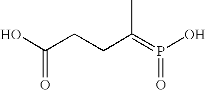 | Metabolite Ruhland et al., 2002 |
| 4-Methylphosphinicobutanoic acid | |
| 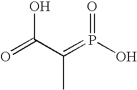 | Metabolite Ruhland at al., 2002 |
| 2-Methylphosphinicoacetic acid | |
| 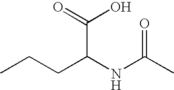 | Metabolite Ruhland et al., 2002 |
| 2-Acetamido-4-methylbutanoic acid | |
| 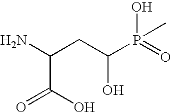 | Synthetic inhibitor Ki = 47 μM (sheep brain) Logusch et al., 1989 Eisenberg et al., 2000 |
| Gamma-hydroxy phosphinothricin | |
| 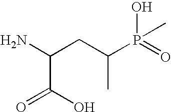 | Synthetic inhibitor Ki = 407 μM (sheep brain) Logusch et al., 1989 Eisenberg et al., 2000 |
| Gamma methyl phosphinothricin | |
| 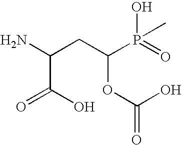 | Synthetic inhibitor Ki = 33 μM (E. coli) Walker et al., 1987 Eisenberg et al., 2000 |
| Gamma-acetoxy phosphinothricin | |
| 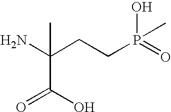 | Synthetic inhibitor Ki = 125 μM (sheep brain) Logusch et al., 1988 Eisenberg et al., 2000 |
| Alpha-methyl phosphinothricin | |
| 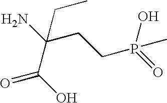 | Synthetic inhibitor Ki = 111 μM (sheep brain) Logusch et al., 1988 Eisenberg et al., 2000 |
| Alpha-ethyl phosphinothricin | |
| 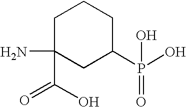 | Synthetic inhibitor Ki = 125 μM (sheep brain) Logusch et at., 1988 Eisenberg et at., 2000 |
| 1-Amino-3-phosphono-cyclohexane- | |
| carboxylic acid | |
| Cyclohexane phosphinothricin | |
| 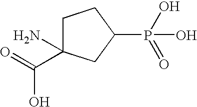 | Synthetic inhibitor Ki = 0.47 μM (mung bean) Johnson et at., 1990 Eisenberg et at., 2000 |
| 1-Amino-3-phosphono-cyclopentane- | |
| carboxylic acid | |
| Cyclopentane phosphinothricin | |
| 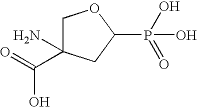 | Synthetic inhibitor Ki = 5 μM (mung bean) Johnson et al., 1990 Eisenberg et at., 2000 |
| 3-Amino-5-phosphono-tetrahydro-furan- | |
| 3-carboxylic acid | |
| Tetrahydrofuran phosphinothricin | |
| 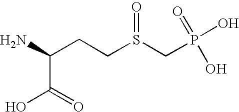 | Synthetic inhibitor Logusch et al., 1988 Eisenberg et at., 2000 |
| s-Phosphonomethyl homocysteine sulfoxide | |
| 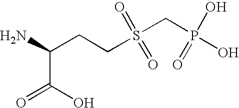 | Synthetic inhibitor Ki = 1.4 mM Logusch et at., 1988 Eisenberg et al., 2000 |
| s-Phosphonomethyl homocysteine sulfone | |
| 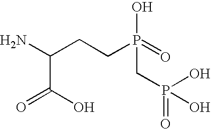 | Synthetic inhibitor Ki = 750 mM (sheep brain) Farrington et al., 1987 Eisenberg et al., 2000 |
| 2-Amino-4-[(phosphonomethyl)hydroxy- | |
| phosphinyl]butanoic acid | |
| 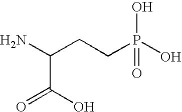 | Synthetic inhibitor Ki = 880 μM (rat liver) Lejczak et al., 1981 Eisenberg et al., 2000 |
| 2-Amino-4-phosphono butanoic acid | |
| 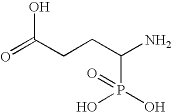 | Synthetic inhibitor Ki = 1.3 mM (rat liver) Lejczak et al., 1981 Eisenberg et al., 2000 |
| 4-Amino-4-phosphono butanoic acid | |
| 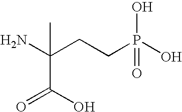 | Synthetic inhibitor with Ki = 6.3 mM (rat liver) Lejczak et al., 1981 Eisenberg et al., 2000 |
| 2-Amino-2-methyl-4-phosphono | |
| butanoic acid | |
| 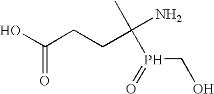 | Synthetic inhibitor Ki = 9.5 mM (rat liver) Lejczak et al., 1981 Eisenberg at al., 2000 |
| 4-Amino-4-(hydroxymethylphosphinyl)- | |
| 4-methyl butanoic acid | |
| 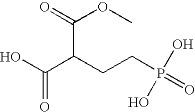 | Synthetic inhibitor Ki = 8.7 mM (rat liver) Lejczak et al., 1981 Eisenberg et al., 2000 |
| 2-Methoxycarbonyl-4-phosphono | |
| butanoic acid | |
| 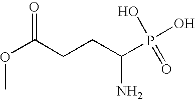 | Synthetic inhibitor Ki = 2.1 mM (rat liver) Lejczak et al., 1981 Eisenberg et al., 2000 |
| Methyl 4-amino-4-phosphono butanate | |
| 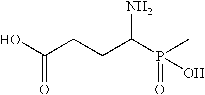 | Synthetic inhibitor Ki = 0.8 mM (rat liver) Lejczak et al., 1981 Eisenberg et al., 2000 |
| 4-Amino-4-(hydroxymethylphosphinyl) | |
| butanoic acid | |
| 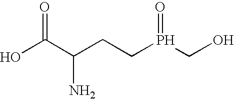 | Lejczak et al., 1981 Eisenberg et al., 2000 |
| 2-Amino-4-(hydroxymethylphosphinyl) | |
| butanoic acid | |
| 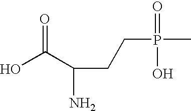 | Bayer et al., 1972 |
| Phosphinothricin | |
| 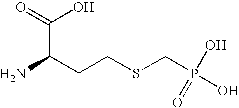 | Logusch et al., 1988 Eisenberg et al., 2000 |
| s-Phosphonomethyl homocysteine | |
| 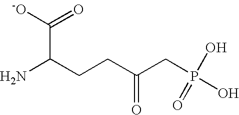 | Wedler et al., 1980 |
| 4-(Phosphonoacetyl)-L-alpha-aminobutyrate | |
| 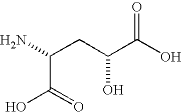 | Firsova et al., 1986 |
| Threo-4-hydroxy-D-glutamic acid | |
| 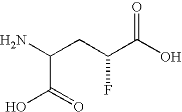 | Firsova et al., 1986 |
| Erythro-4-fluoro-D,L-glutamic acid | |
| 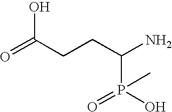 | Lejczak et al., 1981 |
| 4-Amino-4-(hydroxy-methyl-phosphinoyl)- | |
| butyric acid | |
| 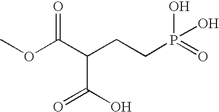 | Lejczak et al., 1981 |
| 2-Methoxycarbonyl-4-phosphono | |
| butanoic acid | |
| 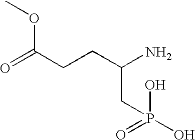 | Lejczak et al., 1981 |
| Methyl 4-amino-4-phosphono butanoate | |
| 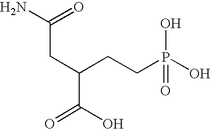 | Synthetic inhibitor Ki = 6.2 mM (rat liver) Eisenberg et al., 2000 |
| 2-Amido-4-phosphono butanoic acid | |
| Phosphinothricylalanylleucine (Phosalacine) | Uri et al., 1988 |

TABLE 10
MS and analogs

| | |
|---|---|
| 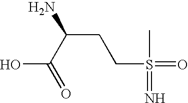 | Eisenberg et al., 2000 |
| Methionine sulfoximine | |
| 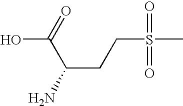 | Very potent ATP-dependent inhibitor of GS activity in a number of species. First discovered in nitrogen chloride treated zein. Eisenberg et al., 2000 |
| Methionine sulfone | |
| 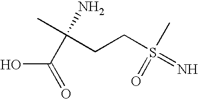 | Slightly less inhibitory synthetic MS derivative Eisenberg et al., 2000 |
| Alpha-methyl methionine sulfoximine | |
| 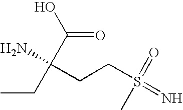 | Eisenberg et al., 2000 |
| Alpha-ethyl methionine sulfoximine | |
| 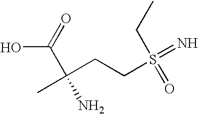 | Eisenberg et al., 2000 |
| Alpha-methyl ethionine sulfoximine | |
| 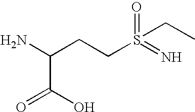 | Eisenberg et al., 2000 |
| Ethionine sulfoximine | |
| 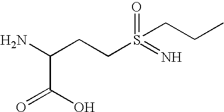 | Eisenberg et al., 2000 |
| Prothionine sulfoximine | |
| 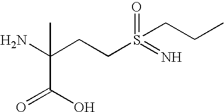 | Eisenberg et al., 2000 |
| Alpha-methyl prothionine sulfoximine | |
| 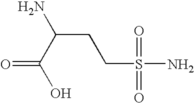 | Eisenberg et al., 2000 |
| 3-Amino-3-carboxypropane-sulfonamide | |
| 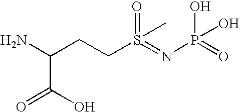 | Rowe et al., 1969 |
| Methionine sulfoximine phosphate | |

TABLE 11
Hydrazines

| | |
|---|---|
| H₂N—NH₂ | |
| Hydrazine | |
| 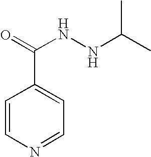 | Ito et al., 1992 |
| Iproniazid | |
| 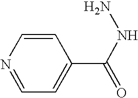 | Ito et al., 1992 |
| Isoniazid | |
| 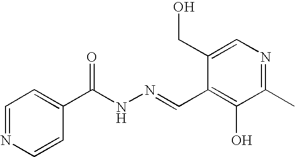 | Buss et al., 2004 |
| Pyridoxal isonicotinoyl hydrazone | |
| 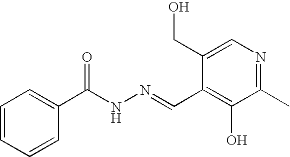 | Buss et at., 2004 |
| Pyridoxal benzoyl hydrazone | |
| 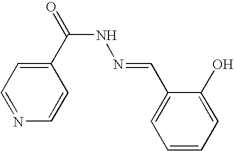 | Buss et al., 2004 |
| Salicylaldehyde isonicotinoyl hydrazone | |
| 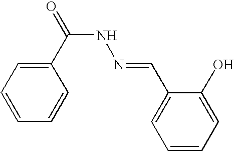 | Buss et al., 2004 |
| Salicylaldehyde benzoyl hydrazone | |
| 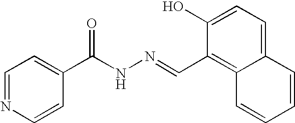 | Ito et al., 1992 |
| 2-Hydroxy-1-naphthaldehyde isonicotinoyl hydrazone | |
| 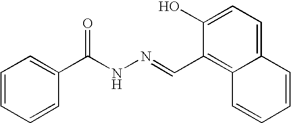 | Ito et al., 1992 |
| 2-Hydroxy-1-naphthaldehyde benzoyl hydrazone | |
| 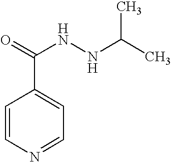 | Ito et al., 1992 |
| Isonicotinic acid N′-isopropyl-hydrazide | |
| 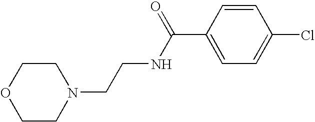 | King 1952 |
| 4-Chloro-N(2-morpholinoethyl)benzamide | |
| 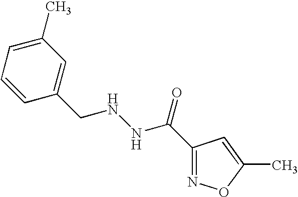 | Darling 1959 |
| 5-Methyl-isoxazole-3-carboxylic acid N′- | |
| 3-methyl-benzyl)-hydrazide | |
| 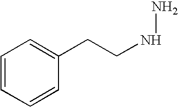 | Phenelzine Roh et al., 1994 |
| Phenethyl-hydrazine | |
| 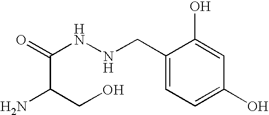 | Benserazide |
| DL-Serine 2-(2,3,4-trihydroxybenzyl)hydrazide | |
| 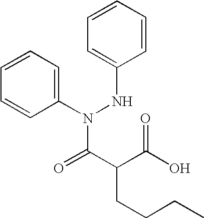 | Bumadizone |
| Butylmalonic acidmono-(1,2-diphenylhydrazide) | |
| 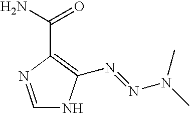 | Dacarbazine |
| 5-(3,3-Dimethyl-1-triazenyl)-1H-imidazole-4- | |
| carboxamide | |
| 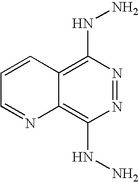 | Dihydralazine |
| 1,4-Dihydrazino-5-azaphthalazine | |
| 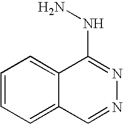 | Hydralazine |
| 1-Hydrazinophthalazine | |
| 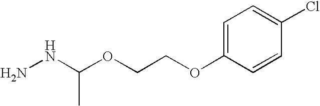 | Iproclozid |
| {1-[2-(4-Chloro-phenoxy)-ethoxy]-ethyl}-hydrazine | |
| 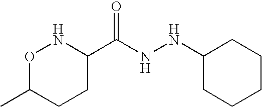 | Isocarboxazid |
| 6-Methyl-[1,2]oxazinane-3-carboxylic acid N′- | |
| cyclohexyl-hydrazide | |
| 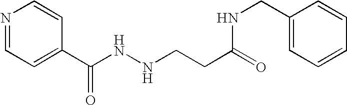 | Nialamide |
| Pyridine-4-carboxylic 2-[2-(benzylcarbamoyl) | |
| ethyl]hydrazide | |
| 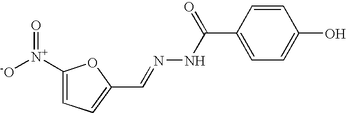 | Nifuroxazide |
| 5-Nitro-2-furaldehyde p-hydroxybenzoylhydrazone | |
| 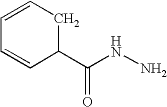 | Phenicarbazide |
| Cyclohexa-2,4-dienecarboxylic acid hydrazide | |
| 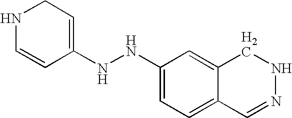 | Picodralazine |
| N-(3,4-Dihydro-phthalazin-6-yl)-N′-(1,2-dihydro-pyridin- | |
| 4-yl)-hydrazine | |
| 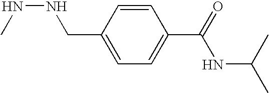 | Procarbazine |
| N-Isopropyl[(methyl-2hydrazino)methyl]-p-toluamide | |

TABLE 12
Bisphosphonates

| | |
|---|---|
| 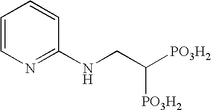 | Obojska et al., 2004 |
| [1-Phosphono-2-(pyridin-2-ylamino)-ethyl]- | |
| phosphonic acid | |
| 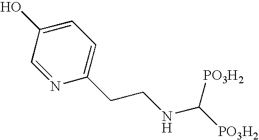 | Obojska et al., 2004 |
| {[2-(5-Hydroxy-pyridin-2-yl)-ethylamino]- | |
| phosphono-methyl}-phosphonic acid | |
| 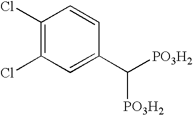 | Obojska et al., 2004 |
| [(3,4-Dichloro-phenyl)-phosphono-methyl]- | |
| phosphonic acid | |
| 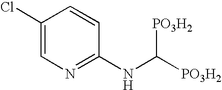 | Kafarski et al., 2001 |
| [(5-Chloro-pyridin-2-ylamino)-phosphono-methyl]- | |
| phosphonic acid | |
| 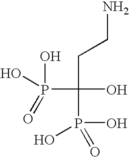 | Pamidronate Dunford et al., 2001 |
| 3-Amino-1-hydroxy-1-phosphonopropyl phosphonic | |
| acid | |
| 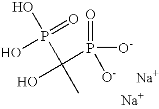 | Etidronate Reitsma et al., 1980 |
| Dihydrogen (1-hydroxyethylidene)bisphosphonate | |
| disodium | |
| 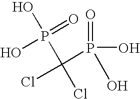 | Clodronate Wronski 1991 |
| Dichloromethylene bisphosphonate | |
| 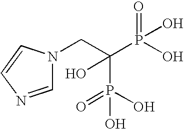 | Zolendronate Neville-Webbe et al., 2002 |
| (1-Hydroxy-2-imidazol-1-yl-1-phosphono-ethyl)- | |
| phosphonic acid | |
| 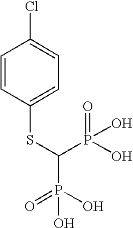 | Tiludronate Morales-Piga 1999 |
| [(4-Chloro-phenylsulfanyl)-phosphono-methyl]- | |
| phosphonic acid | |
| 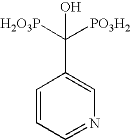 | Risedronate Wronski et at., 1991 |
| (Hydroxy-phosphono-pyridin-3-yl-methyl)- | |
| phosphonic acid | |
| 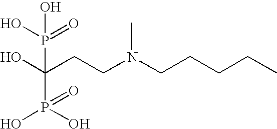 | Ibandronate Muhlbauer et al., 1991 |
| (Hydroxy-phosphono-pyridin-3-yl-methyl)- | |
| phosphonic acid | |
| 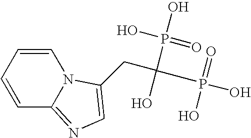 | Minodronate Takeuchi et al., 1998 |
| [1-hydroxy-2-(imidazo[1,2-a]pyridin-3-yl) | |
| ethylidene]bisphosphonic acid | |
| 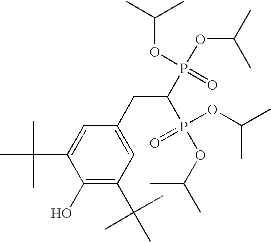 | Apomine Jackson et al., 2000 |
| Tetra-iso-propyl 2-(3,5-di-tert-butyl-4- | |
| hydroxyphenyl)ethyl-1,1-diphosphonate | |

TABLE 13
Other structural analogs of glutamate

| | |
|---|---|
| 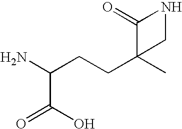 | Langston-Unkefer et al., 1987 |
| Tabtoxinine-β-lactam | |
| 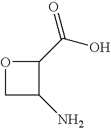 | Omura et al., 1984 |
| Oxetin | |
| 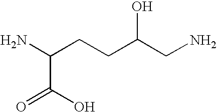 | Rabinovitz et al., 1957 |
| 5-Hydroxylysine | |
| 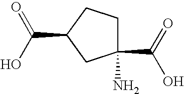 | Group I/II glutamate metabotropic receptor agonist Olverman et al., 1983 |
| 1-Aminocyclopentane-cis-1,3-dicarboxylic acid | |
| 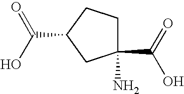 | Monaghan et al., 1989 |
| 1-Aminocyclopentane-trans-1,3-dicarboxylic | |
| acid | |
| 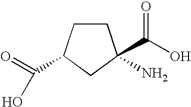 | Irving et al., 1990 |
| (1S,3R)-Aminocyclopentane-1,3-dicarboxylic | |
| acid | |
| 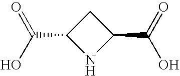 | Group I selective glutamate metabotropic receptor agonist. (activates mGlu₁ and mGlu₅) Kozikowski et al., 1993 |
| Trans-Azetidine-2,4-dicarboxylic acid | |
| 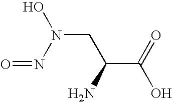 | Antibiotic produced by Streptomyces alansinicus. Anandaraj et al., 1980 |
| Alanosine | |
| 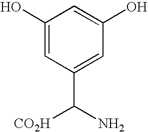 | Group I selective glutamate metabotropic receptor agonist (activates mGlu₁ and mGlu₅) Schoepp et al., 1994 |
| (RS)-3,5-Dihydroxyphenylglycine | |
| 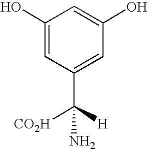 | Schoepp et al., 1994 |
| (S)-3,5-Dihydroxyphenylglycine | |
| 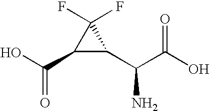 | Glutamate metabotropic receptor agonist Shinozaki et al., 1996 |
| (2S,1′S,2′S)-2-(2′-Carboxy-3′,3′- | |
| difluorocyclopropyl)glycine | |
| 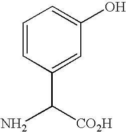 | Glutamate metabotropic receptor agonist Thomsen and Suzdak, 1993 |
| (RS)-3-Hydroxyphenylglycine | |
| 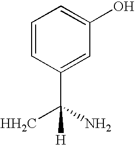 | Selective mGlu₁ glutamate metabotropic receptor agonist Thomsen et al., 1993 |
| S-3-Hydroxyphenylglycine | |
| 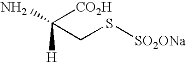 | mGlu₁ₐ and mGlu₅ₐ glutamate metabotropic receptor agonist Mewett et al., 1983 |
| S-Sulfo-L-cysteine sodium salt | |
| 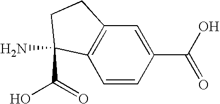 | mGlu₁ glutamate metabotropic receptor antagonist Pellicciari et al.,1995 |
| (RS)-1-Aminoindan-1,5-dicarboxylic acid | |
| 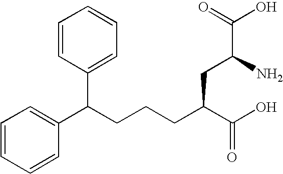 | Selective and competitive mGlu₂ glutamate metabotropic receptor antagonist Wermuth et al., 1996 |
| (4S)-4-(4,4-Diphenylbutyl)-L-glutamic acid | |
| 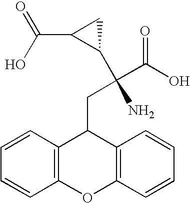 | Highly potent mGlu₂ glutamate metabotropic receptor antagonist Ornstein et al., 1998 |
| (2S)-2-Amino-2-[(1S,2S)-2-carboxycycloprop- | |
| 1-yl]-3-(xanth-9-yl) propanoic acid | |
| 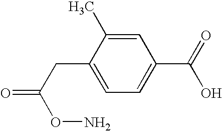 | Selective mGlu₁ₐ glutamate metabotropic receptor antagonist Clarke et al., 1997 |
| (S)-(+)-Amino-4-carboxy-2- | |
| methylbenzeneacetic acid | |
| 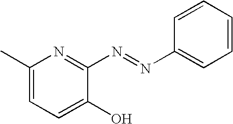 | Selective mGlu₅ glutamate metabotropic receptor antagonist Varney et al., 1999 |
| 6-Methyl-2-(phenylazo)-3-pyridinol | |
| 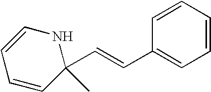 | Selective mGlu₅ glutamate metabotropic receptor antagonist Varney et al., 1999 |
| 2-Methyl-2-(phenylethenyl)pyridine | |
| 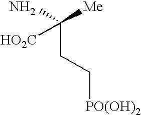 | Selective mGlu₃ glutamate metabotropic receptor antagonist Sekiyama et al., 1996 |
| 2-Amino-2-methyl-4-phosphono-butyric acid | |
| 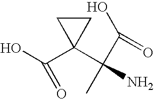 | mGlu₂ glutamate metabotropic receptor antagonist Jane et al., 1994 |
| (2S,3S,4S)-2-Methyl-2-(carboxycyclopropyl) | |
| glycine | |
| 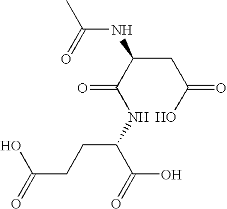 | Highly selective mGlu₃ glutamate metabotropic receptor agonist Morris et al., 1965 |
| N-Acetyl-L-aspartyl-L-glutamic acid | |
| 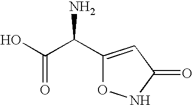 | Glutamate metabotropic receptor agonist Johnston, 1968 |
| Ibotenic acid | |
| 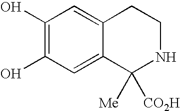 | Natural amino acid in the CNS affecting glutamate receptors Siggins et al., 1982 |
| Salsolinol-1-carboxylic acid | |
| 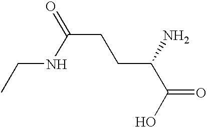 | Sugiyama and Sadzuka, 1998 |
| Theanine | |

TABLE 14
Conditions and disorders already claimed or proposed as clinical applications of NF
(reported according to the MedDRA classification; MedDRA Version 7.1).

At the present time, NF is only used for the treatment of venous insufficiency and venous varices
(HLGT). However, due to its interesting pharmacological profile i.e. protective effects against
lysosomial disruption and lipid peroxydation, platelet anti-aggregant properties and blunting effects
on glutamate release, NF has been claimed/proposed to be used for the prevention and/or treatment
of a large number of other conditions and disorders. These include:
Vascular disorders (SOC) conditions and disorders from Arteriosclerosis, stenosis, vascular
insufficiency and necrosis (HLGT), Embolism and thrombosis (HLGT), Vascular disorders
NEC (HLGT), Vascular haemorrhagic disorders (HLGT), Vascular inflammations (HLGT) and
venous varices (HLGT) groups, and
Nervous system disorders (SOC) from the Central nervous system vascular disorders (HLGT)
and other groups of conditions and disorders that are believed or have been demonstrated to be
associated with an excessive release of glutamate e.g. from Encephalopathies (HLGT), Mental
impairment disorders (HLGT), Movement disorders (incl Parkinsonism), Neurological
disorders of the eye (HLGT), Neuromuscular disorders (HLGT) and Seizures (incl subtypes)
groups such as acute and chronic neurodegenerative diseases, Alzheimer's, Huntington's,
Parkinson's diseases, multiple sclerosis, amyotrophic lateral sclerosis, spinal muscular atrophy,
retinopathy, and traumatic brain injury, drug-induced neurotoxicity, pain, hormonal balance,
blood pressure, thermoregulation, respiration, learning, pattern recognition, memory, and
disorders subsequent to hypoxia or hypoglycaemia.
Abbreviations:
HLGT: high level group term;
HLT: high level term;
NEC: not elsewhere classified;
PT: preferred term.

TABLE 15
Naftazone and its derivatives.

| | |
|---|---|
| 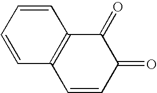 | U.S. Pat. No. 20020115617 A1 |
| 1,2-naphthoquinone | |
| 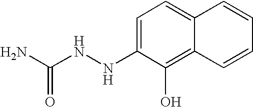 | U.S. Pat. No. 20020115617 A1 |
| 1-(1-hydroxy,2-naphthyl)semicarbazide | |
| 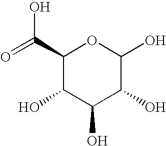 | U.S. Pat. No. 20020115617 A1 |
| gluco-pyranosiduronic acid | |
| 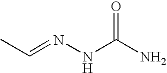 | U.S. Pat. No. 20020115617 A1 |
| Ethanone Semicarbazone | |

N.B. Other derivatives are described in US 20020115617 A1

### REFERENCES

ABCOUWER, et al. (1995). Surgery, 118, 325-34.
ABCOUWER, et al. (1996). Am J Physiol, 270, L141-51.
ABELL, et al. (1995). Biochemistry, 34, 16695-702.
ABELL, L. M. & VILLAFRANCA, J. J. (1991). Biochemistry, 30, 6135-41.
AGHA, A. M. & GAD, M Z. (1995). Pharmacol Res. 32, 279-85.
ALBRECHT, J. (2003). Gastroenterology, 125, 976-78.
ANAND, et al. (2000). Arch Gen Psychiatry, 57, 270-6.
ANANDARAJ, et al. (1980). Biochem Pharmacol, 29, 227-45.
ARAD, et al. (1976). Cell, 8, 95-101.
ARAD, G. & KULKA, R. G. (1978). Biochim Biophys Acta, 544, 153-62.
ARDAWI, M. S. (1990) Biochem J, 270, 829-32.
ARDAWI, M. S. & MAJZOUB, M. F. (1991). Metabolism, 40, 155-64.
ARDAWI, M. S. & NEWSHOLME, E. A. (1983). Biochem J, 212, 835-42.
AULT, B. & HILDEBRAND, L. M. (1993). Agents Actions, 39 Spec No, C142-4.
BABIJ, et al. (1986). J Physiol Lond, 347, 35P.
LE BACQUER, et al. (2001). Am J Physiol Gastrointest Liver Physiol, 281, G1340-7.
BAINS, J. S. & SHAW, C. A. (1997). Brain Res Brain Res Rev, 25, 335-58.
BARNETT, et al. (2000). Glia, 30, 64-73.
BAYER, et al. (1972). Helv Chim Acta, 55, 224-39.
BEAL, M. F. (1992). FASEB J, 6, 3338-44.
BERTRAND, et al. (1993). Eur J Pharmacol, 237, 45-50.
BEUTLER, E. (1989). Annu Rev Nutr, 9, 287-302.
BHANGU, et al. (2001). Bone, 29, 16-23.
BIRCH, et al. (1997). J Bone Miner Res, 12, O10.
BJARNASON, et al. (1995). Gastroenterology, 108, 1566-81.
BOELENS, et al. (2003). Am J Clin Nutr, 77, 250-6.
BRECHLINA, et al. (2000). Physiologia Plantarum, 108, 263-69.
BREMNER, et al. (1995). J Trauma Stress, 8, 527-53.
BRONSTEIN, et al. (1993). Brain Res Brain Res Rev, 18, 135-47.
BUSS, et al. (2004). Arch Biochem Biophys, 421, 1-9.
BUTTERWORTH, R. F. (1993). Dev Neurosci, 15, 313-9.
CAMPBELL, et al. (1950). Nature (London), 165: 34546.
CAO, et al. (1998). JPEN J Parenter Enteral Nutr, 22, 224-7.
CARLTON, et al. (1995). Neurosci Lett, 197, 25-8.
CASTANON, et al. (2001). Psychopharmacology (Berl), 154, 50-60.
CASTANON, et al. (2003). Psychoneuroendocrinology, 28, 19-34.
CASTILHO, et al. (995). Free Radic Biol Med, 18, 479-86.
CEYHAN, et al. (2005). J Psychiatric Res, 39, 191-196.
CHANDRASEKHAR, et al. (1999). Am J Physiol, 276, L319-31.
CHAUDHAR, et al. (2000). Nat Neurosci, 3, 113-9.
CHENU, C. (2002). Microsc Res Tech, 58, 70-6.
CHENU, et al. (1998). Bone, 22, 295-9.
CLARKE, et al. (1997). Nature, 389, 599-603.
COCK, et al. (1991). Plant Mol Biol, 17, 761-71.
CONRAD, et al. (1996). Behav Neurosci, 110, 1321-34.
CONVIT, et al. (1997). Neurobiol Aging, 18, 131-8.
COOPER, et al. (1985). J Neurochem, 44, 1716-23.
COOPER, A. J. & PLUM, F. (1987). Physiol Rev, 67, 440-519.
COSENTINO, et al. (1995). Neurosci Lett, 183, 139-42.
CURI, et al. (1999). Braz J Med Biol Res, 32, 15-21.
CUTHBERTSON, D. P. (1979). JPEN J Parenter Enteral Nutr, 3, 108-29.
DANTZER et al. (1998). Ann N Y Acad Sci, 840, 586-90.
DANTZER, et al. (1999). Mol Psychiatry, 4, 328-32.
DARLING, H. F. (1959). Am J Psychiatry, 116, 355-6.
DAVID, et al. (1988). Exp Eye Res, 46, 657-62.
DAWSON, et al. (1993). J Neurosci, 13, 2651-61.
DAWSON, et al. (1991). Proc Natl Acad Sci USA, 88, 6368-71.
DE GRAAF, et al. (1991). NMR Biomed, 4, 31-7.
DEITCH, E. A. (1992). Ann Surg, 216, 117-34.
DELBENDE, et al. (1991). Eur J Pharmacol, 202, 391-6.
DENG, et al. (2002). J Am Soc Nephrol, 13, 1381-4.
DICKMAN, et al. (2004). Am J Respir Cell Mol Biol, 30, 139-44.
DROGE, et al. (1988). J Cancer Res Clin Oncol, 114, 124-8.
DUNFORD, et al. (2001). J Pharmacol Exp Ther, 296, 235-4. Durand, et al. (1996) J Pharm Pharmacol 48, 566-72.
EATON, S. (2003). Proc Nutr Soc, 62, 745-51.
ECK, et al. (1989). Int Immunol, 1, 367-72.
EDWARDS, et al. (1990). Proc Natl Acad Sci USA, 87, 3459-63.
EID, et al. (2004). Lancet, 363, 28-37.
EISENBERG, et al. (2000). Biochim Biophys Acta, 1477, 12245.
EVANS, P. H. (1993). Br Med Bull, 49, 577-87.
EVSTIGNEEVA, et al. (2003). Prikl Biokhim Mikrobiol, 39, 613-8.
FALDUTO, et al. (1992). Am J Physiol, 262, C214-20.
FAROOQUI, et al. (1995). Mol Chem Neuropathol, 25, 155-73.
FARRINGTON, et al. (1987). J Med Chem, 30, 2062-7.
FELIPO, V. & BUTTERWORTH, R. F. (2002a). Neurochem Int, 40, 487-91.
FELIPO, V. BBUTTERWORTH, R. F. (2002b). Prog Neurobiol, 67, 259-79.
FENG, et al. (1990). J Cell Physiol, 145, 376-80.
FERGUS, A. & LEE, K. S. (1997). Brain Res, 754, 35-45.
FERRIER, et al. (1999). Biochem J. 337 (Pt 3), 543-50.
FINCH, C. E. & COHEN, D. M. (1997). Exp Neurol, 143, 82-102.
FIRSOVA, et al. (1986). Biokhimiia, 51, 850-5.
FLARING, et al. (2003). Clin Sci (Lond), 104, 275-82.
FOYER, C. H. & FERRARIO, S. (1994). Biochem Soc Trans, 22, 909-15.
FRANCONI, et al. (1996). Thromb Haemost, 76, 84-7.
FROST, H. M. (2002). J Bone Miner Metab, 20, 319-25.
FUENTES, et al. (2001). J Exp Bot, 52, 1071-81.
FURUKAWA, et al. (2000). Nutrition, 16, 323-9.
FUZIWARA, et al. (2003). J Invest Dermatol, 120, 1023-9.
GABBOTT, P. L. & SOMOGYI, J. (1984). J Neurosci Methods, 11, 221-30.
GALLARDO, et al. (1999). Planta, 210, 19-26.
GARCIA, et al. (1999). Clin Sci (Lond), 96, 549-55.
GARTHWAITE, et al. (1986). Neuroscience, 18, 437-47.
GASIC, G. P. & HOLLMANN, M. (1992). Ann Rev Physiol, 54, 507-36.
GENEVER, et al. (1999a). J Invest Dermatol, 112, 337-42.
GENEVER, et al. (1999b). Blood, 93, 2876-83.
GENEVER, P. G. & SKERRY, T. M. (2001). Faseb J, 15, 1586-8.
GIARONI, et al. (1999). Gastroenterology, 117, 1438-58.
GIARONI, et al. (2003). Eur J Pharmacol, 476, 63-9.
GILL, et al. (1998). Brain Res Bull, 46, 429-34.
GILL, H. S. & EISENBERG, D. (2001). Biochemistry, 40, 1903-12.
GLEVAREC, et al. (2004). Planta, 219, 286-97.
GONOI, et al. (1994). J Biol Chem, 269, 16989-92.
GONZALEZCADAVID, et al. (2000). J Androl, 21, 566-78.
GOROVITS, et al. (1997). Proc Natl Acad Sci USA, 94, 7024-9.
GOVITRAPONG et al. (1986). J Pineal Res, 3, 223-34.
GRIFFITH, et al. (1979). J Biol Chem, 254, 1205-10.
GURVITS, T et al. (1996). Biol Psychiatry, 40, 1091-9.
HACK, et al. (1994). Food Chem Toxicol, 32, 461-70.
HALESTRAP, et al. (1997). J Biol Chem, 272, 3346-54.
HARDER, et al. (1998). Stroke, 29, 229-34.
HAUSSINGER, et al. (1985). J Hepatol, 1, 3-14.
HAWKES, et al. (2001). Postgard Med J, 77, 316-7.
HAWKINS, et al. (1993). J Neurochem, 60, 1000-6.
HAYASHI, et al. (2003). J Biol Chem, 278, 1966-74.
HERBER, et al. (1995). Drug Metab Dispos, 23, 1305-14. HERTZ, L. (1979). Prog Neurobiol, 13, 277-323.
HERTZ, et al. (1999). J Neurosci Res, 57, 417-28.
HERTZ, L. & ZIELKE, H. R. (2004). Trends Neurosci, 27, 735-43.
HICKSON, et al. (1996). Am J Physiol, 270, E912-7.
HIGUCHI, et al. (2001). Hepatology, 34, 320-8.
HINOI, et al. (2002c) J Neurosci Res, 68, 36-45.
HINOI et al. (2002a). Biochem Biophys Res Commun, 297, 452-8.
HINOI, et al. (2003). FASEB J, 17, 1532-4.
HINOI, et al. (2002b). J Neurosci Res, 70, 161-71.
HINOI, et al. (2004). Eur J Biochem, 271, 1-13.
HINOI, E. & YONEDA, Y. (2001). Neurochem Int, 38, 539-47.
HITCHCOCK, et al. (2003). Blood, 102, 1254-9.
HOLLMANN, M. & HEINEMANN, S. (1994). Annu Rev Neurosci, 17, 31-108.
HONG, et al. (1992). Ann Surg, 215, 114-9.
HOUDIJK, et al. (1999). JPEN J Parenter Enteral Nutr, 23, S52-8.
HOUDJIK, et al. (1998). Lancet, 352, 772-6.
HOURANI, et al. (1971). Arch Intern Med, 127, 1033-6.
HOYER, S. (1994). Adv Exp Med Biol, 368, 197-205.
HREBICEK, J. & KOLOUSEK, J. (1968). Epilepsia, 9, 145-62.
HUNDAL, et al. (1990). Am J Physiol, 259, E148-54.
INAGAKI, et al. (1995). Faseb J, 9, 686-91.
IRVING, et al. (1990). Eur J Pharmacol, 186, 363-5.
ISAACKS, et al. (1999). Neurochem Res, 24, 51-9.
ISHIYAMA, et al. (2004). Plant Cell Physiol, 45, 1640-7.
ISRAELI, et al. (2004). Dig Dis Sci, 49, 1705-12.
ITO, et al. (1992). Biochemistry, 31, 11606-13.
IZUMI, et al. (1992). Neurosci Lett, 135, 227-30.
JACKSON, et al. (2000). Arzneimittelforschung, 50, 380-6.
JANE, et al. (1994). Br J Pharmacol, 112, 809-16.
JAYAKUMAR, et al. (2004). Glia, 46, 296-301.
JOHNSON, et al. (1990) Bioorg Chem, 18, 154-9.
JOHNSTON, G. A. (1968). J Neurochem, 15, 1013-7.
JONES, E. A. & WEISSENBORN, K. (1997). J Neurol Neurosurg Psychiatry, 63, 279-93.
JOU, et al. (2002). J Biomed Sci, 9, 507-16.
KAFARSKI, et al. (2000). Heteroatom Chemistry, 11, 449-53.
KAFARSKI, P. & LEJCZAK, B. (2001). Curr Med Chem Anti-Canc Agents, 1, 301-12.
KANAMORI, et al. (1997). Adv Exp Med Biol, 420, 185-94.
KARINCH, et al. (2001). J Nutr, 131, 2535S-8S.
KING, D. S. (1952). Tuberculosis. N Engl J Med, 247, 718-25.
KIRCHGESSNER, A. L. (2001). Curr Opin Pharmacol, 1, 591-6.
KIRCHGESSNER, et al. (1997). J Neurosci, 17, 8804-16.
KIYAMA, H et al. (1993). Brain Res Mol Brain Res, 19, 262-8.
KLEIN-SOYER et al. (1995). Nouv Rev Fr Hematol, 37, 187-92.
KOLLEGGER, et al. (1991). Neurosci Lett, 130, 95-8.
KOSENKO, et al. (2003). Neurochem Int, 43, 493-9.
KOSOWER, N. S. & KOSOWER, E. M. (1978). Int Rev Cytol, 54, 109-60.
KOSTANYAN, I et al. (1997). Immunol Lett, 58, 177-80.
KOYAMA, et al. (1992). Adv Exp Med Biol, 315, 355-9.
KOZIKOWSKI, et al. (1993). J Med Chem, 36, 2706-8.
KRAKOFF, I. H. (1961). Clin Pharm Ther, 2, 599-04.
KRISTENSEN, P. (1993). FEBS Lett, 332, 14-8.
KRIZBAI, et al., (1998). J Neurosci Res, 54, 814-9.
KVAMME, E. (1998). Prog Brain Res, 116, 73-85.
LABOW, B et al. (1998). Am J Physiol, 275, L877-86.
LABOW, et al. (1999). Am J Physiol, 276, E1136-45.
LABOW, et al. (2001). J Nutr, 131, 2467S-74S.
LACOSTE, et al. (1982). J Neurochem, 39, 78-85.
LAM, et al. (1995). Plant Cell, 7, 887-98.
LAM, et al. (1996). Annu Rev Plant Physiol Plant Mol Biol, 47, 569-93.
LAMBERTY, Y. & KLITGAARD, H. (2000). Epilepsy Behav, 1, 256-61.
LANGSTON-UNKEFER, et al. (1987). J Biol Chem, 262, 1608-13.
LANYON, L. & SKERRY, T. (2001). J Bone Miner Res, 16, 1937-47.
LAPOUBLE, et al. (2002). Brain Res, 957, 46-52.
LARA, et al. (1983). L. Planta, 157, 254-258.
LEA, P. J. & RIDDLEY S. M. (1989). In: Herbicides and plant metabolism (Seminar series/Society for Experimental Biology; 38). A D Dodge (ed) Cambridge University Press, Cambridge, UK, 137-70.
LEA, P. J. (1997). In: Plant biochemistry. P M Dey, J B Harborne (eds) Academic Press, London, UK, 273-306.
LECHIN, et al. (1998). Clin Pharmacol Ther, 64, 223-32.
LEE, et al. (1998). Comb Chem High Throughput Screen, 1, 35-46.
LEJCZAK, et al. (1981). Experientia, 37, 461-2.
LEONARD, J. V. & MORRIS, A. A. (2002). Semin Neonatol, 7, 27-35.
LEUNG, et al. (2002). Am J Physiol Regul Integr Comp Physiol, 283, R964-71.
LI, et al. (2004) Am J Physiol Gastrointest Liver Physiol, 287, G726-33.
LIMAMI, et al. (1999) Planta, 209, 495-502.
LIMAMI, et al. (2002). Plant Physiology, 130, 1860-70.
LIPTON, S. A. & GENDELMAN, H. E. (1995). N Engl J Med, 332, 934-40.
LIPTON, S. A. & ROSENBERG, P. A. (1994). N Engl J Med, 330, 613-22.
LIU, et al. (1997b). Pancreas, 14, 360-68.
LIU, et al. (1997a). J Neurosci, 17, 4764-84.
LODIN, Z. (1958). Cesk Fysiol, 7, 122-8.
LODIN, Z. & KOLOUSEK, J. (1956). Cesk Neurol, 19, 83-9.
LOGUSCH, E. W. (1988). Tetrahedron Lett, 29, 6055-58.
LOGUSCH, et al. (1988). J Org. Chem., 53, 40694074.
LOGUSCH, et al. (1989). Biochemistry, 28, 3043-51.
LOGUSCH, et al. (1990). Biochemistry, 29, 366-72.
LOMBARDI, et al. (2001). Characterization of ionotropic glutamate receptors in human lymphocytes. Br J Pharmacol, 133, 936-44.
LOUZADA-JUNIOR, et al. (1992) J. Neurochem. 59, 358-363.
LUINE (1992). Br J Psychiatry Suppl, 6-75.
LUKASZEWICZ, et al. (1997). Shock, 7, 332-8.
MAINOUS, et al. (1994). New Horiz, 2, 193-201.
MAGARINOS, A. M. & MCEWEN, B. S. (1995). Neuroscience, 69, 89-98.
MANHART, et al. (2001). Ann Surg, 234, 92-7.
MARCINKEWICZ, et al. (1995). J Leukoc Biol, 58, 667-74.
MASON, et al. (1997). Bone, 20, 199-205.
MASSEY, S. C. & MILLER, R. F. (1990). J Neurophysiol, 63, 16-30.
MATTEI, et al. (1999). Neurosci Lett, 271, 183-186.
MATSUMURA et al. (2001). Neurosci Lett, 304, 123-5.
MCCAULEY, et al. (1998). JPEN J Parenter Enteral Nutr, 22, 105-11.
MCEWEN, et al. (1993). Eur Psychiatr, 8 suppl2, 41S-48S.
MCEWEN, B. S. & CHATTARJI, S. (2004). Eur Neuropsychopharmacol, 14, S497-S502.
MCGRATH-MORROW, S. A. & STAHL, J. (2002). Am J Respir Cell Mol Biol, 27, 99-106.
MCNEARNEY, et al. (2000). J Rheumatol, 27, 739-45.
MCROBERTS, et al. (2001). Gastroenterology, 120, 173748.
MEISTER, A. (1985). Methods Enzymol, 113, 185-99.
MEISTER, A. (1991). Pharmacol Ther, 51, 155-94.
MEISTER, A. & TATE, S. S. (1976). Annu Rev Biochem, 45, 559-604.
MELIS, et al. (2004). Curr Opin Clin Nutr Metab Care, 7, 59-70.
MENG, et al. (1995). Stroke, 26, 857-62.
MERLE, et al. (2003). J Cell Biochem, 90, 424-36.
MEWETT, et al. (1983). Adv Biochem Psychopharmacol, 37, 163-74.
MIFLIN, B. J. & HABASH, D. Z. (2002). J Exp Bot, 53, 979-87.
MIFLIN B. J. & LEA P. J. (1980). In: The biochemistry of plants. Vol. 5. Amino acids and their derivatives. B J Miflin (ed) Academic Press, New York, USA, 169-202.
MIGGE, et al. (2000). Planta, 210, 252-60.
MILLER, A. L. (1999). Altern Med Rev, 4, 239-48.
MILMAN, et al. (1975). J Biol Chem, 250, 1393-9.
MONAGHAN, et al. (1989). Annu Rev Pharmacol Toxicol, 29, 365-402.
MORALES-PIGA, A. (1999). Expert Opin Pharmacother, 1, 157-70.
MORRIS, D. & STRAUGHAN, D. W. (1965). Biochem Pharmacol, 14, 1679-81.
MOSINGER, et al. (1991). Exp Neurol, 113, 10-7.
MUHLBAUER, et al. (1991). J Bone Miner Res, 6, 1003-11.
MURTHY, et al. (2001). J Neurosci Res, 66, 282-8.
NAGATA, Y., HOMMA, H., LEE, J. A. & IMAI, K. (1999). FEBS Lett, 444, 160-4.
NAKAKI, et al. (2000). Neurosci Lett, 290, 209-12.
NEAL, et al. (1994). J Neurochem, 62, 1025-33.
NEU, et al. (2002). Curr Opin Clin Nutr Metab Care, 5, 69-75.
NEVILLE-WEBBE, et al. (2001). J Nutr, 131, 2515S-22S,
NORENBERG, M. D. (1981). Adv Cell Neurobiol, 2: 303-51.
NORENBERG, M. D. (1986). In Astrocytes. S Fedoroff, A Vemadakis (eds) Academic Press, New York, USA, 425-460.
NORENBERG, M. D. (1987). Neurochem Pathol, 6, 13-33.
NORENBERG, M. D. & BENDER, A. S. (1994). Acta Neurochir Suppl (Wien), 60, 24-7.
NORENBERG, M. D. & MARTINEZ-HERNANDEZ, A. (1979). Brain Res, 161, 303-10.
NORENBERG, et al. (1992). Prog Brain Res, 94, 261-9.
NORENBERG, et al. (2004). J Bioenerg Biomembr, 36, 303-7.
OBOJSKA, et al. (2004). J Agric Food Chem, 52, 3337-44.
OBRENOVITCH, T. P. & URENJAK, J. (1997). Prog Neurobiol, 51, 39-87.
OBRENOVITCH, et al. (2000). Int J Dev Neurosci, 18, 281-7.
OHTAKE, et al. (2001). Hum Exp Toxicol, 20, 429-34.
OLIVEIRA, et al. (2002). Plant Physiology, 129, 1170-80.
OLKKU, et al. (2004). Bone, 34, 320-9.
OLNEY, J. W. (1990). Prog Brain Res, 86, 37-51.
OLVERMAN, H. J. & SHARMAN, D. (1983). Comp Biochem Physiol C, 75, 223-5.
OMURA, et al. (1984). J Antibiot (Tokyo), 37, 1324-32.
ORNSTEIN, et al. (1998). J Med Chem, 41, 358-78.
ORREGO, F. & VILLANUEVA, S. (1993). Neuroscience, 56, 539-55.
ORZECHOWSKI et al. (2000). Horm Metab Res, 32, 174-80.
PATEL, et al. (1986). Brain Res, 391, 229-38.
PATTON, et al. (1998). Bone, 22, 645-9.
PAULSEN, et al. (1988). J Neurochem, 51, 1294-9.
PEET, et al. (1999). Faseb J, 13, 2179-85.
PELLICCIARI, et al. (1995). J Med Chem, 38, 3717-9.
PITHON-CURI, et al. (2003). Am J Physiol Cell Physiol, 284, C1355-61.
PLAISANT, et al. (2003). Neuropharmacology, 44, 801-9.
PLANAS, et al. (1993). JPEN J Parenter Enteral Nutr, 17, 299-300.
POTSIC, et al. (2002). Pediatr Res, 52, 430-6.
PUSHKIN, et al. (1974). Fiziol. Rast.(Moscow), 21, 512-7.
PUSHKIN, et al. (1975). Biokhimiya (Moscow), 40, 53-6.
RABINOVITZ, et al. (1957). Cancer Res, 17, 885-9.
RAO, K. V. & NORENBERG, M. D. (2004). J Biol Chem, 279, 32333-8.
RAO, et al. (2003). Neurochemistry International, 43, 517-23.
RAO, S. L. & MEISTER, A. (1972). Biochemistry, 11, 1123-7.
RASTOGI, et al. (1998). Plant Cell Physiol, 39, 443-6.
REAGAN, et al. (2004). Proc Natl Acad Sci USA, 101, 2179-84.
REIS, et al. (2000). Neuroscience, 101, 237-42.
REITER, R. J. (1995). Faseb J, 9, 526-33.
REITSMA, et al. (1980). N Engl J Med, 303, 162-3.
ROH, et al. (1994). Biosci Biotechnol Biochem, 58, 1652-6.
RHOADS, J et al. (1995). J Pharmacol Exp Ther, 274, 404-12.
RICHIE, et al. (1987). Proc Soc Exp Biol Med, 184, 113-7.
ROBINSON, et al. (1992). JPEN J Parenter Enteral Nutr, 16, 413-8.
ROSS, et al. (1996). Dig Dis, 14 Suppl 1, 30-9.
ROTH, et al. (2002). Nutrition, 18, 217-21.
ROTHWELL, et al. (1997). J Clin Invest, 100, 2648-52.
ROWE, et al. (1969). Biochemistry, 8, 2674-80.
RUEPPEL, et al. (1972). Biochemistry, 11, 2839-44.
RUHLAND, et al. (2002). Environ Biosafety Res, 1, 29-37.
SADUNISHVILI, et al. (1996). Ecotoxicol Environ Saf, 34, 70-5.
SAID, et al., (1996). Proc Natl Acad Sci USA, 93, 4688-92.
SAID, V (2000). Free Radic Biol Med, 28, 1300-2.
SANCHEZ-MATEO, et al. (2003). Arzneimittelforschung, 53, 12-20.
SARIN, et al. (1981). Anal Biochem, 117, 147-57.
SATO, et al. (1993). Neuroendocrinology, 58, 77-9.
SCHELTINGA, et al. (1991). Ann Surg, 214, 385-93.
SCHEPPACH, et al. (1999). Eur J Cancer Prev, 8, 57-62.
SCHEPPACH, et al. (1994). Gastroenterology, 107, 429-34.
SCHOEPP, et al. (1994). J Neurochem, 63, 769-72.
SEILER, N. (1993). Neurochem Res, 18, 235-45.
SEKIYAMA, et al. (1996). Br J Pharmacol, 117, 1493-503.
SHAKED, et al. (2002). J Neurochem, 83, 574-80.
SHANNON, H. E. & SAWYER, B. D. (1989). J Pharmacol Exp Ther, 251, 518-23.
SHAW, et al. (1999). Can J Physiol Pharmacol, 77, 871-7.
SHELDRICK, W. F. (1987). World Nitrogen Survey. Technical paper no. 59, World Bank, Washington, D.C.
SHELINE, et al. (1996). Proc Natl Acad Sci USA, 93, 3908-13.
SHIGEMOTO, et al. (1992). Neurosci Lett, 144, 229-32.
SHINOZAKI, et al. (1996). Biosci Biotechnol Biochem, 60, 903-5.
SHOHAMI, et al. (1999). J Neurotrauma, 16, 365-76.
SIBSON, et al. (1997). Proc Natl Acad Sci USA, 94, 2699-704.
SIGGINS, et al. (1982). Prog Clin Biol Res, 90, 275-87.
SINSKY, M. & DONNERER, J. (1998). Neurosci Lett, 258, 109-12.
SKERRY, T. M. & GENEVER, P. G. (2001). Trends Pharmacol Sci, 22, 174-81.
SLUKA, et al. (1994). Brain Res, 664, 77-84.
SNINSKY, et al. (1994) Gastroenterology, 106, A569 (abstr).
SOGNI, P., et al. (1998). Eur J Pharmacol 344, 37-43.
SPENCER, G. J. & GENEVER, P. G. (2003). BMC Cell Biol, 4, 9.
SPENCER, et al. (2004). FEBS Lett, 559, 6-12.
SPITTLER, et al. (2001). Clin Nutr, 20, 37-42.
STITT, et al. (2002). J Exp Bot, 53, 959-70.
STORTO, et al. (2000a). J Neuroimmunol, 109, 112-20.
STORTO, et al. (2000b). Hepatology, 31, 649-55.
STORTO, et al. (1999). FEBS Letters, 444, 160-4.
STORTO, et al. (2001). J Endocrinol, 170, 71-8.
SUGIYAMA, T. & SADZUKA, Y. (1998). Cancer Lett, 133, 19-26.
SWANSON, et al. (1990). et al. Stroke, 21, 322-7.
TAKEUCHI, et al. (1998). Chem Pharm Bull (Tokyo), 46, 1703-9.
TAKAHASHI, et al. (1991). Am J Physiol, 261, H825-9.
TAKAHASHI, et al. (1992). Circ Res, 71, 1220-30.
TEITELBAUM, et al. (1990). N Engl J Med, 322, 1781-7.
THOMSEN, C. & SUZDAK, P. D. (1993). Eur J Pharmacol, 245, 299-301.
TOBIN, A. K. & YAMAYA, T. (2001). J Exp Bot, 52, 591-604.
TONG, et al. (2001). Neuroreport, 12, 3929-34.
TONG, et al. (2002). Am J Physiol Endocrinol Metab, 282, E1324-33.
TSACOPOULOS, et al. (1997). Glia, 21, 84-91.
TUMANI, et al. (2000). Glia, 30, 11-8.
VARNEY, et al. (1999). J Pharmacol Exp Ther, 290, 170-81.
VITOUSEK, et al. (1997). Ecol Appl, 7, 737-50.
VOGELS, et al. (1997). J Hepatol, 26, 387-95.
WADA, et al. (1967). Neurology, 17, 854-68.
WALKER, et al. (1987). J Chem Soc Chem Commun, 1710-11.
WATANABE, et al. (1994). Neurosci Lett, 165, 183-6.
WATANABE, et al. (1992). Eur J Pharmacol, 222, 157-62.
WEAVER, et al. (1996). J Biol Chem, 271, 12977-84.
WEAVER, et al. (1998). Mol Pharmacol, 54, 639-46.
WEDLER, et al. (1980). Arch Biochem Biophys, 202, 482-90.
WEISS, et al. (1999). JPEN J Parenter Enteral Nutr, 23, 140-6.
WELBOURNE, T. C. & DASS, P. D. (1982). Life Sci, 30, 793-801.
WERMUTH, et al. (1996). J Med Chem, 39, 814-6.
WHITE, et al. (1981). J Neurochem, 36, 328-32.
WILEY, J. W., et al. (1991). Am J Physiol, 261, G693-700.
WILLARD-MACK, et al. (1996). Neuroscience, 71, 589-99.
WILMORE, D. W. (2001). J Nutr, 131, 2543S-9S.
WIREN, et al. (1998). Int J Biochem Cell Biol, 30, 1331-6.
WRONSKI, et al. (1991). J Bone Miner Res, 6, 387-94.
YAMADA, et al. (1998). J Neurosci, 18, 2056-62.
YATSUSHIRO, et al. (2000). J Neurochem, 75, 288-97.
YONEDA, Y. & OGITA, K. (1986). Brain Res, 383, 387-91.
YOSHIDA, et al. (2001). Acta Med Okayama, 55, 349-55.
YOUNG, V. R. & AJAMI, A. M. (2001). J Nutr, 131, 2449S-59S.
ZALKIN, H. & SMITH, J. L. (1998). Adv Enzymol Relat Areas Mol Biol, 72, 87-144.
ZEEVALK, et al. (1989). J Neurochem, 53, 1610-9.
ZEEVALK, G. D. & NICKLAS, W. J. (1990). J Pharmacol Exp Ther, 253, 1285-92.
ZELLER, E. A. & BARSKY, J. (1952). Proc Soc Exp Biol Med, 81, 459-61.
ZHLOBA, et al. (1968). Lab Delo, 10, 621-2.
ZIEGLER, et al. (2000). Curr Opin Clin Nutr Metab Care, 3, 355-62.
ZIEGLER, et al. (2003). Annu Rev Nutr, 23, 229-61.
ZWINGMANN, et al. (1998). Dev Neurosci, 20, 417-26.
ZWINGMANN, et al. (2003). In: Encephalopathy and Nitrogen Metabolism in Liver Failure. E A Jones, R A F M Chamuleau, A J Meijer (eds) Kluwer, Dordrecht, Netherlands, 299-311.
ZWINGMANN, et al. (2003). Hepatology, 37, 420-28.

1-29. (canceled)

30. A method for treating conditions and disorders associated with glutamine synthetase (GS) activity comprising administering to a subject an effective amount of a compound selected from the group consisting of tianeptine, salts, isomers, pro-drugs, metabolites and structural analogs thereof, wherein said conditions and disorders are not those listed in Table 5.

31. The method according to claim 30, wherein said conditions or disorders are selected from those disclosed in Tables 1-4.

32. The method according to claim 31, wherein said isomers, pro-drugs, metabolites or structural analogs of tianeptine are selected from the compounds in Table 8.

33. The method according to claim 31, wherein said tianeptine or salts, isomers, pro-drugs, metabolites or structural analogs thereof, is used in combination with another drug used to treat diseases or disorders associated with GS activity.

34. A method for treating conditions and disorders associated with glutamine synthetase (GS) activity comprising administering to said subject an effective amount of a compound selected from the group consisting of naftazone (NF), salts, isomers, pro-drugs, metabolites and structural analogs thereof, wherein said conditions and disorders are not those listed in Table 14.

35. The method according to claim 34, wherein said conditions or disorders are selected from those disclosed in Tables 1-4.

36. The method according to claim 34, wherein said isomers, pro-drugs, metabolites and structural analogs of NF are selected from the compounds in Table 15.

37. The method according to claim 34, wherein said NF or salt, isomer, pro-drug, metabolite or structural analog thereof, is used in combination with another drug used to treat diseases or disorders associated with GS activity.

38. A method for treating conditions and disorders selected from the group consisting of asthma, cough, depression, irritable bowel syndrome, mnemo-cognitive disorders, neurodegenerative diseases, non-ulcer dyspepsia, pain, psychoneurotic disorders, seizure, stress and stroke, arteriosclerosis, stenosis, vascular insufficiency and necrosis, embolism and thrombosis, vascular disorders, nec, vascular haemorrhagic disorders, vascular inflammations and venous varices groups, encephalopathies, mental impairment disorders, movement disorders, neurological disorders of the eye, neuromuscular disorders and seizures, Alzheimer's, Huntington's disease, Parkinson's disease, multiple sclerosis, amyotrophic lateral sclerosis, spinal muscular atrophy, retinopathy, and traumatic brain injury, drug-induced neurotoxicity, pain, hormonal balance, blood pressure, thermoregulation, respiration, learning, pattern recognition, memory, and disorders subsequent to hypoxia or hypoglycaemia in a subject comprising administering an effective amount of a glutamine synthetase (GS) ligand, wherein said GS ligand is not of tianeptine and NF;
wherein said GS ligand is selected from the group consisting of glufosinate (GF), L-methionine sulfoximine (MS), hydrazines and bisphosphonates, and salts, isomers, pro-drugs, metabolites and structural analogs thereof, provided that:
if said GS ligand is GF or MS, the conditions and disorders related to cerebral ischemia, hyperammonemia marked in “double-underlined” in Table 2, bacterial, viral and fungal infectious disorders, neoplasm, neurogenerative diseases selected from Alzheimer disease, Huntington's and other polyglutamine disorders and pain are excluded;
if said GS ligand is a hydrazine, the conditions and disorders disclosed in Table 6 are excluded; and
if said GS ligand is a bisphosphonate, the conditions and disorders disclosed in Table 7 are excluded.

39. The method according to claim 38, wherein the GS ligand is used in an amount resulting in activation or inhibition of GS activity in the target cells/tissues/organs/organism, without any strict inhibition of GS.

40. A method for treating conditions and disorders associated with GS activity comprising administering a glutamine synthetase (GS) ligand in an amount resulting in activation or inhibition of GS activity in the target cells/tissues/organs/organism, wherein said GS ligand is not tianeptine or NF.

41. The method according to claim 40, wherein the conditions and disorders are selected from those disclosed in Tables 2 and 3, provided that:
if said GS ligand is GF or MS, the conditions and disorders related to cerebral ischemia, hyperammonemia (marked in “double-underlined” in Table 2), bacterial, viral and fungal infectious disorders, neoplasm, neurogenerative diseases, Alzheimer disease, Huntington's or other polyglutamine disorders and pain are excluded;
if said GS ligand is a hydrazine, the conditions and disorders disclosed in Table 6 are excluded; and
if said GS ligand is a bisphosphonate, the conditions and disorders disclosed in Table 7 are excluded.

42. The method according to claim 40, wherein the GS ligand is used in an amount that results in activation of GS activity in the target cells/tissues/organs/organism.

43. A method for facilitating the growth of non-vertebrate organisms with eukaryotic cells or prokaryotic cells, for reducing the growth of non-vertebrate organisms or for protecting non-vertebrate organisms from stresses and disorders comprising administering an efficient amount of a compound selected from the group consisting of tianeptine, naftazone (NF), salts, isomers, pro-drugs, metabolites and structural analogs thereof.

44. A method for facilitating the growth of non-vertebrate organisms with eukaryotic cells or for protecting non-vertebrate organisms from stresses and disorders, comprising administering an efficient amount of a glutamine synthetase (GS) ligand selected from the group consisting of glufosinate (GF), L-methionine sulfoximine (MS), hydrazines and bisphosphonates, and salts, isomers, pro-drugs, metabolites and structural analogs thereof, wherein said GS ligand is used at an amount that regulates GS activity, provided that if said GS ligand is GF, MS, or a bisphosphonate, plants are excluded as eukaryotic cells.

45. A method for monitoring treatments with a drug selected from the group consisting of NF, tianeptine, GF, hydrazines, bisphosphonates, strontium, salts, isomers, pro-drugs, metabolites and structural analogs thereof, comprising determining biological markers related to the glutamine synthetase (GS) enzyme like GS activity, GS expression, glutamate, glutamine and NH₄.

46. A method for increasing the efficiency or decreasing the side effect of (gluco)corticoid treatment in a subject, comprising administering to said subject an efficient amount of a glutamine synthetase (GS) ligand.

47. The method according to claim 46, wherein the GS ligand is selected from the group consisting of tianeptine, NF, GF and MS as well as hydrazines, bisphosphonates and metal ions, isomers thereof, pro-drugs thereof, metabolites thereof and structural analogs thereof.

48. A method for screening, identifying and/or developing a new active compound, regulating GS activity comprising a competition assay on glutamine synthetase or a fragment thereof with a candidate compound and tianeptine or naftazone (NF), or salts thereof, isomers thereof, pro-drugs thereof, metabolites thereof and structural analogs thereof.

49. The method according to claim 48, comprising an assay on glutamine synthetase or a fragment thereof with a candidate compound and an antibody raised against a fragment comprising or consisting of the amino acid sequence ITGTNAEVMPAQWEFQIGPCEGIR (SEQ ID NO:1).
